(19) United States
(12) Patent Application Publication
(10) Pub. No.: US 2023/0398211 A1
(43) Pub. Date: Dec. 14, 2023

(54) Liposomes Containing TLR4 Agonist, Preparation and Uses Thereof

(71) Applicant: Sanofi Pasteur, Lyon (FR)

(72) Inventors: Marie Garinot, Paris (FR); Jean Haensler, Paris (FR); Fabienne Piras, Paris (FR); Patrick Syntin, Paris (FR); Sophie Ruiz, Paris (FR)

(73) Assignee: Sanofi Pasteur, Lyon (FR)

(21) Appl. No.: 18/034,242

(22) PCT Filed: Oct. 28, 2021
(86) PCT No.: PCT/EP2021/079918

§ 371 Date: Apr. 27, 2023

(30) Foreign Application Priority Data
Oct. 28, 2020 (EP) 20306291.4

(51) Int. Cl. A61K 39/39; A61K 31/688; A61K 9/127; A61K 39/245; A61P 31/22

(52) U.S. Cl. CPC A61K 39/39; A61K 31/688; A61K 9/127; A61K 9/1277; A61K 39/245; A61K 9/1272; A61P 31/22; A61K 2039/55555

(57) ABSTRACT

The present invention relates to a liposome comprising a saponin, a sterol, a phospholipid and a Toll-like receptor 4 (TLR4) agonist of formula (I), to methods of preparing liposomes, to compositions comprising them and to uses thereof, and to immunogenic compositions comprising such liposomes as adjuvant.

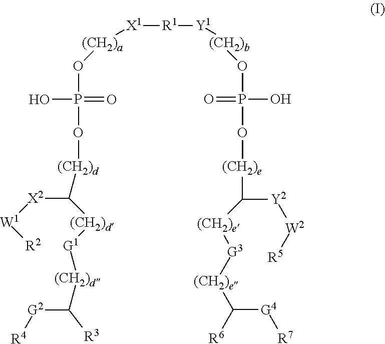

### FIELD OF THE DISCLOSURE

This disclosure relates to the field of novel liposome formulations which may be used as an adjuvant in vaccine compositions. It also relates to methods of producing the liposomes and to their use in medicine.

The present disclosure further relates to immunogenic compositions comprising a CMV (Cytomegalovirus) gB antigen, a CMV gH/gL/UL128/UL130/UL131 pentameric complex antigen, and a TLR-4 agonist-containing adjuvant. Further, it relates to CMV antigens-containing compositions endowed with low reactogenicity. It further relates to immunogenic compositions for use as a CMV vaccine.

### BACKGROUND

Adjuvant formulations have been used for many years in vaccine compositions to help enhance the immune response to a given antigen by enhancing antigen presentation to immune cells with the aim to confer long-term protection against targeted pathogens. Adjuvants may also find useful application to reduce the needed amount of a given antigen, while maintaining an efficient level of immune response of the vaccine. This sparing of antigens may be useful for increasing vaccine manufacturing volume capacity while the available amount of antigen needed stays constant. This antigen sparing may be very useful for instance in a pandemic situation.

Some adjuvants are specific to certain antigens, while others have a broader range of action and are effective in combination with antigens of different chemical natures and against different kinds of diseases. Adjuvants with balanced Th1/Th2 profile may have a broader range of action.

These last types of adjuvants have the advantage of being manufactured in advance and rapidly available to a pharmaceutical company or a medical practitioner for their combination with a broad selection of antigens at hand. They can be directly administered to an individual in need thereof. This characteristic may also be of particular interest during a pandemic.

Among adjuvant systems recognized in the art, mention may be made of the AS01 adjuvant commercialized by GlaxoSmithKline. AS01 is a liposome-based vaccine adjuvant system containing two immunostimulants: the TLR4 agonist 3-O-desacyl-4′-monophosphoryl lipid A (MPL) and the saponin QS-21 (WO2007/068907 A1, EP 0 955 059B1).

However, because of its low solubility in solvents such as ethanol, the presence of MPL in adjuvant systems, notably in liposome adjuvant systems, limits the methods that can be used for their production, which can be a serious impediment for industrial development and scale-up. Furthermore, the amount of MPL required to obtain satisfactory immunopotentiating or adjuvant properties in a formulation is relatively high, which makes its use quite costly. These drawbacks necessarily make these adjuvants more complex to manufacture and also increase their production cost.

Other TLR4 agonists are known in the art, many of which were proposed as vaccine adjuvants (Fox et al., Subcell Biochem. 2010; 53:303-21). As known TLR4 agonists, mention may be made of opioids such as buprenorphine, oxycodone, methadone, fentanyl, curcumin, glycyrrhizin, paclitaxel, morphine (Peri et al., J Med Chem. 2014; 57(9):3612-3622) of natural lipopolysaccharides such as monophosphoryl lipid A (MPL), or of synthetic TLR4 agonists such as aminoalkyl glucosaminide phosphates (AGPs) (Alderson et al., J Endotoxin Res. 2006; 12(5):313-9), GLA-60, ER112022, or ONO-4007 (Peri et al., J Med Chem. 2014; 57(9):3612-3622), the compounds described in WO 2019/157509, or E6020 (Ishizaka and al., 2007, Future Drugs). However, it is challenging to identify TLR4 agonists that may be easily formulated in liposomes, notably in industrial manufacturing processes, while still maintaining satisfactory immunopotentiating and adjuvanting responses.

Another concern when formulating adjuvants which are meant to be used by humans or animals, is that as many of the preparation steps as possible should be performed using products that are generally accepted by Health Agencies. For example, certain solvents should be avoided, and others, which have better acceptance from a pharmaceutical standpoint, should be preferred.

Thus, there remains a need for new formulations, such as adjuvant compositions, which are at least as effective in terms of immune response enhancement as the formulations that are available on the market.

There also remains a need for adjuvant compositions having a good safety profile and no or reduced reactogenic effects.

Furthermore, there is a need for immunopotentiating and adjuvant formulations which are easy to manufacture, notably at industrial scale, and have a low production cost. There is a need to be able to manufacture adjuvant-based liposomes containing TLR4 agonists at an industrial scale and at low cost. There is also a need to provide adjuvant formulations which are as pharmaceutically innocuous as possible, using raw materials and manufacturing intermediates, that are considered safe by most Health Authorities.

There is a need to have adjuvants which may be used for antigens sparing.

Finally, there remains a need for formulations which induce a more balanced Th1/Th2 response, in particular compared to certain adjuvant formulations known in the art.

The present disclosure provides these and other related advantages.

The Human cytomegalovirus (HCMV) is a ubiquitous virus belonging to the Herpes virus family. The virus is composed of a linear double-stranded deoxyribonucleic acid (DNA) contained in a capsid surrounded by a tegument and enveloped in a lipid bilayer carrying glycoprotein spikes on its surface. Like other members of this family, HCMV possesses the characteristics of latency and reactivation.

In the immunocompetent host, most HCMV infections are asymptomatic or very mild with a few nonspecific symptoms, such as fatigue, malaise, moderate fever, lymphadenopathy, hepatomegaly or a slight increase in liver enzymes. Heterophil-negative mononucleosis is however observed in approximately 10% of previously healthy individuals. In contrast, clinical manifestations can be very severe in newborns infected in utero and in adults immunosuppressed by AIDS or in the context of solid organ or bone marrow transplantation.

The prevalence of HCMV infection increases with age and is affected by socioeconomic factors. Serological surveys have shown a higher prevalence in developing countries and in lower socioeconomic groups of developed countries. For women of child-bearing age, the proportion of HCMV seropositive women ranges from approximately 50% in upper and middle incomes groups of developed countries to over 80% in low-income populations. Surveys performed in different western European countries showed globally that HCMV seroprevalence in toddlers and adolescents ranges between 40 and 50% while in older subjects (40 years and over), HCMV seroprevalence is higher than 80%.

HCMV is the most common cause of congenital infection in the developed world. Congenital infection refers to infection transmitted from mother to fetus prior to birth of the newborn. Overall, a primary HCMV infection during pregnancy is associated with a 40% risk of transmission to the fetus. As a result of congenital HCMV infection, infants may suffer disabilities, including mental retardation, blindness and sensorineural deafness. Among congenitally infected newborns, 5% to 10% have major manifestations at birth such as microcephaly, chorioretinitis, intracranial calcifications, hepatosplenomegaly, hepatitis, jaundice, direct hyperbilirubinemia, thrombocytopenia, petechiae, and anemia. Among these newborns with symptomatic congenital HCMV disease, the mortality rate is approximately 10% in early infancy and among survivors, 50-90% will have sequelae such as mental retardation, cerebral palsy, sensorineural hearing loss or visual impairment. Besides, many infants with congenital HCMV infection are asymptomatic at birth. Nonetheless, follow-up studies have shown that approximately 15% of infants who are HCMV seropositive in the newborn period by virological screening and asymptomatic at birth will have sequelae such as hearing loss or central nervous system abnormalities. As a whole, approximately 17,000 infants born each year in Europe and in the USA will have permanent sequelae.

HCMV is also an important viral pathogen in organ and bone marrow transplant recipients and in AIDS patients. The rate of HCMV-associated morbidity in HCMV seronegative solid organ transplant recipients approaches 60%. In solid organ transplant the disease is the most severe when seronegative patients receive a graft from a HCMV positive donor. In contrast, in bone marrow or stem cell transplantation the disease is most severe in HCMV seropositive subjects receiving cells from a seronegative donor showing that the origin of HCMV infection is reactivation of endogenous infection. HCMV causes pneumonitis, hepatitis, gastrointestinal disease, bone marrow suppression, and retinitis in approximately 15% of allograft recipients. In addition to these direct end-organ diseases, HCMV has been associated with indirect effects such as graft rejection, accelerated atherosclerosis and immunosuppression that can lead to bacterial or fungal infection.

Effective means of preventing or treating HCMV infection during pregnancy or congenital HCMV infection, or in organ and bone marrow transplant recipients and in AIDS patients, are currently not available.

Development of an HCMV vaccine is therefore considered a major public health objective in Institute of Medicine vaccine prioritization reports (Institute of Medicine (US) Committee to Study Priorities for Vaccine Development, Stratton K R, Durch J S, Lawrence R S, eds. Vaccines for the 21st Century: A Tool for Decision making. Washington (DC): National Academies Press (US); 2000). Many candidate vaccines have been described, as for example in WO 20090/37359A1, WO 2017/070613A1 or WO 2019/052975, but, so far, none has been licensed (Plotkin et al., Vaccines, 6th edition, Ed. Elsevier, 2013, Schleiss et al., Cytomegalovirus vaccines, pages 1032-1041; Permar et al., J Virol. 2018 Mar. 14; 92(7):e00030-18).

A cytomegalovirus glycoprotein-B vaccine with MF59 adjuvant showed promising results in a phase 2 randomized placebo-controlled trial in transplant recipients (Griffiths et al., Lancet. 2011; 377(9773):1256-1263). A phase 2, placebo-controlled, randomized, double-blind trial in women of child-bearing age, evaluated the same vaccine consisting of recombinant HCMV envelope glycoprotein B with MF59 adjuvant, as compared with placebo. The results showed 50% efficacy in preventing HCMV acquisition of primary HCMV. However, the immunogenicity results showed that the level of neutralizing antibodies (Ab) induced by the gB/MF59 formulation are at the peak level one month after the administration of the 3rd dose, and then rapidly decline (Pass et al., N Engl J Med. 2009; 360(12):1191-1199).

As a consequence, there is a need to have a CMV vaccine with improved efficacy, notably a vaccine able to increase neutralizing antibody levels and induce long-lasting protection by inducing a persistent immune response.

There is also a need to have a CMV vaccine able to induce a broad immune response.

There is a need to have an adjuvanted CMV vaccine able to induce a protecting level of antibodies neutralizing CMV.

There is a need to have an adjuvanted CMV vaccine able to induce long lasting antibodies able to neutralize CMV in individuals.

In addition to their expected benefic effects towards individuals' health, vaccines may sometimes, transiently, locally or systematically, induce reactogenic effects (Hervé et al., NPJ Vaccines. 2019; 4:39). Those effects reflect the physical manifestation of the inflammatory response that results from the injection of a vaccine. They may be, for example, injection-site pain or induration, redness, swelling, or even systemic symptoms, such as fever, myalgia, or headache. Those reactogenic effects may induce negative behavior towards vaccines uses and recommendation, and a low level of adhesion to vaccine schedules. In regard of the perception that an individual may have of the reactogenicity of a given vaccine, he or she may refuse to be vaccinated. Even healthcare professionals may decide to recommend the vaccine or not. As a consequence, poor adherence to vaccination or poor coverage of individuals to a given vaccine may happen, which may dramatically affect the global beneficial effect which can be draw from vaccination.

Adjuvants are immunostimulants that enhance the immune response and/or orient the kind of response (Th1 versus Th2) to the antigen. On the downside, it is acknowledged that adjuvant's type and dose may increase vaccines' reactogenicity compared to non-adjuvanted vaccines (Hervé et al., NPJ Vaccines. 2019; 4:39). For a same antigen, the use of different adjuvants may induce different levels of reactogenicity and different reactogenic response types. For instance, a study reporting hepatitis B antigen (HBsAg) formulated with different antigens, i.e. alum or Adjuvant Systems AS01B, AS01E, AS03A or AS04 showed that formulations with AS01, in particular with AS01B, were inducing the highest local and general reactogenicity (Leroux-Roels et al., Clin Immunol. 2016; 169:16-27). AS01 is in the formulations of various marketed vaccines and contains the TLR4 agonist 3-O-desacyl-4′-monophosphoryl lipid A (MPL) as adjuvant.

Other TLR4 agonists are known in the art and many of which were proposed as vaccine adjuvants (Fox et al., Subcell Biochem. 2010; 53:303-21). As known TLR4 agonists mention may be made of opioids such as buprenorphine, oxycodone, methadone, fentanyl, curcumin, glycyrrhizin, paclitaxel, morphine (Peri et al., J Med Chem. 2014; 57(9):3612-3622), of natural lipopolysaccharides such as monophosphoryl lipid A (MPL), or of synthetic TLR4 agonists such as aminoalkyl glucosaminide phosphates (AGPs) (Alderson et al., J Endotoxin Res. 2006; 12(5):313-9), GLA-60, ER112022, or ONO-4007 (Peri et al., J Med Chem. 2014; 57(9):3612-3622), the compounds described in WO 2019/157509, or E6020 (Ishizaka and al., 2007, Future Drugs).

It is challenging to identify adjuvants containing-TLR4 agonists that may be used to formulate vaccines, while inducing low-level of reactogenicity. It is further challenging to identify adjuvanted CMV-antigens containing-vaccines with low-level of reactogenicity.

Therefore, there is a need to select adjuvants that, while still being good immunostimulants, also induce low or mild reactogenicity to the vaccine in the subject.

Therefore, in addition of the need to have an efficient adjuvanted vaccine towards CMV infection, there is a need that this vaccine induces low reactogenicity in the subject.

There is a need to have an adjuvanted CMV vaccine, for example with a TLR4 agonist, to be used in a multiple doses vaccine schedule which induces low reactogenicity at subsequent doses whatever the regimen schedule.

There is a need to have an adjuvanted CMV vaccine, for example with a TLR4 agonist, favoring compliance and acceptance of subsequent doses administration.

There is a need to have an adjuvanted CMV vaccine, for example with TLR4 agonist, to be used in a multiple doses vaccine schedule which induces low increase of inflammatory serum biomarkers, such as C-reactive protein (CRP), fibrinogen, neutrophil counts, and/or globulin, at subsequent doses following the first dose.

The present disclosure has for purpose to satisfy all or part of those needs.

### SUMMARY OF THE DISCLOSURE

### Liposomes Containing TLR4 Agonist, Preparation And Uses Thereof

The present disclosure relates to a liposome comprising a saponin, a sterol, a phospholipid and a Toll-like receptor 4 (TLR4) agonist (such as a single type of liposome), or
a combination of liposomes comprising at least two types of liposomes, wherein a first type of liposome comprises a saponin, a sterol, and a phospholipid and a second type of liposome comprises a sterol, a phospholipid, and a Toll-like receptor 4 (TLR4) agonist,
wherein the Toll-like receptor 4 (TLR4) agonist is of formula (I):

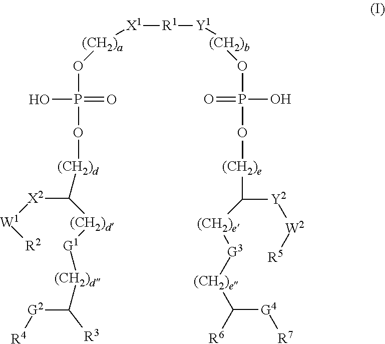

wherein R¹ is selected from the group consisting of:
   a) C(O);
   b) C(O)—(C₁-C₁₄ alkyl)-C(O), in which said C₁-C₁₄ alkyl is optionally substituted with a hydroxyl, a C₁-C₅ alkoxy, a C₁-C₅ alkylenedioxy, a (C₁-C₅ alkyl)amino or a (C₁-C₅ alkyl)aryl, in which said aryl moiety of said (C₁-C₅ alkyl)aryl is optionally substituted with a C₁-C₅ alkoxy, a (C₁-C₅ alkyl)amino, a (C₁-C₅ alkoxy)amino, a (C₁-C₅ alkyl)-amino(C₁-C₅ alkoxy), —O—(C₁-C₅ alkyl)amino(C₁-C₅ alkoxy), O(C₁-C₅ alkyl)amino-C(O)—(C₁-C₅ alkyl)-C(O)OH, or —O—(C₁-C₅ alkyl)amino-C(O)—(C₁-C₅ alkyl)-C(O)—(C₁-C₅)alkyl;
   c) an alkyl comprising a C₂-C₁₅ linear or branched chain, optionally substituted with a hydroxyl or an alkoxy; and
   d) —C(O)—(C₆-C₁₂ arylene)-C(O)— in which said arylene is optionally substituted with a hydroxyl, a halogen, a nitro or an amino;
a and b are independently 0, 1, 2, 3 or 4;
d, d′, d″, e, e′ and e″ are independently 0, 1, 2, 3 or 4;
X₁, X₂, Y₁ and Y₂ are independently selected from the group consisting of null, an oxygen, NH and N(C(O)(C₁-C₄ alkyl)), and N(C₁-C₄ alkyl);
W₁ and W₂ are independently selected from the group consisting of a carbonyl, a methylene, a sulfone and a sulfoxide;
R² and R⁵ are independently selected from the group consisting of:
   a) a C₂ to C₂₀ straight chain or branched chain alkyl, which is optionally substituted with an oxo, a hydroxyl or an alkoxy;
   b) a C₂ to C₂₀ straight chain or branched chain alkenyl or dialkenyl, which is optionally substituted with an oxo, a hydroxyl or an alkoxy;
   c) a C₂ to C₂₀ straight chain or branched chain alkoxy, which is optionally substituted with an oxo, a hydroxyl or an alkoxy;
   d) NH—(C₂ to C₂₀ straight chain or branched chain alkyl), in which said alkyl group is optionally substituted with an oxo, a hydroxy or an alkoxy; and
   e)

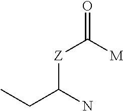

in which Z is selected from the group consisting of an O and NH, and M and N are independently selected from the group consisting of an alkyl, an alkenyl, an alkoxy, an acyloxy, an alkylamino and an acylamino comprising a C₂-C₂₀ linear or branched chain;
R³ and R⁶ are independently selected from the group consisting of a C₂ to C₂₀ straight chain or branched chain alkyl or alkenyl, optionally substituted with an oxo or a fluoro;
R⁴ and R⁷ are independently selected from the group consisting of a C(O)—(C₂ to C₂₀ straight chain or branched chain alkyl or alkenyl), a C₂ to C₂₀ straight chain or branched chain alkyl, a C₂ to C₂₀ straight chain or branched chain alkoxy, and a C₂ to C₂₀ straight chain or branched chain alkenyl; in which said alkyl, alkenyl or alkoxy groups can be independently and optionally substituted with a hydroxyl, a fluoro or a C₁-C₅ alkoxy;
G¹, G², G³ and G⁴ are independently selected from the group consisting of an oxygen, a methylene, an amino, a thiol, —C(O)NH—, —NHC(O)—, and —N(C(O)(C₁-C₄ alkyl))-;
   or G²R⁴ or G⁴R⁷ can together be a hydrogen atom or a hydroxyl;
   or a pharmaceutically acceptable salt of this compound,
   wherein the TLR4 agonist and the saponin are present in a weight:weight ratio of TLR4 agonist:saponin ranging from 1:1 to about 1:50, or from about 1:25 to about 1:35, or in a weight ratio of TLR4 agonist:saponin of about 1:10.

In one embodiment, a TLR4 agonist as disclosed herein has a solubility parameter in ethanol, measured at 25° C., of at least about 0.2 mg/mL.

In some embodiment, the first type of liposomes may be devoid of TLR4 agonist. In some embodiments, the second type of liposomes may be devoid of saponin.

As surprisingly observed by the inventors, and detailed in the Examples, the liposomes, such as a single type of liposomes, or combinations of at least two types of liposomes as disclosed herein are endowed with a strong immunopotentiating activity, a Th1/Th2 balanced response, and are able to adjuvant numerous antigens, including CMV antigens, Flu antigens and RSV antigens. Further, the liposomes or combinations of at least two types of liposomes present the advantages to be able to be manufactured according to a simple and efficient process. Advantageously, a manufacturing process may implement ethanol-only solvent as organic solvent to be used in the steps of manufacturing the liposomes. Further, the liposomes or combinations of at least two types of liposomes of the invention contain low amounts of TLR4 agonist while they are able to induce a strong adjuvant effect. This ease of production associated with low amount of TLR4 agonist results in advantageous reduced costs of production, and make the adjuvant as disclosed herein useful for sparing antigens in vaccine production. Further, compared to similar adjuvants, such as AS01B, the liposomes or combinations of at least two types of liposomes present an adjuvant effect with a more balanced Th1/Th2 effect to a wide range of antigens, which confers to the adjuvant a broader spectrum of application for the vaccination. Further, as shown in the Examples, the liposomes or combinations of at least two types of liposomes as disclosed herein comprising QS7 as saponin present advantageously a good safety profile and a good adjuvanting effect.

Further, the inventors have surprisingly observed that it was not necessary to have a TLR4 agonist and a saponin in single type of liposomes, but that a combination of at least two types of liposomes where a first type of liposomes comprises a saponin, a sterol, and a phospholipid, but no TLR4 agonist, and a second type of liposomes comprises a sterol, a phospholipid, and a Toll-like receptor 4 (TLR4) agonist, but no saponin, was able to induce a similar adjuvanting effect as a single type of liposomes comprising a sterol, a phospholipid, a saponin and a Toll-like receptor 4 (TLR4) agonist. In some embodiment, the first type of liposomes may be devoid of any TLR4 agonist and the second type of liposomes may be devoid of any saponin.

In the specification, the expression “a liposome” may interchangeably refers to a “single type” of liposomes comprising a sterol, a phospholipid, a saponin and a Toll-like receptor 4 (TLR4) agonist or to any one of the “first and/or second types” of liposomes comprising either (i) a saponin, a sterol, and a phospholipid, or (ii) a sterol, a phospholipid, and a Toll-like receptor 4 (TLR4) agonist, except if the context dictates otherwise. A “type of liposome” intends to refer to a liposome defined by the nature and amounts of its constituents, such as sterols, phospholipids, saponins, or TLR4 agonists.

In the specification, a first and a second types of liposomes intends to refer to a first and a second types of liposomes differing by their compositions as described herein.

In another embodiment, a suitable TLR4 agonist is of formula (II):

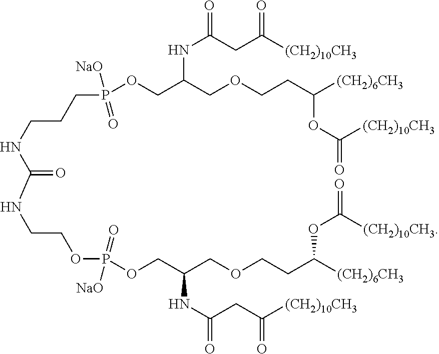

In another embodiment, a suitable TLR4 agonist is E6020 of formula (III):

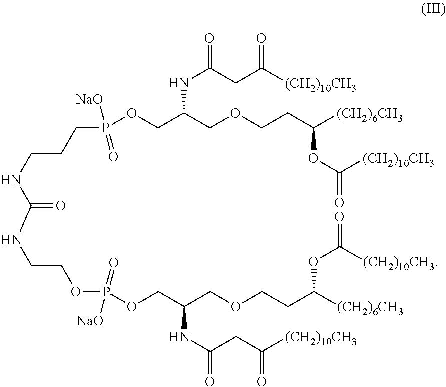

In another embodiment, a liposome (e.g., a single type of liposome) or a liposome of a combination of at least two types of liposomes may comprise as saponin a Quillaja saponaria saponin.

In another embodiment a liposome (e.g., a single type of liposome) or liposomes of a combination of at least two types of liposomes may comprise as saponin a saponin extracted from the bark of Quillaja saponaria Molina.

In another embodiment, a liposome (e.g., a single type of liposome) or liposomes of a combination of at least two types of liposomes may comprise as saponin a saponin selected among QS7, QS17, QS18, QS21, and combinations thereof.

In another embodiment, a saponin may be QS21 or QS7.

In another embodiment a liposome (e.g., a single type of liposome) or liposomes of a combination of at least two types of liposomes may comprise as saponin a QS21.

In another embodiment, a liposome (e.g., a single type of liposome) or liposomes of a combination of at least two types of liposomes may comprise as saponin a QS7.

In another embodiment, a liposome (e.g., a single type of liposome) or liposomes of a combination of at least two types of liposomes may comprise as sterol a sterol selected from cholesterol or its derivatives, ergosterol, desmosterol (3β-hydroxy-5,24-cholestadiene), stigmasterol (stigmasta-5,22-dien-3-ol), lanosterol (8,24-lanostadien-3b-ol), 7-dehydrocholesterol (Δ5,7-cholesterol), dihydrolanosterol (24,25-dihydrolanosterol), zymosterol (5α-cholesta-8,24-dien-3β-ol), lathosterol (5α-cholest-7-en-3β-ol), diosgenin ((3β,25R)-spirost-5-en-3-ol), sitosterol (22,23-dihydrostigmasterol), sitostanol, campesterol (campest-5-en-3β-ol), campestanol (5a-campestan-3b-ol), 24-methylene cholesterol (5,24(28)-cholestadien-24-methylen-3β-ol), cholesteryl margarate (cholest-5-en-3β-yl heptadecanoate), cholesteryl oleate, cholesteryl stearate, and mixtures thereof.

In another embodiment, a liposome (e.g., a single type of liposome) or liposomes of a combination of at least two types of liposomes may comprise as sterol a sterol from cholesterol or its derivatives, such as cholesterol.

In another embodiment, a saponin and a sterol may be present, in a liposome (e.g., a single type of liposome) or liposomes of a combination of at least two types of liposomes, in a weight:weight ratio of saponin:sterol ranging from 1:100 to 1:1, ranging from 1:50 to 1:2, or ranging from 1:10 to 1:5, or in a weight:weight ratio of saponin:sterol of about 1:2, or in a weight:weight ratio of saponin:sterol of about 1:5.

In another embodiment, a phospholipid suitable for a liposome (e.g., a single type of liposome) or liposomes of a combination of at least two types of liposomes may be selected from phosphatidylcholines, phosphatidic acids, phosphatidylethanolamines, phosphatidylglycerols, phosphatidylserines, phosphatidylinositols, and mixtures thereof.

In another embodiment, a phospholipid suitable for a liposome (e.g., a single type of liposome) or liposomes of a combination of at least two types of liposomes may be a phosphatidylcholine selected from DSPC (1,2-distearoyl-sn-glycero-3-phosphocholine), DPPC (1,2-dipalmitoyl-sn-glycero-3-phosphocholine), DMPC (1,2-dimyristoyl-sn-glycero-3-phosphocholine), POPC (1-palmitoyl-2-oleoyl-sn-glycero-3-phosphocholine), DOPC (1,2-dioleoyl-sn-glycero-3-phosphocholine), SOPC (1-stearoyl-2-oleoyl-sn-glycero-3-phosphocholine), and mixtures thereof. In one exemplary embodiment, a phospholipid may be DOPC.

In another embodiment, the disclosure is directed to a method for manufacturing a liposome comprising at least the steps of:
(a) solubilizing, in an organic water-miscible solvent, a TLR4 agonist of formula (I) having a solubility parameter in ethanol, measured at 25° C., of at least about 0.2 mg/mL, a sterol, and a phospholipid,
(b) processing the mixture obtained at step (a) into a liposome,
   wherein a saponin is added either at step (a), at step b) or after step (b), and
   wherein the TLR4 agonist and the saponin are present in a weight:weight ratio of TLR4-agonist:saponin ranging from about 1:1 to about 1:400, ranging from about 1:2 to about 1:200, ranging from about 1:2.5 to about 1:100, ranging from about 1:3 to about 1:40, or ranging from about 1:5 to about 1:25. Such a method allows obtaining a single type of liposomes as disclosed herein.

In one embodiment, a saponin is added after step b), i.e., in the liposome containing suspension obtained at step b).

In another embodiment, the disclosure is directed to a method for manufacturing a liposome comprising at least the steps of:
(a) solubilizing, in an organic water-miscible solvent, a TLR4 agonist of formula (I) having a solubility parameter in ethanol, measured at 25° C., of at least about 0.2 mg/mL, a sterol, and a phospholipid,
(b) processing the mixture obtained at step (a) into a liposome. Such method may allow obtaining a second type of liposome as disclosed herein.

In one embodiment, a method as disclosed herein for manufacturing a liposome may further comprise a step, prior to above step (a), of selecting a TLR4 agonist of formula (I) having a solubility parameter in ethanol, measured at 25° C., of at least about 0.2 mg/mL.

In another embodiment, the disclosure is directed to a method for manufacturing a liposome comprising at least the steps of:
(a) solubilizing, in an organic water-miscible solvent a sterol, and a phospholipid,
(b) processing the mixture obtained at step (a) into a liposome,
   wherein a saponin is added either at step (a), at step b) or after step (b). Such a method may allow obtaining a first type of liposome as disclosed herein.

In one embodiment, step (b) of processing the mixture obtained at step (a) into a liposome, of a method as disclosed herein, is carried out by using the solvent injection method.

In an embodiment, step (b) of processing the mixture obtained at step (a) into a liposome includes the steps of:
(b1) injecting and/or diluting the solution obtained at step (a) into an aqueous buffer, and
(b2) removing the organic water-miscible solvent.

In an embodiment, the organic water-miscible solvent is selected from ethanol, isopropanol, or mixtures thereof. In an embodiment, the organic water-miscible solvent is ethanol only solvent.

In an embodiment, the method further may comprise a step (c) of filtering the liposomes obtained in step (b) and recovering the liposomes having an average diameter lower than 200 nm.

Alternatively, in one embodiment, the method may comprise a step (c) of filtering, as for example a sterilizing filtration of, the liposomes obtained in step (b) and recovering the filtered liposomes.

In another embodiment, the disclosure is directed to a method for manufacturing a combination of at least two types of liposomes, wherein a first type of liposomes comprises a saponin, a sterol, and a phospholipid and a second type of liposomes comprises a sterol, a phospholipid, and a Toll-like receptor 4 (TLR4) agonist, the method comprising at least a step of mixing the first and second liposomes.

In another embodiment, the disclosure is directed to an adjuvant composition comprising at least one liposome, such as a single type of liposomes as disclosed herein, or a combination of at least two types of liposomes as disclosed herein or at least one liposome or a combination of at least two types of liposomes obtained by the methods as disclosed herein.

In another embodiment, the disclosure is directed to an immunopotentiating agent comprising at least one liposome, such as a single type of liposomes, or a combination of at least two types of liposomes as disclosed herein or at least one liposome or a combination of at least two types of liposomes obtained by a method as disclosed herein.

In another embodiment, the disclosure is directed to an immunogenic composition, such as a vaccine composition, comprising at least one liposome (e.g., a single type of liposomes as disclosed herein) or a combination of at least two types of liposomes as disclosed herein or at least one liposome or a combination of at least two types of liposomes obtained by methods as disclosed herein, or an adjuvant composition as disclosed herein, and at least one antigen.

In another embodiment, an immunogenic composition may comprise an antigen selected from bacterial antigens, protozoan antigens, viral antigens, fungal antigens, parasite antigens and tumour antigens.

In another embodiment, the disclosure is directed to a kit-of-parts comprising:
a first container comprising a first composition comprising a liposome as disclosed herein or at least one liposome obtained by a method as disclosed herein or an adjuvant composition as disclosed herein, and
a second container comprising a second composition comprising at least one antigen. In such embodiment, a liposome may be a single type of liposomes. An adjuvant composition may comprise a single type of liposome or a combination of at least two types of liposomes.

In another embodiment, the disclosure is directed to a kit-of-parts comprising:
a first container comprising a first composition comprising a first type of liposomes comprising a saponin, a sterol, and a phospholipid,
a second container comprising a second type of liposomes comprising a sterol, a phospholipid, and a Toll-like receptor 4 (TLR4) agonist, and
a third container comprising a third composition comprising at least one antigen.

In another embodiment, the disclosure is directed to a method for manufacturing an immunogenic composition, such as a vaccine, comprising at least a step of mixing at least one liposome (e.g., a single type of liposomes as disclosed herein) or a combination of at least two types of liposomes as disclosed herein, or at least one liposome (e.g., a single type of liposomes as disclosed herein) or a combination of at least two types of liposomes obtained by a method as disclosed herein or an adjuvant composition as disclosed herein with at least one antigen.

In another embodiment, the disclosure is directed to an immunogenic composition obtainable according to a method as disclosed herein.

In another embodiment, the disclosure is directed to a method for adjuvanting at least one antigen comprising at least a step of combining said at least one antigen with at least one liposome (e.g., a single type of liposomes as disclosed herein) or a combination of at least two types of liposomes as disclosed herein, or at least one liposome or a combination of at least two types of liposomes obtained by a method disclosed herein or an adjuvant composition as disclosed herein.

In another embodiment, the disclosure is directed to a method for adjuvanting an immunogenic response against at least one antigen in an individual in need thereof, comprising administering to said individual said at least one antigen with at least one liposome (e.g., a single type of liposomes as disclosed herein) or a combination of at least two types of liposomes as disclosed herein, or at least one liposome or a combination of at least two types of liposomes obtained by a method disclosed herein or an adjuvant composition as disclosed herein.

In another embodiment, the disclosure is directed to a method for inducing an immune response against at least one antigen in an individual in need thereof, comprising at least one step of administering to said individual said at least one antigen with at least one liposome (e.g., a single type of liposomes as disclosed herein), or a combination of at least two types of liposomes, as disclosed herein, or at least one liposome, or a combination of at least two types of liposomes, obtained by a method disclosed herein or an adjuvant composition as disclosed herein.

In another embodiment, in a method for inducing an immune response in accordance with the invention, the liposome (e.g., a single type of liposomes as disclosed herein), or the combination of at least two types of liposomes, or the adjuvant composition and the antigen may be administered simultaneously, separately, or sequentially. In some embodiment, the first and second types of liposomes of a combination of liposomes as disclosed herein may be administered simultaneously, separately, or sequentially.

In another embodiment, a method for inducing an immune response may further comprise increasing the cytokine and/or chemokine response of said individual. In some embodiments, a method for inducing an immune response may comprise an increase of a cytokine and/or chemokine selected among IL-2, IL-4, IL-5, IL-6, IL-8, IL-12, IL-17, IFN-γ, IP-10, MCP-1, MIP-1β, KC and/or TNF-α. In another embodiment, a method for inducing an immune response may comprise an increase of IFNγ, IL-2, IL-4, IL-5 and IL-17.

### Adjuvanted CMV Antigens-Containing Immunogenic Composition, and Uses Thereof

According to one of its objects, the disclosure relates to an immunogenic composition comprising at least:
one CMV gB antigen;
one CMV gH/gL/UL128/UL130/UL131 pentameric complex antigen; and
one adjuvant comprising:
   either at least one liposome comprising a saponin, a sterol, a phospholipid and a Toll-like receptor 4 (TLR4) agonist or
   at least a combination of at least two types of liposomes, wherein a first type of liposomes comprises a saponin, a sterol, and a phospholipid and a second type of liposomes comprises a sterol, a phospholipid, and a Toll-like receptor 4 (TLR4) agonist.

According to another of its objects, the disclosure relates to an immunogenic composition comprising at least:
one CMV gB antigen;
one CMV gH/gL/UL128/UL130/UL131 pentameric complex antigen; and
one adjuvant comprising:
   either at least one liposome comprising a saponin, a sterol, a phospholipid and a Toll-like receptor 4 (TLR4) agonist, or
   a combination of at least two types of liposomes, wherein a first type of liposomes comprises a saponin, a sterol, and a phospholipid and a second type of liposomes comprises a sterol, a phospholipid, and a Toll-like receptor 4 (TLR4) agonist,
wherein the Toll-like receptor 4 (TLR4) agonist is of formula (I):

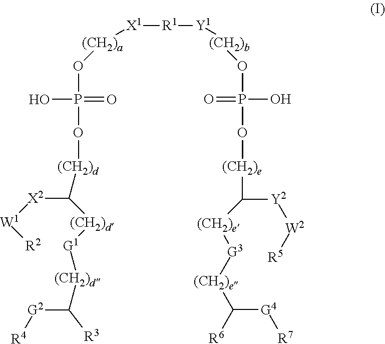

wherein R¹ is selected from the group consisting of:
   a) C(O);
   b) C(O)—(C₁-C₁₄ alkyl)-C(O), in which said C₁-C₁₄ alkyl is optionally substituted with a hydroxyl, a C₁-C₅ alkoxy, a C₁-C₅ alkylenedioxy, a (C₁-C₅ alkyl)amino or a (C₁-C₅ alkyl)aryl, in which said aryl moiety of said (C₁-C₅ alkyl)aryl is optionally substituted with a C₁-C₅ alkoxy, a (C₁-C₅ alkyl)amino, a (C₁-C₅ alkoxy)amino, a (C₁-C₅ alkyl)-amino(C₁-C₅ alkoxy), —O—(C₁-C₅ alkyl)amino(C₁-C₅ alkoxy), —O—(C₁-C₅ alkyl)amino-C(O)—(C₁-C₅ alkyl)-C(O)OH, or —O—(C₁-C₅ alkyl)amino-C(O)—(C₁-C₅ alkyl)-C(O)—(C₁-C₅)alkyl;
   c) an alkyl comprising a C₂-C₁₅ linear or branched chain, optionally substituted with a hydroxyl or an alkoxy; and
   d) —C(O)—(C₆-C₁₂ arylene)-C(O)— in which said arylene is optionally substituted with a hydroxyl, a halogen, a nitro or an amino;
a and b are independently 0, 1, 2, 3 or 4;
d, d′, d″, e, e′ and e″ are independently 0, 1, 2, 3 or 4;
X₁, X₂, Y₁ and Y₂ are independently selected from the group consisting of null, an oxygen, —NH— and —N(C(O)(C₁-C₄ alkyl))-, and —N(C₁-C₄ alkyl)-;
W₁ and W₂ are independently selected from the group consisting of a carbonyl, a methylene, a sulfone and a sulfoxide;
R² and R⁵ are independently selected from the group consisting of:
   a) a C₂ to C₂₀ straight chain or branched chain alkyl, which is optionally substituted with an oxo, a hydroxyl or an alkoxy;
   b) a C₂ to C₂₀ straight chain or branched chain alkenyl or dialkenyl, which is optionally substituted with an oxo, a hydroxyl or an alkoxy;
   c) a C₂ to C₂₀ straight chain or branched chain alkoxy, which is optionally substituted with an oxo, a hydroxyl or an alkoxy;
   d) —NH—(C₂ to C₂₀ straight chain or branched chain alkyl), in which said alkyl group is optionally substituted with an oxo, a hydroxy or an alkoxy; and
   e)

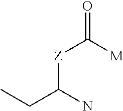

in which Z is selected from the group consisting of an O and NH, and M and N are independently selected from the group consisting of an alkyl, an alkenyl, an alkoxy, an acyloxy, an alkylamino and an acylamino comprising a C₂-C₂₀ linear or branched chain;
R³ and R⁶ are independently selected from the group consisting of a C₂ to C₂₀ straight chain or branched chain alkyl or alkenyl, optionally substituted with an oxo or a fluoro;
R⁴ and R⁷ are independently selected from the group consisting of a C(O)—(C₂ to C₂₀ straight chain or branched chain alkyl or alkenyl), a C₂ to C₂₀ straight chain or branched chain alkyl, a C₂ to C₂₀ straight chain or branched chain alkoxy, and a C₂ to C₂₀ straight chain or branched chain alkenyl; in which said alkyl, alkenyl or alkoxy groups can be independently and optionally substituted with a hydroxyl, a fluoro or a C₁-C₅ alkoxy;
G¹, G², G³ and G⁴ are independently selected from the group consisting of an oxygen, a methylene, an amino, a thiol, —C(O)NH—, —NHC(O)—, and —N(C(O)(C₁-C₄ alkyl))-;
or G²R⁴ or G⁴R⁷ can together be a hydrogen atom or a hydroxyl;
or a pharmaceutically acceptable salt of this compound,
wherein the TLR4 agonist and the saponin are present in a weight:weight ratio of TLR4 agonist:saponin ranging from about 1:50 to about 1:1, or from about 1:35 to about 1:25, or in a weight ratio of TLR4 agonist:saponin of about 1:10.

The adjuvant is comprised of one single type of liposomes or a combination of at least two types of liposomes as described herein.

In some embodiment, the first type of liposomes may be devoid of TLR4 agonist. In some embodiments, the second type of liposomes may be devoid of saponin.

In one exemplary embodiment, a CMV considered in the present disclosure is a Human Cytomegalovirus (HCMV). The gB antigen and CMV gH/gL/UL128/UL130/UL131 pentameric complex antigen may be from a HCMV.

As shown in the Examples section, it was surprisingly observed that an immunogenic composition as disclosed herein, containing HCMV antigens and an adjuvant as disclosed herein (SPA14), was able to elicit long lasting neutralizing antibodies as compared with other HCMV-containing adjuvanted immunogenic compositions.

Furthermore, the adjuvanted immunogenic compositions as disclosed herein presented less reactogenic effect, such as measured with the inflammatory serum biomarkers, such as CRP, neutrophil count or globulin (Example 4), than a composition containing the same antigens but the AS01 adjuvant system used as benchmark adjuvant. Further, an immunogenic composition as disclosed herein was shown as presenting even less reactogenic effect at the second dose than at the prime dose. Further, an immunogenic composition as disclosed herein was shown to be as efficient as an AS01 adjuvanted composition in terms of induction of neutralizing antibodies.

The results presented herein show that an immunogenic composition as disclosed herein may be useful as vaccine against CMV infection as it combines immunogenic efficiency and low reactogenicity. Therefore, such immunogenic composition would favor patient behavior towards acceptance of subsequent doses administration in a multi-dose regimen, and vaccine schedule compliance.

Further, the inventors have surprisingly observed that a combination of at least two types of liposomes where a first type of liposomes comprises a saponin, a sterol, and a phospholipid, but no TLR4 agonists, and a second type of liposomes comprises a sterol, a phospholipid, and a Toll-like receptor 4 (TLR4) agonist, but no saponins, each comprising hCMV antigens, such as gB and pentamer, was able to induce a similar adjuvanting effect than a single type of liposomes comprising a sterol, a phospholipid, a saponin and a Toll-like receptor 4 (TLR4) agonist and the hCMV antigens. Further, as shown in the Examples, the liposomes or combinations of at least two types of liposomes as disclosed herein comprising QS7 as saponin present advantageously a good safety profile and a good adjuvanting effect with hCMV antigens.

According to one embodiment, an immunogenic composition as disclosed herein may comprise a CMV gB antigen selected in a group comprising a full length CMV gB antigen, a truncated CMV gB antigen deleted from at least a part of the transmembrane domain, a truncated CMV gB antigen substantially deleted from all the transmembrane domain, a truncated CMV gB antigen deleted from at least a part of the intracellular domain, a truncated CMV gB antigen substantially deleted from all the intracellular domain, and a truncated CMV gB antigen deleted substantially from both the transmembrane domain and the intracellular domain.

According to one exemplary embodiment, an CMV gB antigen may be the gBdTM antigen.

According to another exemplary embodiment, a CMV gH antigen from a pentameric complex antigen may be deleted from at least a part of the transmembrane domain or from substantially all the transmembrane domain.

According to another exemplary embodiment, a CMV gH antigen from a pentameric complex antigen may comprise the ectodomain of the full length gH polypeptide encoded by CMV UL75 gene.

According to one embodiment, a CMV gB antigen and a CMV gH/gL/UL128/UL130/UL131 pentameric complex antigen may be the only CMV antigens present in an immunogenic composition as disclosed herein.

According to one embodiment, a TLR4 agonist may have a solubility parameter in ethanol, measured at 25° C., of at least about 0.2 mg/ml.

According to one exemplary embodiment, a TLR4 agonist may be of formula (II):

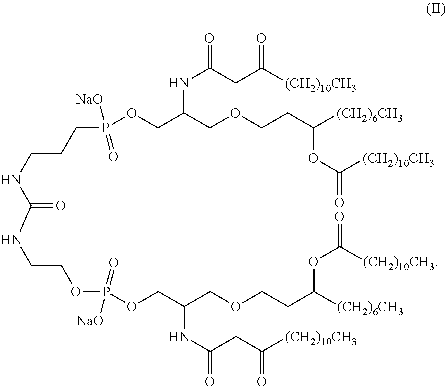

According to another exemplary embodiment, a TLR4 agonist may be of formula (III):

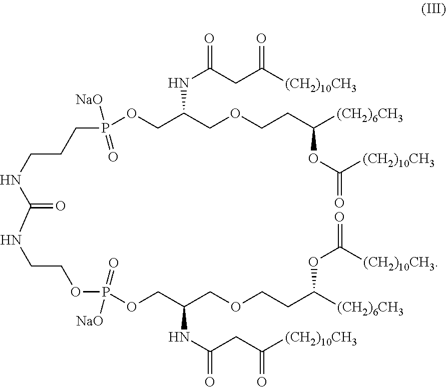

According to one embodiment, a saponin may be a Quillaja saponaria saponin.

According to another embodiment, a saponin is extracted from the bark of Quillaja saponaria Molina.

In another embodiment, a saponin may be selected among QS7, QS17, QS18, QS21, and combinations thereof. A saponin may be QS7 or QS21.

According to another embodiment, a saponin may be QS21.

According to another embodiment, a saponin may be QS7.

According to one embodiment, a sterol may be selected from cholesterol or its derivatives, ergosterol, desmosterol (3β-hydroxy-5,24-cholestadiene), stigmasterol (stigmasta-5,22-dien-3-ol), lanosterol (8,24-lanostadien-3b-ol), 7-dehydrocholesterol (Δ5,7-cholesterol), dihydrolanosterol (24,25-dihydrolanosterol), zymosterol (5α-cholesta-8,24-dien-33-ol), lathosterol (5α-cholest-7-en-3β-ol), diosgenin ((3β,25R)-spirost-5-en-3-ol), sitosterol (22,23-dihydrostigmasterol), sitostanol, campesterol (campest-5-en-3β-ol), campestanol (5a-campestan-3b-ol), 24-methylene cholesterol (5,24(28)-cholestadien-24-methylen-3β-ol), cholesteryl margarate (cholest-5-en-3β-yl heptadecanoate), cholesteryl oleate, cholesteryl stearate, and mixtures thereof.

According to another embodiment, a sterol may be selected from cholesterol or its derivatives, in particular is cholesterol.

According to one embodiment, a saponin and a sterol may be present in a weight:weight ratio of saponin:sterol ranging from 1:100 to 1:1, ranging from 1:50 to 1:2, or ranging from 1:10 to 1:5, or in a weight:weight ratio of saponin:sterol of about 1:2, or in a weight:weight ratio of saponin:sterol of about 1:5.

According to one embodiment, a phospholipid may be selected from phosphatidylcholines, phosphatidic acids, phosphatidylethanolamines, phosphatidylglycerols, phosphatidylserines, phosphatidylinositols, and mixtures thereof.

According to another embodiment, a phospholipid may be a phosphatidylcholine selected from DSPC (1,2-distearoyl-sn-glycero-3-phosphocholine), DPPC (1,2-dipalmitoyl-sn-glycero-3-phosphocholine), DMPC (1,2-dimyristoyl-sn-glycero-3-phosphocholine), POPC (1-palmitoyl-2-oleoyl-sn-glycero-3-phosphocholine), DOPC (1,2-dioleoyl-sn-glycero-3-phosphocholine), SOPC (1-stearoyl-2-oleoyl-sn-glycero-3-phosphocholine), and mixtures thereof.

According to one embodiment, an immunogenic composition as disclosed herein may be for use as a CMV vaccine, such as an HCMV vaccine.

According to one embodiment, an immunogenic composition as disclosed herein may be for use in a method for inducing neutralizing antibodies against a CMV, said method comprising administering to a subject at least a first and a second doses of said composition, the at least first and second doses being administered at least one month-apart, wherein the second dose induces to said subject less reactogenicity than the first dose, said reactogenicity being measured with a method comprising at least the steps of (a) dosing at least a biomarker selected among CRP, globulin and fibrinogen (i) in a first blood sample taken from said subject having been administered with said first dose of said composition and before being administered with said second dose of said composition to obtain a first measured amount of said biomarker, and (ii) in a second blood sample taken from said subject having been administered with said second dose of said composition to obtain a second measured amount of said biomarker, and (b) comparing said first measured amount with said second measured amount wherein said comparison is informative as to the reactogenicity elicited by said administered composition.

In some embodiments, an increased measured amount of at least biomarker in the second measure compared to the first measure may be indicative of a reactogenic composition. In some embodiments, an absence of increased measured amount of at least biomarker in the second measure compared to the first measure may be indicative of a no or reduced reactogenic composition.

According to one embodiment, it is disclosed a kit-of-parts comprising:
a first container comprising a first composition comprising an adjuvant as disclosed herein, and
a second container comprising a second composition comprising at least one CMV gB antigen and at least one CMV gH/gL/UL128/UL130/UL131 pentameric complex antigen as disclosed herein.

In another embodiment, the disclosure is directed to a kit-of-parts comprising:
a first container comprising a first composition comprising a single type of liposomes as disclosed herein or at least one single type of liposomes obtained by a method as disclosed herein or an adjuvant composition as disclosed herein, and
a second container comprising a second composition comprising at least one CMV gB antigen and at least one CMV gH/gL/UL128/UL130/UL131 pentameric complex antigen as disclosed herein. In such embodiment, the liposome may be a single type of liposomes.

According to one embodiment, it is disclosed a kit-of-parts comprising:
a first container comprising a first composition comprising a first type of liposomes comprising a saponin, a sterol, and a phospholipid,
a second container comprising a second type of liposomes comprising a sterol, a phospholipid, and a Toll-like receptor 4 (TLR4) agonist, and
a third container container comprising a third composition comprising at least one CMV gB antigen and at least one CMV gH/gL/UL128/UL130/UL131 pentameric complex antigen as disclosed herein.

According to one embodiment, it is disclosed a method for inducing an immune response against a CMV in a subject, comprising at least one step of administering to said subject at least one immunogenic composition as disclosed herein.

According to another embodiment, a method as disclosed herein may comprise administering to said subject a first and a second doses of said composition, at least one month-apart, wherein the second dose induces less reactogenicity than the first dose, said reactogenicity being measured with a method comprising at least the steps of (a) dosing at least one biomarker selected among CRP, globulin and fibrinogen (i) in a first blood sample taken from said subject after being administered with said first dose of said composition and before being administered with said second dose of said composition to obtain a first measured amount of said biomarker, and (ii) in a second blood sample taken from said subject after being administered with said second dose of said composition to obtain a second measured amount of said biomarker, and (b) comparing said first measured amount with said second measured amount wherein said comparison is informative as to the reactogenicity elicited by said administered composition.

In some embodiments, an increased measured amount of at least biomarker in the second measure compared to the first measure may be indicative of a reactogenic composition. In some embodiments, an absence of increased measured amount of at least biomarker in the second measure compared to the first measure may be indicative of a no or reduced reactogenic composition.

### DESCRIPTION OF THE FIGURES

FIG. 1: Variation of the Relative nephelometry unit (RNU) (ordinate) for E6020 solution (●) and for MPL solution (♦) in ethanol for increasing Ethanol concentrations (abscissa): 0.5, 1.0, 2.0 and 10 mg/ml on a UV 96-well microplate.

FIG. 2: Cell viability (%) (ordinate) measured via flow cytometry in a MIMIC®PTE system (Modular Immune In vitro Construct—Peripheral tissue equivalent) in the following situations 48h after administration, from left to right on the abscissa axis: mock condition (M—Mock), in the presence of a mixture of 100 ng/mL LPS (from Pseudomonas aeruginosa, Cat #L8643, Millipore Sigma, Burlington, MA), and 10 μg/mL R848 (Cat #TLRL-R848, InvivoGen, San Diego, CA), in the presence of SPA14-8 (diluted 1:40, 1:400, 1:4000 and 1:40000), in the presence of QS21 liposome (SPA14-0) (diluted 1:40, 1:400, 1:4000 and 1:40000), and in the presence of E6020-Eq-1:40. The mock condition for each donor was normalized to 100% and the treatment conditions were calculated against this value. Bars represent Geo. Mean±95% Cl; n=8-20 donors.

FIG. 3: Amount of CD86-positive APCs (Antigene presenting cells) (% HLA-DR+CD11c+CD86+) (ordinate) measured via flow cytometry in a MIMIC®PTE system in the following situations 48h after administration, from left to right on the abscissa axis: mock condition (M—Mock), in the presence of a mixture of 100 ng/mL LPS (from Pseudomonas aeruginosa, Cat #L8643, Millipore Sigma, Burlington, MA), and 10 μg/mL R848 (Cat #TLRL-R848, InvivoGen, San Diego, CA), in the presence of SPA14-20 (diluted 1:20, 1:40, 1:80 and 1:160), and in the presence of SPA14-8 (diluted 1:20, 1:40, 1:80 and 1:160) Bars represent Geo. Mean±95% Cl; n=8-20 donors. ANOVA with Tukey post-test. Mock vs. SPA14-20, 1:20: ****, Mock vs SPA14-8, 1:20: ****, SPA14-20 vs SPA14-8: NS (**** is indicative of a p-value<0.05).

FIG. 4: HCMV neutralizing antibody responses in sera from immunized rabbits. μPRNT50 on epithelial cells MRC-5 in absence of complement at D15, D24 and D36 (A) and μPRNT50 on fibroblast ARPE-19 in presence of complement at D24 and D36 (B). Rabbits were immunized twice (DO, D21) with gB+pentamer (●), gB+pentamer+SPA14 (0 μg of E6020) (▾), gB+pentamer+SPA14 (1 μg of E6020) (▾), gB+pentamer+SPA14 (2 μg of E6020) (▾), gB+pentamer+SPA14 (5 μg of E6020) (▾), and gB+pentamer+AS01B (▪). (see Examples 1 and 9).

FIG. 5: HAI titers obtained for Fluzone® QIV (0.1 and 0.5 μg HA) against A/Hong Kong/4801/2014 (H3N2) strain (ordinate) after administration of (from left to right): SPA14+0.1 μg HA Fluzone®, AS01B+0.1 μg HA Fluzone®, SPA14+0.5 μg HA Fluzone®, AS01B+0.5 μg HA Fluzone®, 0.1 μg HA Fluzone® alone and 0.5 μg HA Fluzone® alone (abscissa) at D35 in the sera of mice.

FIG. 6: HAI titers obtained for Fluzone® QIV 0.5 μg HA against HK/2014 strain, Michigan/2015 strain, Brisbanne/08 strain, Singapore/2016 strain and Colorado/2017 strain (ordinate) after administration of (from left to right) adjuvanted formulations with SPA14 or AS01B and Fluzone® QIV 0.5 μg HA alone (abscissa) at D35 in the sera of mice.

FIG. 7: HAI titers obtained for Flublok® QIV 1 μg HA against Michigan/2015 (H1N1) strain and Brisbanne/08 strain (ordinate) after administration of (from left to right) adjuvanted formulations with SPA14 or AS01B and Flublok® QIV 1 μg HA alone (abscissa) at D35 in the sera of mice.

FIG. 8: increase of IFNγ, IL-5, TNFα, MCP-1, KC, and IL-6 secretion in response to immunization with Fluzone® and Flublok® adjuvanted formulations. Amount of cytokine/chemokine (pg/mL) (ordinate) in the presence of (from left to right): no antigens (prebleed), Fluzone® alone (Fzone), Flublok® alone (Fblok), SPA14 alone, Fzone+SPA14, Fblok+SPA14, AS01B, Fzone+AS01B and Fblok+AS01B (abscissa) in sera of immunized mice 6 hrs after immunization.

FIG. 9: Th1 (IFNγ)/Th2 (IL-5) cytokine secretion in splenocytes of immunized mice two weeks after boost immunization (Day 35) measured by ELISPOT. Ratio Th1/Th2 (ordinate) after administration of (from left to right) Fluzone alone (∘), Fluzone+SPA14 (▪), Fluzone+AS01B (▪), Flublok alone (Δ), Flublok+SPA14 (▾) and Flublok+AS01B (▴) (abscissa).

FIG. 10: Adjuvanted gB plus pentamer neutralizing antibody response to human CMV virus strain. Human BADrUL131-Y4 CMV virus strain neutralizing titers (PRNT50) (ordinate) measured on D20 and D35 on ARPE-19 epithelial cell line without (A) and on MRC-5 fibroblast cell line with (B) additional complement following intramuscular administration of 8 C57BL/6 mice without adjuvant, with SPA14 or with AS01B on DO and D21 (abscissa). Mouse data are shown as scattered plots and geometric mean of neutralizing titers (GMT) for each group. Tukey adjustment and one-way ANOVA (p<0.05)

FIG. 11: hCMVgB and pentamer IgG1 and IgG2c-secreting B cells in spleen cells from immunized mice.

hCMV gB-specific IgG1 and IgG2c-secreting B cells (A and B) and hCMV pentamer-specific IgG1 and IgG2c-secreting B cells measured on D35 following IM administrations of C57BL/6 mice on DO and D21 with non adjuvanted hCMV gB plus pentamer vaccine, with SPA14-adjuvanted hCMV gB plus pentamer or AS01B-adjuvanted hCMV gB plus pentamer. (A) gB-specific IgG1- and IgG2c-secreting B cells frequencies per 10⁶ spleen cells. (B) Ratio of IgG1- and IgG2c-secreting B cells specific to gB. (C) Pentamer-specific IgG1- and IgG2c-secreting B cells frequencies per 10⁶ spleen cells. (D) Ratio of IgG1- and IgG2c-secreting B cells specific to pentamer. Bar=Geometric mean, scattered dot=individual mouse response, dotted line in (A) and (C)=responder cut-off, dotted line in (B) and (D)=balanced Th1/Th2 ratio=1. Tukey adjustment and one-way ANOVA (p<0.05)

FIG. 12: Characterization of T cell responses in spleen cells from immunized mice. hCMV gB-specific IFN-γ- and IL-5-secreting cells (A and B) and hCMV pentamer-specific IFN-γ- and IL-5-secreting cells measured on D35 following IM administrations of C57BL/6 mice on DO and D21 with non adjuvanted hCMV gB plus pentamer vaccine, with SPA14-adjuvanted hCMV gB plus pentamer and AS01B-adjuvanted hCMV gB plus pentamer. (A) gB-specific IFNγ-secreting cells frequencies (per 10⁶ spleen cells). (B) gB-specific IL-5-secreting cells frequencies per 10⁶ spleen cells (C) Ratio of IFNγ- and IL-5-secreting cells specific to gB. (D) Pentamer-specific IFNγ-secreting cells frequencies per 10⁶ spleen cells. (E) Pentamer-specific IL-5-secreting cells frequencies per 10⁶ spleen cells. (F) Ratio of IFNγ- and IL-5-secreting cells specific to pentamer. Bar=Geometric mean, scattered dot=individual mouse response, dotted line in (A) (B) (D) and (E)=responder cut-off, dotted line in (C) and (F)=balanced Th1/Th2 ratio=1. Tukey adjustment and one-way ANOVA (p<0.05).

FIG. 13: SPA14 enhances F-specific IgG ELISA responses in sera from pre-F-ferritin vaccinated NHPs. Individual monkey data are shown for each group. Dotted lines=limit of quantification.

F-specific IgG titers (sera) (ordinate) after administration of Pre-F ferritin+SPA14 (left graph) or Pre-F ferritin alone (right graph) over time, in days, for four different macaques: macaque #1 (●) macaque #2 (▪) macaque #3 (▴) and macaque #4 (▾) (abscissa).

FIG. 14: RSV-A2 neutralizing antibody response to the Pre-F-ferritin. RSV-A2 neutralizing titers (PRNT60) (ordinate) without (A) and with (B) complement over time (in days) (abscissa) following intramuscular vaccination of four cynomolgus macaques (macaque #1 (●) macaque #2 (▪) macaque #3 (▴) and macaque #4 (▾)) without adjuvant or with SPA14 on day 0 and day 28. Individual monkey data are shown for each group. Dotted lines=limit of quantification. (ANOVA **P-value<0.01).

FIG. 15: Pre-F-ferritin+SPA14 induces cross-neutralizing antibodies to RSV B strain in NHPs. RSV-A2 neutralizing titers (PRNT60) (ordinate) without complement over time (in days) (abscissa) following intramuscular vaccination of four cynomolgus macaques (macaque #1 (0) macaque #2 (n) macaque #3 (A) and macaque #4 (V)) without adjuvant or with SPA14. Individual monkey data are shown for each group. Dotted lines=limit of quantification.

FIG. 16: F-specific IgG memory B-cell ELISpot responses in PBMCs from immunized macaques. F-specific memory B cell ELISpot results at baseline, day 119 and day 161 following IM vaccination of cynomolgus macaques with Pre-F-NP adjuvanted or not with SPA14 on day 0 and day 28. (A) F-specific memory IgG-secreting cells/10⁶ cells. (B) % F-specific memory IgG-secreting cells/total IgG secreting cells. Bar=Geometric mean; dotted line=responder cut-off; ANOVA **P-value<0.01.

FIG. 17: Characterization of cellular immune responses in macaques following vaccination. (A) F-specific IFNγ ELISpot responses and (B) F-specific IL-2 ELISpot responses at D7 (7 days post-dose 1) and D35 (7 days post-dose 2) in PBMCs from immunized macaques. **P-value<0.01. Bar=Geometric mean; dotted line=responder cut-off.

FIG. 18: represents the results of a micro-plaque reduction neutralization test (μPRNT) carried on epithelial cell line ARPE-19 in presence of complement with sera obtained from mice injected with saline buffer (Δ) or immunized with an immunogenic composition comprising 20 μg/dose HCMV gB+20 μg/dose HCMV gH/gL/UL128/UL130/UL131A in buffer (e.g. PBS pH 7.4, NaCl 140 mM; - -▾- -), or formulated with SPA14 (▾), AF04 (♦), AF03 (●) or AS01E (□) (see Examples 1 and 2). The animals were injected with the immunogenic composition at day 0, 21, and 221 (month 7). In abscissa axis is given the day of blood sampling, i.e. Day (D) 19, month (M) 1, M2, M3, M4, M5, M6, M7 and M8, and in ordinate axis is given the μPRNT neutralizing antibody titers (log 10).

FIG. 19: Neutralizing antibody titers specific to gB and pentamer. Panel A: Neutralizing antibody on epithelial cells MRC-5 in absence of complement. Panel B: Neutralizing antibody on fibroblasts ARPE-19 in presence of complement. Neutralizing antibodies were measured at months 1 and 8 (*p-values<0.05, **p-value<0.001 when compared to AF03). Sera were obtained from mice immunized with an immunogenic composition comprising 20 μg/dose HCMV gB+20 μg/dose HCMV gH/gL/UL128/UL130/UL131A in buffer (e.g. PBS pH 7.4, NaCl 140 mM), adjuvanted with SPA14, AF04, AF03 or AS01E (see Examples 1 and 2). The animals were injected with the immunogenic composition at day 0, 21, and 221 (month 7).

FIG. 20: IFN-γ(panel A) and IL-5 (panel B) secreting cells frequencies upon CMV pentamer stimulation measured by ELISPOT at months 1, 7 and 8. Sera were obtained from mice immunized with an immunogenic composition comprising 20 μg/dose HCMV gB+20 μg/dose HCMV gH/gL/UL128/UL130/UL131A in buffer (e.g. PBS pH 7.4, NaCl 140 mM), adjuvanted with SPA14, AF04, AF03 or AS01E (see Examples 1 and 2). The animals were injected with the immunogenic composition at day 0, 21, and 221 (month 7).

FIG. 21 shows the haemolytic effect of QS21 or QS7 (from 0.8 μM to 100 μM) or with citrate buffer used as control on sheep red blood cells.

FIGS. 22A, 22B, 22C and 22D show hCMVgB and pentamer IgG1 and IgG2c-induced responses in mice immunized with CMV gB and CMV pentamer (2 μg each/dose) formulated with DOPC-Chol liposomes containing QS21 (5 μg) without E6020 (“QS21 LIP” (0:200 μg/mL)), DOPC-Chol liposomes containing E6020 without QS21 or QS7 (“E6020 LIP” (20:0 μg/mL)), SPA14 containing QS21 (DOPC-Chol liposomes containing 5 μg QS21 and 0.5 μg E6020/dose (“SPA14” (20:200 μg/mL)), SPA14-like containing QS7 (DOPC-Chol liposomes containing 5, 15 or 45 μg QS7 and 0 or 0.5 μg of E6020/dose) (“QS7 LIP” (0:200 μg/mL)”, (0:600 μg/mL) or (0:1800 μg/mL), “LIP [QS7+E6020 20]” (20:200 μg/mL), (20:600 μg/mL) or (20:1800 μg/mL)).

FIGS. 23A & 23B show the IgG1/IgG2c responses ratios induced in mice immunized with CMV gB and CMV pentamer (2 μg each/dose) formulated in DOPC-Chol liposomes containing QS21 (5 μg) without E6020 (“QS21 LIP” (0:200)), DOPC-Chol liposomes containing E6020 without QS21 or QS7 (“E6020 LIP” (20:0)), SPA14 containing QS21 (DOPC-Chol liposomes containing 5 μg QS21 and 0.5 μg E6020/dose(“SPA14” (20:200)), and SPA14-like formulation containing QS7 (DOPC-Chol liposomes containing 5, 15 or 45 μg QS7 and 0 or 0.5 μg E6020/dose (“QS7 LIP” (0:200)”, (0:600) or (0:1800), “LIP [QS7+E6020 20]” (20:200), (20:600) or (20:1800)).

FIG. 24 shows the serum neutralizing titer response induced in mice immunized with CMV gB and CMV pentamer (2 μg each/dose) formulated in DOPC-Chol liposomes containing QS21 (5 μg) without E6020 (“QS21 LIP” (0:200)), DOPC-Chol liposomes containing E6020 without QS21 or QS7 (“E6020 LIP” (20:0)), SPA14 containing QS21 (DOPC-Chol liposomes containing 5 μg QS21 and 0.5 μg E6020/dose) (“SPA14” (20:200)), and SPA14-like formulation containing QS7 (DOPC-Chol liposomes containing 5, 15 or 45 μg QS7 and 0 or 0.5 μg E6020/dose (“QS7 LIP” (0:200)”, (0:600) or (0:1800), “LIP [QS7+E6020 20]” (20:200), (20:600) or (20:1800)).

FIGS. 25A & 25B show the IFN-γ- and IL-5-secreted responses ratios induced induced in mice immunized with CMV gB and CMV pentamer (2 μg each/dose) formulated in in DOPC-Chol liposomes containing QS21 (5 μg) without E6020 (“QS21 LIP” (0:200)), DOPC-Chol liposomes containing E6020 without QS21 or QS7 (“E6020 LIP” (20:0)), SPA14 containing QS21 (DOPC-Chol liposomes containing 5 μg QS21 and 0.5 μg E6020/dose (“SPA14” (20:200)), and SPA14-like formulation containing QS7 (DOPC-Chol liposomes containing 5, 15 or 45 μg QS7 and 0 or 0.5 μg E6020/dose [please confirm]) (“QS7 LIP” (0:200)”, (0:600) or (0:1800), “LIP [QS7+E6020 20]” (20:200), (20:600) or (20:1800)).

FIGS. 26A & 26B show hCMVgB and pentamer IgG1 and IgG2c-induced responses in mice immunized with CMV gB and CMV pentamer (2 μg each/dose) formulated with DOPC-Chol liposomes containing QS21 (5 μg) without E6020 (“QS21 LIP” (0:200)), DOPC-Chol liposomes containing E6020 without QS21 (“E6020 LIP” (20:0)), SPA14 containing QS21 (DOPC-Chol liposomes containing 5 μg QS21 and 0.5 μg E6020/dose) (“SPA14h20)” (20:200)), and a combination of “QS21 LIP” and “E6020 LIP” (QS21 LIP”+“E6020 LIP”) with QS21 and E6020 injected at the same doses than those found in SPA14.

FIGS. 27A & 27B show the IgG1/IgG2c responses ratios induced in mice immunized with CMV gB and CMV pentamer (2 μg each/dose) formulated in DOPC-Chol liposomes containing QS21 (5 μg) without E6020 (“QS21 LIP” (0:200)), DOPC-Chol liposomes containing E6020 without QS21 (“E6020 LIP” (20:0)), SPA14 containing QS21 (DOPC-Chol liposomes containing 5 μg QS21 and 0.5 μg E6020/dose and a combination of “QS21 LIP” and “E6020 LIP” (QS21 LIP”+“E6020 LIP”) with QS21 and E6020 injected at the same doses than those found in SPA14.

FIGS. 28A & 28B show the IFN-γ- and IL-5-secreted responses ratios induced in mice immunized with CMV gB and CMV pentamer (2 μg each/dose) formulated in DOPC-Chol liposomes containing QS21 (5 μg) without E6020 (“QS21 LIP” (0:200)), DOPC-Chol liposomes containing E6020 without QS21 (“E6020 LIP” (20:0)), SPA14 containing QS21 (DOPC-Chol liposomes containing 5 μg QS21 and 0.5 μg E6020/dose), and a combination of “QS21 LIP” and “E6020 LIP” (QS21 LIP”+“E6020 LIP”) with QS21 and E6020 injected at the same doses than those found in SPA14.

FIGS. 29A & 29B show the results of a micro-plaque reduction neutralization test (μPRNT) carried on epithelial cell lines MRC5 (B) in absence of complement and ARPE-19 (A) in presence of complement, with sera obtained from mice immunized with CMV gB and CMV pentamer (2 μg each/dose) formulated in DOPC-Chol liposomes containing QS21 (5 μg) without E6020 (“QS21 LIP” (0:200)), DOPC-Chol liposomes containing E6020 without Qs21 (“E6020 LIP” (20:0)), SPA14 containing QS21 (DOPC-Chol liposomes containing 5 μg QS21 and 0.5 μg E6020/dose) and a combination of “QS21 LIP” and “E6020 LIP” (QS21 LIP”+“E6020 LIP”) with QS21 and E6020 injected at the same doses than those found in SPA14.

### DETAILED DESCRIPTION

### Definitions

The terms used in this specification generally have their ordinary meanings in the art. Certain terms are discussed below, or elsewhere in the present disclosure, to provide additional guidance in describing the products and methods of the presently disclosed subject matter.

The following definitions apply in the context of the present disclosure:

As used in this specification and the appended claims, the singular forms “a”, “an” and “the” include plural referents unless the content clearly dictates otherwise.

The terms “about” or “approximately” as used herein refer to the usual error range for the respective value readily known to the skilled person in this technical field. Reference to “about” a value or parameter herein includes (and describes) embodiments that are directed to that value or parameter per se. In some embodiments, the term “about” refers to ±10% of a given value. However, whenever the value in question refers to an indivisible object, such as a molecule or other object that would lose its identity once subdivided, then “about” refers to ±1 of the indivisible object.

It is understood that aspects and embodiments of the present disclosure described herein include “having,” “comprising,” “consisting of,” and “consisting essentially of” aspects and embodiments. The words “have” and “comprise,” or variations such as “has,” “having,” “comprises,” or “comprising,” will be understood to imply the inclusion of the stated element(s) (such as a composition of matter or a method step) but not the exclusion of any other elements. The term “consisting of” implies the inclusion of the stated element(s), to the exclusion of any additional elements. The term “consisting essentially of” implies the inclusion of the stated elements, and possibly other element(s) where the other element(s) do not materially affect the basic and novel characteristic(s) of the disclosure. It is understood that the different embodiments of the disclosure using the term “comprising” or equivalent cover the embodiments where this term is replaced with “consisting of” or “consisting essentially of”.

As used herein, the terms “immunologically effective amount” used with regard to an antigen or a combination of an antigen and an adjuvant, intend to refer to an amount which, when administered to a subject, is effective for eliciting an immune response against the antigen. This amount may vary depending various factors, such as the health or physical condition of the subject, its age, the capacity of the subject's immune system to produce antibodies, the degree of protection desired, the formulation of the composition containing the antigen, the treating doctor's assessment of the medical situation. This amount may be determined by routine methods known to the skilled person.

As used herein, in the context of an immune response elicitation, the terms “treat”, “treatment”, “therapy” and the like refer to the administration or consumption of a composition as disclosed herein with the purpose to cure, heal, alleviate, relieve, alter, remedy, ameliorate, improve, or affect a disease or a disorder, the symptoms of the condition, or to prevent or delay the onset of the symptoms, complications, or otherwise arrest or inhibit further development of the disorder in a statistically significant manner.

Also, as used herein, in the context of the present disclosure, the terms “treat”, “treatment” and the like refer to relief from or alleviation of pathological processes mediated by CMV infection. In the context of the present disclosure, insofar as it relates to any of the other conditions recited herein, the terms “treat”, “treatment”, and the like refer to relieving or alleviating one or more symptoms associated with such condition.

As used herein, the terms “prevent”, “preventing” or “delay progression of” (and grammatical variants thereof) with respect to a disease or disorder relate to prophylactic treatment of the disease or the disorder, e.g., in an individual suspected to have the disease, or at risk for developing the disease. Prevention may include, but is not limited to, preventing or delaying onset or progression of the disease and/or maintaining one or more symptoms of the disease or disorder at a desired or sub-pathological level. The term “prevent” does not require the 100% elimination of the possibility or likelihood of occurrence of the event. Rather, it denotes that the likelihood of the occurrence of the event has been reduced in the presence of a composition or method as described herein.

As used herein, the terms “effective amount”, “therapeutically effective amount” and “prophylactically effective amount” refer to an amount that provides a therapeutic benefit in the treatment, prevention, or management of the disease or disorder considered. The specific amount that is therapeutically effective can be readily determined by an ordinary medical practitioner and may vary depending on factors such as the type and stage of the disease or disorder considered, the patient's medical history and age, and the administration of other therapeutic agents.

As used herein, the terms “individual” or “subject” or “patient” are used interchangeably and intends to refer to a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In some exemplary embodiments, the individual or subject is a human.

In the context of the disclosure, the expression “neutralizing antibody” has the meaning known to a skilled person and is intended to cover an antibody that directly neutralizes its target pathogen, for example by blocking a virus entry into a host cell or by blocking the virus dissemination from cell to cell. Neutralizing antibodies are functional antibodies that are able to induce an immune protection into a subject with regard to their pathogen target. Some illustration of the methods available to determine presence and/or increase and/or amount of a neutralizing antibody level and/or neutralizing antibody persistence is provided in the experimental part of the present disclosure.

In the context of the disclosure, the expression “pharmaceutically acceptable carrier” refers to a carrier or vehicle that is physiologically acceptable for administration to a mammal, such as a human being, while retaining the physiological activity of the immunogenic composition as disclosed herein, i.e., its ability to induce an immune response with a low reactogenic effect.

The term “pharmaceutically acceptable salts” includes addition salts of compounds as disclosed herein derived from the combination of such compounds with for example non-toxic acid addition salts.

The term “antigen” comprises any molecule, for example a peptide, a protein, a polysaccharide or a glycoconjugate, which comprises at least one epitope that will elicit an immune response and/or against which an immune response is directed. For example, an antigen is a molecule which, optionally after processing, induces an immune response, which is for example specific for the antigen or cells expressing the antigen. After processing, an antigen may be presented by MHC molecules and reacts specifically with T lymphocytes (T cells). Thus, an antigen or fragments thereof should be recognizable by a T cell receptor and should be able to induce in the presence of appropriate co-stimulatory signals, clonal expansion of the T cell carrying the T cell receptor specifically recognizing the antigen or fragment, which results in an immune response against the antigen or cells expressing the antigen. According to the present disclosure, any suitable antigen may be envisioned which is a candidate for an immune response. An antigen may correspond to or may be derived from a naturally occurring antigen. Such naturally occurring antigens may include or may be derived from allergens, viruses, bacteria, fungi, parasites and other infectious agents and pathogens or an antigen may also be a tumor antigen. Said antigens may be proteins or peptides antigens, polysaccharide antigens or glycocongugate antigens. Antigens suitable herein are discussed further in the disclosure.

Within the context of the disclosure and vaccines, “reactogenicity” intends to refer to a subset of symptoms occurring shortly after vaccination, and which are a physical manifestation of the inflammatory response to vaccination. Those symptoms may be local (injection site) or systemic symptoms and may include at least one of: pain, redness, swelling, site-injection induration as local symptoms and, and fever, myalgia, headache, or rash as systemic symptoms. The reactogenicity of a vaccine or an immunogenic composition may also be determined by the measure of a level of some biomarkers such as globulin, CRP, fibrinogen or neutrophil counts and the comparison of the measure level with a level of reference. Within the context of the present disclosure, “low reactogenicity” or “reduced reactogenicity” is used to qualify a level of a reactogenic response elicited by an immunogenic or vaccine composition used for a given therapeutic indication in an individual receiving a dose of this first composition that is inferior to the level of a reactogenic response elicited in the same or another individual receiving or having received an equivalent dose of a second immunogenic or vaccine composition used for the same given therapeutic indication, the second immunogenic being different in its formulation relatively to the first one. Also, “low reactogenicity” or “reduced reactogenicity” may qualify a level of reactogenic response elicited by an immunogenic or vaccine composition used for a given therapeutic indication in an individual receiving a dose of this composition that is inferior to the level of a reactogenic response elicited in the same individual having received a previous identical dose of this composition or receiving a subsequent identical dose of this composition. A level of reactogenic response may be determined by the measure of at least one symptom or of at least one biomarker usually considered as a reactogenic symptom or biomarker. A biomarker of reactogenicity may be CRP, globulin or fibrinogen dosed in a blood or sera sample.

The term “sterol” or “steroid alcohol” refers to a group of lipids comprised of a sterane core bearing a hydroxyl moiety which may be free or esterified. As example of steroid alcohol with a free hydroxyl moiety, one may cite cholesterol, campesterol, sitosterol, stigmasterol and ergosterol. Esters of steroid alcohol or of sterol refer to ester of carboxylic acid with the hydroxyl group of the steroid alcohol. Suitable carboxylic acid comprises, further to the carboxyl moiety, a saturated or unsaturated, linear, or branched, alkyl group. In some embodiments the alkyl group may be a C₁-C₂₀ alkyl group. In other embodiments, the carboxylic acid may be a fatty acid.

Within the disclosure, the term “significantly” used with respect to change intends to mean that the observe change is noticeable and/or it has a statistic meaning.

Within the disclosure, the term “substantially” used in conjunction with a feature of the disclosure intends to define a set of embodiments related to this feature which are largely but not wholly similar to this feature. The difference between the set of embodiments related to a given feature and the given feature is such that in the set of embodiments, the nature and function of the given feature is not materially affected.

As used herein, the term “immunopotentiating” refers to a compound or composition which has the ability to trigger and/or enhance an immune response by activating components of the immune system in an individual to whom it is administered to.

Within the disclosure, the term “adjuvant” or “adjuvant effect” is used to qualify a compound or composition which is added to an antigen-containing vaccine compositions to help trigger or enhance an immune response to the antigen by, e.g., enhancing antigen presentation to antigen-specific immune cells and by activating these cells with the aim to confer long-term protection against targeted pathogens.

As used herein, the term “vaccine” is intended to mean an immunogenic composition directed to a pathogen agent which is administered to a subject to induce an immune response with the intent to protect or treat the subject from an illness caused by the pathogen agent. A vaccine as disclosed herein is intended for use as a preventive (prophylactic) vaccine, for administration to a subject prior to infection, with the intent to prevent, or reduced the likelihood of occurrence of, initial (and/or recurrent) infection. In case of congenital CMV infection, a composition as disclosed herein may be intended for use as a preventive vaccine for adolescent girls and women of child-bearing age, before pregnancy in order to prevent, or reduce the likelihood of occurrence of, the vertical CMV transmission from mother to fetus or infant.

The list of sources, ingredients, and components as described hereinafter are listed such that combinations and mixtures thereof are also contemplated and within the scope herein.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

All lists of items, such as, for example, lists of ingredients, are intended to and should be interpreted as Markush groups. Thus, all lists can be read and interpreted as items “selected from the group consisting of’ . . . list of items . . . “and combinations and mixtures thereof.”

Referenced herein may be trade names for components including various ingredients utilized in the present disclosure. The inventors herein do not intend to be limited by materials under any particular trade name. Equivalent materials (e.g., those obtained from a different source under a different name or reference number) to those referenced by trade name may be substituted and utilized in the descriptions herein.

### Toll-Like Receptor 4 (TLR4) Agonist

The Toll-like receptor (TLR4) agonist suitable for the disclosure is the compound of formula (I):

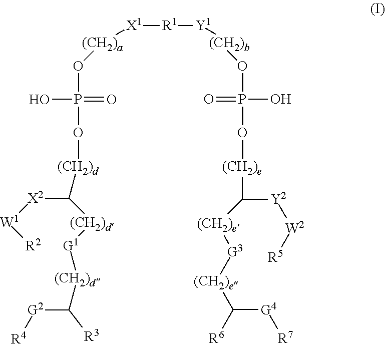

wherein R¹ is selected from the group consisting of:
   a) C(O);
   b) C(O)—(C₁-C₁₄ alkyl)-C(O), in which said C₁-C₁₄ alkyl is optionally substituted with a hydroxyl, a C₁-C₅ alkoxy, a C₁-C₅ alkylenedioxy, a (C₁-C₅ alkyl)amino or a (C₁-C₅ alkyl)aryl, in which said aryl moiety of said (C₁-C₅ alkyl)aryl is optionally substituted with a C₁-C₅ alkoxy, a (C₁-C₅ alkyl)amino, a (C₁-C₅ alkoxy)amino, a (C₁-C₅ alkyl)-amino(C₁-C₅ alkoxy), —O—(C₁-C₅ alkyl)amino (C₁-C₅ alkoxy), —O—(C₁-C₅ alkyl)amino-C(O)—(C₁-C₅ alkyl)-C(O)OH, or —O—(C₁-C₅ alkyl)amino-C(O)—(C₁-C₅ alkyl)-C(O)—(C₁-C₅)alkyl;
   c) an alkyl comprising a C₂-C₁₅ linear or branched chain, optionally substituted with a hydroxyl or an alkoxy; and
   d) —C(O)—(C₆-C₁₂ arylene)-C(O)— in which said arylene is optionally substituted with a hydroxyl, a halogen, a nitro or an amino;
a and b are independently 0, 1, 2, 3 or 4;
d, d′, d″, e, e′ and e″ are independently 0, 1, 2, 3 or 4;
X₁, X₂, Y₁ and Y₂ are independently selected from the group consisting of null, an oxygen, —NH— and —N(C(O)(C₁-C₄ alkyl))-, and —N(C₁-C₄ alkyl)-;
W₁ and W₂ are independently selected from the group consisting of a carbonyl, a methylene, a sulfone and a sulfoxide;
R² and R⁵ are independently selected from the group consisting of:
   a) a C₂ to C₂₀ straight chain or branched chain alkyl, which is optionally substituted with an oxo, a hydroxyl or an alkoxy;
   b) a C₂ to C₂₀ straight chain or branched chain alkenyl or dialkenyl, which is optionally substituted with an oxo, a hydroxyl or an alkoxy;
   c) a C₂ to C₂₀ straight chain or branched chain alkoxy, which is optionally substituted with an oxo, a hydroxyl or an alkoxy;
   d) NH—(C₂ to C₂₀ straight chain or branched chain alkyl), in which said alkyl group is optionally substituted with an oxo, a hydroxy or an alkoxy; and
   e)

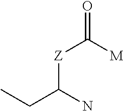

in which Z is selected from the group consisting of an O and NH, and M and N are independently selected from the group consisting of an alkyl, an alkenyl, an alkoxy, an acyloxy, an alkylamino and an acylamino comprising a C₂-C₂₀ linear or branched chain;
R³ and R⁶ are independently selected from the group consisting of a C₂ to C₂₀ straight chain or branched chain alkyl or alkenyl, optionally substituted with an oxo or a fluoro;
R⁴ and R⁷ are independently selected from the group consisting of a C(O)—(C₂ to C₂₀ straight chain or branched chain alkyl or alkenyl), a C₂ to C₂₀ straight chain or branched chain alkyl, a C₂ to C₂₀ straight chain or branched chain alkoxy, and a C₂ to C₂₀ straight chain or branched chain alkenyl; in which said alkyl, alkenyl or alkoxy groups can be independently and optionally substituted with a hydroxyl, a fluoro or a C₁-C₅ alkoxy;
G¹, G², G³ and G⁴ are independently selected from the group consisting of an oxygen, a methylene, an amino, a thiol, —C(O)NH—, —NHC(O)—, and —N(C(O)(C₁-C₄ alkyl))-;
   or G²R⁴ or G⁴R⁷ can together be a hydrogen atom or a hydroxyl;
   or a pharmaceutically acceptable salt of this compound.

A pharmaceutically acceptable salt of compounds of formula (I) may be a salt of organic or inorganic base of those compounds. For example, an organic or inorganic base may be from the group consisting of: hydroxides of alkali metals such as sodium, potassium and lithium; hydroxides of alkaline-earth metals such as calcium and magnesium; hydroxides of other metals, such as aluminum and zinc; ammonia and organic amines such as unsubstituted or hydroxy-substituted mono-, di- or trialkylamines; dicyclohexylamines; tributylamines; pyridine; N-methyl-N-ethylamine; diethylamine; triethylamine; mono-, bis- or tris(2-hydroxyalkylamines) such as mono-, bis- or tris(2-hydroxyethyl)amine, 2-hydroxy-tert-butylamine, or tris-(hydroxymethyl)methylamine, N,N-dialkyl-N-(hydroxyalkyl)amines, such as N,N-dimethyl-N-(2-hydroxyethyl)amine or tris(2-hydroxyethyl)amine; N-methyl-D-glucamine; and amino acids such as arginine and lysine.

In an embodiment, a TLR4 agonist suitable for the invention may be a compound of formula (I) as described above, wherein
R¹ is —C(O)— or —C(O)—(CH₂)ₙ—C(O)—, n being 1, 2, 3 or 4,
a, b, d, d′, d″, e, e′ and e″ are independently 1 or 2,
X¹, X², Y¹ and Y² are NH,
W¹ and W² are —C(O)—,
R² and R⁵ are independently selected from the group consisting of a C₁₀-C₁₅ straight chain alkyl optionally substituted with an oxo, an NH—(C₁₀-C₁₅ straight chain alkyl), and

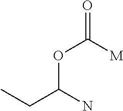

in which M and N are independently a C₂ to C₂₀ straight chain alkyl or alkenyl,
R³ and R⁶ are C₅-C₁₀ straight chain alkyls,
R⁴ and R⁷ are selected from the group consisting of a hydrogen, C(O)—(C₈-C₁₂ straight chain alkyl) or C(O) (C₈-C₁₂ straight chain alkenyl),
G¹ and G³ are an oxygen or —NH(CO)—,
G² and G⁴ are an oxygen.

In the context of the present disclosure, the terms below have the following definitions unless otherwise mentioned throughout the instant specification:
a halogen atom: a fluorine, a chlorine, a bromine or an iodine atom;
an oxo: a “═O” group;
a hydroxyl or hydroxy group: a OH group;
an alkyl group: a linear or branched saturated hydrocarbon-based aliphatic group comprising, unless otherwise mentioned, from 1 to 6 carbon atoms (noted “(C₁-C₆)-alkyl”). By way of examples, mention may be made of, but not limited to: methyl, ethyl, propyl, n-propyl, isopropyl, butyl, isobutyl, sec-butyl, tert-butyl, pentyl, isopentyl, hexyl and isohexyl groups, and the like;
an alkoxy group: an —O-alkyl group where the alkyl group is as previously defined. By way of example, mention may be made of, but not limited to: methoxy, ethoxy, propoxy, isopropoxy, linear, secondary or tertiary butoxy, isobutoxy, pentoxy or hexoxy groups, and the like;
an alkylene group: a bivalent saturated hydrocarbon radical which is either branched or linear. Unless otherwise indicated, the alkylene group comprises from 1 to 6 carbon atoms (noted “(C₁-C₆)-alkylene”).
an alkylendioxy group: a —ORO— group where R is an alkylene group, as defined herein.
an acyl group: a carbonyl group bonded to a carbon group.
an aryl group: a functional group or substituent derived from an aromatic ring, usually an aromatic hydrocarbon, such as phenyl and naphthyl;
an alkenyl group: a fragment, containing an open point of attachment on a carbon atom, that would form if a hydrogen atom bonded to a doubly bonded carbon is removed from the molecule of an alkene. Unless otherwise indicated, the alkenyl group comprises from 1 to 6 carbon atoms (noted “(C₁-C₆)-alkenyl”).
an acyloxy group: a R—COO—, derived from a carboxylic acid. Unless otherwise indicated, the acyloxy group comprises from 1 to 6 carbon atoms (noted “(C₁-C₆)-acyloxy”).
an alkylamino group: contains both an alkyl and an amino group, as defined herein;
an acylamino group: contains both an acyl and an amino group, as defined herein;
an amino group: a NH₂ group;
a carbonyl group: a (C═O) group;
a fluoro group: a —F;
a thiol: any organosulfur compound of the form R—SH, where R represents an alkyl, as defined herein;
a nitro group: a —NO₂;
a sulfone: contains a sulfonyl functional group attached to two carbon atoms. The central hexavalent sulfur atom is double-bonded to each of two oxygen atoms and has a single bond to each of two carbon atoms, usually in two separate hydrocarbon substituents;
a sulfoxide: a sulfinyl (SO) functional group attached to two carbon atoms, usually in two separate hydrocarbon substituents.

In an embodiment, a suitable TLR4 agonist may be the compound of formula (II):

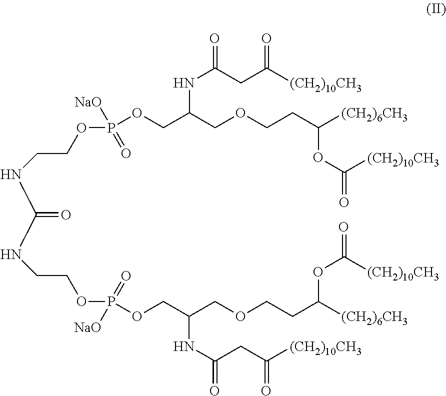

In an embodiment, a suitable TLR4 agonist may be E6020 of following formula (III):

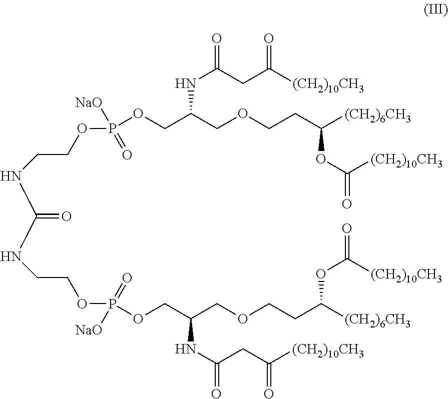

Compounds of formula (II) and (Ill) are potent TLR-4 receptor agonists (Ishizaka et al., Expert review of vaccines, 2007, 6: 773-84), and thus may be useful in liposomes of the disclosure to give an immunological adjuvant when the liposomes are co-administered with antigens such as vaccines for bacterial, viral, fungal, or parasitic diseases or with tumour antigens such as cancer vaccines.

Suitable TLR4-agonists may be obtained as described in WO 2007/005583 A1.

The IUPAC name of E6020 is Disodium (1R,6R,22R,27R)-1,27-diheptyl-9,19-dioxido-9,14,19,29-tetraoxo-6,22-bis[(3-oxotetradecanoyl)amino]-4,8,10,18,20,24,28-heptaoxa-13,15-diaza-9,19-diphosphatetracont-1-yl dodecanoate. Its CAS number is 287180-63-6.

Suitable TLR4 agonists according to the present disclosure present a solubility parameter in ethanol of at least about 0.2 mg/mL, measured at 25° C.

A suitable TLR4 agonist may have a solubility parameter in ethanol, measured at 25° C., of at least about 0.5 mg/mL, of at least about 1 mg/mL, of at least 2 mg/mL, of at least 4 mg/mL, of at least 6 mg/mL, of at least 10 mg/mL, of at least 12 mg/mL, of at least 15 mg/mL, of at least 20 mg/mL, of at least 25 mg/mL, or of at least 30 mg/mL.

A suitable TLR4 agonist may have a solubility parameter in ethanol, measured at 25° C., of from about 0.1 to about 50 mg/mL, of about 0.2 to about 45 mg/mL, of about 1 to about 40 mg/mL, of about 2 to about 35 mg/mL, of about 6 to about 30 mg/mL, or of about 10 to about 25 mg/mL.

A suitable TLR4 agonist may have a solubility parameter in ethanol, measured at 25° C., ranging from about at least about 0.2 mg/mL to about 20 mg/ml from about at least about 0.5 mg/mL to about 15 mg/ml, from about at least about 1 mg/mL to about 12 mg/ml, from about at least about 2 mg/mL to about 10 mg/ml, from about at least about 4 mg/mL to about 10 mg/ml.

In an exemplary embodiment, the TLR4 agonist has a solubility parameter in ethanol of at least about 10 mg/mL. The solubility parameters provided herein are measured at about 25° C. and at an atmospheric pressure of about 1013 hPa.

Solubility indicates the maximum amount of a substance, here the TLR4 agonist, that can be dissolved in a solvent, here ethanol, at a given temperature and pressure. The extent of the solubility of a substance in a specific solvent is measured as the saturation concentration, where adding more solute does not increase the concentration of the solution and begins to precipitate the excess amount of solute.

The solubility of a TLR4 agonist in ethanol may be determined by any methods known in the art. The solubility may be measured experimentally. For example, a method suitable for determining the solubility parameter of a given TLR4 agonist in ethanol, such as a TLR4 agonist suitable according to the present disclosure, is by performing nephelometry, as provided further below in the examples. Other methods for determining the solubility parameter of a given TLR4 agonist in ethanol may include the methods described in Veseli et al. (Drug Dev Ind Pharm. 2019 November; 45(11):1717-1724).

Ethanol, as opposed to other available organic solvents or mixtures of organic solvents, such as isopropanol or ethanol/isopropanol, is considered as a safe compound and its use in the manufacturing process of pharmaceutical products is usually not challenged by Health Agencies.

Because of the specific displayed range of solubility in ethanol of the selected TLR4 agonists, those compounds may be advantageously implemented in a liposome manufacturing method based on the solvent injection method. Such method presents the advantage of being able to be scaled up at industrial scale. Therefore, the liposome-based adjuvant as disclosed may be easily and cost-effectively produced at industrial scale.

Suitable TLR4-agonists may be used in combination with proteins or peptides antigens, with polysaccharide antigens and/or with glycoconjugate antigens to give immunogenic compositions, such as vaccine compositions.

The TLR4-agonists as disclosed herein are used in the liposomes, such as single type of liposomes or second type of liposomes of a combination as disclosed herein, in an amount effective to confer to the liposomes, or to the combination of liposomes, in association with the other components of the liposomes, or with the components of the other type of liposomes of the combination of liposomes, such as the saponin and the phospholipid, an immunopotentiating effect when administered to an individual. The TLR-4 agonists are used in the liposomes, as single type of liposomes or second type of liposomes of a combination as disclosed herein, in an amount effective to confer to the liposomes, or the combination of liposomes, in association with the other components of the liposomes, or with the components of the other type of liposomes of the combination of liposomes, such as the saponin and the phospholipid, an adjuvant effect to an antigen.

An amount of TLR4 agonist may range from about 0.5 μg/ml to about 200 μg/ml, from about 1 μg/ml to about 150 μg/ml, from about 1.5 μg/ml to about 100 μg/ml, from about 2.0 μg/ml to about 50 μg/ml, such as from about 2.5 μg/ml to about 20 μg/ml, such as from about 4 μg/ml to about 10 μg/ml of TLR4 agonist in weight/volume in the vaccine composition in which the liposomes may be comprised.

In an embodiment, a TLR4 agonist may be present in the liposomes, as single type of liposomes or second type of liposomes of a combination as disclosed herein, with the saponin in a weight:weight ratio of TLR4 agonist:saponin ranging from about 1:1 to about 1:500, from about 1:1 to about 1:400, ranging from about 1:2 to about 1:200, ranging from about 1:2.5 to about 1:100, ranging from about 1:3 to about 1:40, or ranging from about 1:5 to about 1:25.

In a combination of liposomes as disclosed herein, the content of the different components, i.e., TLR4-agonist, saponin, sterol or sterol ester, and phospholipid may be expressed per type of liposomes or per the combination of liposomes, or per the composition comprising the liposomes. In some embodiments, the content of the different components, i.e., TLR4-agonist, saponin, sterol or sterol ester, and phospholipid is expressed per the combination of liposomes or per the composition comprising the liposomes. For example, in a combination of liposomes as disclosed herein, when the amounts of TLR4 agonist and saponin are expressed in a weight:weight ratio, that refers to the amount of TLR-4 agonist in a first type of liposomes and to the amount of saponin in the second type of liposomes. As other example, in a combination of liposomes as disclosed herein, when the amount of TLR-4 agonist is expressed in weight/volume, that refers to the total amount of TLR-4 agonist in the combination of liposomes per volume unit of composition containing this combination. As for example also, in a combination of liposomes as disclosed herein, when the amounts of TLR4 agonist and e.g., phospholipids are expressed in a weight:weight ratio, that refers to the amount of TLR-4 agonist in a first type of liposomes and to the total amount of phospholipds in the first and in the second type of liposomes.

In an embodiment, a TLR4 agonist may be present in the liposomes, as single type of liposomes or in a second type of liposomes of a combination as disclosed herein, with the saponin in a weight:weight ratio of TLR4 agonist:saponin ranging from about 1:1 to about 1:50, or from about 1:25 to about 1:35, or in a weight ratio of TLR4 agonist:saponin of about 1:10.

A TLR4 agonist may be present in the liposomes, as single type of liposomes or in a second type of liposomes of a combination as disclosed herein, with the saponin in a weight:weight ratio of TLR4 agonist:saponin of about 1:10.

As shown in the Examples, compared to other TLR4 agonists, for example the MPLA, the TLR4 agonists as disclosed herein display an enhanced efficacy to elicit an immune response and therefore may be used in a lower amount compared to other TLR4 agonists. Hence, starting with a same absolute amount of material, with the TLR4 agonists as disclosed herein it may be possible to manufacture more adjuvant compositions at less expense than with MPLA.

Also, compared to other TLR4 agonists, for instance the MPL as shown in the Examples, the TLR4 agonists as disclosed herein and formulated in liposomes display a better tolerability and a lower reactogenicity than other TLR4 agonists or than the same TLR4 agonists but not formulated in liposomes.

### Saponin

A liposome of the disclosure, such as a single type of liposome or a first type of liposomes of a combination as disclosed herein, may include at least one saponin. The presence of saponin, such as in combination with the TLR4 agonist, imparts an immunopotentiating effect to the liposomes.

Saponins may be useful in liposomes, such as a single type of liposome or a first type of liposomes of a combination as disclosed herein, in combination with the TLR4 agonists, to impart an immunological adjuvant effect when the liposomes are co-administered with antigens, such as vaccines for bacterial, viral, fungal or parasitic diseases or with tumour antigens such as cancer vaccines.

“Saponin” refers to a group of surface-active amphiphile glycosides found in abundance in various plant species which are composed of a hydrophilic region (usually several sugar chains) combined with a hydrophobic region of either steroid or triterpenoid structure.

It is known in the art (Fleck et al., Molecules. 2019; 24(1):171; Wang et al., ACS Infect Dis. 2019; 5(6):974-981) that, when non-formulated in presence of cholesterol, saponins, such as Quillaja saponins, may induce undesirable haemolytic effect and may be unstable in aqueous phase. Further, it is admitted that a correlation between adjuvant activity and haemolytic effect of saponin may exist. Formulation of saponins in presence of cholesterol advantageously reduces the haemolytic effect while at the same time maintains the adjuvanting effect. Haemolytic effects may be involved in some adverse reactions after administration.

Saponins as referred to in the disclosure my be prepared by chemical synthesis as described for instance in Wang P. et al., J Org Chem, 2013 Nov. 15; 78(22): 11525-11534, in Kim Y J et al., J Am Chem Soc, 2006; 128:11906-11915 or in Deng K et al., Angew Chem Int Ed Engl. 2008; 47(34): 6395-6398.

Saponins useful for the disclosure may be Quillaja saponaria saponins. A “Quillaja saponaria saponin” as used herein intends to refer to a saponin that is structurally and functionally identical to a saponin that can be found in the Quillaja saponaria Molina tree, for example in the bark of the Quillaja saponaria Molina tree, but which may be obtained either from another vegetal source or by synthetisis means. Synthetisis means can be chemical synthesis means or in vitro biological production means such as production in isolated recombinant cells grown in fermentor, or even in vitro reconstituted artificial cells. Culture cells may be isolated cells grown in vitro, such as vegetal cells, either from Quillaja saponaria Molina tree or from another vegetal but modified (recombinant isolated cells) to produce saponins that can be found in Quillaja saponaria Molina tree.

In an embodiment, a Quillaja saponaria saponin may be obtained by extraction from Quillaja Saponaria Molina.

Immunologically active saponin fractions having adjuvant activity derived from the bark of the South American tree Quillaja Saponaria Molina are known in the art. For example, QS21, also known as QA21, an HPLC purified fraction from the Quillaja Saponaria Molina tree and its method of production are disclosed (as QA21) in U.S. Pat. No. 5,057,540. Quillaja saponin has also been disclosed as an adjuvant by Scott et al, 1985, Int Archs. Allergy Appl. Immun., 77, 409.

Any method known to one skilled in the art for extracting components from plants may be used to extract saponins from Quillaja saponaria Molina. Methods for manufacturing saponin extracts from Quillaja Saponaria Molina are described for example in WO 2019/106192 A1. Saponins may be obtained by further fractionation of Quil A, the saponin fraction from the bark of Quillaja saponaria Molina.

Saponins may be used as mixtures or as purified individual components. Suitable saponins include QS-7, QS-17, QS-18, and QS-21, all fractioned from QuilA.

In some embodiments, a liposome may a comprise a saponin selected among QS-7, QS-17, QS-18, QS-21, and combinations thereof.

In one embodiment, a liposome may comprise as saponin a QS-21, also known as QS21 or QA21.

In one embodiment, a liposome may comprise as saponin a QS-7. QS7 has an haemolytic effect far below the haemolytic effect of QS21. As shown in the Examples, when formulated in liposomes of the disclosure, QS7 is able to induce an adjuvanting effect as good as the adjuvanting effect of QS21. This may be advantageously implemented for increasing the amount of QS7, for example comparatively to QS21, to further enhance the adjuvanting effect without increasing possible risks of adverse reactions after administration to an individual.

Other suitable saponins are Momordica cochichinensis Spreng saponins. A “Momordica cochichinensis Spreng saponin” as used herein intends to refer to a saponin that is structurally and functionally identical to a saponin that can be found in the Momordica cochichinensis Spreng fruit, but which is obtained either from another vegetal source or by synthetisis means as above disclosed.

Such saponins are described by P. Wang et al. (J. Med. Chem. 2020, 63, 3290-3297).

An amount of saponin may range from 1 μg/ml to 1 000 μg/ml, such as from 25 μg/ml to 750 μg/ml, such as from 50 μg/ml to 500 μg/ml of saponin in weight/volume in the vaccine composition in which the liposomes (as single type or as a combination of different types of liposomes) may be comprised. The saponin may be present in the vaccine composition in an amount of about 100 μg/ml.

In an embodiment, saponins may be present in the liposomes with the TLR4 agonist, or in a combination of liposomes as described herein, in a weight:weight ratio of saponin:TLR4-agonist ranging from about 1:1 to about 400:1, ranging from about 2:1 to about 200:1, ranging from about 2.5:1 to about 100:1, ranging from about 3:1 to about 40:1, or ranging from about 5:1 to about 25:1.

Saponins may be present in the liposomes, such as single type of liposomes or liposomes of a combination as disclosed herein, with the TLR4 agonist in a weight:weight ratio of saponin:TLR4 agonist of about 10:1.

Saponins, such as QS21 or QS7, may be present in a liposome, as a single type of liposomes or in a combination of liposomes as disclosed herein, with the TLR4 agonist, such as E6020, in an amount, expressed in μg/mL, TLR4 agonist:saponin of about 20:200, or about 20:600, or about 20:1800.

A saponin QS21 may be present in a liposome, as a single type of liposomes or in a combination as disclosed herein, with E6020 in an amount, expressed in μg/mL, E6020:QS21 of about 20:200, or about 20:600, or about 20:1800, and for example of about 20:200.

A saponin QS7 may be present in a liposome, as a single type of liposomes or in a combination as disclosed herein, with E6020 in an amount, expressed in μg/mL, E6020:QS7 of about 20:200, or about 20:600, or about 20:1800, and for example of about 20:600.

Saponins may be present in a liposome, as a single type of liposomes or in a combination as disclosed herein, in a weight:weight ratio of saponin:sterol ranging from 1:100 to 1:1, ranging from 1:50 to 1:2, or ranging from 1:10 to 1:5, or in a weight:weight ratio of saponin:sterol of about 1:2, or in a weight:weight ratio of saponin:sterol of about 1:5.

### Sterols

A liposome of the disclosure, as a single type of liposome and/or as a first or second types of liposomes of a combination as disclosed herein, may include a sterol or an ester thereof. The presence of sterol or ester of sterol may improve structural stability of the liposomes.

Sterols useful herein may be selected from the group consisting of cholesterol or its derivatives, ergosterol, desmosterol (3B-hydroxy-5,24-cholestadiene), stigmasterol (stigmasta-5,22-dien-3-ol), lanosterol (8,24-lanostadien-3b-ol), 7-dehydrocholesterol (Δ5,7-cholesterol), dihydrolanosterol (24,25-dihydrolanosterol), zymosterol (5α-cholesta-8,24-dien-3β-ol), lathosterol (5α-cholest-7-en-3β-ol), diosgenin ((3p,25R)-spirost-5-en-3-ol), sitosterol (22,23-dihydrostigmasterol), sitostanol, campesterol (campest-5-en-3β-ol), campestanol (5a-campestan-3b-ol), 24-methylene cholesterol (5,24(28)-cholestadien-24-methylen-3β-ol), and mixture thereof.

Esters of sterol refer to esters of carboxylic acid with the hydroxyl group of the steroid alcohol. Suitable carboxylic acid comprises, further to the carboxyl moiety, a saturated or unsaturated, linear or branched, alkyl group. In some embodiments the alkyl group may be a C₁-C₂₀ saturated or unsaturated, linear or branched, alkyl group, such as a C₂-C₁₈, such as a C₄-C₁₆, such as C₈-C₁₂ saturated or unsaturated, linear or branched, alkyl group, In other embodiments, the carboxylic acid may be a fatty acid. For example, a fatty acid may be caprylic acid, capric acid, lauric acid, stearic acid, margaric acid, oleic acid, linoleic acid, or arachidic acid.

In one embodiment, an ester of sterol may be a cholesteryl ester.

Esters of sterol useful herein may be selected from the group consisting of cholesteryl margarate (cholest-5-en-3β-yl heptadecanoate), cholesteryl oleate, and cholesteryl stearate, and mixture thereof.

Sterols or esters thereof may selected from the group consisting of cholesterol or its derivatives, ergosterol, desmosterol (3β-hydroxy-5,24-cholestadiene), stigmasterol (stigmasta-5,22-dien-3-ol), lanosterol (8,24-lanostadien-3b-ol), 7-dehydrocholesterol (Δ5,7-cholesterol), dihydrolanosterol (24,25-dihydrolanosterol), zymosterol (5α-cholesta-8,24-dien-3β-ol), lathosterol (5α-cholest-7-en-3β-ol), diosgenin ((3p,25R)-spirost-5-en-3-ol), sitosterol (22,23-dihydrostigmasterol), sitostanol, campesterol (campest-5-en-3β-ol), campestanol (5a-campestan-3b-ol), 24-methylene cholesterol (5,24(28)-cholestadien-24-methylen-3β-ol), cholesteryl margarate (cholest-5-en-3β-yl heptadecanoate), cholesteryl oleate, and cholesteryl stearate, and mixture thereof.

Alternatively, a useful sterol may be a cholesterol derivative such as an oxidized cholesterol.

Suitable oxidized cholesterols may be 25-hydroxycholesterol, 27-hydroxycholesterol, 20α-hydroxycholesterol, 6-keto-5α-hydroxycholesterol, 7-keto-cholesterol, 7β,25-hydroxycholesterol and 7β-hydroxycholesterol. Oxidized cholesterols may be 25-hydroxycholesterol and 20a-hydroxycholesterol, and mixture thereof, and for example it may be 20α-hydroxycholesterol.

In one embodiment, a sterol or ester thereof, may be cholesterol, a cholesteryl ester, or a cholesterol derivative, such as an oxidized cholesterol. In one embodiment, a sterol or steroid alcohol may be cholesterol or a cholesteryl ester. In a further embodiment, a sterol or steroid alcohol is cholesterol.

In a combination of liposomes as disclosed herein, the content of sterol in the different types of liposomes, e.g., the first and second types of liposomes, may be identical or different. In some embodiments, the content of sterol in the different types of liposomes, e.g., the first and second types of liposomes, is identical.

Sterols or esters thereof may be present in a molar amount ranging from about 0.1 mM to about 10 mM, in a molar amount ranging from about 0.2 mM to about 7 mM, in a molar amount ranging from about 0.5 mM to about 5 mM, or in a molar amount ranging from about 0.8 mM to about 4 mM, or in a molar amount ranging from about 1 mM to about 3 mM, or in a molar amount ranging from about 1.2 mM to about 2 mM in the vaccine composition in which the liposomes may be comprised. In one exemplary embodiment, sterols or esters thereof may be present in a molar amount of about 1.3 mM in the vaccine composition in which the liposomes may be comprised.

Sterols or esters thereof may be present in a liposome of the disclosure, as a single type of liposome, or as a first and/or a second types of liposomes of a combination as disclosed herein, in a weight:weight ratio of saponin:sterol ranging from 1:100 to 1:1, ranging from 1:50 to 1:2, or ranging from 1:10 to 1:5, or in a weight:weight ratio of saponin:sterol of about 1:2, or in a weight:weight ratio of saponin:sterol of about 1:5.

### Phospholipids

A liposome of the disclosure, as a single type of liposome, and/or as a first and/or second types of liposomes of a combination as disclosed herein, may include at least one phospholipid. The presence of phospholipids may improve structural stability of the liposomes.

Suitable phospholipids may be selected from the group consisting of phosphatidylcholines, phosphatidic acids, phosphatidylethanolamines, phosphatidylglycerols, phosphatidylserines, phosphatidylinositols, and mixtures thereof.

As examples of useful phosphatidylcholines, one may mention DSPC (1,2-distearoyl-sn-glycero-3-phosphocholine), DPPC (1,2-dipalmitoyl-sn-glycero-3-phosphocholine), DMPC (1,2-dimyristoyl-sn-glycero-3-phosphocholine), POPC (1-palmitoyl-2-oleoyl-sn-glycero-3-phosphocholine), DOPC (1,2-dioleoyl-sn-glycero-3-phosphocholine), SOPC (1-stearoyl-2-oleoyl-sn-glycero-3-phosphocholine), and mixtures thereof.

As examples of useful phosphatidylethanolamines, one may mention DSPE (1,2-distearoyl-sn-glycero-3-phosphoethanolamine), DPPE (1,2-dipalmitoyl-sn-glycero-3-phosphoethanolamine), DMPE (1,2-dimyristoyl-sn-glycero-3-phosphoethanolamine), POPE (1-palmitoyl-2-oleoyl-sn-glycero-3-phosphoethanolamine) DOPE (1,2-dioleyl-sn-glycero-3-phosphoethanolamine), SOPE (1-stearoyl-2-oleoyl-sn-glycero-phosphatidyethanolamine), and mixtures thereof.

As examples of useful phosphatidic acids, one may mention DSPA (1,2-distearoyl-sn-glycero-3-phosphatidic acid), DPPA (1,2-dipalmitoyl-sn-glycero-3-phosphatidic acid), DMPA (1,2-dimyristoyl-sn-glycero-3-phosphatidic acid), POPA (1-palmitoyl-2-oleoyl-sn-glycero-3-phosphatidic acid), DOPA (1,2-dioleoyl-sn-glycero-3-phosphatidic acid), SOPA (1-stearoyl-2-oleoyl-sn-glycero-phosphatidic acid), and mixtures thereof. Pharmaceutically acceptable salts of these phosphatidic acids may also be useful.

As examples of useful phosphatidylglycerols one may mention DSPG (1,2-distearoyl-sn-glycero-3-phosphatidylglycerol), DPPG (1,2-dipalmitoyl-sn-glycero-3-phosphatidylglycerol), DMPG (1,2-dimyristoyl-sn-glycero-3-phosphatidylglycerol), POPG (1-palmitoyl-2-oleoyl-sn-glycero-3-phosphatidylglycerol), DOPG (1,2-dioleoyl-sn-glycero-3-phosphatidylglycerol), SOPG (1-stearoyl-2-oleoyl-sn-glycero-phosphatidylglycerol), and mixtures thereof.

As examples of useful phosphatidylserines, one may mention DSPS (1,2-distearoyl-sn-glycero-3-phosphatidylserine), DPPS (1,2-dipalmitoyl-sn-glycero-3-phosphatidylserine), DMPS (1,2-dimyristoyl-sn-glycero-3-phosphatidylserine), POPS (1-palmitoyl-2-oleoyl-sn-glycero-3-phosphatidylserine), DOPS (1,2-dioleoyl-sn-glycero-3-phosphatidylserine), SOPS (1-stearoyl-2-oleoyl-sn-glycero-phosphatidylserine), and mixtures thereof.

As examples of useful phosphatidylinositols, one may mention DSPI (1,2-distearoyl-sn-glycero-3-phosphatidylinositol), DPPI (1,2-dipalmitoyl-sn-glycero-3-phosphatidylinositol), DMPI (1,2-dimyristoyl-sn-glycero-3-phosphatidylinositol), POPI (1-palmitoyl-2-oleoyl-sn-glycero-3-phosphatidylinositol), DOPI (1,2-dioleoyl-sn-glycero-3-phosphatidylinositol), SOPI (1-stearoyl-2-oleoyl-sn-glycero-phosphatidylinositol), and mixtures thereof.

A phospholipid may be selected from the group consisting of phosphatidylcholines, such as DSPC, DPPC, DMPC, POPC, DOPC; SOPC and phosphatidylethanolamines, such as DSPE, DPPE, DMPE, POPE, DOPE, SOPE; and mixtures thereof.

In one embodiment, a suitable phospholipid may be DSPC, DOPC, and DOPE, and may be DSPC or DOPE, and mixtures thereof.

In a combination of liposomes as disclosed herein, the content of phospholipids in the different types of liposomes, e.g., first and second types of liposomes, may be identical or different. In some embodiments, the content of phospholipids in the different types of liposomes, e.g., the first and second types of liposomes, is identical.

Phospholipids may be present in a molar amount ranging from about 0.1 mM to about 20 mM, in a molar amount ranging from about 0.2 mM to about 15 mM, in a molar amount ranging from about 0.5 mM to about 10 mM, in a molar amount ranging from about 0.8 mM to about 7 mM, in a molar amount ranging from about 1 mM to about 5 mM, or in a molar amount ranging from about 1.2 mM to about 2.5 mM in the vaccine composition in which the liposomes, as a single type of liposome, or as a first and/or second types of liposomes of a combination as disclosed herein, may be comprised. In one exemplary embodiment, phospholipids may be present in a molar amount of about 1.25 mM in the vaccine composition in which the liposomes may be comprised.

Phospholipids may be present in liposomes, as a single type of liposome, or as a first and/or second types of liposomes of a combination as disclosed herein, in a weight:weight ratio of saponin:phospholipid ranging from 1:400 to 1:4, ranging from 1:200 to 1:8, ranging from 1:100 to 1:10, ranging from 1:50 to 1:10, of about 1:8, or of about 1:20.

Phospholipids may be present in a liposome of the disclosure, as a single type of liposome, or as a first and/or second types of liposomes of a combination as disclosed herein, in aweight:weight ratio of sterol:phospholipid ranging from 100:1 to 1:200, ranging from 50:1 to 1:100, ranging from 10:1 to 20:1, of about 1:1, of about 1:2, or of about 1:4.

### Antigens

According to one embodiment, liposomes of the disclosure may be used to adjuvant wild type or recombinant antigens, or fragments or subunits thereof. Said antigens may be proteins, peptides, polysaccharides and/or glycocongugates.

In embodiments where a combination of at least two liposomes is implemented, an antigen may be present in the first and/or the second types of liposomes of a combination as disclosed herein.

Liposome/antigen-containing compositions of the disclosure may vary in their valency. Valency refers to the number of antigenic components, i.e., the number of different antigens, in the composition. In some embodiments, the compositions are monovalent. They may also be compositions comprising more than one valence such as divalent, trivalent or multivalent composition. Multivalent compositions may comprise 2, 3, 4, 5, 6, 7, 8, 9, 10, 11, 12, 13, 14, 15, 16, 17, 18, 19, 20, or more antigens or antigenic moieties (e.g., antigenic peptides, etc.).

Liposome/antigen-containing compositions of the disclosure may be used as immunogenic compositions, such as vaccine compositions, to protect, treat or cure infection arising from contact with an infectious agent, such as bacteria, viruses, fungi, protozoa and parasites. Liposome/antigen-containing compositions may be used to protect, treat or cure cancer diseases.

According to one embodiment, an antigen suitable herein may be selected in the group consisting of bacterial antigens, protozoan antigens, viral antigens, fungal antigens, parasite antigens or tumour antigens.

### Bacterial Antigens

The bacterial antigen may be from Gram-positive bacteria or Gram-negative bactera. Bacterial antigens may be obtained from Acinetobacter baumannii, Bacillus anthracis, Bacillus subtilis, Bordetella pertussis, Borrelia burgdorferi, Brucella abortus, Brucella canis, Brucella melitensis, Brucella suis, Campylobacter jejuni, Chlamydia pneumoniae, Chlamydia triachomatis, Chlamydophila psittaci, Clostridium botulinum, Clostridium difficile, Clostridium perfringens, Clostridium tetani, coagulase Negative Staphylococcus, Corynebacterium diphtheria, Enterococcus faecalis, Enterococcus faecium, Escherichia coli, enterotoxigenic Escherichia coli (ETEC), enteropathogenic E. coli, E. coli 0157:H7, Enterobacter sp., Francisella tularensis, Haemophilus influenzae, Helicobacter pylori, Klebsiella pneumoniae, Legionella pneumophila, Leptospira interrogans, Listeria monocytogenes, Moraxella catarralis, Mycobacterium leprae, Mycobacterium tuberculosis, Mycoplasma pneumoniae, Neisseria gonorrhoeae, Neisseria meningitides, Proteus mirabilis, Proteus sps., Pseudomonas aeruginosa, Rickettsia rickettsii, Salmonella typhi, Salmonella typhimurium, Serratia marcesens, Shigella flexneri, Shigella sonnei, Staphylococcus aureus, Staphylococcus epidermidis, Staphylococcus saprophyticus, Streptococcus agalactiae, Streptococcus mutans, Streptococcus pneumoniae, Streptococcus pyogenes, Treponema pallidum, Vibrio cholerae, or Yersinia pestis.

### Viral Antigens

Viral antigens may be obtained from adenovirus; Herpes simplex, type 1; Herpes simplex, type 2; encephalitis virus, papillomavirus, Varicella-zoster virus; Epstein-barr virus; Human cytomegalovirus (CMV); Human herpesvirus, type 8; Human papillomavirus; BK virus; JC virus; Smallpox; polio virus, Hepatitis B virus; Human bocavirus; Parvovirus B19; Human astrovirus; Norwalk virus; coxsackievirus; hepatitis A virus; poliovirus; rhinovirus; Severe acute respiratory syndrome virus; Hepatitis C virus; yellow fever virus; dengue virus; West Nile virus; Rubella virus; Hepatitis E virus; Human immunodeficiency virus (HIV); Influenza virus, type A or B; Guanarito virus; Junin virus; Lassa virus; Machupo virus; Sabia virus; Crimean-Congo hemorrhagic fever virus; Ebola virus; Marburg virus; Measles virus; Mumps virus; Parainfluenza virus; Respiratory syncytial virus (RSV); Human metapneumovirus; Hendra virus; Nipah virus; Rabies virus; Hepatitis D; Rotavirus; Orbivirus; Coltivirus; Hantavirus, Middle East Respiratory Coronavirus; SARS-Cov-2 virus; Chikungunya virus; Zika virus; parainfluenza virus; Human Enterovirus; Hanta virus; Japanese encephalitis virus; Vesicular exanthernavirus; Eastern equine encephalitisor; or Banna virus.

In an embodiment, the antigen is from a strain of Influenza A or Influenza B virus or combinations thereof. The strain of Influenza A or Influenza B may be associated with birds, pigs, horses, dogs, humans or non-human primates.

The nucleic acid may encode a hemagglutinin protein or fragment thereof. The hemagglutinin protein may be H1, H2, H3, H4, H5, H6, H7, H8, H9, H10, HI 11, H12, H13, H14, H15, H16, H17, H18, or a fragment thereof. The hemagglutinin protein may or may not comprise a head domain (HA1). Alternatively, the hemagglutinin protein may or may not comprise a cytoplasmic domain.

In certain embodiments, the hemagglutinin protein is a truncated hemagglutinin protein. The truncated hemagglutinin protein may comprise a portion of the transmembrane domain.

In some embodiments, the virus may be selected from the group consisting of H1N1, H3N2, H7N9, H5N1 and H10N8 virus or a B strain virus.

In another embodiment, the antigen may be from CMV. The antigen may be from HCMV. The antigen may be a combination of a pentamer (gH/gL/pUL128/pUL130/pUL131) and a gB. In another embodiment, the antigen is not from CMV. The antigen is not from HCMV. The antigen is not a combination of a pentamer (gH/gL/pUL128/pUL130/pUL131) and a gB.

In another embodiment, the antigen is from a coronavirus such as SARS-Cov-1 virus, SARS-Cov-2 virus, or MERS-Cov virus.

In another embodiment, the antigen may be from RSV. The antigen may be PreF-ferritin. A prefusion RSV F antigen suitable may be as disclosed in WO 2014/160463 A1 or in WO 2019/195316 A1.

In an embodiment, an antigen suitable herein may be an antigen from human CMV such as a combination of a pentamer (gH/gL/pUL128/pUL130/pUL131) and gB, an antigen from human Influenza strains such as A/H1N1, A/H3N2, and Influenza B strains, an antigen from RSV such as the F antigen in its prefusion conformation (preF) fused or not to a ferritin moiety (preF-ferritin).

### CMV Antigens

The CMV antigen(s) which can be used in an immunogenic composition according to the disclosure may be a CMV gB antigen and a CMV gH/gL/UL128/UL130/UL131 pentameric complex antigen.

In one exemplary embodiment, the CMV antigens may be from Human Cytomegalovirus (HCMV), and therefore may be HCMV antigens.

### CMV gB Antigen

A CMV gB antigen according to the disclosure may be a full length gB polypeptide or a gB-derived polypeptide that induces neutralizing antibodies. A gB-derived polypeptide is a polypeptide obtained from the full-length gB in which some modifications such as amino acid additions, deletions, and/or substitutions were introduced, and which still induces neutralizing antibodies towards CMV. As exemplary of gB-derived polypeptides one may mention truncated gB antigen and/or mutated gB antigens containing some amino acids substitutions, for example in the furin site. A truncated gB, as disclosed herein, refers to a gB from which one or a plurality of region(s) or domain(s) has/have been deleted, in all or in part, such as the transmembrane region.

The gB polypeptide is encoded by the UL55 gene of CMV genome. The size of the native form of gB (or gp130) depends on the size of the open reading frame (ORF), which may vary according to the strain considered. For example, the ORF of AD 69 strain, which is 2717 bp long, encodes a full length gB of 906 amino acids, whereas the ORF of Towne strain encodes a native gB of 907 amino acids. The protein sequences of these two strains are described in US 2002/0102562, incorporated by reference in its entirety. The native form of gB contains an amino acid signal sequence that may be 22 to 25 amino acids long, followed by an extracellular domain, or ectodomain, spanning from amino acid 26 to 706 or 707, and which contains an endoproteolytic cleavage site (furin site, RTRR, residues 456-459 in strain AD169 or RTKR in strain Towne) leading to a cut between residues arginine 459 (or 460 in strain Towne—numbering may vary depending on the strain) and serine 460 (or 461 in strain Towne—numbering may vary depending on the strain), followed by a membrane proximal region (from amino acid 707 or 708 to 750) and a transmembrane domain (from amino acid 750 or 751 to 772) and then terminated by an intracellular domain spanning from amino acids 772 or 773 to 906 or 907 (Sharma et al., Virology. 2013; 435(2):239-249 and Burke et al., PLOS Pathogen. 2015; 11(10): e1005227). Once processed the full length gB is deleted from the amino acid signal sequence as a consequence of posttranslational mechanisms occurring in infected cells. Exemplary of full length gB antigen for the purpose of the disclosure encompasses both the full length gB of CMV strains Towne and AD169, as well as other equivalent strains. Several antigenic domains (AD) inducing neutralizing antibodies have been described in the gB polypeptide sequence. As exemplary antigenic domains, one may mention the domain extending from amino acid residues 461 to 680. This domain may be subdivided into two discontinuous domains, a first one extending from residues 461 to 619 and a second one extending from residues 620 to 680 (U.S. Pat. No. 5,547,834). As other antigenic domains identified one may cite the antigenic domain 1 (AD-1) located from amino acid residues 560 to 640 (Schoppel K. et al., Virology, 1996, 216:133-45) or the antigenic domain 2 (AD-2) located from amino acid residues 65 to 84 (Axelsson F et al., Vaccine, 2007, 26:41-6) or from amino acid residues 27 to 84 (Burke H G et al., PLoS pathogens, 2015, 11(10):e1005227). Consequently, a polypeptide comprising in its sequence a sequence homologous to one or several of the above cited antigenic domains may also be suitable for the purpose of the disclosure. The term “a sequence homologous to” is intended to mean an amino acid sequence in which there is at least 80% identity with the amino acid sequence of the antigenic domain being considered of the native gB originating from the Towne or AD169 strain (which are described in US 2002/0102562). Typically, the sequence homology is based on a sequence identity of at least 90% and, even more specifically, the sequence homology is complete (sequence identity of 100%).

As used herein, a first sequence having at least x % identity with a second sequence means that x % represents the number of amino acids in the first sequence which are identical to their matched amino acids of the second sequence when both sequences are optimally aligned via a global alignment, relative to the total length of the second amino acid sequence. Both sequences are optimally aligned when x is maximum. The alignment and the determination of the percentage of identity may be carried out manually or automatically using a global alignment algorithm, for instance the Needleman and Wunsch algorithm, described in Needleman and Wunsch, J. Mol Biol., 48, 443-453 (1970), with for example the following parameters for polypeptide sequence comparison: comparison matrix: BLOSUM62 from Henikoff and Henikoff, Proc. Natl. Acad. Sci. USA., 89, 10915-10919 (1992), gap penalty: 8 and gap length penalty: 2; and the following parameters for polynucleotide sequence comparison: comparison matrix: matches=+10, mismatch=0; gap penalty: 50 and gap length penalty: 3.

A program which may be used with the above parameters is publicly available as the “gap” program from Genetics Computer Group, Madison WI. The aforementioned parameters are the default parameters respectively for peptide comparisons (along with no penalty for end gaps) and for nucleic acid comparisons.

Among the gB-derived polypeptides useful for the purpose of the disclosure, one may mention gp 55 as described in U.S. Pat. No. 5,547,834. It is derived from the cleavage of gB at the endoproteolytic cleavage site; its amino acid sequence corresponds to the sequence extending from serine residue 461 to the C-terminal end. Truncated forms of gp 55 can also be used, such as a gp 55 deleted from all or part of the transmembrane sequence and from all or part of the intracellular C-terminal domain. Examples of such gB-truncated antigens may be a peptide having a sequence homologous to the amino acid sequence of the gB ranging from residues 461 to 646 or a gp 55 deleted of all or part of the intracellular C-terminal domain, such as a peptide having a sequence homologous to the amino acid sequence of the gB ranging from residues 461 to 680. Such truncated forms of gp 55 are also described in U.S. Pat. No. 5,547,834, incorporated by reference in its entirety.

It is also possible to use a mutated form of a full length gB that may carry one or several amino acid substitutions at the endoproteolytic cleavage site such that the latter is made ineffectual. As exemplary embodiment, amino acid substitutions may be located between residues 457 and 460 of a sequence of a gp130 and, such as for example at arginine 460 and/or lysine 459 and/or arginine 457. Such mutated form of a full length gB may carry the entire extracellular domain with all the domains that are targets for neutralizing antibodies. Such mutated forms can be secondarily truncated from all or part of the transmembrane sequence (extending from aa 752 to 773) and/or from all or part of the intracellular C-terminal domain (extending from aa 774 to 907) in order to allow their secretion in the host when produced as recombinant proteins and their easy downstream purification. Such gB-derivatives are useful in so far as substantially all the domains that are targets for neutralizing antibodies are conserved.

In one exemplary embodiment, a CMV gB antigen may be selected in a group comprising a full length CMV gB antigen, a truncated CMV gB antigen deleted from at least a part of the transmembrane domain, a truncated CMV gB antigen substantially deleted from all the transmembrane domain, a truncated CMV gB antigen deleted from at least a part of the intracellular domain, a truncated CMV gB antigen substantially deleted from all the intracellular domain, and a truncated CMV gB antigen deleted substantially from both the transmembrane domain and the intracellular domain.

In another embodiment, in combination or not with the preceding one, a CMV gB antigen may comprise one or several mutations, such as amino acid substitutions in the endoproteolytic cleavage site.

The expression “substantially deleted from all the intracellular domain” or “substantially deleted from all the transmembrane domain” means that at least 80% of the amino acid sequence of said domain is deleted. Therefore, a truncated gB antigen substantially deleted from all of a given domain may comprise from 0% to about 20%, for example from about 5% to about 10% of the length of the sequence of said domain, for example the intracellular domain.

As disclosed herein, by “deleted of at least a part of a domain”, is meant deleted of at least 10%, at least 20%, at least 30%, at least 40%, at least 50%, at least 60% or at least 70%, but of less than 80%, of the domain. Therefore, a truncated gB antigen deleted from at least a part of a given domain may comprise about from about 20% to about 95%, for example from about 30% to about 90%, for example from about 40% to about 60%, or for example 50% of the length of the sequence of said domain, for example the intracellular domain.

In one embodiment, a CMV gB antigen may consist in the ectodomain of a gB polypeptide, i.e., a full length gB deleted from all the transmembrane sequence, possibly including the proximal membrane domain, and from all the intracellular C-terminal domain. The “ectodomain” is the portion of a transmembrane anchored protein that extends beyond the membrane into the extracellular space. For example, the ectodomain of the full-length gB polypeptide from the AD169 strain is spanning from amino acid 26 to amino acid 707.

A CMV gB antigen as disclosed herein may also contain other mutations and/or deletions and/or additions. For instance, a CMV gB antigen may contain at least one amino acid deletion or substitution in at least one of the fusion loop 1 (FL1) domain and fusion loop 2 (FL2) domain located in the extracellular domain as described in EP 2 627 352. Alternatively, or in addition, it may contain a deletion of at least a portion of the leader sequence as described in EP 2 627 352. CMV gB antigens as disclosed herein may also comprise a mutation introducing a glycosylation site within the hydrophobic surface 1 (domain comprised of amino acid residues 154-160 and 236-243) as described in WO 2016/092460. Such glycosylation site may be an N-glycosylation site comprising an N-X-S/T/C motif, wherein X may be any amino acid residue (usually not proline). A CMV gB antigen may comprise a mutation introducing a glycosylation site. In such embodiment, the glycosylation site may be (1) within the hydrophobic surface 2 (domain comprised of amino acid residues 145-167 and 230-252); or (2) at a residue that is within 20 angstroms from fusion loop 1 (FL1) (domain comprised of amino acid residues 155-157) and/or fusion loop 2 (FL2) (amino acid residues 240-242), as described in WO 2016/092460.

In another embodiment, a CMV gB antigen may comprise a heterologous sequence which may be at least 12 residues long at the C-terminus as described in WO 2016/092460. In such embodiment, the gB protein may be a fusion protein where the heterologous sequence may be fused at the C-terminus of the ectodomain.

CMV gB has been postulated to assemble as a homotrimer, based on the 3D crystallography structure of gB proteins in related viruses, Herpes Simplex Virus 1 (HSV-1) gB and Epstein Barr Virus (EBV) gB, which are homotrimers (Heldwein et al., Science, 2006, 313:217-220; Backovic et al., PNAS, 2009, 106(8):2880-2885). A CMV gB antigen as disclosed herein may be in a trimeric form, and/or in a hexameric form (dimer of the trimeric form), and/or in a dodecameric form (dimer of hexamer). For example, a CMV gB antigen of an immunogenic composition as disclosed herein may substantially be not in a monomeric form. The expression “substantially not in a monomeric form” means that less than 20%, for example less than 10%, for example less than 5%, of the CMV gB antigen may be in a monomeric form.

According to one embodiment, a gB antigen may comprise, or consist in, an amino acid sequence which has at least 80% identity with SEQ ID NO: 1. For example, said gB antigen comprises an amino acid sequence which has at least 85% identity, at least 90% identity, at least 95% identity, at least 97% identity, at least 98% identity, at least 99% identity or even 100% identity with SEQ ID NO: 1:

STRGTSATHSHHSSHTTSAAHSRSGSVSQRVTSSQTVSHGVNETIYNTT
LKYGDVVGVNTTKYPYRVCSMAQGTDLIRFERNIVCTSMKPINEDLDEG
IMVVYKRNIVAHTFKVRVYQKVLTFRRSYAYIHTTYLLGSNTEYVAPPM
WEIHHINSHSQCYSSYSRVIAGTVFVAYHRDSYENKTMQLMPDDYSNTH
STRYVTVKDQWHSRGSTWLYRETCNLNCMVTITTARSKYPYHFFATSTG
DVVDISPFYNGTNRNASYFGENADKFFIFPNYTIVSDFGRPNSALETHR
LVAFLERADSVISWDIQDEKNVTCQLTFWEASERTIRSEAEDSYHFSSA
KMTATFLSKKQEVNMSDSALDCVRDEAINKLQQIFNTSYNQTYEKYGNV
SVFETTGGLVVFWQGIKQKSLVELERLANRSSLNLTHNTTQTSTDGNNA
THLSNMESVHNLVYAQLQFTYDTLRGYINRALAQIAEAWCVDQRRTLEV
FKELSKINPSAILSAIYNKPIAARFMGDVLGLASCVTINQTSVKVLRDM
NVKESPGRCYSRPVVIFNFANSSYVQYGQLGEDNEILLGNHRTEECQLP
SLKIFIAGNSAYEYVDYLFKRMIDLSSISTVDSMIALDIDPLENTDFRV
LELYSQKELRSSNVFDLEEIMREFNSYKQRVKYVEDKRLCMQPLQNLFP
YLVSADGTTVTSGNTKDTSLQAPPSYEESVYNSGRKGPGPPSSDASTAA
PPYTNEQAYQMLLALVRLDAEQRAQQNGTDSLDGQTGTQDKGQKPNLLD
RLRHRKNGYRHLKDSDEEENV

In an exemplary embodiment the gB antigen may comprise, or consist in, an amino acid sequence which has 100% identity with SEQ ID NO: 1.

A CMV gB antigen suitable for the present disclosure may be a truncated gB polypeptide obtained from the full length gB in which all or part of the C-terminal domain and/or all or part of the transmembrane sequence have been removed and in which the cleavage site is ineffectual. An exemplary truncated form of such a gB antigen may be the one described in U.S. Pat. No. 6,100,064, called gBd™, incorporated by reference in its entirety. In U.S. Pat. No. 6,100,064 the signal sequence of the gB was hypothesized as being 24 amino acids long. In fact, the signal sequence is 25 amino acids long. Therefore, the numbering of the amino acid of the gB indicated in U.S. Pat. No. 6,100,064 should be shifted by 1. Considering this, the gBdTM described in U.S. Pat. No. 6,100,064 carries three mutations at the cleavage site: Arginine 432 is substituted by Threonine, Lysine 434 is substituted by Glutamine and Arginine 435 is substituted by Threonine (taking into account the renumbered positions and without counting the signal sequence); and a deletion in the transmembrane region between amino acid residues valine 676 and arginine 751 (taking into account the renumbered positions), such that the extracellular domain is directly connected to the cytoplasmic domain. Such gB antigen is easier to purify as it is produced by recombinant cells expressing this product under a secreted form. The resulting form is an 806 amino acid long polypeptide deleted of its signal sequence and of its transmembrane region when it is derived from the gB Towne strain. In one exemplary embodiment, a gB antigen may be gBdTM, as disclosed herein.

A CMV gB antigen described herein may be prepared according to any method well-known to the man skilled in the art. Such methods may include conventional chemical synthesis, in solid phase (R. B. Merrifield, J. Am. Chem. Soc., 85 (14), 2149-2154 (1963)), or in liquid phase, enzymatic synthesis (K. Morihara, Trends in Biotechnology, 5(6), 164-170 (1987)) from constitutive amino acids or derivatives thereof, cell-free protein synthesis (Katzen et al., Trends in Biotechnology, 23(3), 150-156 (2005)), as well as biological production methods by recombinant technology.

For example, a CMV gB antigen may be obtained using a biological production process with a recombinant host cell. In such a process, an expression cassette, containing a nucleic acid encoding a CMV gB antigen as described herein, is transferred into a host cell, which is cultured in conditions enabling expression of the corresponding protein. The protein thereby produced can then be recovered and purified. Methods for the purification of proteins are well-known to the skilled person. The obtained recombinant protein can be purified from lysates and cell extracts or from the culture medium supernatant, by methods used individually or in combination, such as fractionation, chromatographic methods, immunoaffinity methods using specific mono- or polyclonal antibodies, etc. In one embodiment, the obtained recombinant protein may be purified from the culture medium supernatant.

CMV gB antigens may usually be obtained by recombinant DNA techniques and purified according to methods well known to those skilled in the art. The methods described in U.S. Pat. No. 6,100,064 and in US 2002/0102562, incorporated by reference in their entirety, can for example be used.

For example, a CMV gB antigen as disclosed herein may be a recombinant glycoprotein, which may be produced in Chinese hamster ovary (CHO) cell cultures. The gB gene from the Towne strain of CMV may be mutagenized to remove the cleavage site and the transmembrane part of the molecule in order to facilitate secretion in cell culture as described in U.S. Pat. No. 6,100,064. The secreted molecule may be a polypeptide of 806 amino acids, retaining 19 potential N-linked glycosylation sites, and is also called gBdTm. The purification process may involve affinity and ion-exchange chromatography steps.

A CMV gB antigen may be present in a composition in an immunologically active amount, that is in an amount suitable to induce an immune response in the intended recipient. As example of immunologically active amount of the gB antigen suitable for the present disclosure, one may cite an amount ranging from about 1 μg/ml to about 500 μg/ml, or from about 10 μg/ml to about 400 μg/ml, or from about 20 μg/ml to about 350 μg/ml, or from about 40 μg/ml to about 300 μg/ml or from about 50 μg/ml to about 280 μg/ml, or from about 80 μg/ml to about 240 μg/ml.

CMV gH/gL/UL128/UL130/UL131 Pentameric Complex Antigen

Another antigen of the immunogenic composition as disclosed herein is the CMV gH/gL/UL128/UL130/UL131 pentameric complex antigen.

Such pentameric complex is assembled through disulfide bonds and non-covalent interactions among the five components to form a functional complex able to present conformational epitopes (Ciferri et al., PNAS, 2015, 112(6):1767-1772; Wen et al., Vaccine, 2014, 32(30):3796-3804).

Suitable pentameric complex for the present disclosure has already been described and is known by the man skilled in the art. For example, such pentameric complex is described in Ryckman et al. (Journal of Virology, January 2008, p. 60-70) and in patent application WO 2014/005959 or WO 2019/052975.

### gH Antigen

A CMV gH/gL/UL128/UL130/UL131 pentameric complex may comprise a modified CMV gH polypeptide. A modified CMV gH polypeptide may be deleted from at least a part of the transmembrane (TM) domain. In some embodiments, the modified gH polypeptide may retain a part of the TM domain, but not enough to let the protein stay in a lipid bilayer. In an exemplary embodiment, a gH polypeptide may be deleted from substantially all the transmembrane domain. In another exemplary embodiment, the gH polypeptide may be deleted from all of the TM domain.

In one embodiment, a CMV glycoprotein H (gH) polypeptide may contain up to 10 amino acids (e.g. 1, 2, 3, 4, 5, 6, 7, 8, 9 or 10 amino acids) of the gH TM domain. In another embodiment, a gH polypeptide may contain no more than 10 amino acids (e.g. 1, 2, 3, 4, 5, 6, 7, 8, 9 or 10 amino acids) of the gHTM domain.

In one embodiment, a gH antigen may be deleted from at least a part of the transmembrane domain or from substantially all the transmembrane domain

In the context of the present invention, by “deleted of at least a part of a domain”, is meant deleted of at least 10%, at least 20%, at least 30%, at least 40%, at least 50%, at least 60% or at least 70%, but of less than 80%, of the domain. Therefore, a truncated gH antigen deleted from at least a part of a given domain may comprise about from about 20% to about 95%, for example from about 30% to about 90%, for example from about 40% to about 60%, or for example 50% of the length of the sequence of said domain, for example the transmembrane domain.

The expression “deleted from substantially all the intracellular domain” or “deleted from substantially all the transmembrane domain” means that at least 80% of the amino acid sequence of the corresponding domain is deleted. Therefore, a truncated gH antigen substantially deleted from all of a given domain may comprise from 0% to about 20%, for example from about 5% to about 10% of the length of the sequence of the domain, for example the transmembrane domain.

Alternatively, or in addition of being deleted from at least a part, from substantially all, or from all of the TM domain, a gH polypeptide may be deleted from a part, from substantially all, or from all of the intracellular domain of CMV gH.

In one embodiment, a gH antigen may be deleted from a part of the intracellular domain of CMV gH. In another embodiment, a gH antigen may be deleted from substantially all the intracellular domain. In another embodiment, a gH polypeptide may be deleted from all the intracellular domain.

In one embodiment, a gH polypeptide may be deleted from all the TM domain and from all the intracellular domain.

In one embodiment, a gH antigen may comprise, or consist in, the ectodomain of the full length gH polypeptide encoded by CMV UL75 gene.

A gH antigen, which is encoded by the UL75 gene, is a virion glycoprotein that is essential for infectivity and which is conserved among members of the alpha-, beta- and gamma-herpes viruses. It forms a stable complex with gL, and the formation of this complex facilitates the cell surface expression of gH. Based on the crystal structures of HSV-2 and EBV gH/gL complexes, the gL subunit and N-terminal residues of gH form a globular domain at one end of the structure (the ‘head’), which is implicated in interactions with gB and activation of membrane fusion. The C-terminal domain of gH, proximal to the viral membrane (the ‘tail’), is also implicated in membrane fusion.

In one embodiment, a gH polypeptide in the pentameric complex described herein may comprise, or consist in, an amino acid sequence which has at least 80% identity with SEQ ID NO: 2. In another embodiment, a gH antigen may comprise, or consist in, an amino acid sequence which has at least 85% identity, at least 90% identity, at least 95% identity, at least 97% identity, at least 98% identity, at least 99% identity or even 100% identity with SEQ ID NO: 2:

RYGAEAVSEPLDKAFHLLLNTYGRPIRFLRENTTQCTYNNSLRNSTVVR
ENAISFNFFQSYNQYYVFHMPRCLFAGPLAEQFLNQVDLTETLERYQQR
LNTYALVSKDLASYRSFSQQLKAQDSLGEQPTTVPPPIDLSIPHVWMPP
QTTPHGWTESHTTSGLHRPHFNQTCILFDGHDLLFSTVTPCLHQGFYLI
DELRYVKITLTEDFFVVTVSIDDDTPMLLIFGHLPRVLFKAPYQRDNFI
LRQTEKHELLVLVKKDQLNRHSYLKDPDFLDAALDFNYLDLSALLRNSF
HRYAVDVLKSGRCQMLDRRTVEMAFAYALALFAAARQEEAGAQVSVPRA
LDRQAALLQIQEFMITCLSQTPPRTTLLLYPTAVDLAKRALWTPNQITD
ITSLVRLVYILSKQNQQHLIPQWALRQIADFALKLHKTHLASFLSAFAR
QELYLMGSLVHSMLVHTTERREIFIVETGLCSLAELSHFTQLLAHPHHE
YLSDLYTPCSSSGRRDHSLERLTRLFPDATVPATVPAALSILSTMQPST
LETFPDLFCLPLGESFSALTVSEHVSYVVTNQYLIKGISYPVSTTVVGQ
SLIITQTDSQTKCELTRNMHTTHSITAALNISLENCAFCQSALLEYDDT
QGVINIMYMHDSDDVLFALDPYNEVVVSSPRTHYLMLLKNGTVLEVTDV
VVDATDSR

In another embodiment, a gH polypeptide may comprise, or consist in, an amino acid sequence which has 100% identity with SEQ ID NO: 2.

### gL Antigen

CMV glycoprotein L (gL) is encoded by the UL115 gene. gL antigen is thought to be essential for viral replication and all known functional properties of gL are directly associated with its dimerization with gH. The gL/gH complex is required for the fusion of viral and plasma membranes leading to virus entry into the host cell.

According to one embodiment, a gL polypeptide of a pentameric complex described herein may comprise, or consist in, an amino acid sequence which has at least 80% identity with SEQ ID NO: 3. In another embodiment, a gL antigen may comprise, consist in, an amino acid sequence which has at least 85% identity, at least 90% identity, at least 95% identity, at least 97% identity, at least 98% identity, at least 99% identity or even 100% identity with SEQ ID NO: 3.

In an exemplary embodiment, a gL polypeptide may comprise, or consist in, an amino acid sequence which has 100% identity with SEQ ID NO: 3:

AAVSVAPTAAEKVPAECPELTRRCLLGEVFQGDKYESWLRPLVNVTGRD
GPLSQLIRYRPVTPEAANSVLLDEAFLDTLALLYNNPDQLRALLTLLSS
DTAPRWMTVMRGYSECGDGSPAVYTCVDDLCRGYDLTRLSYERSIFTEH
VLGFELVPPSLFNVVVAIRNEATRTNRAVRLPVSTAAAPEGITLFYGLY
NAVKEFCLRHQLDPPLLRHLDKYYAGLPPELKQTRVNLPAHSRYGPQAV
DAR

### UL128 Antigen

According to an embodiment, a UL128 polypeptide in a pentameric complex described herein may comprise, or consist in, an amino acid sequence which has at least 80% identity with SEQ ID NO: 4. In one embodiment, a UL128 antigen may comprise, or consist in, an amino acid sequence which has at least 85% identity, at least 90% identity, at least 95% identity, at least 97% identity, at least 98% identity, at least 99% identity or even 100% identity with SEQ ID NO: 4.

In an exemplary embodiment, a UL128 polypeptide may comprise, or consist in, an amino acid sequence which has 100% identity with SEQ ID NO: 4:

EECCEFINVNHPPERCYDFKMCNRFTVALRCPDGEVCYSPEKTAEIRGI
VTTMTHSLTRQVVHNKLTSCNYNPLYLEADGRIRCGKVNDKAQYLLGAA
GSVPYRWINLEYDKITRIVGLDQYLESVKKHKRLDVCRAKMGYMLQ

### UL130 Antigen

UL130 is the central and the largest (214 codons) gene of the UL131A-128 locus. Conceptual translation of the gene predicts a long (25 amino acids) N-terminal signal sequence that precedes a hydrophilic protein containing two potential N-linked glycosylation sites (Asn85 and Asn118) within a putative chemokine domain (amino acids 46 to 120) and an additional N-glycosylation site (Asn201) close to the end of a unique C-terminal region. UL130 is predicted to be devoid of a TM domain.

It has been reported to be a luminal glycoprotein that is inefficiently secreted from infected cells but is incorporated into the virion envelope as a Golgi-matured form (Patrone, et al., Journal of Virology. 79 (2005): 8361-8373).

According to an embodiment, a UL130 polypeptide in a pentameric complex described herein may comprise, or consist in, an amino acid sequence which has at least 80% identity with SEQ ID NO: 5. In one embodiment, the UL130 antigen may comprise, or consist in, an amino acid sequence which has at least 85% identity, at least 90% identity, at least 95% identity, at least 97% identity, at least 98% identity, at least 99% identity or even 100% identity with SEQ ID NO: 5.

In an exemplary embodiment, a UL130 polypeptide may comprise, or consist in, an amino acid sequence which has 100% identity with SEQ ID NO: 5:

SPWSTLTANQNPSPLWSKLTYSKPHDAATFYCPFIYPSPPRSPLQFSGF
QRVLTGPECRNETLYLLYNREGQTLVERSSTWVKKVIWYLSGRNQTILQ
RMPRTASKPSDGNVQISVEDAKIFGAHMVPKQTKLLRFVVNDGTRYQMC
VMKLESWAHVFRDYSVSFQVRLTFTEANNQTYTFCTHPNLIV

### UL131A Antigen

UL131, also called UL131A, function is required for CMV replication not only in endothelial cells but also in epithelial cells. According to an embodiment, a UL131A polypeptide in a pentameric complex described herein may comprise, or consist in, an amino acid sequence which has at least 80% identity with SEQ ID NO: 6. In one embodiment, the UL131A antigen may comprise, or consist in, an amino acid sequence which has at least 85% identity, at least 90% identity, at least 95% identity, at least 97% identity, at least 98% identity, at least 99% identity or even 100% identity with SEQ ID NO: 6.

In an exemplary embodiment, a UL131 polypeptide may comprise, or consist in, an amino acid sequence which has 100% identity with SEQ ID NO: 6:

QCQRETAEKNDYYRVPHYWDACSRALPDQTRYKYVEQLVDLTLNYHYDA
SHGLDNFDVLKRINVTEVSLLISDFRRQNRRGGTNKRTTFNAAGSLAPH
ARSLEFSVRLFAN SEQ ID NO: 2 to 6 are from the
strain BE/28/2011 (Genbank ID KP745669).

### Pentameric Complex Antigen

In a pentameric complex antigen of an immunogenic composition as disclosed herein, gH, gL and UL128 can be linked through disulfide bonds, but UL130 and UL131A can be incorporated into the pentameric complex by non-covalent interactions. For example, the UL130 protein and/or UL131A protein may be incorporated into a pentameric complex by non-covalent interactions. Furthermore, a UL130 protein and/or a UL131A protein may be inter-linked by non-covalent interactions.

A range of conformational epitopes for the pentameric complex are known. For example, Macagno et al. (Macagno et al., Journal of Virology. 84 (2010): 1005-13) isolated a panel of human monoclonal antibodies that neutralized CMV infection of endothelial, epithelial, and myeloid cells. In one embodiment, a pentameric complex antigen of an immunogenic composition as disclosed herein may display one or more of the conformational epitopes identified by Macagno et al. (2010).

Each protein of a pentameric complex antigen may contain mutations, such as insertions, deletions and substitutions, so long as these mutations are not detrimental to the use of the proteins as antigens. In addition, such mutations should not prevent the capacity of the proteins to form a pentameric complex according to the invention. The ability to form a pentameric complex as disclosed herein can be tested by performing protein purification, and analyzing the proteins by non-reducing PAGE, Western blot and/or size exclusion chromatography. If the proteins form part of a complex, they may all be present in a single band on a native PAGE gel and/or be present in a single peak in a size exclusion chromatogram.

Expression of said pentameric complex can be realized according to methods known by the man skilled in the art. Mention can be made for example of the method described in Hofmann et al. (Biotechnology and Bioengineering, 2015).

Suitable expression systems for use in the context of the present disclosure are well known to the man skilled in the art and many are described in detail in Doyle (Doyle, ed. High Throughput Protein Expression and Purification: Methods and Protocols, in Methods in Molecular Biology, Ed. Humana Press, 2008). Generally, any system or vector that is suitable to maintain, propagate and express nucleic acid molecules to produce a polypeptide in the required host may be used. The appropriate nucleotide sequence may be inserted into an expression system by any of a variety of well-known and routine techniques, such as, for example, those described in Sambrook (Sambrook, J. Molecular Cloning: A Laboratory Manual. 3rd. Ed. Cold Spring Harbor Laboratory Press, 2000). Generally, the encoding gene can be placed under the control of a control element such as a promoter, and, optionally, an operator, so that the DNA sequence encoding the desired peptide is transcribed into RNA in the transformed host cell. Examples of suitable expression systems include, for example, chromosomal, episomal and virus-derived systems, including, for example, vectors derived from: bacterial plasmids, bacteriophage, transposons, yeast episomes, insertion elements, yeast chromosomal elements, viruses such as baculoviruses such as described in patent application WO 2015/170287, papova viruses such as SV40, vaccinia viruses, adenoviruses, fowl pox viruses, pseudorabies viruses and retroviruses, or combinations thereof, such as those derived from plasmid and bacteriophage genetic elements, including cosmids and phagemids. Human artificial chromosomes (HACs) may also be employed to deliver larger fragments of DNA than can be contained and expressed in a plasmid.

In order to express the five different recombinant proteins of a CMV gH/gL/UL128/UL130/UL131 pentameric complex antigen simultaneously and in an equimolar way, there are several possibilities. A first possibility (1) may be to build a single vector containing all five ORFs under the control of the same or similar regulations elements (promoter, enhancer, splice signal, termination signal, . . . ) and optionally a selection system for cell line selection. The vector may contain five expression cassettes (for instance as described in Albers et al., J. Clin. Invest., 2015, 125(4): 1603-1619; or in Cheshenko et al., Gene Ther., 2001, 8(11): 846-854), or the five components (gH, gL, UL128, UL130 and UL131) may be fused in a single ORF with elements triggering the proper polyprotein maturation into the five proteins of a CMV gH/gL/UL128/UL130/UL131 pentameric complex antigen (for instance self-cleavable sequences as described in Szymczak-Workman et al., Cold Spring Harb. Protoc., 2012, 2012 (2): 199-204). In that second case, the equimolarity is guaranteed, assuming all cleavage occur correctly. Another possibility (2) for expressing a CMV gH/gL/UL1 28/UL130/UL131 pentameric complex may be to build five vectors each expressing one component of the CMV gH/gL/UL128/UL130/UL131 pentameric complex antigen and optionally a selection system for cell line selection. The five vectors may be co-transfected in the target cell line. Any intermediate system between possibility (1) and possibility (2) could also be designed to minimize the number of vectors required and maintain each vector to a reasonable size (less than 12 kb, for example).

Suitable expression systems include microorganisms such as bacteria transformed with recombinant bacteriophage, plasmid or cosmid DNA expression vectors; yeast transformed with yeast expression vectors; insect cell systems infected or transfected with virus expression vectors (for example, baculovirus such as described in patent application WO 2015/170287); plant cell systems transformed with virus expression vectors (for example, cauliflower mosaic virus, CaMV; tobacco mosaic virus, TMV) or with bacterial expression vectors (for example, Ti or pBR322 plasmids); or animal cell systems. Cell-free translation systems can also be employed to produce the proteins.

Examples of suitable plant cellular genetic expression systems may include those described in U.S. Pat. Nos. 5,693,506; 5,659,122; 5,608,143 and Zenk, Phytochemistry, 1991, 30(12): 3861-3863. For example, all plants from which protoplasts can be isolated and cultured to give whole regenerated plants can be used, so that whole plants are recovered which contain the transferred gene. Practically all plants can be regenerated from cultured cells or tissues, including but not limited to all major species of sugar cane, sugar beet, cotton, fruit and other trees, legumes and vegetables.

HEK293 cells may be suitable for transient expression of CMV proteins of a pentamer complex as disclosed herein due to their high transfectability by various techniques, including the calcium phosphate and polyethylenimine (PEI) methods. A useful cell line of HEK293 may be the one that expresses the EBNA1 protein of EBV, such as 293-6E (Loignon, et al., BMC Biotechnology, 2008; 8: 65). Transformed HEK293 cells have been shown to secrete high levels of the protein into the growth medium, thus allowing the purification of such protein complexes directly from the growth medium.

CHO cells may be suitable mammalian hosts for industrial production of CMV proteins, as for example industrial production of a CMV gH/gL/UL128/UL130/UL131 pentameric complex antigen part of the immunogenic composition according to the invention. Transfection can be carried out by a range of methods well known in the art including using calcium phosphate, electroporation, or by mixing a cationic lipid with the material to produce liposomes which fuse with the cell membrane and deposit their cargo inside.

Methods for purifying recombinant proteins from cell supernatant or from inclusion bodies are well known in the art. In an exemplary embodiment, a CMV gH/gL/UL128/UL130/UL131 pentameric complex antigen may be purified by size-exclusion chromatography.

A CMV gH/gL/UL128/UL130/UL131 pentameric complex antigen may be present in a composition in an immunologically active amount, that is in an amount suitable to induce an immune response in the intend recipient. As example of immunologically active amount of gH/gL/UL128/UL130/UL131 pentameric complex antigen suitable for the present disclosure, one may cite an amount ranging from about 1 μg/ml to about 500 μg/ml, or from about 10 μg/ml to about 400 μg/ml, or from about 20 μg/ml to about 350 μg/ml, or from about 40 μg/ml to about 300 μg/ml or from about 50 μg/ml to about 280 μg/ml, or from about 80 μg/ml to about 240 μg/ml.

In one embodiment, immunogenic compositions as disclosed herein does not comprise any complete CMV virus.

In one embodiment, immunogenic compositions as disclosed herein may comprise further antigens that the CMV antigens described herein. As example of further antigens which may added to a composition as disclosed herein, one may cite antigen from: Bordetella pertussis, Corynebacterium diptheriae, Clostridium tetani, Mycobacterium tuberculosis, Plasmodium spp., Bacillus anthracis, Vibrio cholera, Salmonella typhi, Borrelia spp., Streptococcus pneumoniae, Staphylococcus aureus, Escherichia coli, Clostridium spp., Mycobacterium leprae, Yersinia pestis, influenza virus, varicella zoster virus, human immunodeficiency virus (HIV), respiratory syncytial virus (RSV), SARS-Cov-2 virus, polio virus, variola virus, rabies virus, rotavirus, human papillomavirus, Ebola virus, hepatitis A virus, hepatitis B virus, hepatitis C virus, lyssavirus, measles virus, mumps virus, and Rubella virus. In an exemplary embodiment, immunogenic compositions as disclosed herein may comprise an CMV gB antigen and a CMV gH/gL/UL128/UL130/UL131 pentameric complex antigen as the only CMV antigens of the composition.

In an exemplary embodiment, immunogenic compositions as disclosed herein may comprise an CMV gB antigen and a CMV gH/gL/UL128/UL130/UL131 pentameric complex antigen as the only CMV antigens of the composition.

### Fungal Antigens

Fungal antigens may be obtained from Ascomycota (e.g., Fusarium oxysporum, Pneumocystis jiroviecii, Aspergillus spp., Coccidioides immitis/posadasii, Candida albicians), Basidiomycota (e.g., Filobasidiella neoformans, Trichosporon), Microsporidia (e.g., Encephalitozoon cuniculi, Enterocytozoon bieneusi), or Mucoromycotina (e.g., Mucor circinelloides, Rhizopus oryzae, Lichtheimia corymbifera).

### Protozoan Antigens

Protozoan antigens may be obtained from Entamoeba histolytica, Giardia lambila, Trichomonas vaginalis, Trypanosoma brucei, T. cruzi, Leishmania donovani, Balantidium coli, Toxoplasma gondii, Plasmodium spp., or Babesia microti.

### Parasitic Antigens

Parasitic antigens may be obtained from Acanthamoeba, Anisakis, Ascaris lumbricoides, botfly, Balantidium coli, bedbug, Cestoda, chiggers, Cochliomyia hominivorax, Entamoeba histolytica, Fasciola hepatica, Giardia lamblia, hookworm, Leishmania, Linguatula serrata, liver fluke, Loa loa, Paragonimus, pinworm, Plasmodium falciparum, Schistosoma, Strongyloides stercoralis, mite, tapeworm, Toxoplasma gondii, Trypanosoma, whipworm, or Wuchereria bancrofti.

### Tumour Antigens

In one embodiment, an antigen may be a tumor antigen, i.e., a constituent of cancer cells such as a protein or peptide expressed in a cancer cell. The term “tumor antigen” relates to proteins that are under normal conditions specifically expressed in a limited number of tissues and/or organs or in specific developmental stages and are expressed or aberrantly expressed in one or more tumor or cancer tissues. Tumor antigens include, for example, differentiation antigens, such as cell type specific differentiation antigens, i.e., proteins that are under normal conditions specifically expressed in a certain cell type at a certain differentiation stage and germ line specific antigens. For example, a tumor antigen is presented by a cancer cell in which it is expressed.

For example, tumor antigens include the carcinoembryonal antigen, a 1-fetoprotein, isoferritin, and fetal sulphoglycoprotein, cc2-H-ferroprotein and γ-fetoprotein.

Other examples for tumor antigens that may be useful in the present invention are p53, ART-4, BAGE, beta-catenin/m, Bcr-abL CAMEL, CAP-1, CASP-8, CDC27/m, CD 4/m, CEA, the cell surface proteins of the claudin family, such as CLAUDIN-6, CLAUDIN-18.2 and CLAUDIN-12, c-MYC, CT, Cyp-B, DAM, ELF2M, ETV6-AML1, G250, GAGE, GnT-V, Gapl OO, HAGE, HER-2/neu, HPV-E7, HPV-E6, HAST-2, hTERT (or hTRT), LAGE, LDLR/FUT, MAGE-A, such as MAGE-A1, MAGE-A2, MAGE-A3, MAGE-A4, MAGE-A5, MAGE-A6, MAGE-A7, MAGE-A8, MAGE-A9, MAGE-A10, MAGE-A11, or MAGE-A12, MAGE-B, MAGE-C, MART-1/Melan-A, MC1R, Myosin/m, MUC1, MUM-1, -2, -3, NA88-A, NF1, NY-ESO-1, NY-BR-1, pl 90 minor BCR-abL, Pm I/RARa, PRAME, proteinase 3, PSA, PSM, RAGE, RUI or RU2, SAGE, SART-1 or SART-3, SCGB3A2, SCP 1, SCP2, SCP3, SSX, SURVrVIN, TEL/AMLI, TPI/m, TRP-1, TRP-2, TRP-2/1 NT2, TPTE and WT, such as WT-1.

### Liposomes and Methods for Manufacturing Thereof

The present disclosure also relates to a method for manufacturing a liposome comprising at least the steps of:
(a) solubilizing, in an organic water-miscible solvent, a TLR4 agonist of formula (I) having a solubility parameter in ethanol, measured at 25° C., of at least about 0.2 mg/mL, a sterol, and a phospholipid,
(b) processing the solution obtained at step (a) into a liposome,
   wherein a saponin is added either at step (a), at step (b), or after step (b) and
   wherein the TLR4 agonist and the saponin are present in a weight:weight ratio of saponin:TLR4-agonist ranging from about 1:1 to about 400:1, ranging from about 2:1 to about 200:1, ranging from about 2.5:1 to about 100:1, ranging from about 3:1 to about 40:1, or ranging from about 5:1 to about 25:1. Such method may allow obtaining a single type of liposomes as disclosed herein.

In another embodiment, the disclosure is directed to a method for manufacturing a liposome, for example a second type of liposome, comprising at least the steps of:
(a) solubilizing, in an organic water-miscible solvent, a TLR4 agonist of formula (I) having a solubility parameter in ethanol, measured at 25° C., of at least about 0.2 mg/mL, a sterol, and a phospholipid,
(b) processing the mixture obtained at step (a) into a liposome. In such embodiment, the method does not comprise the addition of a saponin at step (a) and/or (b). In such embodiment, the obtained liposomes may be devoid of saponin. Such method may allow obtaining a second type of liposomes as disclosed herein.

In one embodiment, a method as disclosed herein for manufacturing a liposome may comprise a step, prior to step (a), of selecting a TLR4 agonist of formula (I) having a solubility parameter in ethanol, measured at 25° C., of at least about 0.2 mg/mL.

The present disclosure also relates to a method for manufacturing a liposome as disclosed herein comprising at least the steps of:
(a1) selecting a TLR4 agonist of formula (I) which has a solubility parameter in ethanol, measured at 25° C., of at least about 0.2 mg/mL,
(a2) solubilizing, in an organic water-miscible solvent, the TLR4 agonist selected as step (a1), a sterol, and a phospholipid, and
(b) processing the solution obtained at step (a2) into a liposome,
   wherein a saponin is added either at step (a2), at step (b), or after step (b), and
   wherein the TLR4 agonist and the saponin are present in a weight:weight ratio of saponin:TLR4-agonist ranging from about 1:1 to about 400:1, ranging from about 2:1 to about 200:1, ranging from about 2.5:1 to about 100:1, ranging from about 3:1 to about 40:1, or ranging from about 5:1 to about 25:1.

In another embodiment, the disclosure is directed to a method for manufacturing a liposome, for example a second type of liposome, comprising at least the steps of:
(a1) selecting a TLR4 agonist of formula (I) which has a solubility parameter in ethanol, measured at 25° C., of at least about 0.2 mg/mL,
(a2) solubilizing, in an organic water-miscible solvent, the TLR4 agonist selected as step (a1), a sterol, and a phospholipid, and,
(b) processing the mixture obtained at step (a) into a liposome. Such a method may not comprise the addition of a saponin at steps (a) and/or (b). In such embodiment, the obtained liposomes may be devoid of saponin. Such method may allow obtaining a second type of liposomes as disclosed herein.

In one embodiment, a method as disclosed herein for manufacturing a liposome may comprise a step, prior to step (a1), of determining the solubility parameter in ethanol of a TLR4 agonist of formula (I) at a temperature of about 25° C. and at an atmospheric pressure of about 1 013 hPa.

The present disclosure further relates to a method for manufacturing a liposome as disclosed herein comprising at least the steps of:
(a1) determining the solubility parameter in ethanol of a TLR4 agonist of formula (I) at a temperature of about 25° C. and at an atmospheric pressure of about 1 013 hPa;
(a2) selecting a TLR4 agonist of formula (I) which has a solubility parameter measured at step (a1) of at least about 0.2 mg/mL;
(a3) solubilizing, in an organic water-miscible solvent, a TLR4 agonist, a sterol, and a phospholipid,
wherein the TLR4 agonist is a TLR4 agonist of formula (I) having a solubility parameter in ethanol, measured at 25° C., of at least about 0.2 mg/mL, and
(b) processing the solution obtained at step (a3) into a liposome,
   wherein a saponin is added either at step (a3), at step (b), or after step (b), and
   wherein the TLR4 agonist and the saponin are present in a weight:weight ratio of saponin:TLR4-agonist ranging from about 1:1 to about 400:1, ranging from about 2:1 to about 200:1, ranging from about 2.5:1 to about 100:1, ranging from about 3:1 to about 40:1, or ranging from about 5:1 to about 25:1.

In another embodiment, the disclosure is directed to a method for manufacturing a liposome, for example a second type of liposome, comprising at least the steps of:
(a1) determining the solubility parameter in ethanol of a TLR4 agonist of formula (I) at a temperature of about 25° C. and at an atmospheric pressure of about 1 013 hPa;
(a2) selecting a TLR4 agonist of formula (I) which has a solubility parameter measured at step (a1) of at least about 0.2 mg/mL;
(a3) solubilizing, in an organic water-miscible solvent, a TLR4 agonist, a sterol, and a phospholipid,
wherein the TLR4 agonist is a TLR4 agonist of formula (I) having a solubility parameter in ethanol, measured at 25° C., of at least about 0.2 mg/mL, and
(b) processing the mixture obtained at step (a) into a liposome. Such a method may not comprise the addition of a saponin at steps (a) and/or (b). In such embodiment, the obtained liposomes may be devoid of saponin. Such method may allow obtaining a second type of liposomes as disclosed herein.

The TLR4 agonists, the saponins, the sterols and the phospholipids that are suitable for manufacturing a liposome according to the methods disclosed herein have been described above. The amounts and ratios in which these compounds may be mixed have also been described above.

The selected TLR4 agonist may have a solubility parameter in ethanol of at least about 0.2 mg/mL.

A selected TLR4 agonist may have a solubility parameter in ethanol of at least about 0.5 mg/mL, of at least about 1 mg/mL, of at least 2 mg/mL, of at least 4 mg/mL, of at least 6 mg/mL, of at least 10 mg/mL, of at least 12 mg/mL, of at least 15 mg/mL, of at least 20 mg/mL, of at least 25 mg/mL, or of at least 30 mg/mL.

A selected TLR4 agonist may have a solubility parameter in ethanol of about 0.1 to about 50 mg/mL, of about 0.2 to about 45 mg/mL, of about 1 to about 40 mg/mL, of about 2 to about 35 mg/mL, of about 6 to about 30 mg/mL, or of about 10 to about 25 mg/mL.

A selected TLR4 agonist may have a solubility parameter in ethanol ranging from about at least about 0.2 mg/mL to about 20 mg/ml from about at least about 0.5 mg/mL to about 15 mg/ml, from about at least about 1 mg/mL to about 12 mg/ml, from about at least about 2 mg/mL to about 10 mg/ml, from about at least about 4 mg/mL to about 10 mg/ml.

In one embodiment, the selected TLR4 agonist has a solubility parameter in ethanol of at least about 10 mg/mL.

The solubility parameter is measured at a temperature of about 25° C. and at an atmospheric pressure of about 1 013 hPa. The solubility parameter may be measured by nephelometry.

Methods of determining the solubility parameter of a molecule such as a TLR4 agonist of formula (I) are well known to one skilled in the art. Examples of such methods include performing a nephelometry measure of the molecule in ethanol at different concentrations of said molecule. For example, nephelometry may be performed on a BMG-Labtech Nephelostar with 0.200 ml of each solution including a different concentration of the molecule to be tested on a UV 96-well microplate (Thermo UV Flat Bottom 96 Ref 8404) with a blank in ethanol. RNU (Relative nephelometry Unit) of each solution may be recorded. Other methods of determining the solubility parameter of a molecule are described in Veseli et al. (Drug Dev Ind Pharm. 2019 November; 45(11):1717-1724.

Methods for processing a solution obtained at step (a) in liposomes are known in the art (Wagner A et al. J Drug Deliv. 2011; 591325). As exemplary embodiments, mention may be made of the “thin film method” or of the “solvent injection method”.

The “thin film method”, detailed for instance in Liposomes: A practical approach. Edited by RRC New. Oxford University Press, 1990, consists in obtaining a solution of the lipidic compounds, i.e. the TLR4 agonist, the sterol, the phospholipid and optionally the saponin, in an appropriate organic solvent or organic solvent mixture, according to step (a). This method is used for instance in the preparation of liposomes in WO2007/068907 A1.

A suitable organic solvent or solvent mixture may be chloroform, dichloromethane, chloroform/ethanol, dichloromethane/ethanol, isopropanol, isopropanol/ethanol, chloroform/methanol, dichloromethane/methanol, isopropanol, or isopropanol/methanol.

The obtained solution is then dried to evaporate the organic solvent to obtain a lipidic dry matter, as a thin lipid film or lipid cakes. Evaporation may be made by using a dry nitrogen or argon stream in a fume hood or by rotary evaporation on the walls of a glass vessel.

The obtained lipidic dry matter is then hydrated by resuspension in an appropriate aqueous medium or aqueous buffer. Hydration time may differ slightly among lipid species and structure. A suitable aqueous medium or buffer may be PBS at pH 6.1, or a citrate buffer at pH 6.3, to obtain liposomes.

In the methods disclosed herein, if the saponin is not added at step (a) but is added at step (b), then, in the thin film method, it may be added at a step of hydration of the dry lipid matter, by addition and solubilization in the aqueous medium or aqueous buffer used for the hydration step. Alternatively, it may be added after step b) as a solution of saponin to a suspension of liposomes obtained at step (b).

In another embodiment, the disclosure is directed to a method for manufacturing a liposome, for example a first type of liposome, comprising at least the steps of:
(a) solubilizing, in an organic water-miscible solvent a sterol, and a phospholipid,
(b) processing the mixture obtained at step (a) into a liposome,
   wherein a saponin is added either at step (a), at step b) or after step (b). In such embodiment, step a) does not comprising a step of solubilizing, in an organic water-miscible solvent, a TLR4 agonist. In such embodiment, the obtained liposome may be devoid of TLR4 agonist. Such method may allow obtaining a first type of liposomes as disclosed herein.

The resulting liposomes or liposomal suspension are/is then sized by treatment with ultrasonication, microfluidization or extrusion so as to reduce the diameter of the liposomes in order to enable sterilization by filtration through a 0.2 μm pore size membrane.

The thin film method often uses chlorinated organic solvents which are usually difficult to manipulate. Furthermore, the thin film method relies upon a lipid drying step to obtain a thin lipid film on the walls of glass vessels. This step poses many hurdles to scale up, such as at an industrial level. As such, other methods may prove more advantageous when preparing liposomes.

The “solvent injection method”, detailed for instance in Liposomes: A practical approach. Edited by RRC New. Oxford University Press, 1990, consists in obtaining a solution lipidic compounds, i.e. the TLR4 agonist, the sterol, the phospholipid and, optionally, the saponin in a selected ratio into an organic water-miscible solvent or an organic water-miscible solvent mixture.

A suitable organic water-miscible solvent or organic water-miscible solvent mixture may be ethanol, isopropanol, or isopropanol/ethanol. In one exemplary embodiment, a suitable organic water-miscible solvent may be ethanol. Ethanol, as opposed to other available solvents or mixtures of solvents, such as isopropanol, is considered as one of the safest compounds to be used in the manufacturing process of pharmaceutical products by the Health Agencies.

The solvent injection method imposes a step of solubilizing lipid compounds in an appropriate organic water-miscible solvent or organic water-miscible solvent mixture, such as ethanol. The use of the method to manufacture the liposomes as disclosed herein is made possible because of a selection of specific TLR4 agonists having a specific threshold of solubility in organic water-miscible solvents. In one embodiment, the selected TLR4 agonists have a specific threshold of solubility in ethanol, as disclosed herein.

The solvent injection method has the advantage to be easy to scale-up at industrial level compared to other possible liposome manufacturing methods, as for example the thin-film method

In an embodiment, the step (b) of processing the solution obtained at step (a) into a liposome is performed by using the solvent injection method.

In another embodiment, step (b) of processing the solution obtained at step (a) into a liposome includes the step of:
(b1) injecting and/or diluting the solution obtained at step (a) into an aqueous buffer.

The obtained solution at step (a) is then injected or diluted into an excess of aqueous medium or aqueous buffer. A suitable buffer may be PBS at pH 6.1, a citrate buffer at pH 6.3. The solvent is then eliminated by dialysis or diafiltration. A dilution may be performed by crossflow mixing by using a T connector or a crossflow injection device as described in Wagner et al., J Liposome Res. 2006; 16(3):311-9 or in Wagner et al., J Drug Deliv. 2011; 2011:591325 or by using a microfluidic device. A suitable microfluidic device may be NanoAssemblR from Precison Nanosystems, Vancouver, Canada. By using the solvent injection method, small liposomes compatible with sterilization by filtration on a 0.2 μm pore size membrane can be obtained directly by adequate selection of the process parameters (volumes and ratio solvent/buffer, speed of mixing, etc. . . . ).

In one embodiment, the injection step may be carried out by dilution steps, injection with a syringe, or cross-flow injection system.

In another embodiment, the step (b) of processing the solution obtained at step (a) into a liposome may further include the step of:
(b2) removing the organic water-miscible solvent.

Removing of organic water-miscible solvent may be made by dialysis, diafiltration or tangential flow filtration.

In another embodiment, the step (b) of processing the solution obtained at step (a) into a liposome may include the steps of:
(b1) injecting and/or diluting the solution obtained at step (a) into an aqueous buffer, and
(b2) removing the organic water-miscible solvent.

In one embodiment, a method for manufacturing a liposome may comprise at least the steps of:
(a) solubilizing, in an organic water-miscible solvent, a TLR4 agonist of formula (I) having a solubility parameter in ethanol, measured at 25° C., of at least about 0.2 mg/mL, a sterol, and a phospholipid,
(b1) injecting and/or diluting the solution obtained at step (a) into an aqueous buffer, and
(b2) removing the organic water-miscible solvent,
   wherein a saponin is added either at step (a), at step (b1), at step (b2), or after step (b2), and
   wherein the TLR4 agonist and the saponin are present in a weight:weight ratio of saponin:TLR4-agonist ranging from about 1:1 to about 400:1, ranging from about 2:1 to about 200:1, ranging from about 2.5:1 to about 100:1, ranging from about 3:1 to about 40:1, or ranging from about 5:1 to about 25:1.

When added at step (b1), the saponin is solubilized in the aqueous buffer.

When added at step (b2), the saponin is solubilized in an aqueous buffer used for dialyzing the suspension containing the liposomes to remove the organic water-miscible solvent.

When added after step (b2), the saponin is solubilized in an aqueous buffer and then mixed to the suspension of liposomes obtained after step (b2).

When the saponin displays a high affinity for the sterol, such as when using QS21 and cholesterol, the saponin may be incorporated into the liposomes by post-addition to the preformed sterol-containing liposomes. In this case the sterol-containing liposomes are prepared as described above and the saponin is incorporated by simple mixing of a saponin solution (in water or acidic buffer such as PBS pH 6.1 or citrate pH 6.3) with the suspension of sterol-containing liposomes.

In another embodiment, the disclosure is directed to a method for manufacturing a liposome, for example a first type of liposome, comprising at least the steps of:
(a) solubilizing, in an organic water-miscible solvent a sterol, and a phospholipid,
(b1) injecting and/or diluting the solution obtained at step (a) into an aqueous buffer, and
(b2) removing the organic water-miscible solvent,
wherein a saponin is added either at step (a), at step (b1), at step (b2), or after step (b2). In such embodiment, step a) does not comprising a step of solubilizing, in an organic water-miscible solvent, a TLR4 agonist. In such embodiment, the obtained liposome may be devoid of TLR4 agonist. Such method may allow obtaining a first type of liposomes as disclosed herein.

In another embodiment, the disclosure is directed to a method for manufacturing a liposome, for example a second type of liposome, comprising at least the steps of:
(a) solubilizing, in an organic water-miscible solvent, a TLR4 agonist of formula (I) having a solubility parameter in ethanol, measured at 25° C., of at least about 0.2 mg/mL, a sterol, and a phospholipid,
(b1) injecting and/or diluting the solution obtained at step (a) into an aqueous buffer, and
(b2) removing the organic water-miscible solvent. In such embodiment, the method does not comprise the addition of a saponin at step (a) and/or (b). In such embodiment, the obtained liposomes may be devoid of saponin. Such method may allow obtaining a second type of liposomes as disclosed herein.

Step (a) of a method as disclosed herein may be broken down in steps (a1) and (a2) or (a1), (a2) and (a3) as above described.

Liposomes of the present disclosure are mixtures of small unilamellar vesicles and small multilamellar vesicles having an average diameter of around 100 nm, when measured by dynamic light scattering using a Zetasizer Nano ZS (Malvern Instrument; Malvern, UK) by following the recommended operating instructions of the instrument.

In another embodiment, the disclosure is directed to a method for manufacturing a combination of at least two types of liposomes, wherein a first type of liposomes comprises a saponin, a sterol, and a phospholipid and a second type of liposomes comprises a sterol, a phospholipid, and a Toll-like receptor 4 (TLR4) agonist, the method comprising at least a step of mixing the first and second liposomes.

In some embodiments, the method for manufacturing a liposome as disclosed herein, further comprises a step (c) of filtering the liposomes obtained in step (b) and recovering the liposomes having an average diameter lower than 200 nm. In one exemplary embodiment, the liposomes as disclosed herein may have an average diameter ranging from about 80 nm to about 200 nm or ranging from about 120 nm to about 180 nm.

In case of a combination of at least two types of liposomes, the step of filtering may be carried on the liposomes before and/or after the step of mixing the at least two types of liposomes.

In another embodiment, step (c) includes recovering liposomes having an average diameter lower than 175 nm, lower than 150 nm, or of about 100 nm. As such, step (c) of filtering the liposomes obtained in step (b) may be performed on a 0.22 μm pore size membrane.

In one exemplary embodiment, the method for manufacturing liposomes as disclosed herein, further comprises a step (c) of filtering the liposomes obtained in step (b) on a sterilizing filter. A sterilizing filter may have a 0.22 μm pore size membrane. In such embodiment, the method comprises a step of recovering the liposomes having an average diameter compatible with a sterilizing filtration on a 0.22 μm pore size membrane.

When analyzed by electron microscopic examination, a suspension of liposomes obtained as disclosed herein comprises a mixture of unilamellar liposomes, as well as some multilamellar and multivesicular liposomes.

The liposomes as disclosed herein, or obtained according to the methods herein, may further be combined with an antigen. In a combination of at least two types of liposomes, the first or the second or both types of liposomes may contain at least one antigen. The first and second types of liposomes may contain same or different antigens.

Therefore, a method as disclosed herein may comprise a further step of mixing the liposomes obtained after step b) or after step c) with at least one antigen. A suitable antigen may be as disclosed above. The mixing may be done by adding at least one antigen with a suspension of liposomes. The volume and concentration of each antigen and of the suspension of liposomes before mixing are adjusted so as to obtain the desired concentration of each component, e.g., antigen, TLR-4 agonist, QS21 or QS7, cholesterol (or the like), and phospholipids, in the final composition.

Alternatively, an antigen may be added at one of the steps a) or b) of the disclosed methods, provided that does not alter the nature and function of the antigen.

The antigens may be provided in liquid, semi-liquid, e.g., a gel, or a solid, e.g. a powder, form. In one exemplary embodiment, an antigen is added to the liposomes in a liquid form, as a solution.

The methods may further comprise steps of purification, filtration, and/or sterilization as usually practiced in the field. The obtained composition may be packaged in vials or syringe for further storage and use.

In a combination of liposomes as disclosed herein, the content of the different components, i.e., TLR4-agonist, saponin, sterol or sterol ester, and phospholipid may be expressed per type of liposomes or per the combination of liposomes, or per the composition comprising the liposomes. In some embodiments, the contents of the different components, i.e., TLR4-agonist, saponin, sterol or sterol ester, and phospholipid, are expressed per the combination of liposomes or per the composition comprising the liposomes. For example, in a combination of liposomes as disclosed herein, when the amount of a given component is expressed in weight/volume, that refers to the total amount of this component in the combination of liposomes per volume unit of composition containing this combination. As other example, in a combination of liposomes as disclosed herein, when the amounts of the given components are expressed in a weight:weight ratio, that refers to the amount of each component in the first and second types of liposomes.

In a combination of liposomes as disclosed herein, the contents of sterol and phospholipids in the different types of liposomes, e.g., the first and second types of liposomes, may be identical or different. In some embodiments, the contents of sterol phospholipids in the different types of liposomes, e.g., the first and second types of liposomes, are identical.

In one embodiment, liposomes adjuvant as disclosed herein, i.e., single type of liposomes or a combination of at least two types of liposomes, may comprise:
a weight:weight ratio of TLR4 agonist:saponin ranging from about 1:1 to about 1:500, from about 1:1 to about 1:400, ranging from about 1:2 to about 1:200, ranging from about 1:2.5 to about 1:100, ranging from about 1:2.5 to about 1:90, ranging from about 1:3 to about 1:40, ranging from about 1:3 to about 1:30, or ranging from about 1:5 to about 1:25, or ranging from about 1:5 to about 1:10,
a weight:weight ratio of saponin:sterol ranging from 1:100 to 1:1, ranging from 1:50 to 1:2, or ranging from 1:10 to 1:5, of about 1:2, or of about 1:5,
a weight:weight ratio of sterol:phospholipid ranging from 100:1 to 1:200, ranging from 50:1 to 1:100, ranging from 10:1 to 20:1, of about 1:1, of about 1:2, or of about 1:4.

In one embodiment, liposomes adjuvant as disclosed herein, i.e., single type of liposomes or a combination of at least two types of liposomes, may comprise:
a weight:weight ratio TLR4 agonist:saponin ranging from about 1:1 to about 1:500, from about 1:1 to about 1:400, ranging from about 1:2 to about 1:200, ranging from about 1:2.5 to about 1:100, ranging from about 1:2.5 to about 1:90, ranging from about 1:3 to about 1:40, ranging from about 1:3 to about 1:30,or ranging from about 1:5 to about 1:25, or ranging from about 1:5 to about 1:10,
a weight:weight ratio of saponin:sterol ranging from 1:100 to 1:1, ranging from 1:50 to 1:2, or ranging from 1:10 to 1:5, of about 1:2, or of about 1:5,
a weight:weight ratio of saponin:phospholipid ranging from 1:400 to 1:4, ranging from 1:200 to 1:8, ranging from 1:100 to 1:10, ranging from 1:50 to 1:10, of about 1:8, or of about 1:20.

In one embodiment, liposomes adjuvant as disclosed herein may comprise:
a weight:weight ratio of E6020:QS21 ranging from about 1:1 to about 1:500, from about 1:1 to about 1:400, ranging from about 1:2 to about 1:200, ranging from about 1:2.5 to about 1:100, ranging from about 1:3 to about 1:40, or ranging from about 1:5 to about 1:25, or ranging from about 1:5 to about 1:10,
a weight:weight ratio of QS21:cholesterol ranging from 1:100 to 1:1, ranging from 1:50 to 1:2, or ranging from 1:10 to 1:5, of about 1:2, or of about 1:5,
a weight:weight ratio of cholesterol:DOPC ranging from 100:1 to 1:200, ranging from 50:1 to 1:100, ranging from 10:1 to 20:1, of about 1:1, of about 1:2, or of about 1:4.

In one embodiment, liposomes adjuvant as disclosed herein may comprise:
a weight:weight ratio of E6020:QS21 ranging from about 1:1 to about 1:500, from about 1:1 to about 1:400, ranging from about 1:2 to about 1:200, ranging from about 1:2.5 to about 1:100, ranging from about 1:3 to about 1:40, or ranging from about 1:5 to about 1:25, or ranging from about 1:5 to about 1:10,
a weight:weight ratio of QS21:cholesterol ranging from 1:100 to 1:1, ranging from 1:50 to 1:2, or ranging from 1:10 to 1:5, of about 1:2, or of about 1:5,
a weight:weight ratio of QS21:DOPC ranging from 1:400 to 1:4, ranging from 1:200 to 1:8, ranging from 1:100 to 1:10, ranging from 1:50 to 1:10, of about 1:8, or of about 1:20.

In one embodiment, liposomes adjuvant as disclosed herein may comprise:
a weight:weight ratio of E6020:QS7 ranging from about 1:1 to about 1:500, from about 1:1 to about 1:400, ranging from about 1:2 to about 1:200, ranging from about 1:2.5 to about 1:100, ranging from about 1:3 to about 1:90, or ranging from about 1:5 to about 1:30, or ranging from about 1:5 to about 1:10,
a weight:weight ratio of QS7:cholesterol ranging from 1:100 to 1:1, ranging from 1:50 to 1:2, or ranging from 1:10 to 1:5, of about 1:2, or of about 1:5,
a weight:weight ratio of cholesterol:DOPC ranging from 100:1 to 1:200, ranging from 50:1 to 1:100, ranging from 10:1 to 20:1, of about 1:1, of about 1:2, or of about 1:4.

In one embodiment, liposomes adjuvant as disclosed herein may comprise:
a weight:weight ratio of E6020:QS7 ranging from about 1:1 to about 1:500, from about 1:1 to about 1:400, ranging from about 1:2 to about 1:200, ranging from about 1:2.5 to about 1:100, ranging from about 1:3 to about 1:90, or ranging from about 1:5 to about 1:30, or ranging from about 1:5 to about 1:10,
a weight:weight ratio of QS7:cholesterol ranging from 1:100 to 1:1, ranging from 1:50 to 1:2, or ranging from 1:10 to 1:5, of about 1:2, or of about 1:5,
a weight:weight ratio of QS7:DOPC ranging from 1:400 to 1:4, ranging from 1:200 to 1:8, ranging from 1:100 to 1:10, ranging from 1:50 to 1:10, of about 1:8, or of about 1:20.

In one embodiment, liposome adjuvants as disclosed herein may comprise phospholipid/sterol or ester thereof/saponin/TLR-4 agonist as disclosed herein in weight:weight ratio ranging from about 2:0.5:0.05:X mg/ml to about 8:1.5:1.8:X mg/ml with X ranging from 0.001 mg/ml to 0.05 mg/ml.

In one embodiment, liposome adjuvants as disclosed herein may comprise phospholipid/sterol or ester thereof/saponin/TLR-4 agonist as disclosed herein in weight:weight ratio ranging from about 2:0.5:0.05:X mg/ml to about 8:1.5:0.8:X mg/ml with X ranging from 0.001 mg/ml to 0.05 mg/ml.

In one embodiment, liposomes adjuvant as disclosed herein may comprise phospholipid/sterol or ester thereof/saponin/TLR-4 agonist as disclosed herein in weight:weight ratio of 4:1:0.2:X mg/ml with X being 0.004 mg/ml, 0.008 mg/ml, or 0.02 mg/ml.

In one embodiment, liposomes adjuvant as disclosed herein may comprise phospholipid/sterol or ester thereof/saponin/TLR-4 agonist as disclosed herein in weight:weight ratio of 4:1:0.6:X mg/ml with X being 0.004 mg/ml, 0.008 mg/ml, or 0.02 mg/ml.

In one embodiment, liposomes adjuvant as disclosed herein may comprise DOPC/Chol/QS21/E6020 in weight:weight ratio ranging from about 2:0.5:0.05:X mg/ml to about 8:1.5:0.8:X mg/ml with X ranging from 0.001 mg/ml to 0.05 mg/ml.

In one embodiment, liposomes adjuvant as disclosed herein may comprise DOPC/Chol/QS21/E6020 in weight:weight ratio of 4:1:0.2:X mg/ml with X being 0.004 mg/ml, 0.008 mg/ml, or 0.02 mg/ml.

In one embodiment, liposomes adjuvant as disclosed herein may comprise DOPC/Chol/QS7/E6020 in weight:weight ratio ranging from about 2:0.5:0.05:X mg/ml to about 8:1.5:1.8:X mg/ml with X ranging from 0.001 mg/ml to 0.05 mg/ml.

In one embodiment, liposomes adjuvant as disclosed herein may comprise DOPC/Chol/QS7/E6020 in weight:weight ratio of 4:1:0.2:X mg/ml with X being 0.004 mg/ml, 0.008 mg/ml, or 0.02 mg/ml.

In one embodiment, liposomes adjuvant as disclosed herein may comprise DOPC/Chol/QS7/E6020 in weight:weight ratio of 4:1:0.6:X mg/ml with X being 0.004 mg/ml, 0.008 mg/ml, or 0.02 mg/ml.

In one embodiment, liposomes adjuvant as disclosed herein may comprise DOPC/Chol/QS7/E6020 in weight:weight ratio of 4:1:1.8:X mg/ml with X being 0.004 mg/ml, 0.008 mg/ml, or 0.02 mg/ml.

In one embodiment, liposomes adjuvant as disclosed herein may comprise DOPC/Chol/QS21/E6020 in weight:weight ratio of 4:1:0.2:0.020 mg/ml.

In one embodiment, liposomes adjuvant as disclosed herein may comprise DOPC/Chol/QS7/E6020 in weight:weight ratio of 4:1:0.2:0.020 mg/ml.

In one embodiment, liposomes adjuvant as disclosed herein may comprise DOPC/Chol/QS7/E6020 in weight:weight ratio of 4:1:0.6:0.020 mg/ml.

In one embodiment, liposomes adjuvant as disclosed herein may comprise DOPC/Chol/QS7/E6020 in weight:weight ratio of 4:1:1.8:0.020 mg/ml.

In particular, an antigen that may be used in the liposome adjuvants of the embodiments provided above, is a CMV antigen.

The ratios provided in these embodiments may be particularly beneficial in that they allow the liposomes to be endowed with a low reactogenicity and to induce a high and persistent level of neutralizing antibodies against a given antigen, while simultaneously requiring less TLR4 agonist than other known liposomes adjuvant and therefore reducing production costs.

### Compositions Comprising Liposomes

According to some embodiments, the disclosure relates to compositions comprising liposomes, e.g., single type of liposomes, or a combination of at least two types of liposomes as disclosed herein, or liposome-comprising compositions. The liposomes, as referred to in this section, include liposomes as described above and liposomes obtained by the methods of manufacturing a liposome described above, as well as a combination of at least two types of liposomes as disclosed herein or obtained by methods as disclosed herein.

In another embodiment, the disclosure relates to an adjuvant composition comprising at least one liposome, e.g., single type of liposomes, as described herein, or a combination of at least two types of liposomes as disclosed herein.

Said adjuvant composition may further comprise other compounds which are known in the art to have adjuvant properties.

In an embodiment, the disclosure relates to an immunopotentiating agent comprising at least one liposome, e.g., single type of liposomes, as described herein or at least a combination of at least two types of liposomes as disclosed herein. The liposome described herein, e.g., single type of liposomes, or a combination of at least two types of liposomes as disclosed herein, may also be used alone as an immunopotentiating agent.

In an embodiment, a composition comprising liposomes described herein, e.g., single type of liposomes, or a combination of at least two types of liposomes as disclosed herein, may further comprise a buffer solution in which the liposomes are suspended. A buffer solution suitable herein includes aqueous buffered solutions, for example acidic buffers, such as citrate buffer, sodium acetate buffer, histidine buffer, succinate buffer, borate buffer or a phosphate buffer. For example, an aqueous buffer may be a citrate buffered solution or an acetate buffered solution, or else a histidine buffer.

A buffer solution may further comprise a stabilizing agent. Suitable stabilizing agents include carbohydrates, surfactants, polymers such as polyvinylalcohol, amino acids, cyclodextrins, and small molecular weight excipients such as urea.

A composition comprising liposomes described herein, e.g., single type of liposomes, or a combination of at least two types of liposomes as disclosed herein, may be lyophilised. Lyophilisation is a low temperature dehydration process that involves freezing the liposome, lowering pressure, then removing the ice by sublimation. Methods of lyophilization that are suitable for liposomes, and avoid their degradation, are well-known to one skilled in the art. Lyophilised compositions present the advantage of increasing the shelf life of the liposomes.

A composition as disclosed herein may be sterilized. Methods of sterilisation that are suitable for liposomes, and avoid their degradation, are well-known to one skilled in the art. Sterilized compositions are particularly advantageous for administration to individuals.

### Immunogenic Compositions

In another embodiment, the disclosure relates to an immunogenic composition, such as a vaccine composition, comprising at least one liposome described herein, e.g., single type of liposomes, or a combination of at least two types of liposomes as disclosed herein, or a liposome-comprising composition as described herein, or an adjuvant composition as described above, and at least one antigen. The liposomes, as referred to in this section, include liposomes as described above and liposomes obtained by the methods of manufacturing a liposome described above, as well as a combination of at least two types of liposomes as disclosed herein or obtained by methods as disclosed herein.

A vaccine composition is a composition which is used to elicit a protective immune response to a given antigen. A vaccine is usually used as a prevention tool, but may also, in certain cases, be used as a treatment.

The presence of liposomes as disclosed herein in an immunogenic composition, such as a vaccine composition, acts as an adjuvant, by increasing the immune response elicited by the antigen in the composition.

Suitable antigens that may be used in an immunogenic composition, such as a vaccine composition, are described above. In an embodiment, the antigen may be selected from bacterial antigens, protozoan antigens, viral antigens, fungal antigens, parasite antigens and tumour antigens.

Certain aspects of the present disclosure relate to immunogenic compositions comprising a gB antigen, a CMV gH/gL/UL128/UL130/UL131 pentameric complex antigen, as disclosed herein, and an adjuvant comprising at least one liposome comprising a saponin, a sterol, a phospholipid and a Toll-like receptor 4 (TLR4) agonist of formula (I) as described herein. In some embodiments, an immunogenic composition may further comprise a pharmaceutically acceptable carrier. In some embodiments, an immunogenic composition may be useful for preventing and/or treating a CMV infection.

In one aspect, an immunogenic composition as disclosed herein is a subunit immunogenic composition, for example a subunit vaccine composition.

An immunogenic or vaccine composition as disclosed herein may be formulated into preparations in solid, semi-solid, liquid forms, such as tablets, capsules, powders, aerosols, solutions, suspensions, or emulsions. Typical routes of administering such compositions include, without limitation, oral, topical, transdermal, inhalation, parenteral, sublingual, buccal, intranasal. The term parenteral as used herein includes subcutaneous injections, intravenous, intramuscular, intradermal, intrasternal injection or infusion techniques. In some embodiments, a vaccine composition as disclosed herein may be administered by transdermal, subcutaneous, intradermal or intramuscular route. Compositions of the present disclosure are formulated based upon the mode of delivery, including, for example, compositions formulated for delivery via parenteral delivery, such as intramuscular, intradermal, or subcutaneous injection.

An immunogenic composition as disclosed herein may be administered via any suitable route, such as by mucosal administration (e.g., intranasal or sublingual), parenteral administration (e.g., intramuscular, subcutaneous, transcutaneous, or intradermal route), or oral administration. As appreciated by the man skilled in the art, an immunogenic composition may be suitably formulated to be compatible with the intended route of administration. In one embodiment, an immunogenic composition as disclosed herein may be formulated to be administered via the intramuscular route, or the intradermal route, or the subcutaneous route. In one embodiment, an immunogenic composition may be formulated to be administered via the intramuscular route.

Compositions as disclosed herein are formulated so as to allow the active ingredients contained therein to be bioavailable upon administration of the composition to a subject.

Actual methods of preparing such dosage forms are known, or will be apparent, to those skilled in this art; for example, see Remington: The Science and Practice of Pharmacy, 20th Edition (Philadelphia College of Pharmacy and Science, 2000).

Immunogenic compositions as disclosed herein may be formulated with any pharmaceutically acceptable carrier. The compositions may contain at least one inert diluent or carrier. One exemplary pharmaceutically acceptable vehicle is a physiological saline buffer. Other physiologically acceptable vehicles are known to those skilled in the art and are described, for instance, in Remington's Pharmaceutical Sciences (18th edition), ed. A. Gennaro, 1990, Mack Publishing Company, Easton, Pa. An immunogenic composition as described herein may optionally contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, human serum albumin, essential amino acids, nonessential amino acids, L-arginine hydrochlorate, saccharose, D-trehalose dehydrate, sorbitol, tris (hydroxymethyl) aminomethane and/or urea. In addition, the vaccine composition may optionally comprise pharmaceutically acceptable additives including, for example, diluents, binders, stabilizers, and preservatives.

In one embodiment, the composition may be in the form of a liquid, for example, a solution, an emulsion, or a suspension. The liquid may be for delivery by injection. Compositions intended to be administered by injection may contain at least one of: a surfactant, preservative, wetting agent, dispersing agent, suspending agent, buffer, stabilizer, and isotonic agent may be included. The liquid compositions as disclosed herein may include at least one of: sterile diluents such as water for injection, saline solution, such as physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose; agents to act as cryoprotectants such as sucrose or trehalose.

The pH of an immunogenic composition disclosed herein may range from about 5.5 to about 8, for example from about 6.5 to about 7.5, or may be at about 7. Stable pH may be maintained by the use of a buffer. As possible usable buffers, one may cite Tris buffer, citrate buffer, phosphate buffer, Hepes buffer, or histidine buffer. An immunogenic composition as disclosed herein may generally include a buffer. Immunogenic compositions may be isotonic with respect to mammals, such as humans. An immunogenic composition may also comprise one or several additional salts, such as NaCl

The parenteral preparation can be enclosed in ampoules, disposable syringes or multiple dose vials made of glass or plastic. An injectable composition is for example sterile.

Immunogenic compositions as disclosed herein may be sterilized by conventional sterilization techniques, for example with UV or gamma-radiation, or may be sterile filtered. The compositions resulting from sterile filtration of liquid immunogenic compositions as disclosed herein may be packaged and stored in liquid form or lyophilized. A lyophilized composition may be reconstituted with a sterile aqueous carrier prior to administration.

The compositions as disclosed herein may be prepared by methodology well known in the pharmaceutical art. For example, a composition intended to be administered by injection can be prepared by combining the liposomes, a combination of at least two types of liposomes as disclosed herein, or liposome-comprising compositions as disclosed herein with sterile, distilled water or other carrier so as to form a solution. A surfactant may be added to facilitate the formation of a homogeneous solution or suspension.

The compositions as disclosed herein are administered in a therapeutically effective amount, which will vary depending on a variety of factors including the activity of the specific therapeutic agent employed; the metabolic stability and length of action of the therapeutic agent; the age, body weight, general health, sex, and diet of the patient; the mode and time of administration; the rate of excretion; the drug combination; the severity of the specific disorder or condition; and the subject undergoing therapy.

In one embodiment, immunogenic compositions as disclosed herein may be packaged and stored in dry form such as lyophilized compositions or as micropellets obtained via a prilling process as described in WO 2009/109550. In one embodiment the different components of a composition, e.g., the gB antigen, the gH/gL/UL128/UL130/UL131 pentameric complex antigen and the adjuvant, may all be present in the same micropellets. In another embodiment, the components of an immunogenic composition as disclosed herein, e.g., the gB antigen, the gH/gL/UL128/UL130/UL131 pentameric complex antigen and the adjuvant the gB antigen, may each be in distinct micropellets, that is one component per micropellet. In such embodiment, the different micropellets containing separately the different components may be mixed before administration to a subject. In one embodiment, they may be mixed before reconstitution in a liquid carrier. In another embodiment, they may be mixed at the time of reconstitution in liquid carrier by being added in one volume of liquid carrier. In another embodiment, they may be, first, each separately added to distinct volumes of liquid carrier, and second, the different volumes of liquid carrier may be then mixed together to give the final liquid composition to be administered to the subject.

Dry compositions may include stabilizers such as mannitol, sucrose, or dodecyl maltoside, as well as mixtures thereof e.g., lactose/sucrose mixtures, sucrose/mannitol mixtures, etc.

In one embodiment, the adjuvant, and the antigens of an immunogenic composition as disclosed herein may be blended together in a single composition. In such embodiment, an immunogenic composition may be prepared as a ready-to-use mix of the CMV gB antigen, the CMV gH/gL/UL128/UL130/UL131 pentameric complex antigen and the adjuvant.

In one embodiment, the adjuvant and the antigens may be prepared in at least two distinct compositions. The distinct compositions may be then blended together, in an extemporaneous manner, just prior to administration to a patient. In another embodiment, the distinct compositions may be administered separately, that is administered at the same time (in practice only a few seconds or minutes apart, e.g., less than 5 minutes), but via at least two distinct sites of administration, such as at least two distinct sites of injections. In another embodiment, the distinct compositions may be administered sequentially, that is at least two distinct points in time, such as at least 5 minutes apart, or up to hours or 1 or 2 days apart. In such embodiment, the distinct compositions may be administered at the same site of administration, such as the same injection site, or at different sites of administration, such as different injection sites.

In one exemplary embodiment, an immunogenic composition may be prepared extemporaneously, just before administration to a patient. In such embodiment, the different components of a composition as disclosed herein may be provided separately as kit-of-parts. A kit-of-parts as disclosed herein may comprise the different components of an immunogenic composition, each in separate containers, and ready for being mixed.

In an embodiment, the disclosure relates to a kit-of-parts comprising:
a first container comprising a first composition comprising a liposome or an adjuvant composition as disclosed herein, and
a second container comprising a second composition comprising at least one antigen. In such embodiment, a liposome may be a single type of liposomes. An adjuvant composition may comprise a single type of liposome or a combination of at least two types of liposomes.

In another embodiment, the disclosure is directed to a kit-of-parts comprising:
a first container comprising a first composition comprising a first type of liposomes comprising a saponin, a sterol, and a phospholipid,
a second container comprising a second type of liposomes comprising a sterol, a phospholipid, and a Toll-like receptor 4 (TLR4) agonist, and
a third container comprising a third composition comprising at least one antigen.

In one embodiment, at least one of an adjuvant and of an antigen may be in dry form.

In another embodiment, all of the adjuvant and of the antigen may be a dry form in separate containers. In such embodiment, the kit-of-parts may further comprise a container comprising a liquid pharmaceutically carrier for reconstituting in a liquid form the different components of the composition before use.

The containers to be used in a kit-of-parts as disclosed herein may be separate containers, such as vials. In some arrangements, all the components are kept separately until the time of use. The contents of the vials may then be mixed, e.g., by removing the content of one vial and adding it to the other vial, or by separately removing the contents of all the vials and mixing them in a new container.

In one embodiment, a kit-of-parts as disclosed herein may comprise:
a first container comprising a first composition comprising an adjuvant as disclosed herein, and
a second container comprising a second composition comprising at least one gB antigen and at least one CMV gH/gL/UL128/UL130/UL131 pentameric complex antigen as disclosed herein. In such embodiment, a liposome may be a single type of liposomes. An adjuvant composition may comprise a single type of liposome or a combination of at least two types of liposomes.

In another embodiment, the disclosure is directed to a kit-of-parts comprising:
a first container comprising a first composition comprising a first type of liposomes comprising a saponin, a sterol, and a phospholipid,
a second container comprising a second type of liposomes comprising a sterol, a phospholipid, and a Toll-like receptor 4 (TLR4) agonist, and
a third container comprising a third composition comprising at least one gB antigen and at least one CMV gH/gL/UL128/UL130/UL131 pentameric complex antigen as disclosed herein.

In one embodiment the CMV antigens, i.e., the gB antigen and the gH/gL/UL128/UL130/UL131 pentameric complex antigen, may be provided in separate containers. In such embodiment, a kit-of-parts may comprise at least three, four or more containers.

In one embodiment, at least one of an adjuvant and of the CMV antigens, i.e., the gB antigen and the gH/gL/UL128/UL130/UL131 pentameric complex antigen, may be in dry form.

In another embodiment, all of the adjuvant and the CMV antigens, i.e., the gB antigen and the gH/gL/UL128/UL130/UL131 pentameric complex antigen, may be a dry form in separate containers, for example in 2 or 3 containers. In such embodiment, the kit-of-parts may further comprise a container comprising a liquid pharmaceutically carrier for reconstituting in a liquid form the different components of the composition before use.

The containers to be used in a kit-of-parts as disclosed herein may be separate containers, such as vials. In some arrangements, all the components are kept separately until the time of use. For example, the gB antigen and the CMV gH/gL/UL128/UL130/UL131 pentameric complex antigen may be in a same container and the adjuvant may be in another container. The contents of the vials may then be mixed, e.g., by removing the content of one vial and adding it to the other vial, or by separately removing the contents of all the vials and mixing them in a new container.

In one embodiment, at least one container may be a syringe and the other container(s) may be vial(s). The syringe may be used (e.g., with a needle) to insert its contents into another container for mixing, and the mixture can then be withdrawn into the syringe. The mixed contents of the syringe can then be administered to a patient, typically through a new sterile needle.

In another embodiment, the containers of a kit may be separate, contiguous, communicating chambers of a single syringe, such as multi-chambers syringe. In such embodiment, each chamber is in communication with the adjacent chambers, the communication being held close until use. The communication may be open by actuation of the plunger of the syringe which breaks the seals between the chambers, allowing the mixing of the different components. In such embodiment, at least one chamber contains a liquid composition. The other chambers may contain a component either in liquid form or in a dry form such as a lyophilized product or micropellets.

In one exemplary embodiment, an immunogenic composition as disclosed herein may be packaged in a single vial or a single syringe as a ready-to-use mix of the antigen, and the adjuvant.

In one exemplary embodiment, an immunogenic composition as disclosed herein may be packaged in a single vial or a single syringe as a ready-to-use mix of the CMV gB antigen, the CMV gH/gL/UL128/UL130/UL131 pentameric complex antigen, and the adjuvant.

### Uses, Methods of Uses, and Methods of Treatments

The present disclosure further relates to the uses of liposomes, adjuvant compositions, immunopotentiating agents, and immunogenic compositions as described herein. The liposomes, as referred to in this section, include liposomes as described above, e.g., single type of liposomes, and liposomes obtained by the method of manufacturing a liposome described above, as well as a combination of at least two types of liposomes and combinations of liposomes obtained as disclosed herein.

In some embodiments, the disclosure relates to a method for adjuvanting at least one antigen comprising at least a step of combining at least one liposome, e.g., single type of liposomes, or a combination of at least two types of liposomes as disclosed herein, or an adjuvant composition as described herein, with at least one antigen.

In a further embodiment, the disclosure relates to a method for adjuvanting an immunogenic response against at least one antigen in an individual in need thereof, comprising administering to said individual at least one liposome, e.g., single type of liposomes, or a combination of at least two types of liposomes as disclosed herein, or an adjuvant composition as described herein with said antigen.

In another embodiment, the disclosure relates to a method for inducing an immune response against at least one antigen in an individual in need thereof, comprising at least one step of administering to said individual at least one liposome, e.g., single type of liposomes, or a combination of at least two types of liposomes as disclosed herein, or an adjuvant composition as described herein, with said antigen.

Suitable antigens are described above.

In the methods disclosed herein, the liposomes, e.g., single type of liposomes, or combinations of at least two types of liposomes as disclosed herein, or adjuvant compositions and the antigens are administered simultaneously, separately or sequentially.

In an embodiment, the methods enclosed herein further comprise a step of increasing the cytokine and/or chemokine response of the individual in need thereof. In some embodiments, the cytokine and/or chemokine response includes the response of IL-2, IL-4, IL-5, IL-6, IL-8, IL-12, IL-17, IFN-γ, IP-10, MCP-1, MIP-1β, KC and/or TNF-α of the individual in need thereof. In another embodiment, a method enclosed herein comprises a step of increasing the response of IFN-γ, IL-2, IL-4, IL-5 and IL-17 which confers a balanced Th1/Th2 immune response to the individual in need thereof. In another embodiment, a method enclosed herein comprises a step of increasing the response of IL-2, IL-4, IL-5, IL-12, IL-17, IFNγ of the individual in need thereof. By “increasing the cytokine and/or chemokine response” it is meant that the cytokine and/or chemokine response of the individual is higher when compared to the cytokine and/or chemokine response of the individual when the antigen is administered alone, or without the liposome or adjuvant composition.

In one embodiment, an immunogenic composition as disclosed herein, comprising at least one adjuvant as disclosed herein and at least one antigen, is for use in a method for eliciting an immune response against said antigen in a patient receiving said composition, said immune response being a balanced Th1/Th2 immune response.

A Th1 immune response is substantially a cell-mediated immune response. IFN-γ may be used as a biomarker of a Th1 immune response. A Th2 immune response is substantially a humoral-mediated immune response. IL-5 may be a biomarker of a Th2 immune response.

A balanced Th1/Th2 immune response may be an immune response in which the log 10 of the ratio of a number of IFNγ-secreting cells per million of cells to a number of IL-5-secreting cells per million of cells is ranging from about 1 to about 15, preferably from about 2 to about 10, from about 3 to about 8, and is of about 5. IFNγ and IL-5-secreting cells may be measured by ELISPOT as detailed in the Examples section.

The secretion of IL-5 or INFγ may be measured on immune cells, such as spleen cells, obtained from the individual having received an immune composition as disclosed herein.

In some embodiments, the disclosure also relates to a method of preventing and/or treating a disease in an individual in need thereof, wherein the method comprises administering an effective amount of at least one liposome, e.g., single type of liposomes, or a combination of at least two types of liposomes as disclosed herein, at least one adjuvant composition, at least one immunopotentiating agent, or at least one immunogenic composition as described herein, to an individual in need thereof. For example, a liposome, e.g., single type of liposomes, or a combination of at least two types of liposomes as disclosed herein, an adjuvant composition, an immunopotentiating agent or an immunogenic composition as disclosed herein may be for use in a therapeutic method for preventing and/or treating infectious diseases, allergies, autoimmune diseases, rare blood disorders, rare metabolic diseases, rare neurologic diseases, and tumour or cancer diseases.

In some embodiments, the disclosure also relates to a use of at least one liposome, e.g., single type of liposomes, or a combination of at least two types of liposomes as disclosed herein, at least one an adjuvant composition, at least one immunopotentiating agent or at least one immunogenic composition as disclosed herein for the manufacture of a medicament for preventing and/or treating infectious diseases, allergies, autoimmune diseases, rare blood disorders, rare metabolic diseases, rare neurologic diseases, and tumour or cancer diseases. For example, diseases which may be concerned by the disclosure may be infectious diseases such as viral infectious diseases, bacterial infectious diseases, fungal or parasitic infectious diseases. Diseases also concerned by the disclosure may be cancer or tumour diseases.

In some embodiments, the disclosure also relates to at least one liposome, e.g., single type of liposomes, or a combination of at least two types of liposomes as disclosed herein, at least one an adjuvant composition, at least one immunopotentiating agent or at least one immunogenic composition as disclosed herein for their use in the prevention and/or the treatment of an infectious diseases, allergies, autoimmune diseases, rare blood disorders, rare metabolic diseases, rare neurologic diseases, and tumour or cancer diseases.

Viral infectious diseases may be acute febrile pharyngitis, pharyngoconjunctival fever, epidemic keratoconjunctivitis, infantile gastroenteritis, Coxsackie infections, infectious mononucleosis, Burkitt lymphoma, acute hepatitis, chronic hepatitis, hepatic cirrhosis, hepatocellular carcinoma, primary HSV-1 infection (e.g., gingivostomatitis in children, tonsillitis and pharyngitis in adults, keratoconjunctivitis), latent HSV-1 infection (e.g., herpes labialis and cold sores), primary HSV-2 infection, latent HSV-2 infection, aseptic meningitis, infectious mononucleosis, Cytomegalic inclusion disease, Kaposi sarcoma, multicentric Castleman disease, primary effusion lymphoma, AIDS, influenza, Reye syndrome, measles, postinfectious encephalomyelitis, Mumps, hyperplastic epithelial lesions (e.g., common, flat, plantar and anogenital warts, laryngeal papillomas, epidermodysplasia verruciformis), cervical carcinoma, squamous cell carcinomas, croup, pneumonia, bronchiolitis, common cold, Poliomyelitis, Rabies, bronchiolitis, pneumonia, influenza-like syndrome, severe bronchiolitis with pneumonia, German measles, congenital rubella, Varicella, Covid-19, Respiratory Syncytial Virus (RSV) infection, and herpes zoster.

In one embodiment, the disease is influenza, a Respiratory Syncytial Virus (RSV) infection, or Covid-19, and for example is influenza.

In one embodiment, the disease is not a cytomegalovirus infection.

Bacterial infectious diseases may be such as abscesses, actinomycosis, acute prostatitis, Aeromonas hydrophila, annual ryegrass toxicity, anthrax, bacillary peliosis, bacteremia, bacterial gastroenteritis, bacterial meningitis, bacterial pneumonia, bacterial vaginosis, bacterium-related cutaneous conditions, bartonellosis, BCG-oma, botryomycosis, botulism, Brazilian purpuric fever, Brodie abscess, brucellosis, Buruli ulcer, campylobacteriosis, caries, Carrion's disease, cat scratch disease, cellulitis, chlamydia infection, cholera, chronic bacterial prostatitis, chronic recurrent multifocal osteomyelitis, clostridial necrotizing enteritis, combined periodontic-endodontic lesions, contagious bovine pleuropneumonia, diphtheria, diphtheritic stomatitis, ehrlichiosis, erysipelas, piglottitis, erysipelas, Fitz-Hugh-Curtis syndrome, flea-borne spotted fever, foot rot (infectious pododermatitis), Garre's sclerosing osteomyelitis, Gonorrhea, Granuloma inguinale, human granulocytic anaplasmosis, human monocytotropic ehrlichiosis, hundred days' cough, impetigo, late congenital syphilitic oculopathy, legionellosis, Lemierre's syndrome, leprosy (Hansen's Disease), leptospirosis, listeriosis, Lyme disease, lymphadenitis, melioidosis, meningococcal disease, meningococcal septicaemia, methicillin-resistant Staphylococcus aureus (MRS A) infection, Mycobacterium avium-intracellulare (MAI), mycoplasma pneumonia, necrotizing fasciitis, nocardiosis, noma (cancrum oris or gangrenous stomatitis), omphalitis, orbital cellulitis, osteomyelitis, overwhelming post-splenectomy infection (OPSI), ovine brucellosis, pasteurellosis, periorbital cellulitis, pertussis (whooping cough), plague, pneumococcal pneumonia, Pott disease, proctitis, pseudomonas infection, psittacosis, pyaemia, pyomyositis, Q fever, relapsing fever (typhinia), rheumatic fever, Rocky Mountain spotted fever (RMSF), rickettsiosis, salmonellosis, scarlet fever, sepsis, serratia infection, shigellosis, southern tick-associated rash illness, staphylococcal scalded skin syndrome, streptococcal pharyngitis, swimming pool granuloma, swine brucellosis, syphilis, syphilitic aortitis, tetanus, toxic shock syndrome (TSS), trachoma, trench fever, tropical ulcer, tuberculosis, tularemia, typhoid fever, typhus, urogenital tuberculosis, urinary tract infections, vancomycin-resistant Staphylococcus aureus infection, Waterhouse-Friderichsen syndrome, pseudotuberculosis (Yersinia) disease, and yersiniosis.

Parasitic infectious diseases may be amoebiasis, giardiasis, trichomoniasis, African Sleeping Sickness, American Sleeping Sickness, leishmaniasis (Kala-Azar), balantidiasis, toxoplasmosis, malaria, acanthamoeba keratitis, and babesiosis.

Fungal infectious diseases may be aspergilloses, blastomycosis, candidasis, coccidioidomycosis, cryptococcosis, histoplasmosis, mycetomas, paracoccidioidomycosis, and tinea pedis. Furthermore, persons with immuno-deficiencies are susceptible to disease by fungal genera such as Aspergillus, Candida, Cryptoccocus, Histoplasma, and Pneumocystis. Other fungi can attack eyes, nails, hair, and especially skin, the so-called dermatophytic fungi and keratinophilic fungi, and cause a variety of conditions, of which ringworms such as athlete's foot are common. Fungal spores are also a major cause of allergies, and a wide range of fungi from different taxonomic groups can evoke allergic reactions in some people.

Cancer or tumour diseases may be cancer or tumor diseases are for example selected from melanomas, malignant melanomas, colon carcinomas, lymphomas, sarcomas, blastomas, renal carcinomas, gastrointestinal tumors, gliomas, prostate tumors, bladder cancer, rectal tumors, stomach cancer, oesophageal cancer, pancreatic cancer, liver cancer, mammary carcinomas (=breast cancer), uterine cancer, cervical cancer, acute myeloid leukaemia (AML), acute lymphoid leukaemia (ALL), chronic myeloid leukaemia (CML), chronic lymphocytic leukaemia (CLL), hepatomas, various virus-induced tumors such as, for example, papilloma virus-induced carcinomas (e.g. cervical carcinoma=cervical cancer), adenocarcinomas, herpes virus-induced tumors (e.g. Burkitt's lymphoma, EBV-induced B-cell lymphoma), heptatitis B-induced tumors (hepatocell carcinomas), HTLV-1- and HTLV-2-induced lymphomas, acoustic neuroma, lung carcinomas (=lung cancer=bronchial carcinoma), small-cell lung carcinomas, pharyngeal cancer, anal carcinoma, glioblastoma, rectal carcinoma, astrocytoma, brain tumors, retinoblastoma, basalioma, brain metastases, medulloblastomas, vaginal cancer, pancreatic cancer, testicular cancer, Hodgkin's syndrome, meningiomas, Schneeberger disease, hypophysis tumor, Mycosis fungoides, carcinoids, neurinoma, spinalioma, Burkitt's lymphoma, laryngeal cancer, renal cancer, thymoma, corpus carcinoma, bone cancer, non-Hodgkin's lymphomas, urethral cancer, CUP syndrome, head/neck tumors, oligodendroglioma, vulval cancer, intestinal cancer, colon carcinoma, oesophageal carcinoma (=oesophageal cancer), wart involvement, tumors of the small intestine, craniopharyngeomas, ovarian carcinoma, genital tumors, ovarian cancer (=ovarian carcinoma), pancreatic carcinoma (=pancreatic cancer), endometrial carcinoma, liver metastases, penile cancer, tongue cancer, gall bladder cancer, leukaemia, plasmocytoma, lid tumor, prostate cancer (=prostate tumors).

Diseases for which the present disclosure can be useful as a therapeutic intervention include diseases such as SMN1-related spinal muscular atrophy (SMA); amyotrophic lateral sclerosis (ALS); GALT-related galactosemia; Cystic Fibrosis (CF); SLC3A1-related disorders including cystinuria; COL4A5-related disorders including Alport syndrome; galactocerebrosidase deficiencies; X-linked adrenoleukodystrophy and adrenomyeloneuropathy; Friedreich's ataxia; Pelizaeus-Merzbacher disease; TSC1 and TSC2-related tuberous sclerosis; Sanfilippo B syndrome (MPS IIIB); CTNS-related cystinosis; the FMR1-related disorders which include Fragile X syndrome, Fragile X-Associated Tremor/Ataxia Syndrome and Fragile X Premature Ovarian Failure Syndrome; Prader-Willi syndrome; hereditary hemorrhagic telangiectasia (AT); Niemann-Pick disease Type C1; the neuronal ceroid lipofuscinoses-related diseases including Juvenile Neuronal Ceroid Lipofuscinosis (JNCL), Juvenile Batten disease, Santavuori-Haltia disease, Jansky-Bielschowsky disease, and PTT-1 and TPP1 deficiencies; EIF2B1, EIF2B2, EIF2B3, EIF2B4 and EIF2B5-related childhood ataxia with central nervous system hypomyelination/vanishing white matter; CACNA1A and CACNB4-related Episodic Ataxia Type 2; the MECP2-related disorders including Classic Rett Syndrome, MECP2-related Severe Neonatal Encephalopathy and PPM-X Syndrome; CDKL5-related Atypical Rett Syndrome; Kennedy's disease (SBMA); Notch-3 related cerebral autosomal dominant arteriopathy with subcortical infarcts and leukoencephalopathy (CADASIL); SCN1A and SCN1B-related seizure disorders; the Polymerase G-related disorders which include Alpers-Huttenlocher syndrome, POLG-related sensory ataxic neuropathy, dysarthria, and ophthalmoparesis, and autosomal dominant and recessive progressive external ophthalmoplegia with mitochondrial DNA deletions; X-Linked adrenal hypoplasia; X-linked agammaglobulinemia; Fabry disease; and Wilson's disease.

According to one embodiment, a composition of the present disclosure may be administered in dosages sufficient to induce an immune response against the CMV antigens present in the composition. The CMV antigens and the adjuvant are administered in an immunologically active amount.

Usually, for human subjects, a dose of an immunogenic, or vaccine, composition to be administered may have a volume in a range of 0.2 to 1 mL, for example of 0.4 to 0.8 mL. In one exemplary embodiment a dose may be of 0.5 mL.

An immunogenic, or a vaccine, composition may be provided as a single composition or as a kit-of-parts comprising at least two containers, a first one containing the CMV antigens, as for example the CMV gB antigen and the CMV gH/gL/UL128/UL130/UL131 pentameric complex antigen formulated as liquid formulation, and a second one containing the adjuvant composition as a liquid formulation, the content of both containers may be mixed volume to volume before use.

In some embodiment, a kit-of-parts may comprising at least three containers, a first one containing the CMV antigens, as for example the CMV gB antigen and the CMV gH/gL/UL128/UL130/UL131 pentameric complex antigen formulated as liquid formulation, a second one containing a first type of liposomes as disclosed herein as a liquid formulation, a third one containing a second type of liposomes as disclosed herein as a liquid formulation, the content of the three containers may be mixed volume to volume before use.

The amount of CMV antigens and adjuvant to be administered to a subject may vary depending upon various factors well known to the skilled person, such as the age, size, weight, gender, symptoms, or conditions of the subject, as well as the route of administration, and the like. As example, a dose may be calculated according to body weight or body surface area.

According to another embodiment, an immunogenic composition as disclosed herein may be for use as a CMV vaccine, such as a HCMV vaccine.

An immunogenic or a vaccine composition as disclosed herein may be administered by any route commonly used for administering immunogenic or vaccine composition. A regimen leading to the induction of the expected immune response will be used. Usually, an immunization schedule may include several administrations. The amount of the immunogenic composition administered is enough to produce the desired immune response and may be determined by a skilled person.

According to another embodiment, an immunogenic composition as disclosed herein may be for in use in a method, as a medicament, for inducing neutralizing antibodies against a CMV, such as HCMV. The induced neutralizing antibodies may neutralize CMV, such as HCMV. Neutralization of CMV may prevent a CMV disease or infection, or reduce a risk of occurrence of a CMV disease or infection, or may reduce symptoms of a CMV disease. A method as disclosed herein may comprise administering to a subject at least a first and a second dose of said composition, the at least first and second doses being administered at least one week-apart, for example at least one or two month-apart. In a method as disclosed herein a second dose may induce to the subject less reactogenicity than a first dose, the reactogenicity being measured with a method comprising at least the steps of (a) dosing at least a biomarker selected among CRP, globulin and fibrinogen (i) in a first blood sample taken from said subject having been administered with said first dose of said composition and before being administered with said second dose of said composition to obtain a first measure of said biomarker, and (ii) in a second blood sample taken from said subject having been administered with said second dose of said composition to obtain a second measure of said biomarker, and (b) comparing said first measure with said second measure wherein said comparison is informative as to the reactogenicity elicited by said administered composition.

In one embodiment, a method as disclosed herein may comprise an administration of a third dose. A third dose may be administered at least 4, 5, 6 or 7-months apart from the first dose. In one embodiment, a third dose may be administered 6-months apart from the first dose.

In one embodiment, a method as disclosed may comprise an administration of a first dose and of a second dose one or two-months apart from the first dose. In another embodiment, a method as disclosed may comprise an administration of a first dose, of a second dose one or two-months apart from the first dose, and a third dose six-months apart from the first dose.

According to another embodiment, the disclosure relates to a method, as a medicament, for inducing an immune response against a CMV, such as HCMV, in a subject. The induced immune response may prevent a CMV disease or infection, or may reduce a risk of occurrence of a CMV disease or infection, or may reduce symptoms of a CMV disease or infection. A method as disclosed herein may comprise at least one step of administering to a subject at least one immunogenic, or vaccine, composition as disclosed herein.

A CMV infection or disease to be prevented, or for which the likelihood of occurrence is to be reduced, may be a CMV infection in a woman of child-bearing age, a CMV infection during pregnancy, a CMV congenital infection in an infant, or a CMV infection in subject to be subjected to an organ transplant, such as a solid-organ transplant or a bone-marrow transplant.

In one embodiment, a method as disclosed herein is for preventing a CMV disease or infection, such as an HCMV disease or infection, in a subject receiving a composition as disclosed herein.

In one embodiment, a method as disclosed herein may comprise administering to said subject at least a first and a second doses of said composition, at least one week-apart, for example at least one or two month-apart, wherein the second dose induces less reactogenicity than the first dose, the reactogenicity being measured with a method comprising at least the steps of (a) dosing at least a biomarker selected among CRP, globulin and fibrinogen (i) in a first blood sample taken from said subject having been administered with said first dose of said composition and before being administered with said second dose of said composition to obtain a first measured amount of said biomarker, and (ii) in a second blood sample taken from said subject having been administered with said second dose of said composition to obtain a second measured amount of said biomarker, and (b) comparing said first measured amount with said second measured amount wherein said comparison is informative as to the reactogenicity elicited by said administered composition.

In some embodiments, an increased measured amount of at least biomarker in the second measure compared to the first measure may be indicative of a reactogenic composition.

In some embodiments, an absence of increased measured amount of at least biomarker in the second measure compared to the first measure may be indicative of a no or reduced reactogenic composition.

An immunogenic composition as disclosed herein, such as a vaccine composition, may increase neutralizing antibody levels and/or neutralizing antibody persistence in a subject to whom is administer such composition.

In one embodiment, the disclosure relates to a method for preventing, or for reducing the likelihood of occurrence, of a CMV infection or disease in a subject. Such a method may comprise a step of administering of an immunologically effective amount of an immunogenic composition, or vaccine composition, as disclosed herein.

An immunogenic composition of the invention, for example a vaccine composition, may be administered to a subject in a schedule of administration comprising an administration of at least a first and a second dose of the composition. A schedule of administration may comprise 2 or 3 doses, successively administered to a subject in time. Time between 2 successive doses may range from 1 to 2, 3, 4, 5, 6, 7, 8, 9, 10, 11, 12-months, or more.

The first and second doses may be separated by at least about one month, for example about two months, about three months, about four months, about five, about six, about seven or about eight months. In exemplary embodiments, the first and second dose may be separated by at least about one month, two months, or about three months, or about four months. In one exemplary embodiment, the first and second doses are one-month apart.

In one embodiment, the second dose may be followed by further subsequent doses, for example at least one, at least two or at least four subsequent doses. The interval of time separating each subsequent dose(s) may be identical to the period of time separating the first and second dose. In another of embodiment, the period of time separating the subsequent dose(s) may be different. In one embodiment, each period of time separating the subsequent doses may differ from each other. A period of time separating the subsequent doses from each other may range from about 1 to about 8 months, from about 2 about 4 months or may be about 3 months. In one embodiment, a period of time separating a first dose and a third dose may be of about 4 to about 8-months, for example of about 6-months.

In an exemplary embodiment, an immunogenic, or vaccine, composition as disclosed herein may be administered in two or three doses. In one embodiment where a composition may be administered in three doses, the first dose and the third dose may be administered about 4 to about 8-months apart, for example about 6-months apart. For example, a composition may be administered with a first, a second and a third dose. In such embodiment, the second dose may be administered about one to about three months after the first dose, for example at about 1-month or 1 and half-month after the 1^{st} dose, and the third dose may be administered about 4 to 8-months, for example about 6-months, after the first dose.

In another embodiment, a composition as disclosed herein be administered in a single dose.

A vaccine according to the present invention may be administered in two doses. Preferably, the first dose and the second dose are administered approximately about one, two, three, six, eight or nine months apart. In one exemplary embodiment, the first and second doses may be administered two-months apart.

An immunogenic composition as disclosed herein may be administered to any subject in need thereof. As example of subjects concerned by such compositions one may cite: infants, children, teenagers, young adults, adults, or elderly. In one embodiment, a subject may be a new-born child or a woman of child-bearing age. In another embodiment, a subject may be a subject to be subject to an organ transplant, such as solid-organ, bone-marrow, or stem-cells transplant. In an exemplary embodiment, a subject may be a woman of child-bearing age (16-45y) or an adolescent girl (11-15 years).

A composition as disclosed herein may be administered alone, or concomitantly with other immunogenic, or vaccine, compositions. Such compositions may be directed against Bordetella pertussis, Corynebacterium diptheriae, Clostridium tetani, Mycobacterium tuberculosis, Plasmodium spp., Bacillus anthracis, Vibrio cholera, Salmonella typhi, Borrelia spp., Streptococcus pneumoniae, Staphylococcus aureus, Escherichia coli, Clostridium spp., Mycobacterium leprae, Yersinia pestis, influenza virus, varicella zoster virus, human immunodeficiency virus (HIV), respiratory syncytial virus (RSV), SARS-Cov-2 virus, polio virus, variola virus, rabies virus, rotavirus, human papillomavirus, Ebola virus, hepatitis A virus, hepatitis B virus, hepatitis C virus, lyssavirus, measles virus, mumps virus, and Rubella virus.

It is to be understood that the disclosure encompasses all variations, combinations, and permutations in which at least one limitation, element, clauss, descriptive term, etc., from at least one of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists, e.g., in Markush group or similar format, it is to be understood that each subgroup of the elements is also disclosed, and any element(s) can be removed from the group. It should be understood that, in general, where the disclosure, or aspects of the disclosure, is/are referred to as comprising particular elements, features, etc., they also encompass embodiments consisting, or consisting essentially of, such elements, features, etc. For purposes of simplicity those embodiments have not in every case been specifically set forth in so many words herein. It should also be understood that any embodiment or aspect of the disclosure can be explicitly excluded from the claims, regardless of whether the specific exclusion is recited in the specification. The publications and other reference materials referenced herein to describe the background of the disclosure and to provide additional detail regarding its practice are hereby incorporated by reference.

The sequences disclosed in the present specification serve as references. The same sequences are also presented in a sequence listing formatted according to standard requirements for the purpose of patent matters. In case of any sequence discrepancy with the standard sequence listing, the sequences described in the present specification shall be the reference.

Without limiting the present disclosure, a number of embodiments of the present disclosure are described below for the purpose of illustration.

The embodiments of the invention are further detailed in the following items.

According to a first item, the disclosure relates to a liposome comprising a saponin, a sterol, a phospholipid and a Toll-like receptor 4 (TLR4) agonist (such as a single type of liposome), or
a combination of liposomes comprising at least two types of liposomes, wherein a first type of liposome comprises a saponin, a sterol, and a phospholipid and a second type of liposome comprises a sterol, a phospholipid, and a Toll-like receptor 4 (TLR4) agonist,
wherein the Toll-like receptor 4 (TLR4) agonist is of formula (I):

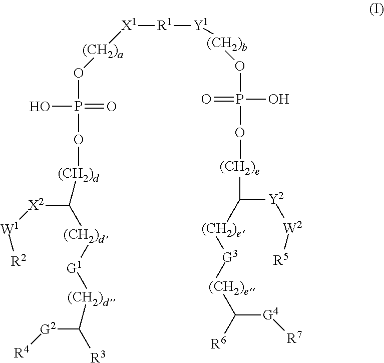

wherein R¹ is selected from the group consisting of:
   a) C(O);
   b) C(O)—(C₁-C₁₄ alkyl)-C(O), in which said C₁-C₁₄ alkyl is optionally substituted with a hydroxyl, a C₁-C₅ alkoxy, a C₁-C₅ alkylenedioxy, a (C₁-C₅ alkyl)amino or a (C₁-C₅ alkyl)aryl, in which said aryl moiety of said (C₁-C₅ alkyl)aryl is optionally substituted with a C₁-C₅ alkoxy, a (C₁-C₅ alkyl)amino, a (C₁-C₅ alkoxy)amino, a (C₁-C₅ alkyl)-amino(C₁-C₅ alkoxy), —O—(C₁-C₅ alkyl)amino(C₁-C₅ alkoxy), —O—(C₁-C₅ alkyl)amino-C(O)—(C₁-C₅ alkyl)-C(O)OH, or —O—(C₁-C₅ alkyl)amino-C(O)—(C₁-C₅ alkyl)-C(O)—(C₁-C₅)alkyl;
   c) an alkyl comprising a C₂-C₁₅ linear or branched chain, optionally substituted with a hydroxyl or an alkoxy; and
   d) —C(O)—(C₆-C₁₂ arylene)-C(O)— in which said arylene is optionally substituted with a hydroxyl, a halogen, a nitro or an amino;
a and b are independently 0, 1, 2, 3 or 4;
d, d′, d″, e, e′ and e″ are independently 0, 1, 2, 3 or 4;
X₁, X₂, Y₁ and Y₂ are independently selected from the group consisting of null, an oxygen, —NH— and —N(C(O)(C₁-C₄ alkyl))-, and —N(C₁-C₄ alkyl)-;
W₁ and W₂ are independently selected from the group consisting of a carbonyl, a methylene, a sulfone and a sulfoxide;
R² and R⁵ are independently selected from the group consisting of:
   a) a C₂ to C₂₀ straight chain or branched chain alkyl, which is optionally substituted with an oxo, a hydroxyl or an alkoxy;
   b) a C₂ to C₂₀ straight chain or branched chain alkenyl or dialkenyl, which is optionally substituted with an oxo, a hydroxyl or an alkoxy;
   c) a C₂ to C₂₀ straight chain or branched chain alkoxy, which is optionally substituted with an oxo, a hydroxyl or an alkoxy;
   d) —NH—(C₂ to C₂₀ straight chain or branched chain alkyl), in which said alkyl group is optionally substituted with an oxo, a hydroxy or an alkoxy; and
   e)

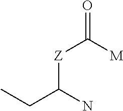

in which Z is selected from the group consisting of an O and NH, and M and N are independently selected from the group consisting of an alkyl, an alkenyl, an alkoxy, an acyloxy, an alkylamino and an acylamino comprising a C₂-C₂₀ linear or branched chain;
R³ and R⁶ are independently selected from the group consisting of a C₂ to C₂₀ straight chain or branched chain alkyl or alkenyl, optionally substituted with an oxo or a fluoro;
R⁴ and R⁷ are independently selected from the group consisting of a C(O)—(C₂ to C₂₀ straight chain or branched chain alkyl or alkenyl), a C₂ to C₂₀ straight chain or branched chain alkyl, a C₂ to C₂₀ straight chain or branched chain alkoxy, and a C₂ to C₂₀ straight chain or branched chain alkenyl; in which said alkyl, alkenyl or alkoxy groups can be independently and optionally substituted with a hydroxyl, a fluoro or a C₁-C₅ alkoxy;
G¹, G², G³ and G⁴ are independently selected from the group consisting of an oxygen, a methylene, an amino, a thiol, —C(O)NH—, —NHC(O)—, and —N(C(O)(C₁-C₄ alkyl))-;
   or G²R⁴ or G⁴R⁷ can together be a hydrogen atom or a hydroxyl;
   or a pharmaceutically acceptable salt of this compound;
   wherein the TLR4 agonist and the saponin are present in a weight:weight ratio of TLR4 agonist:saponin ranging from about 1:50 to about 1:1, or from about 1:35 to about 1:25, or in a weight ratio of TLR4 agonist:saponin of about 1:10.

According to a second item, the disclosure relates to a liposome (e.g., a single type of liposome) or a liposome of a combination of at least two types of liposomes of item 1, wherein the TLR4 agonist has a solubility parameter in ethanol, measured at 25° C., of at least about 0.2 mg/ml.

According to a third item, the disclosure relates to a liposome (e.g., a single type of liposome) or a liposome of a combination of at least two types of liposomes of item 1 or 2, wherein the TLR4 agonist is of formula (II):

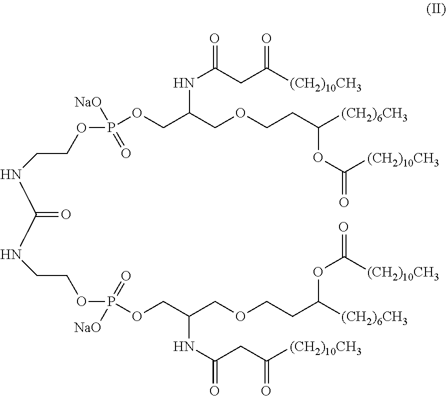

According to a fourth item, the disclosure relates to a liposome (e.g., a single type of liposome) or a liposome of a combination of at least two types of liposomes of any one of items 1 to 3, wherein the TLR4 agonist is E6020 of formula (III):

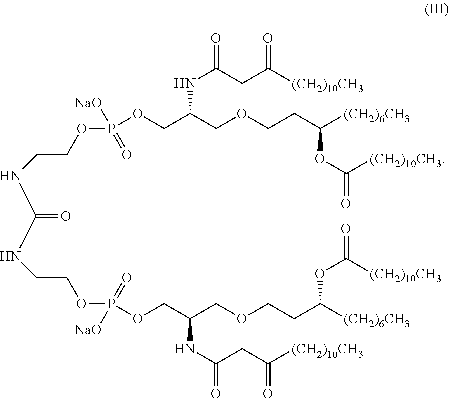

According to a fifth item, the disclosure relates to a liposome (e.g., a single type of liposome) or a liposome of a combination of at least two types of liposomes of any one of items 1 to 4, wherein the saponin is a Quillaja saponaria saponin.

According to a sixth item, the disclosure relates to a liposome (e.g., a single type of liposome) or a liposome of a combination of at least two types of liposomes of any one of items 1 to 5, wherein the saponin is extracted from the bark of Quillaja saponaria Molina.

According to a seventh item, the disclosure relates to a liposome (e.g., a single type of liposome) or a liposome of a combination of at least two types of liposomes of any one of items 1 to 6, wherein the saponin is selected among QS-7, QS-17, QS-18, QS-21, and combinations thereof. In some embodiments, the saponin is QS21 or QS7.

According to an eighth item, the disclosure relates to a liposome (e.g., a single type of liposome) or liposomes of a combination of at least two types of liposomes of any one of items 1 to 7, wherein the sterol is selected from cholesterol or its derivatives, ergosterol, desmosterol (3β-hydroxy-5,24-cholestadiene), stigmasterol (stigmasta-5,22-dien-3-ol), lanosterol (8,24-lanostadien-3b-ol), 7-dehydrocholesterol (Δ5,7-cholesterol), dihydrolanosterol (24,25-dihydrolanosterol), zymosterol (5α-cholesta-8,24-dien-3β-ol), lathosterol (5α-cholest-7-en-3β-ol), diosgenin ((3p,25R)-spirost-5-en-3-ol), sitosterol (22,23-dihydrostigmasterol), sitostanol, campesterol (campest-5-en-3β-ol), campestanol (5a-campestan-3b-ol), 24-methylene cholesterol (5,24(28)-cholestadien-24-methylen-3β-ol), cholesteryl margarate (cholest-5-en-3β-yl heptadecanoate), cholesteryl oleate, cholesteryl stearate, and mixtures thereof.

According to a ninth item, the disclosure relates to a liposome (e.g., a single type of liposome) or liposomes of a combination of at least two types of liposomes of any one of items 1 to 8, wherein the sterol is selected from cholesterol or its derivatives, in particular is cholesterol.

According to a tenth item, the disclosure relates to a liposome (e.g., a single type of liposome) or liposomes of a combination of at least two types of liposomes of any one of items 1 to 9, wherein the saponin and the sterol are present in a weight:weight ratio of saponin:sterol ranging from 1:100 to 1:1, in aweight:weight ratio of saponin:sterol of about 1:2, or in a weight:weight ratio of saponin:sterol of about 1:5.

According to an eleventh item, the disclosure relates to a liposome (e.g., a single type of liposome) or liposomes of a combination of at least two types of liposomes of any one of items 1 to 10, wherein the phospholipid is selected from phosphatidylcholines, phosphatidic acids, phosphatidylethanolamines, phosphatidylglycerols, phosphatidylserines, phosphatidylinositols, and mixtures thereof.

According to a twelfth item, the disclosure relates to a liposome (e.g., a single type of liposome) or liposomes of a combination of at least two types of liposomes of any one of items 1 to 11, wherein the phospholipid is a phosphatidylcholine selected from DSPC (1,2-distearoyl-sn-glycero-3-phosphocholine), DPPC (1,2-dipalmitoyl-sn-glycero-3-phosphocholine), DMPC (1,2-dimyristoyl-sn-glycero-3-phosphocholine), POPC (1-palmitoyl-2-oleoyl-sn-glycero-3-phosphocholine), DOPC (1,2-dioleoyl-sn-glycero-3-phosphocholine), SOPC (1-stearoyl-2-oleoyl-sn-glycero-3-phosphocholine), and mixtures thereof.

According to a thirteenth item, the disclosure relates to a method for manufacturing a liposome comprising at least the steps of:
(a) solubilizing, in an organic water-miscible solvent, a TLR4 agonist of formula (I) having a solubility parameter in ethanol, measured at 25° C., of at least about 0.2 mg/ml, a sterol, and a phospholipid,
(b) processing the mixture obtained at step (a) into a liposome,
   wherein a saponin is added either at step (a), at step (b), or after step b), and
   wherein the TLR4 agonist and the saponin are present in a weight:weight ratio of saponin:TLR4-agonist ranging from about 1:1 to about 400:1, ranging from about 2:1 to about 200:1, ranging from about 2.5:1 to about 100:1, ranging from about 3:1 to about 40:1, or ranging from about 5:1 to about 25:1.

According to a fourteenth item, the disclosure relates to a method of item 13, comprising a step, prior to step (a), of selecting a TLR4 agonist of formula (I) having a solubility parameter in ethanol, measured at 25° C., of at least about 0.2 mg/ml.

According to a fifteenth item, the disclosure relates to a method of any one of items 13 to 14, wherein step (b) of processing the mixture obtained at step (a) into a liposome is carried out by using the solvent injection method.

According to a sixteenth item, the disclosure relates to a method of any one of items 13 to 15, wherein step (b) of processing the mixture obtained at step (a) into a liposome includes the steps of: (b1) injecting and/or diluting the solution obtained at step (a) into an aqueous buffer, and (b2) removing the organic water-miscible solvent.

According to a seventeenth item, the disclosure relates to a method of any one of items 13 to 16, wherein the organic water-miscible solvent is selected from ethanol, isopropanol, or mixtures thereof, or is ethanol.

According to an eighteenth item, the disclosure relates to a method of any one of items 13 to 17, further comprising a step (c) of filtering the liposomes obtained in step (b) and recovering the liposomes having an average diameter lower than 200 nm.

According to a nineteenth item, the disclosure relates to an adjuvant composition comprising at least one liposome or a combination of liposomes of any one of items 1 to 12 or at least one liposome obtained according to the method of any one of items 13 to 18.

According to a twentieth item, the disclosure relates to an immunopotentiating agent comprising at least a liposome (e.g., a single type of liposome) or liposomes of a combination of at least two types of liposomes of any one of items 1 to 12 or at least one liposome obtained according to the method of any one of items 13 to 18.

According to a twenty-first item, the disclosure relates to an immunogenic composition comprising at least a liposome (e.g., a single type of liposome) or liposomes of a combination of at least two types of liposomes of any one of items 1 to 12, or at least one liposome obtained according to the method of any one of items 13 to 18, or an adjuvant composition of item 19, and at least one antigen.

According to a twenty-second item, the disclosure relates to an immunogenic composition of item 21, wherein the antigen is selected from bacterial antigens, protozoan antigens, viral antigens, fungal antigens, parasite antigens and tumour antigens.

According to a twenty-third item, the disclosure relates to a kit-of-parts comprising:
a first container comprising a first composition comprising at least one liposome according to any one of items 1 to 12, or at least one liposome obtained according to the method of any one of items 13 to 18, or an adjuvant composition according to item 19, and
a second container comprising a second composition comprising at least one antigen.

In some embodiments, the disclosure relates to a kit-of-parts comprising:
a first container comprising a first composition comprising at least a first type of liposomes of a combination of liposomes according to any one of item s 1 to 12, and
a second container comprising a second composition comprising at least a second type of liposomes of a combination of liposomes according to any one of item s 1 to 12, and
a third container comprising a third composition comprising at least one antigen.

According to a twenty-fourth item, the disclosure relates to a method for manufacturing an immunogenic composition comprising at least a step of mixing at least a liposome (e.g., a single type of liposome) or liposomes of a combination of at least two types of liposomes of any one of items 1 to 12, or at least one liposome obtained according to the method of any one of items 13 to 18, or an adjuvant composition of item 19, with at least one antigen.

According to a twenty-fifth item, the disclosure relates to a method for adjuvanting at least one antigen comprising at least a step of combining said at least one antigen with at least a liposome (e.g., a single type of liposome) or liposomes of a combination of at least two types of liposomes of any one of items 1 to 12, or at least one liposome obtained according to the method of any one of items 13 to 18, or an adjuvant composition of item 19.

According to a twenty-sixth item, the disclosure relates to a method for adjuvanting an immunogenic response against at least one antigen in an individual in need thereof, comprising administering to said individual said at least one antigen with at least a liposome (e.g., a single type of liposome) or liposomes of a combination of at least two types of liposomes of any one of items 1 to 12, or at least one liposome obtained according to the method of any one of items 13 to 18, or an adjuvant composition of item 19.

According to a twenty-seventh item, the disclosure relates to a method for inducing an immune response against at least one antigen in an individual in need thereof, comprising at least one step of administering to said individual said at least one antigen with at least a liposome (e.g., a single type of liposome) or liposomes of a combination of at least two types of liposomes of any one of items 1 to 12, or at least one liposome obtained according to the method of any one of items 13 to 18, or an adjuvant composition of item 19.

According to a twenty-eighth item, the disclosure relates to a method of item 26 or 27, wherein the liposomes or the adjuvant composition and the antigen are administered simultaneously, separately or sequentially.

According to a twenty-ninth item, the disclosure relates to a method of any one of items 26 to 28, further comprising increasing the cytokine and/chemokine response of said individual.

According to a thirtieth item, the disclosure relates to a method of item 29, comprising an increase of a cytokine and/or chemokine selected among IL-2, IL-4, IL-5, IL-6, IL-8, IL-12, IL-17, IFN-γ, IP-10, MCP-1, MIP-1β, KC and or TNF-α.

According to a thirty-first item, the disclosure relates to an immunogenic composition comprising at least:
one CMV gB antigen;
one CMV gH/gL/UL128/UL130/UL131 pentameric complex antigen; and
one adjuvant comprising at least one liposome comprising a saponin, a sterol, a phospholipid and a Toll-like receptor 4 (TLR4) agonist or at least a combination of liposomes comprising at least two types of liposomes, wherein a first type of liposome comprises a saponin, a sterol, and a phospholipid and a second type of liposome comprises a sterol, a phospholipid, and a Toll-like receptor 4 (TLR4) agonist.

According to a thirty-second item, the present disclosure relates to an immunogenic composition comprising at least:
one CMV gB antigen;
one CMV gH/gL/UL128/UL130/UL131 pentameric complex antigen; and
one adjuvant comprising at least one liposome comprising a saponin, a sterol, a phospholipid and a Toll-like receptor 4 (TLR4) agonist of formula (I), or
   a combination of liposomes comprising at least two types of liposomes, wherein a first type of liposome comprises a saponin, a sterol, and a phospholipid and a second type of liposome comprises a sterol, a phospholipid, and a Toll-like receptor 4 (TLR4) agonist,
wherein the Toll-like receptor 4 (TLR4) agonist is:

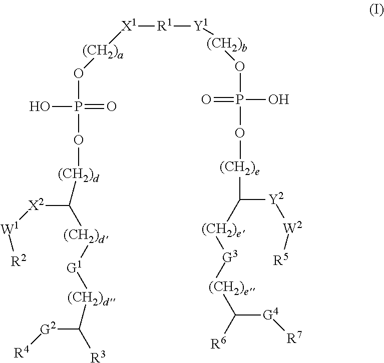

wherein R¹ is selected from the group consisting of:
a) C(O);
b) C(O)—(C₁-C₁₄ alkyl)-C(O), in which said C₁-C₁₄ alkyl is optionally substituted with a hydroxyl, a C₁-C₅ alkoxy, a C₁-C₅ alkylenedioxy, a (C₁-C₅ alkyl)amino or a (C₁-C₅ alkyl)aryl, in which said aryl moiety of said (C₁-C₅ alkyl)aryl is optionally substituted with a C₁-C₅ alkoxy, a (C₁-C₅ alkyl)amino, a (C₁-C₅ alkoxy)amino, a (C₁-C₅ alkyl)-amino(C₁-C₅ alkoxy), —O—(C₁-C₅ alkyl)amino(C₁-C₅ alkoxy), —O—(C₁-C₅ alkyl)amino-C(O)—(C₁-C₅ alkyl)-C(O)OH, or —O—(C₁-C₅ alkyl)amino-C(O)—(C₁-C₅ alkyl)-C(O)—(C₁-C₅)alkyl;

c) an alkyl comprising a C₂-C₁₅ linear or branched chain, optionally substituted with a hydroxyl or an alkoxy; and

d) —C(O)—(C₆-C₁₂ arylene)-C(O)— in which said arylene is optionally substituted with a hydroxyl, a halogen, a nitro or an amino;
a and b are independently 0, 1, 2, 3 or 4;
d, d′, d″, e, e′ and e″ are independently 0, 1, 2, 3 or 4;
X₁, X₂, Y₁ and Y₂ are independently selected from the group consisting of null, an oxygen, —NH— and —N(C(O)(C₁-C₄ alkyl))-, and —N(C₁-C₄ alkyl)-;
W₁ and W₂ are independently selected from the group consisting of a carbonyl, a methylene, a sulfone and a sulfoxide;
R² and R⁵ are independently selected from the group consisting of:
a) a C₂ to C₂₀ straight chain or branched chain alkyl, which is optionally substituted with an oxo, a hydroxyl or an alkoxy;
b) a C₂ to C₂₀ straight chain or branched chain alkenyl or dialkenyl, which is optionally substituted with an oxo, a hydroxyl or an alkoxy;
c) a C₂ to C₂₀ straight chain or branched chain alkoxy, which is optionally substituted with an oxo, a hydroxyl or an alkoxy;
d) —NH—(C₂ to C₂₀ straight chain or branched chain alkyl), in which said alkyl group is optionally substituted with an oxo, a hydroxy or an alkoxy; and
e)

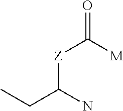

in which Z is selected from the group consisting of an O and NH, and M and N are independently selected from the group consisting of an alkyl, an alkenyl, an alkoxy, an acyloxy, an alkylamino and an acylamino comprising a C₂-C₂₀ linear or branched chain;
R³ and R⁶ are independently selected from the group consisting of a C₂ to C₂₀ straight chain or branched chain alkyl or alkenyl, optionally substituted with an oxo or a fluoro;
R⁴ and R⁷ are independently selected from the group consisting of a C(O)—(C₂ to C₂₀ straight chain or branched chain alkyl or alkenyl), a C₂ to C₂₀ straight chain or branched chain alkyl, a C₂ to C₂₀ straight chain or branched chain alkoxy, and a C₂ to C₂₀ straight chain or branched chain alkenyl; in which said alkyl, alkenyl or alkoxy groups can be independently and optionally substituted with a hydroxyl, a fluoro or a C₁-C₅ alkoxy;
G¹, G², G³ and G⁴ are independently selected from the group consisting of an oxygen, a methylene, an amino, a thiol, —C(O)NH—, —NHC(O)—, and —N(C(O)(C₁-C₄ alkyl))-;
or G²R⁴ or G⁴R⁷ can together be a hydrogen atom or a hydroxyl;
or a pharmaceutically acceptable salt of this compound
wherein the TLR4 agonist and the saponin are present in a weight:weight ratio of TLR4 agonist:saponin ranging from about 1:50 to about 1:1, or from about 1:35 to about 1:25, or in a weight ratio of TLR4 agonist:saponin of about 1:10.

According to a thirty-third item, the immunogenic composition according to item 31 or 32, wherein said CMV gB antigen is selected in a group comprising a full length CMV gB antigen, a truncated CMV gB antigen deleted from at least a part of the transmembrane domain, a truncated CMV gB antigen substantially deleted from all the transmembrane domain, a truncated CMV gB antigen deleted from at least a part of the intracellular domain, a truncated CMV gB antigen substantially deleted from all the intracellular domain, and a truncated CMV gB antigen deleted substantially from both the transmembrane domain and the intracellular domain.

According to a thirty-fourth item, the immunogenic composition according to any one of items 31 to 33, wherein said CMV gB antigen is gBdTm.

According to a thirty-fifth item, the immunogenic composition according to any one of items 31 to 34, wherein said gH is deleted from at least a part of the transmembrane domain or from substantially all the transmembrane domain.

According to a thirty-sixth item, the immunogenic composition according to any one of items 31 to 35, wherein said gH comprises the ectodomain of the full length gH polypeptide encoded by CMV UL75 gene.

According to a thirty-seventh item, the immunogenic composition according to any one of items 31 to 36, wherein the CMV gB antigen and the CMV gH/gL/UL128/UL130/UL131 pentameric complex antigen are the only CMV antigens.

According to a thirty-eighth item, the immunogenic composition according to any one of items 31 to 37, wherein the TLR4 agonist has a solubility parameter in ethanol, measured at 25° C., of at least about 0.2 mg/ml.

According to a thirty-ninth item, the immunogenic composition according to any one of items 31 to 38, wherein the TLR4 agonist is of formula (II):

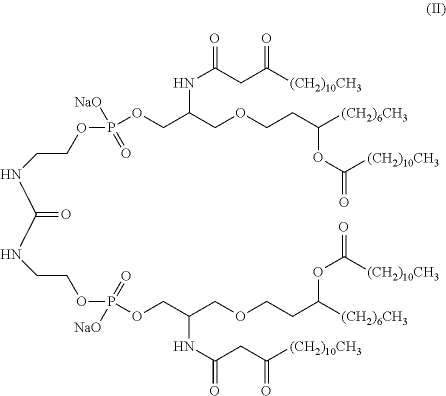

According to a fortieth item, the immunogenic composition according to any one of items 31 to 39, wherein the TLR4 agonist is E6020 of formula (III):

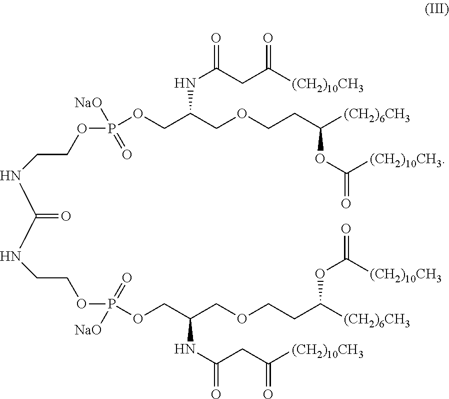

According to a forty-first item, the immunogenic composition according to any one of items 31 to 40, wherein the saponin is a Quillaja saponaria saponin.

According to a forty-second item, the immunogenic composition according to any one of items 31 to 41, wherein the saponin is extracted from the bark of Quillaja saponaria Molina.

According to a forty-third item, the immunogenic composition according to any one of items 31 to 42, wherein the saponin is selected among QS-7, QS-17, QS-18, QS-21, and combinations thereof. In some embodiments, the saponin is QS21 or QS7.

According to a forty-fourth item, the immunogenic composition according to any one of items 31 to 43, wherein the sterol is selected from cholesterol or its derivatives, ergosterol, desmosterol (3β-hydroxy-5,24-cholestadiene), stigmasterol (stigmasta-5,22-dien-3-ol), lanosterol (8,24-lanostadien-3b-ol), 7-dehydrocholesterol (Δ5,7-cholesterol), dihydrolanosterol (24,25-dihydrolanosterol), zymosterol (5α-cholesta-8,24-dien-3β-ol), lathosterol (5α-cholest-7-en-3β-ol), diosgenin ((3β,25R)-spirost-5-en-3-ol), sitosterol (22,23-dihydrostigmasterol), sitostanol, campesterol (campest-5-en-3β-ol), campestanol (5a-campestan-3b-ol), 24-methylene cholesterol (5,24(28)-cholestadien-24-methylen-3β-ol), cholesteryl margarate (cholest-5-en-3β-yl heptadecanoate), cholesteryl oleate, cholesteryl stearate, and mixtures thereof.

According to a forty-fifth item, the immunogenic composition according to any one of items 31 to 44, wherein the sterol is selected from cholesterol or its derivatives, in particular is cholesterol.

According to a forty-sixth item, the immunogenic composition according to any one of items 31 to 45, wherein the saponin and the sterol are present in a weight:weight ratio of saponin:sterol ranging from 1:100 to 1:1, in a weight:weight ratio of saponin:sterol of about 1:2, or in a weight:weight ratio of saponin:sterol of about 1:5.

According to a forty-seventh item, the immunogenic composition according to any one of items 31 to 46, wherein the phospholipid is selected from phosphatidylcholines, phosphatidic acids, phosphatidylethanolamines, phosphatidylglycerols, phosphatidylserines, phosphatidylinositols, and mixtures thereof.

According to a forty-eighth item, the immunogenic composition according to any one of items 31 to 47, wherein the phospholipid is a phosphatidylcholine selected from DSPC (1,2-distearoyl-sn-glycero-3-phosphocholine), DPPC (1,2-dipalmitoyl-sn-glycero-3-phosphocholine), DMPC (1,2-dimyristoyl-sn-glycero-3-phosphocholine), POPC (1-palmitoyl-2-oleoyl-sn-glycero-3-phosphocholine), DOPC (1,2-dioleoyl-sn-glycero-3-phosphocholine), SOPC (1-stearoyl-2-oleoyl-sn-glycero-3-phosphocholine), and mixtures thereof.

According to a forty-ninth item, the present disclosure relates to an immunogenic composition according to any of items 31 to 48, for use as a CMV vaccine.

According to a fiftieth item, the present disclosure relates to an immunogenic composition according to anyone of items 31 to 49, for use in a method for inducing neutralizing antibodies against a CMV, said method comprising administering to a subject at least a first and a second doses of said composition, the at least first and second doses being administered at least one month-apart, wherein the second dose induces to said subject less reactogenicity than the first dose, said reactogenicity being measured with a method comprising at least the steps of (a) dosing at least a biomarker selected among CRP, globulin and fibrinogen (i) in a first blood sample taken from said subject having been administered with said first dose of said composition and before being administered with said second dose of said composition to obtain a first measured amount of said biomarker, and (ii) in a second blood sample taken from said subject having been administered with said second dose of said composition to obtain a second measured amount of said biomarker, and (b) comparing said first measured amount with said second measured amount wherein said comparison is informative as to the reactogenicity elicited by said administered composition. In some embodiments, an increased measured amount of at least biomarker in the second measure compared to the first measure may be indicative of a reactogenic composition. In some embodiments, an absence of increased measured amount of at least biomarker in the second measure compared to the first measure may be indicative of a no or reduced reactogenic composition.

According to a fifty-first item, the present disclosure relates to a kit-of-parts comprising:
a first container comprising a first composition comprising an adjuvant according to any one of items 31 and 38 to 48, and
a second container comprising a second composition comprising at least one CMV gB antigen and at least one CMV gH/gL/UL128/UL130/UL131 pentameric complex antigen according to any one of items 32 to 37.

In some embodiments, the disclosure relates to a kit-of-parts comprising:
a first container comprising a first composition comprising at least a first type of liposomes of a combination of liposomes according to any one of item s 1 to 12, and
a second container comprising a second composition comprising at least a second type of liposomes of a combination of liposomes according to any one of item s 1 to 12, and
a third container comprising a third composition comprising at least one CMV gB antigen and at least one CMV gH/gL/UL128/UL130/UL131 pentameric complex antigen according to any one of items 32 to 37.

According to a fifty-second item, the present disclosure relates to a method for inducing an immune response against a CMV in a subject, comprising at least one step of administering to said subject at least one immunogenic composition according to any one of items 31 to 48.

According to a fifty-third item, the method according to item 52, administering to said subject a first and a second doses of said composition, at least one month-apart, wherein the second dose induces less reactogenicity than the first dose, said reactogenicity being measured with a method comprising at least the steps of (a) dosing at least one biomarker selected among CRP, globulin and fibrinogen (i) in a first blood sample taken from said subject after being administered with said first dose of said composition and before being administered with said second dose of said composition to obtain a first measured amount of said biomarker, and (ii) in a second blood sample taken from said subject after being administered with said second dose of said composition to obtain a second measured amount of said biomarker, and (b) comparing said first measured amount with said second measured amount wherein said comparison is informative as to the reactogenicity elicited by said administered composition. In some embodiments, an increased measured amount of at least biomarker in the second measure compared to the first measure may be indicative of a reactogenic composition. In some embodiments, an absence of increased measured amount of at least biomarker in the second measure compared to the first measure may be indicative of a no or reduced reactogenic composition.

According to a fifty-fourth item, the present disclosure relates to a liposome or a combination of liposomes according to any one of items 1 to 12, a liposome obtained according to the method of any one of items 13 to 18, an immunopotentiating agent of item 19, a adjuvant composition according to item 19, an immunogenic composition of item 21, or an immunogenic composition according to any one of items 31 to 47 for their use in the prevention and/or the treatment of a infectious diseases, allergies, autoimmune diseases, rare blood disorders, rare metabolic diseases, rare neurologic diseases, and tumour or cancer diseases.

### EXAMPLES

### Example 1: Method of Preparation of Liposomes

### I. Materials and Methods

The liposomes were prepared according to the solvent, e.g. ethanol, injection method as follows.

A solution of E6020 in ethanol was prepared at 2 mg/ml by dissolving 2.0 mg of E6020 powder in 0.998 ml of ethanol.

A 4-fold concentrated ethanol solution was prepared at 40 mg/ml of DOPC, 10 mg/ml of cholesterol and 0.200 mg/ml of E6020 by dissolving, in 0.850 ml of ethanol, 40 mg of DOPC and 10 mg of cholesterol and adding 0.100 ml of the previously prepared E6020 solution in ethanol.

The solution was stirred at room temperature (RT) until total dissolution of the product and obtaining a colorless solution.

In a 7 ml Lyo glass vial, 3.0 mL of CBS (Citrate Buffer Solution) pH 6.3 (citrate 10 mM, NaCl 140 mM pH 6.3) were stirred at 1000 rpm at room temperature. 1.0 ml of the lipid solution was slowly added using a Hamilton syringe with a 22 ga needle and a syringe pump at 0.1 ml/min to form liposomes. Liposomes were dialyzed (on 10000 MCWO dialysis cassettes) three times (half a day, one night and one day) against CBS pH 6.3.

Liposomes suspension was sterile filtered on Millex filter PVDF 0.22 μm of 33 mm diameter and stored at +4° C. under nitrogen.

Liposomes components concentrations were estimated according to the dilution factor of the dialysis. For a 1.6 dialysis dilution factor, liposomes components concentrations were at 6.25 mg/ml of DOPC, 1.56 mg/ml of Cholesterol and 0.031 mg/ml of E6020.

Under a flow hood, 3.0 mg of QS21 was re-suspended in 3.0 mL of CBS pH 6.3 to obtain a solution of QS21 at 1.0 mg/ml and sterile filtered on Pall Acrodisc 0.2 μm of 25 mm diameter.

In sterile conditions, SPA14 (liposomes suspension) was formulated by addition of 1.563 ml of the solution of QS21 at 1.0 mg/ml in CBS pH 6.3 to 5.000 ml of the previous liposomal suspension and 1.250 ml of CBS pH 6.3. The mixture was stirred for 10 seconds using a vortex and stored at +4° C. under Nitrogen for a final SPA14 sterile suspension of 4 mg/ml DOPC, 1 mg/ml cholesterol, 0.020 mg/ml E6020 and 0.200 mg/ml QS21.

To do the mixture with antigens, the SPA14 adjuvant was gently turned upside down 5 times to homogenize the product prior mixing with antigen(s) twice concentrated. The immunogenic compositions (adjuvant SPA14+antigen) were then stored at an appropriate temperature (2-8° C.) until further use.

The antigen needed to be prepared 2×C concentrated (e.g. for a dose of 5 μg of antigen under 50 μl injected (C=100 μg/ml), antigen was prepared at 200 μg/ml)

The mixing with the antigen was done volume/volume and the resulting mixture was gently turned upside down 5 times.

The mixtures were prepared just before injection or maximum 3 hours before the injection. In this later case, they had to be placed at 2-8° C. until injection.

Comparative adjuvant AS01B was sampled from the adjuvant vial of Shingrix commercial vaccine.

What is called “vehicle” or “QS21 liposome” throughout the studies in the in vitro MIMIC system (see EXAMPLE 3) was prepared as described for SPA14 without including E6020 in the lipid ethanol solution.

### Example 2: Alcohol Solubility of E6020

### I. Materials and Methods

E6020 (E6020 Eisai) and MPL powder (from Salmonella Minnesota Re 595, Sigma L6895) were solubilized at 0.5, 1.0, 2.0 and 10 mg/ml in absolute ethanol (EtOH) (Carlo Erba). 1 ml of each solution was mixed for 3h at room temperature (about 25° C.).

E6020 solutions were clear but MPL solutions were opalescent, with opalescence increasing with the concentration. The appearance and increase of opalescence, indicative of insolubility was followed by nephelometry.

Nephelometry was performed on a BMG-Labtech Nephelostar with 0.200 ml samples on a UV 96-well microplate (Thermo UV Flat Bottom 96 Ref 8404) with an absolute ethanol blank. RNU (Relative nephelometry Unit) of each solution was recorded and plotted on a graph.

### II. Results

E6020 ethanol solution was perfectly clear up to a concentration of at least 10 mg/ml, whereas MPL ethanol solution was opalescent even at the lowest concentration tested, with opalescence increasing with MPL concentration. (FIG. 1).

As shown by the data, a TLR4 agonist suitable for the present disclosure, such as E6020, had a solubility of at least 10 mg/ml. Such degree of solubility makes the TLR4 agonist advantageous for a use with the ethanol injection process for the manufacturing of liposomes.

The very low solubility in ethanol of MPL makes it non compatible with such liposomes manufacturing process.

### Example 3: Immunopotentiating Effect of E6020-QS21-Containing Liposomes

In this Example, the innate immune profile of SPA14 was assessed using the innate arm of the MIMIC system. The MIMIC system (modular immune in vitro construct) is an artificial system imitating the human immune system. This module, termed the peripheral tissue equivalent (PTE) construct, is a three-dimensional tissue-engineered endothelial cell/collagen matrix culture system that has been previously used to study TLR agonists and vaccines (Ma Y et al., Immunology, 2010, 130: 374-87). Application of TLR agonists to the PTE module not only induces cytokine and chemokine production that can be evaluated by multiplex bead-based array but also promotes dendritic cells (DC) differentiation and maturation that can be examined by flow cytometric analysis (Drake et al., Disruptive Science and Technology, 2012, 1: 28-40; Higbee et al., Altern Lab Anim, 2009, 37 Suppl 1: 19-27). For this analysis, antigen-presenting cells (APC) activation, and cytokine/chemokine profiles were evaluated in cultures left untreated or treated with various doses of SPA14 or QS21-liposomes.

### I. Materials and Methods

### 1. Liposomes Preparation

SPA14 and QS21-liposomes were prepared according to the protocole described in Example 1.

SPA14-20: a liposomal formulation composed of DOPC/Chol/QS21/E6020 (2:0.5:0.1:0.01 mg/ml after dilution ½ with PBS)

SPA14-8: a liposomal formulation composed of DOPC/Chol/QS21/E6020 (2:0.5:0.1:0.004 mg/ml after dilution ½ with PBS)

QS21 liposome (SPA14-0): a liposomal formulation composed of DOPC/Chol/QS21/(2:0.5:0.1 mg/ml after dilution ½ with PBS)

This study, which mainly evaluates the ability of SPA14 to stimulate human immune cells, was designed to test two concentrations of E6020 in SPA14, i.e. 8 and 20 μg/mL, keeping constant all other SPA14 ingredients.

Next, the test items were diluted 1:40-1:4000 in a 10-fold dose curve or 1:20-1:160 in a 2-fold dose curve. To understand the contribution of the QS21 liposome to the innate immune signature induced by SPA14, QS21 liposome (minus any TLR agonist) was also examined in the assay using the same dose scheme as described above.

E6020 (EISAI) (Ishizaka et al., Expert review of vaccines, 2007, 6: 773-84; WO2007005583A1), the TLR-4 agonist in SPA14, was also dosed alone into the assays at the highest concentration of each dose range.

### 2. PBMC Preparation

Apheresis blood products were collected from donors at the OneBlood (Orlando, FL) blood bank. The study protocol and donor program were reviewed and approved by Chesapeake Research Review, Inc. (Columbia, MD). At the time of collection, peripheral blood mononuclear cells (PBMCs) from healthy donors were enriched by Ficoll density gradient separation and cryopreserved in DMSO-containing freezing media as taught in Ma Y. et al. (Assessing the immunopotency of Toll-like receptor agonists in an in vitro tissue-engineered immunological model. Immunology 130: 374-87, 2010).

### 3. MIMIC® PTE Assay

The MIMIC® PTE construct was assembled on a robotic line using the method taught in Ma Y. et al. mentioned above.

Briefly, endothelial cells were grown to confluence atop a collagen matrix (Advanced Biomatrix, San Diego, CA). Thereafter, donor PBMCs prepared from frozen stocks were applied to the assay wells. After an incubation of 90 minutes, non-migrated cells were removed by washing and the test items were added to the cultures at different concentrations, as described above.

A mixture of 100 ng/mL LPS (from Pseudomonas aeruginosa, Cat #L8643, Millipore Sigma, Burlington, MA), and 10 μg/mL R848 (Cat #TLRL-R848, InvivoGen, San Diego, CA) was used as a positive control in these assays (L+R). No Ag/Mock (M—Mock), the negative assay control, was set with culture media without any treatment added.

The culture supernatants were harvested after a 48-hr treatment period and analyzed for cytokines/chemokines by a multiplex assay and PGE2 secretion by ELISA. Cells, harvested at the same time point, were phenotyped for cell viability and APC activation using flow cytometry.

### 4. Cytokine/Chemokine Analysis

MIMIC® culture supernatants were analyzed using the Milliplex® human 12-plex multi-cytokine detection system (Millipore). The kit includes IFN-α2, IFNγ, IL-1β, IL-6, IL-8, IL-10, IL-12p40, IP-10, MCP-1, MIP-1β, RANTES, and TNFα. Analyte concentrations were calculated based on relevant standard curves using the Bio-Plex manager software (Luna et al. PloS one vol. 13,6 e0197478. 6 Jun. 2018, doi:10.1371/journal.pone.0197478).

For run acceptance criteria, the lower limit of quantification (LLOQ) and the upper limit of quantification (ULOQ) for each analyte was established based on the percent recovery (Observed/Expected*100) of each point against a 5-parameter logistic (5PL) curve fit of the standard values. A recovery percentage of 80%-120% was considered acceptable, such that values falling within this range define the lower and upper bounds of the standard curve. The raw data file was reviewed for bead counts; a data point was considered valid when a minimum of 35 beads per region was counted.

### 5. Flow Cytometry

Flow cytometry staining and acquisition was performed as taught in Luna et al. mentioned above.

Briefly, MIMIC PTE-derived cells were washed with PBS and labeled with Live-Dead Aqua (InvitroGen, Carlsbad, CA) for 20 min on ice. After washing and performing an IgG-Fc block (Normal mouse serum; Cat #015-000-120, Jackson ImmunoResearch Laboratories), the cells were incubated with a cocktail of fluorochrome-labeled mAbs, such as anti-CD14, anti-HLA-DR, anti-CD11c, anti-CD86, anti-CD25, anti-CD83, anti-CD3 and anti-CD19, that are specific for non-myeloid lineage cells and immune ligands (BD Biosciences, San Jose, CA). Thereafter, the cells were washed with buffered media and acquired on a BD Fortessa flow cytometer equipped with BD FACS Diva software (BD Biosciences). Data analysis was performed using FlowJo software (Tree Star, Ashland, OR). For flow gating, doublets were first excluded from the live-cell population and then lymphocytes (CD3+, CD19+) cells were removed from the analysis using a dump-channel approach. Next, HLA-DR+cells were gated into CD11c+monocytic DCs and CD123+pDCs. Thereafter, each DC subpopulation was analysed for its expression of HLA-DR and individual activation markers (CD14, CD25, CD86, CD83).

### 6. Data Analysis and Graph Generation

Data were exported to GraphPad Prism (GraphPad Software, San Diego, CA, USA) for statistical analyses and graph preparation. Cytokine data was exported into excel databases. Out-of-range high (>OOR) values (values higher than the ULOQ) were removed from the data table. Out-of-range low (<OOR) values were replaced with a value representing ½ the LLOQ. The different test items were compared via one-way ANOVA test with Tukey post test adjustment. A “p” value p<0.05 was considered significant.

### II. Results

### 1. SPA14 is Minimally Immunotoxic

The assessment of cells viability is critical to establish the potential immunocytotoxic effects of compounds in cellular subpopulations. To perform this analysis in the current study, cells from 48 hr-treated MIMIC-PTE® cultures were harvested, labeled with a live-dead stain, and analyzed for cell viability via flow cytometry.

As can be seen in FIG. 2, which shows each treatment condition normalized to 100% viability based on the Mock condition, SPA14-8 and SPA14-0 (QS21-Liposomes) had a minimal and comparable impact on cell viability at all doses tested. Interestingly, when tested alone, E6020 triggered a 40-50% reduction in cell viability at a dose equivalent to the 1:40 dilution of SPA14. This observation indicates that the liposomal formulation is able to modulate the immunocytotoxic effect of E6020.

The observation that the TLR4 and TLR7/8 agonists combination (LPS+R848:L+R) induced an ˜80% reduction in PTE cell viability at 48 hrs post-treatment was expected and demonstrates that the assays were operating as expected.

### 2. SPA14 Induces APC Activation/Maturation

APCs (antigen-presentins cells) represent a major element of innate immunity that can steer adaptive immunity through their capacity to engage and activate B and T lymphocytes. A major functional feature of TLR4 agonists is that they can trigger APC maturation, which is a complex process involving changes in the expression of surface markers, such as HLA-DR, CD14, and CD80/86, and the altered expression of various cytokines and chemokines. In the MIMIC PTE module, the activation status of the CD11c+(mDC) subpopulation was measured through the analysis of costimulatory markers on the surface of harvested cells and via the production of soluble cytokines that were evaluated in supernatants pulled from untreated and treated cultures. Of note, while other DC subpopulations are generated in the MIMIC PTE construct, this analysis was focused on conventional CD11c+DCs since they are responsive to diverse TLR agonists and constitute one of the major circulating APC sub-population in vivo (Collin et al., Human dendritic cell subsets. Immunology, 2013, 140: 22-30).

The inventors evaluated the expression of maturation and activation markers on the surface of PTE-derived APCs in the absence or presence of adjuvant treatment. Of particular interest for this study were the costimulatory markers CD86 (B7-2) and CD83 since they have been described as important ligands for APC maturation and activation and are critical for driving naïve CD4+ T cell responses (see FIG. 3).

SPA14 was able to trigger an increase in CD86-positive PTE-derived APCs in a dose dependent manner. CD83 followed a similar expression pattern (data not shown).

### 3. SPA14 Induces the Secretion of Immunostimulatory Cytokines in the PTE Assay

Culture supernatants from untreated and treated MIMIC PTE cultures were harvested after 48 hr and analyzed for cytokine/chemokine secretion using a Millipore custom 12-plex array. The following innate chemokines/cytokines were evaluated: IL-6, IL-8, TNFα, MIP-1β and IP-10, since they are critical for innate immune activity and can also drive immuno-cytotoxicity. Results obtained from the highest dose tested (dilutions 1:20) are reported on the Table 1 below.

TABLE 1

| | | Cytokines/Multiplex (pg/mL) | | | | | |
|---|---|---|---|---|---|---|---|
| | Conditions | IL-6 | IL-8 | MCP-1 | MIP-1β | TNF-α | IL-1B |
| Assay | Mock | 12.81 | 1394 | 627.4 | 49.61 | 12.02 | 9.92 |
| controls | LPS + R848 | 15673 | 63740 | 146792 | 18179 | 12428 | 14170 |
| Adjuvant | SPA14-20 | 2397 | 16389 | 17275 | 8052 | 455 | 192.8 |
| formulations | SPA14-8 | 123.1 | 10929 | 10169 | 974.3 | 67.14 | 33.82 |
| | QS21-Lipososmes | 14.2 | 2287 | 2335 | 80.2 | 20.3 | 9.1 |
| | (SPA14-0) | | | | | | |

### Example 4: Adjuvanting Effect of E6020-QS21-Containing Liposomes on CMV Antigens Administered to Rabbits

Immunogenicity Evaluation of SPA14 and AS01B in Rabbits

The objective of the study was to investigate the immune responses induced by CMV antigen-containing vaccine compositions containing as adjuvant either SPA14 or AS01B in the New Zealand White rabbits following two intramuscular injections at three weeks intervals.

### I. Materials and Methods

The CMV gB+CMV pentamer (pentamer (gH/gL/pUL128/pUL130/pUL131) antigens of CMV were prepared by dilution of concentrated antigens in buffer (e.g. PBS pH 7.4, NaCl 140 mM) to obtain a solution two fold concentrated at 80 μg/mL gB+80 μg/mL pentamer, and were used alone (half diluted in PBS at 40 μg/mL gB+40 μg/mL pentamer) or in combinations (mixture volume/volume) with E6020-QS21 liposomes—SPA14 adjuvant. 500 μL of the antigens/adjuvant mixture were administered via the IM route at at the following concentration: 20 μg gB+20 μg pentamer per dose.

The HCMV pentamer gH/gL/pUL128/pUL130/pUL131 was obtained in CHO cell line transfected with 5 plasmids, each plasmid comprising the sequence coding for one of the 5 proteins constituting the HCMV pentamer. The sequences were from the strain BE/28/2011 (Genbank ID KP745669). The gH sequence was without the transmembrane domain for secretion of the recombinant pentamer. An example of expression of pentamer complex is given in Hofmann et al., Biotechnology and Bioengineering, 2015, vol 112, issue 12, pages 2505-2515). gBdTM obtained as described in U.S. Pat. No. 6,100,064, which is a 806 amino acids long polypeptide.

AS01B was obtained from the commercial vaccine Shingrix at 2:0.5:0.1:0.1 mg/ml of respectively DOPC/Chol/QS21/MPL. As it is not two-fold concentrated, it was mixed with concentrated antigens to reach a volume of injection of 550 μl containing 20 μg gB+20 μg pentamer per dose.

SPA14 was prepared as described in Example 1 or as described in Example 10 at 4:1:0.2:X mg/ml of respectively DOPC/Chol/QS21/E6020. Four different concentrations X of E6020 were used: 0 mg/ml, 0.004 mg/ml, 0.008 mg/ml, and 0.02 mg/ml E6020 to obtain the doses of E6020 described in Table 2 below (dilution v/v with the antigens and 500 μl injected).

Fifty-six New Zealand White female rabbits (Charles River Laboratoires France—ESD) 12-14 weeks-old were administered with 2 intramuscular (IM—0.5 mL or 0.55 mL) injections of different adjuvant formulations of CMV-gB and pentamer (Pent) antigens given 3 weeks apart. The rabbits were assigned to 6 different adjuvant formulation groups, each containing 8 rabbits. Each rabbit received 2 IM injections on Days 1 and 22 in 2 different sites of the lumbar region, each site being injected once. Control Group 1 rabbits received sterile physiological saline (0.9% NaCl). Treated rabbits from Groups 2 and 3 received antigens in buffer and antigens in AS01B control adjuvant, respectively. Treated rabbits from Groups 4 to 7 received antigens in SPA14 adjuvant containing E6020 at doses of 0, 1, 2 and 5 μg, respectively.

TABLE 2

| | | Dose volume |
|---|---|---|
| Group | Treatment | (mL) |
| 1 | 0.9% NaCl | 0.5 |
| 2 | 20 μg gB + 20 μg Pent + buffer | 0.5 |
| 3 | 20 μg gB + 20 μg Pent + AS01B | 0.55 |
| 4 | 20 μg gB + 20 μg Pent + SPA14 | 0.5 |
| | (0 μg E6020) | |
| 5 | 20 μg gB + 20 μg Pent + SPA14 | 0.5 |
| | (1 μg E6020) | |
| 6 | 20 μg gB + 20 μg Pent + SPA14 | 0.5 |
| | (2 μg E6020) | |
| 7 | 20 μg gB + 20 μg Pent + SPA14 | 0.5 |
| | (5 μg E6020) | |

### Seroneutralization Assays

Briefly, 2,5×104 MRC5 fibroblasts or ARPE-19 cells were dispensed in 96-well dark plates the day before the microneutralization (MN) assay. On D0, sera were heat-inactivated at 56° C. for 30 min. Serum samples were serially two-fold diluted in DMEM/F12 1% FBS, starting from 1/10 to 1/10240 in a 96-deep-well plate and incubated with 4.2 log FFU/ml of the BADrUL131-Y4 CMV virus strain (as described in Wang et al., J Virol. 2005 August; 79(16):10330-8) for 60 min at 37° C. in a 5% CO2 cell culture incubator. The serum/virus mixtures were then transferred onto the MRC5 or the ARPE-19 cells and incubated at 37° C. in a 5% CO₂ cell culture incubator for 3 days for the MRC5 cells and for 4 days for the ARPE cells.

Culture supernatant was then removed, and cells were fixed with 100 μl of 1% formol in PBS for 1 hour at room temperature. The plates were then washed with PBS and air-dried at room temperature before analysis on the Microvision fluorescent plate reader to count infected cells in each well.

As control, two wells of cell control (without virus) and six wells with cells infected with half of the viral dilution containing the 4.2 log FFU/mL were present on each plate. The mean of these six wells defined the threshold of seroneutralization, determined as 50% of the specific-signal value. Neutralizing end-point titers were defined as the reciprocal of the last dilution that fell below the calculated 50% specific-signal value. Neutralizing titers (μPRNT50) were defined for each individual serum as the last dilution that induced 50% reduction of infected cells, i.e. the last dilution that induced lower infected cells than the calculated 50% specific-signal value. Geometric mean neutralizing antibody titers were calculated for each group.

### II. Results

### Functional Humoral Responses

### HCMVgB+HCMV Pentamer+SPA14 Induced Neutralizing Antibody Titers in Sera

Individual serum samples collected from all animals at days 1, 15, 24 and 36, were tested for their neutralizing activity. Neutralizing antibody titers inhibiting HCMV entry into epithelial cells in absence of baby rabbit complement and neutralizing antibody titers inhibiting HCMV entry into fibroblast in presence of baby rabbit complement are presented hereafter in order to focus on functional antibodies specific to CMV-pentamer and to CMV-gB, respectively.

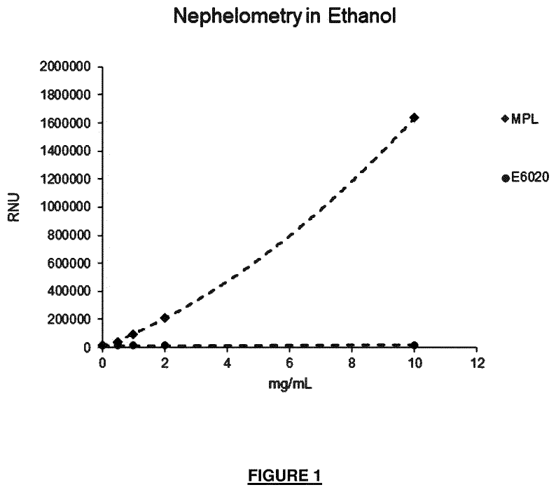

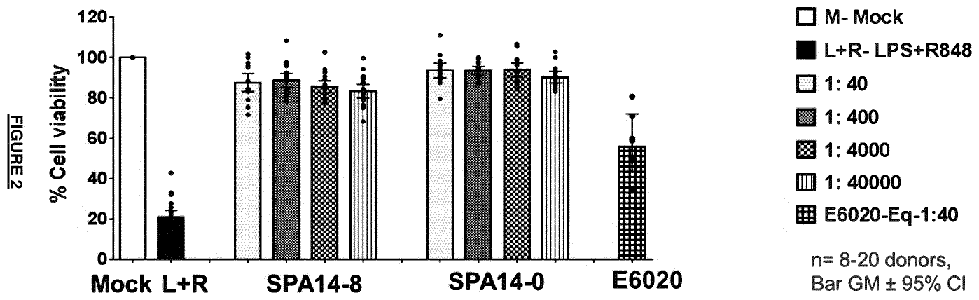

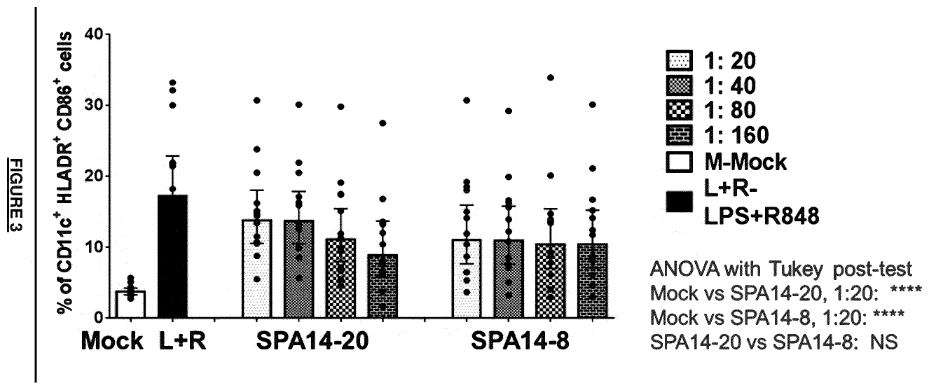

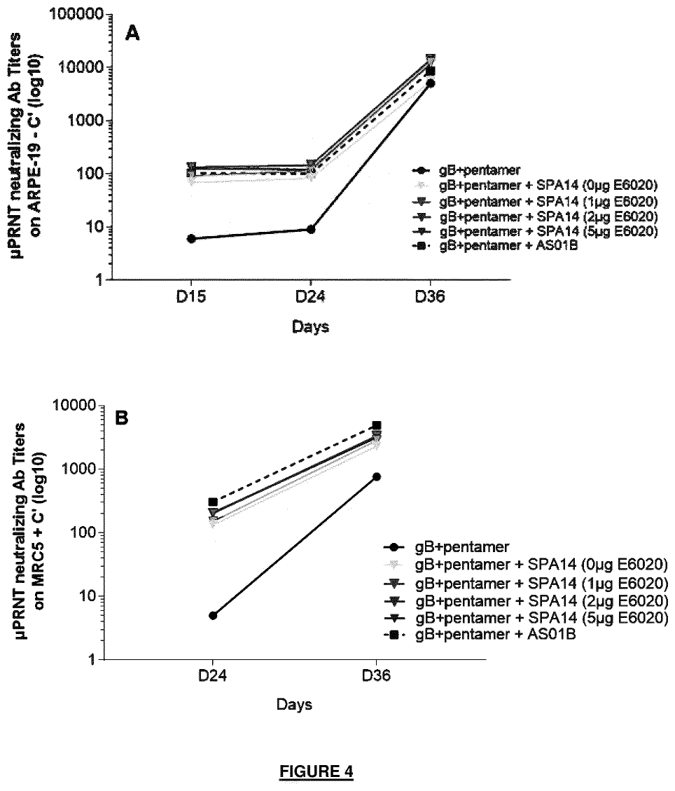

At day 15 as well as at day 24, all the adjuvanted groups developed a functional antibody response (FIGS. 4A and 4B). A highly significant adjuvant effect was observed for all the adjuvanted groups with at least 9-fold higher GMT as compared to the non adjuvanted group (all p-values<0.001, ANOVA, Dunnett adjustment).

At day 24, early after the second vaccine administration, a slight but significant increase of neutralizing antibody titers on epithelial cells was observed as compared to those obtained at day 15 (all p-value s 0.028), whatever the formulations. Similarly, all the adjuvanted groups developed a functional antibody response with mean neutralizing antibody titers ranging from 2.1 to 2.5 log 10 μPRNT50 on fibroblast in presence of complement (FIG. 4B).The neutralizing antibody response increase was further confirmed at day 36, with at least a 15-fold increase as compared to day 24 for all the vaccine formulations, whatever the used seroneutralizing assay.

With respect to the E6020 dose range in SPA14 formulations, no significant impact of the E6020 dosage was observed on the neutralizing antibody response. Addition of 1, 2 or 5 μg E6020 in SPA14 liposomes induced up to 2-fold higher neutralizing antibody titers as compared to the SPA14 liposome without E6020, but none of these differences were statistically significant (all p-values>0.06). With respect to the comparison of SPA14 formulations with the AS01B benchmark, neutralizing antibody titers measured on epithelial cells with and without complement for SPA14 adjuvanted groups were not significantly different from those measured for AS01B adjuvanted group, whatever the E6020 dosage contained in SPA14 and whatever the timepoint (all p-values>0.05, One-side Dunnett test). For GMT measured on fibroblasts (with complement at day 24), neutralizing antibody titers obtained in groups administered with SPA14+0 μg E6020 and SPA14+1 μg E6020 were respectively 2.3 and 2-fold significantly inferior (p-values s 0.02, Oneside Dunnett test) to those measured for AS01B adjuvanted group, whatever the E6020 dosage contained in SPA14 and whatever the timepoint (all p-values>0.05, One-side Dunnett test). For GMT measured on fibroblasts (with complement at day 24), neutralizing antibody titers obtained in groups administered with SPA14+0 μg E6020 and SPA14+1 μg E6020 were respectively 2.3 and 2-fold significantly inferior (p-values s 0.02, One-side Dunnett test) to those measured for AS01B adjuvanted group. Then, with higher E6020 dosage in SPA14, i.e. 2 and 5 μg, no significant difference was observed (p-values>0.18).

Taken as a whole, these results tend to show that SPA14 enhances HCMV neutralizing antibody responses in sera from immunized rabbits no matter the amount of E6020 present in the adjuvant. Further, neutralizing antibody titers measured on fibroblasts with complement for SPA14 adjuvanted groups were not significantly different from those measured for AS01B adjuvanted group, when E6020 content was at least 2 μg/dose which remains far below the concentration of MPL used in AS01B.

The use of E6020 to formulate adjuvant in liposomes with QS21 reveals itself particularly advantageous as it requires 25-times less compound compared to the use of MPLA. This brings advantages in terms of cost and potential reactogenicity.

SPA14 enhances HCMV neutralizing antibody responses in sera from immunized rabbits. μPRNT50 on epithelial cells in absence of complement (FIG. 4A), μPRNT50 on fibroblast in the presence of complement (FIG. 4B).

### Example 5: Adjuvanting Effect of E6020-Qs21-Containing Liposomes on CMV Antigens Administered to Mice

### I. Materials and Methods

In the present mouse study, groups of 7-week old naïve female C57BL/6J mice received three IM immunizations of CMV gB and CMV pentamer (2 μg each/dose) formulated with SPA14 (DOPC-Chol liposomes containing 5 μg QS21 and 1 μg E6020/dose) or AS01E (two-fold dilution of AS01B as described in example 3 obtained from the commercial vaccine Shingrix) adjuvants via the IM route on days 0, 21 and 221 (month 7). Blood samples were collected at months 1, 2, 3, 4, 5, 6, 7 and 8 for monitoring of the seroneutralizing antibody response (the seroneutralization assays were as described in Example 4). Additionally, at months 1, 7 and 8, blood and spleens were collected from 10 mice per group to monitor the CMV gB- and CMV pentamer specific IgG antibody subclasses, the Antibody Secreting Cell (ASC) frequencies as well as the IL-5 and IFN-γ secretions.

### II. Results

Up to month 7 (prior to the late booster), an adjuvant effect was demonstrated on the CMV gB- and CMV pentamer-specific immune response for all tested formulations.

The results of SPA14 vs AS01E are shown below in Table 3 below.

For all timepoints and parameters, the antibody responses, such as neutralizing antibody as well as B memory secreting cell frequencies, obtained with the AS01E adjuvant control were similar or tended to be lower (but not significant different) than the responses obtained with SPA14. Some differences between SPA14 and AS01E were observed with respect the Th1/Th2 cytokine profiles resulting to SPA14 inducing a more balanced Th1/Th2 cytokine profile.

TABLE 3

| | Month 1 | Month 7 | Month 8 |
|---|---|---|---|
| | (15 days | (7 month after | (28 days |
| SPA14 properties as | after the 2^{nd} | the primary | after the 3^{rd} |
| compared to AS01E | injection) | series injection | injection) |
| Induce higher neutralizing | 1. fold | 1.5 fold increase | 1.1 fold |
| Ab titers (fibroblasts in | increase | | increase |
| presence of complement) | | | |
| Induce higher neutralizing | 1.3 fold | 0.7 fold increase | 1.0 fold |
| Ab titers | increase | | increase |
| (epithelial cells in presence | | | |
| of complement) | | | |
| T IFN-γ response (upon | 1.4 fold | 3.2 fold lower* | 2.6 fold |
| pentamer stimulation) | higher | | lower |
| T IL-5 response (upon | 2.2 higher | 2 fold lower | 2.9 fold |
| pentamer stimulation) | | | higher * |
| Induces higher frequencies | 1.9fold | 1.9fold increase | 1.0 fold |
| of memory B cells | increase | | increase |
| (gB specificIgG2c) | | | |
| Induces higher frequencies | 1.0 fold | 1.1 fold increase | 1.3 fold |
| of memory B cells | increase | | increase |
| (Pentamer specificIgG2c) | | | |

In conclusion, analyses conducted up to month 8 in the mouse models allow to conclude that SPA14 was at least as effective as AS01 to improve the immune response to the CMV antigens.

### Example 6: Adjuvanting Effect of E6020-Qs21-Containing Liposomes on Flu Antigens Administered to Mice

Immunogenicity studies with Seasonal Quadrivalent Influenza Vaccines (QIV) extratemporaneously mixed with SPA14 were performed to test the benefits of the adjuvant in naïve BALB/c mice.

GlaxoSmithKline's adjuvant AS01B was used as a comparator control in this study. Commercial lots of Northern Hemisphere 2017-2018 seasonal QIV's Fluzone® and Flublok®containing A/Michigan/45/2015 (H1N1), A/Hong Kong/4801/2014 (H3N2), B/Brisbane/60/2008 (Victoria lineage) and B/Phuket/3073/2013 (Yamagata lineage) strains were used.

The antigen and adjuvant batches were prepared as provided in Tables 4 and 5 below.

The objective of this study was to evaluate immunogenicity of SPA14 adjuvanted Fluzone® and Flublok® vaccines (+/−SPA14) in a mouse model. Here was assessed in naïve BALB/c mice, the immunogenicity of SPA14 adjuvanted-seasonal quadrivalent influenza vaccines (QIV's).

### I. Materials and Methods

Groups of mice (n=8) were immunized with SPA14 or AS01B adjuvanted Fluzone® and Flublok® QIV's and tested for hemagglutination inhibition (HAI or HI) responses, innate responses, and Th1/Th2 ratio. The adjuvants were mixed with the commercially available formulations. The final amounts of antigens, TLR4 agonists and QS21 are indicated in Tables 4 and 5 below.

The tests were performed with BALB/c female mice 6-8 weeks old and minimally 20 g of weight at DO. Animals were immunized via the intramuscular route into the right thigh muscle using a 28 g needle, 0.5 mL syringe (BD #329461). 50 μL of the tested compositions were injected per mouse.

TABLE 4

| | | Amount of |
|---|---|---|
| Vaccine | Antigens | antigens |
| Fluzone ® | A/Michigan/45/2015 (H1N1) | 15 μg (HA) per |
| | A/Hong Kong /4801/2014 (H3N2) | strain in 0.5 mL |
| | B/Brisbane/60/2008 (Victoria lineage), | |
| | B/Phuket/3073/2013 (Yamagata lineage) | |
| Flublock ® | A/Michigan/45/2015 (H1N1) | 45 μg HA per |
| | A/Hong Kong /4801/2014 (H3N2) | strain in 0.5 mL |
| | B/Brisbane/60/2008 (Victoria lineage), | |
| | B/Phuket/3073/2013 (Yamagata lineage) | |
| Hongkong | H3A/Hong Kong/4801/2014 | 705 μg/mL |
| 2014 bulk | | |
| antigen | | |

TABLE 5

| Adjuvant | Components | Amounts |
|---|---|---|
| AS01B | MPL + QS21 + | 5 μg MPL + 5 μg |
| | DOPC + Chol | QS21 + 100 μg |
| | | DOPC + 25 μg Chol |
| | | per mouse |
| SPA14 | E6020 + QS21 + | 1 pg E6020 + 5 μg |
| | DOPC + Chol | QS21 + 100 μg |
| | | DOPC + 25 μg Chol |
| | | per mouse |

### Biological Sampling

At day 21, prior to dose 2 of tested composition being administered, 75 μL of blood were collected via submandibular bleed. Terminal blood was collected on Day 35 into a gel tube by cardiac puncture. Subsequently, it was incubated for at least 30 min at room temperature followed by centrifugation at 10 000 g for 5 minutes, at 23° C. Supernatant was aliquoted into a microvial and stored at −20° C. freezer.

### Hemagglutinin Inhibition Assay:

The sera were diluted 1:5 in RDE (RDE (II) “Seiken” (receptor-destroying enzyme), cat. UCC-340-122, Accurate Chemical) and placed in 37° C. water bath overnight (18-20 hours). Sera were heat-inactivated at 56° C. for 40 minutes. An additional 1:2 dilution with PBS was performed, leading to a final serum dilution of 1:10. Turkey red blood cells (TRBC) were prepared by mixing 0.75% TRBC in PBS/0.75% BSA. The antigens were diluted in PBS/0.75% BSA to contain 4 hemagglutinating units (HAU) in 25 μl and verified as follows: Three rows of a 96-well plate were filled with 50 μl PBS. The first wells of two rows were filled with an additional 50 μl of virus and titrated to the last well in two-fold dilutions. Fifty microliters (50 μl) TRBC were added, the plates were agitated, and the HAU was read after 1 hour incubation at room temperature.

Each well of a 96-well V bottom assay plate was filled with 25 μl PBS. Sera were added across the top row and diluted down the columns in two-fold dilutions. Each sample was tested in duplicate. The second to last column contained the positive control sera and the last column contained the negative control (PBS) and the virus back-titration. 25 μL of virus was added to each well except the last column. The plates were agitated and incubated for 1 hour at room temperature. 50 μL of TRBC were then added to each well followed by a 1-hour incubation, after which the hemagglutination patterns were read by tilting the plates at a slight angle.

For the current study, homologous virus panel including A/Michigan/45/2015 (H1N1), A/Hong Kong/4801/2014 (H3N2), B/Brisbane/60/2008 and heterologous virus panel including A/Singapore/INFIMH-16-0019/2016 and B/Colorado/06/2017 strains were grown in eggs.

### 18-Plex Cytokine/Chemokine Assessment by Luminex-Based Assay

The cytokine profile induced in mice after immunization was evaluated by quantification of serum cytokine/chemokine levels using the Milliplex MAP Kit: Mouse High Sensitivity T Cell Magnetic Bead Panel (EMD Millipore: MHSTCMAG-70KPMX). The following cytokines/chemokines were quantified: GM-CSF, IFNγ, IL-1α, IL-1β, IL-2, IL-4, IL-5, IL-6, IL-7, IL-10, IL-12(p70), IL-13, IL-17A, KC/CXCL1, LIX, MCP-1, MIP-2, and TNF-α.

The Mouse High Sensitivity T Cell Magnetic Bead Panel Assay Kit assay protocol was followed. Within a biosafety cabinet, 200 μl per well of wash buffer was dispensed in 96-well plate provided in the kit. The plate was covered with the provided plate sealer kit and placed on an orbital plate shaker for 10 minutes at 500 rpm-800 rpm at room temperature. Wash buffer was decanted, and any residual liquid was tapped out on absorbent paper. 25 μL of serum matrix was added into standard and controls wells; 25 μL of assay buffer was added to each of sample wells. 25 μL of serum was added to designated wells of each plate.

Samples were assessed in duplicate. 50 μL of standard and quality control was added to appropriate wells and 50 μL of serum matrix was used for Blank (BL). The pre-mixed 18-plex beads were vortexed for 1 minute prior to addition to the plate. The beads were mixed up and down with the pipette before addition of 25 μL of beads per well. The plate was sealed with an adhesive aluminum plate sealer and incubated overnight at 2-8° C. on an orbital plate shaker at 500 rpm-800 rpm.

The following day, the Bio-Plex Wash Station Pro was turned on and primed. The prime function filled the wash station channels with wash buffer and removed any air bubbles prior to use. The detection antibody and streptavidin-phycoerythrin was removed from storage at 2-8° C., 30 minutes prior to use so that the reagents could reach ambient temperature. The plates were washed three times using the “Mag 3×” wash program. 25 μL of the detection antibody solution was added to each well. The plate was covered with an adhesive aluminum plate sealer and incubated on an orbital plate shaker at 500 rpm-800 rpm for 1 hour at room temperature.

The Bio-Plex Luminex plate reader system was calibrated during this detection antibody incubation. A calibration kit with a known low and high analyte concentration bead set was used to measure that the machine was operating correctly and reading within the set parameters defined by the calibration kit. After the incubation, 25 μL of streptavidin-phycoerythrin was added to each well (without washing). The plate was sealed with an adhesive aluminum plate sealer and incubated on an orbital plate shaker at 500 rpm-800 rpm for 30 minutes at room temperature. The plate was washed three times using the “Mag 3×” wash program.

150 μL of sheath fluid was added to all wells. The plate was covered with an adhesive aluminum plate sealer and was allowed to shake on an orbital plate shaker for at least 5 minutes at 500 rpm-800 rpm at 2-8° C. to ensure suspension of the beads. A protocol for reading the plate was set up which included a sample plate map with dilution factors for samples, standards and controls in the Bioplex Manager Software Protocol. The dilution factor of samples was set to 2 as the samples were diluted 1:2 in assay buffer. The dilution of the standard and controls were also set. The plates were read on the Bio-Plex Luminex 200 Plate Reader or the CS 1000 (Perkin Elmer) at low PMT RP1 setting.

### IL-5 and INF-γ Cytokines Assessment by ELISPOT

Anti-mouse IFN-γ (Cat #3321-3-1000, MABTECH) and IL-5 mAbs (Cat #3391-3-1000, MABTECH) were diluted in sterile PBS pH 7.4 (Cat #10010023, Thermo Fisher Scientific) to 15 μg/mL and 96-well ELISpot PVDF-membrane plates (Cat #MSIPS4W10, EMD Millipore) were coated with 100 μL/well of these diluted mAbs at 4° C. overnight.

Each mouse spleen was harvested in 10 ml of ice cold complete medium and transferred into a GentleMACS C tube (Cat #130-096-334, MACS Miltenyi Biotec) for homogenization. The tubes were centrifuged at 1,200 rpm for 6 minutes. Supernatant was discarded, cell pellet resuspended in 4 ml of ACK lysis buffer (Cat #A10492-01, Gibco) and incubated at room temperature (RT) for 3 minutes to lyse red blood cells. Cell suspension was filtered using a 40 μm cell strainer (Cat #352340, BD Falcon) and centrifuged at 1,200 rpm for 6 minutes. Supernatant was discarded and cell pellet was resuspended with 10 mL of the complete medium.

The resuspended cells were diluted to 1:20 with Guava solution (Cat #4000-0041, EMD Millipore Co.). The cell number and viability were determined using Guava® easyCyte cell counter and cell concentration was adjusted to 1×10⁷ cells/mL (5×10⁵ cells/50 μL/well).

The coating antibody solution was removed, plates were washed 3 times with 200 μL/well of sterile PBS and 200 μl/well of blocking solution (complete medium) was added to plates for 2 hours at RT.

The solutions with stimulating agents, recombinant A/Hong Kong/4801/2014 (H3N2) protein or peptide pool (specific stimulation) and Con A (positive control), were diluted in complete medium to 2×final concentration. The final concentration of recombinant protein was 5 μg/mL. The peptide pool contained 122 peptides. The length of each peptide was 15 amino acids with 11 amino acids overlap. The final concentration of each peptide was 2 μg/mL. The concentration of Con A was 2.5 μg/ml. The complete medium was used as a negative control. After 2-hour incubation with blocking solution the plates were emptied out and 50 μL of diluted stimulating agent was added to the plates followed by 50 μL of the cell suspension and mixed by gently tapping the sides of the plate. The plates were then incubated at 37° C. supplied with 5% CO2 for 20 hours.

The cells were removed from the plates, 200 μL/well of water was added and plates incubated at RT for 3 minutes to lyse the cells attached to the plates. The plates were then washed 5 times with 200 μL/well of PBS and 100 μL/well of 1 μg/mL biotinylated anti-mouse IFN-γ (Cat #3321-6-1000, MABTECH) or IL-5 mAbs (Cat #3391-6-1000, MABTECH) diluted with complete medium are added. The plates were incubated at RT for 2 hours and then washed 5 times with 200 μl/well of PBS. One hundred μl/well of 1:1000 diluted alkaline phosphatase-conjugated streptavidin (Cat #7100-04, SouthernBiotech) was added to the plates and incubated at RT for 1 hour. Plates were washed 5 times with 200 μl/well of PBS and 100 μl/well of NBT/BCIP substrate solution (Cat #P134042, Thermo Fisher Scientific) was added for color development. Plates were incubated at RT in the dark for 30 minutes or until spots developed. Plates were rinsed 5 times with water and air-dried in the dark at RT for 24 hours. The spots were counted and analyzed using the CTL-Immunospot plate reader (ImmunoSpot 7.0.23.2 Analyzer Professional DC\ImmunoSpot 7, Cellular Technology Limited) and software (CTL Switchboard 2.7.2). Number of spot forming cells per million was reported.

### Data Analysis

### For the HAI Titers:

The aim was to determine HAI titers against homologous (Michigan/2015, Hong Kong/2014, Brisbane/2008) and heterologous (Singapore/2016, Colorado/2017) influenza strains for 136 mouse sera (136 sera x 3 virus strains; 136 sera x 2 virus strains). Individual animal sera from the D35 time point were tested for HAI for all treatment groups. HAI titers were log 2-transformed and a descriptive analysis to compute means by group was performed, for the homologous (Michigan/2015, Hong Kong/2014, Brisbane/2008) and heterologous (A/Singapore/INFIMH-16-0019/2016 and B/Colorado/06/2017) influenza strains. Values below the limit of detection (<10) were replaced by half that limit prior to log 2-transformation.

### For Cytokine Assessment:

There were 9 groups (8 animals/group) tested in the Mouse High Sensitivity T Cell magnetic bead panel kit (EMD Millipore). This kit assesses 18 analytes. The pre-bleed group was collected 4 days prior to immunization from a limited number of animals. Therefore, there are no paired prebleed and 6 hours post immunization samples. The pre-bleed samples were treated as a separate group in terms of analysis.

The mean fluorescence intensity (MFI) values for samples, controls and standards were measured using the Bio-Plex Luminex 200 Plate Reader (Biorad) or the CS 1000 (Perkin Elmer). The data were analyzed using the Bio-Plex Manager software. Acceptance criterion for quality controls was that the calculated concentration for both high- and low-quality controls were within the lot specific concentration range set by the manufacturer. If the control values were within the range, then the controls passed, and the assay results were accepted. A five-parameters logistic regression curve was plotted using the Bio-Plex Manager software. The specific cytokine concentrations for each sample were interpolated from the standard curve and reported in pg/ml. Any result that was below the limit of quantification (LOQ) set by the assay kit parameters/manufacturer for each cytokine or was “OOR<=Out of Range Below” was changed to the LOQ value of the assay for analysis.

### II. Results

SPA14 was found to enhance immunogenicity by inducing high titers of antigen specific antibodies as monitored by functional HA assay. The increase in homologous HI titers was pronounced for all the adjuvanted vaccine groups.

SPA14 also enhanced heterologous HAI titers in the Fluzone® vaccinated group for H3 and B heterologous virus tested in this study. SPA14 shifted the response towards Th1 response. In addition, the two adjuvants resulted in a measurable increase in IFNγ, IL-5, TNFα, MCP-1, KC, and IL-6 analytes compared to the unadjuvanted formulations. The performance of SPA14 was comparable to AS01B.

Immunogenicity with SPA14 Adjuvanted Fluzone® and Flublok® Vaccines

The ability of the SPA14 adjuvanted Fluzone® and Flublok® QIVs (+/−SPA14) to induce homologous and heterologous immune response was evaluated in BALB/c mice. For that purpose, 25 groups of 8 female BALB/c mice were immunized twice, 3 weeks apart (on DO and D21), by the IM route with the commercial Fluzone® and Flublok® seasonal influenza vaccines. Doses of 0.1 μg and 0.5 μg HA for Fluzone® vaccine and 0.1 μg and 1.0 μg HA for Flublok® vaccine were selected to evaluate immunogenicity of SPA14 adjuvants.

A control group received 0.5 μg dosage of recombinant HA (rHA) from the H3A/Hong Kong/4801/2014 strain (n=8 mice). The serological antibody responses elicited in animals 35 days post-immunization were measured by HAI assays performed with chicken Red Blood Cells (RBCs) against the homologous panel [A strains: A/Michigan/45/2015 (H1N1), A/Hong Kong/4801/2014 (H3N2); B strains: B/Brisbane/60/2008 (Victoria lineage)] and heterologous panel (A/Singapore/INFIMH-16-0019/2016 and B/Colorado/06/2017) of influenza strains.

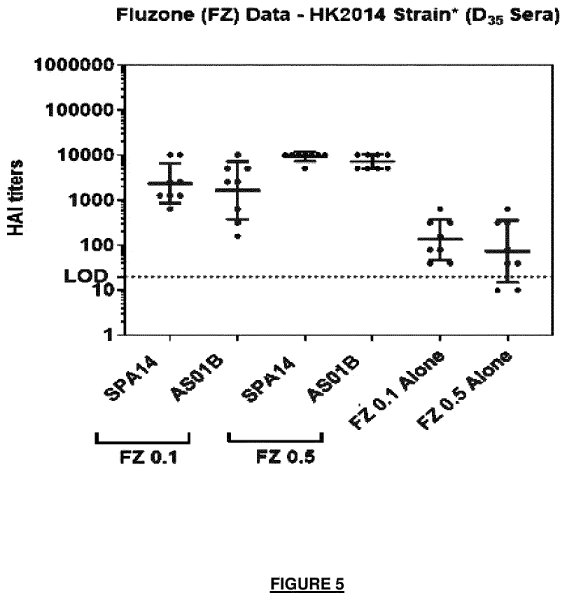

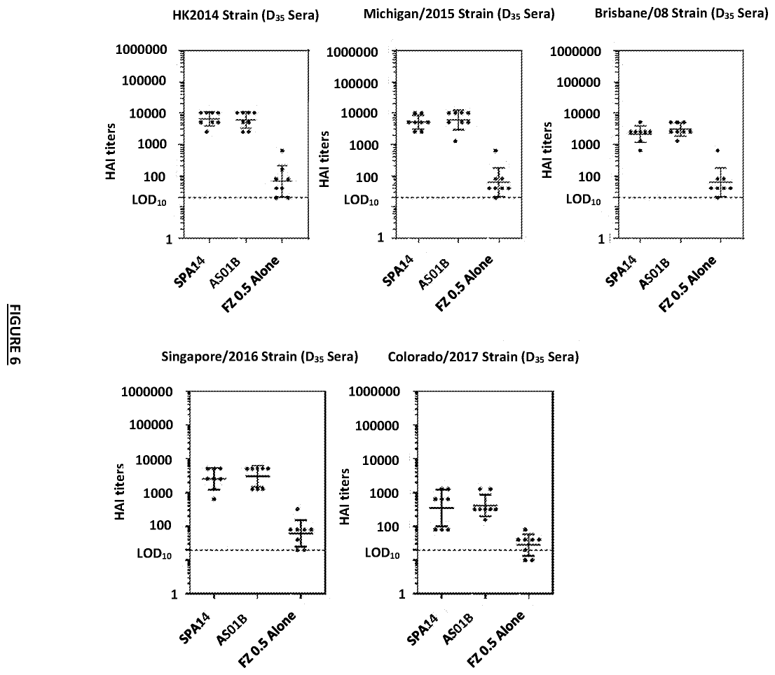

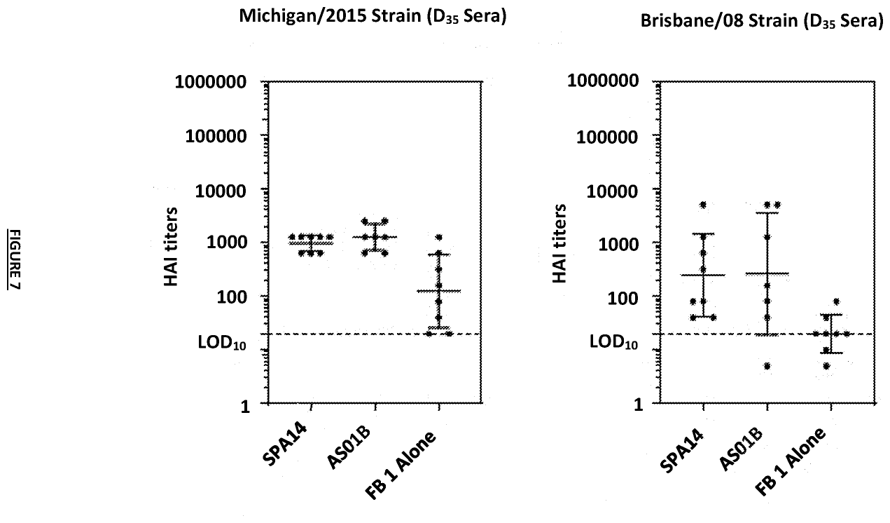

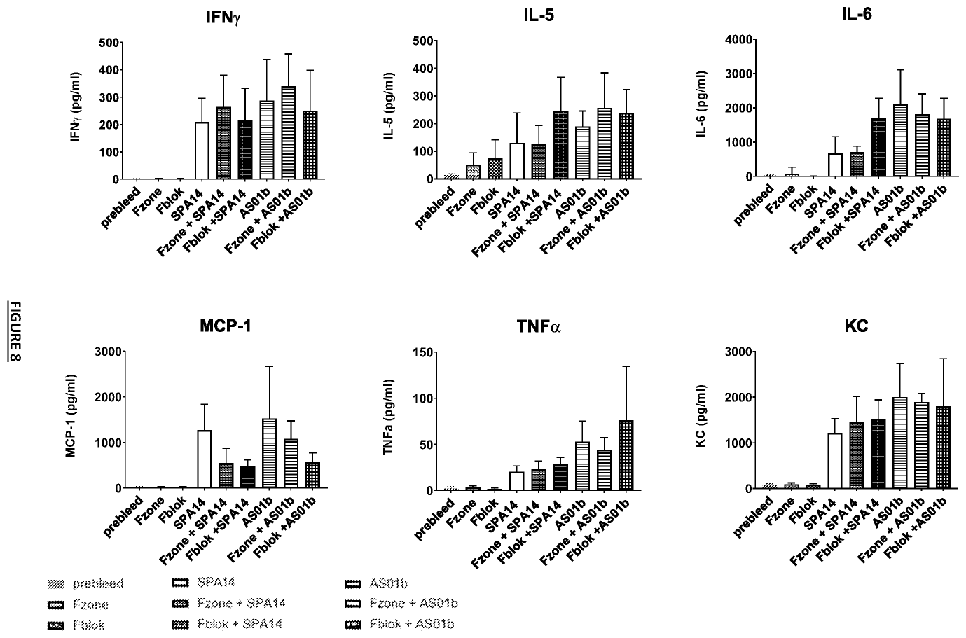

Results in FIG. 5 depicted preliminary HAI titers obtained for Fluzone® QIV (0.1 μg HA (FZ 0.1 in FIG. 5) and 0.5 μg HA (FZ 0.5 in FIG. 5)) against A/Hong Kong/4801/2014 (H3N2) strain. At both the doses of Fluzone® QIV (0.1 and 0.5 μg HA), adjuvants SPA14 and AS01B enhanced HAI titers when compared with antigen alone. Based on these results obtained for Fluzone® QIV the inventors chose higher dose of 0.5 μg HA for subsequent testing against expanded panel of homologous and heterologous influenza strains.

Results depicted in FIG. 6 for Fluzone® QIV (0.5 μg HA) (FZ 0.5 in FIG. 6) showed that both the adjuvants SPA14 and AS01B enhanced HAI titers when compared with antigen alone for the 3 homologous strains. SPA14 and AS01B adjuvants also enhanced HAI titers able to cross-react with the heterologous strains (Singapore/2016 and Colorado). Results obtained for Fluzone® QIV indicated that SPA14 adjuvants performed on par with AS01B.

Results depicted in FIG. 7 for Flublok® QIV (1 μg HA) (FB 1 in FIG. 7) showed that both the adjuvants SPA14 and AS01B enhanced HAI titers when compared with antigen alone for A/Michigan/45/2015 (H1N1) and Brisbane strains.

As expected, no antibody response was induced by SPA14 and AS01B adjuvants alone for heterologous strains.

### Innate Cytokine Response to SPA14-Adjuvanted Vaccines

Cytokine/chemokine profiling in FIG. 8 assessed in sera of immunized animals 6 hrs after immunization demonstrated a measurable increase of IFNγ, IL-5, TNFα, MCP-1, KC, and IL-6 secretion in response to immunization with Fluzone® (Fzon in FIG. 8) and Flublok® (Fblok in FIG. 8) adjuvanted formulations. Immunization with non-adjuvanted formulations yielded a similar profile to prebleed. SPA14 and AS01B adjuvants induced similar increases in cytokine responses when used with Flublok®. Similar or apparently higher responses are observed for AS01B and for SPA14 with Fluzone®.

### Adaptive Cellular Response to SPA14-Adjuvanted Vaccines

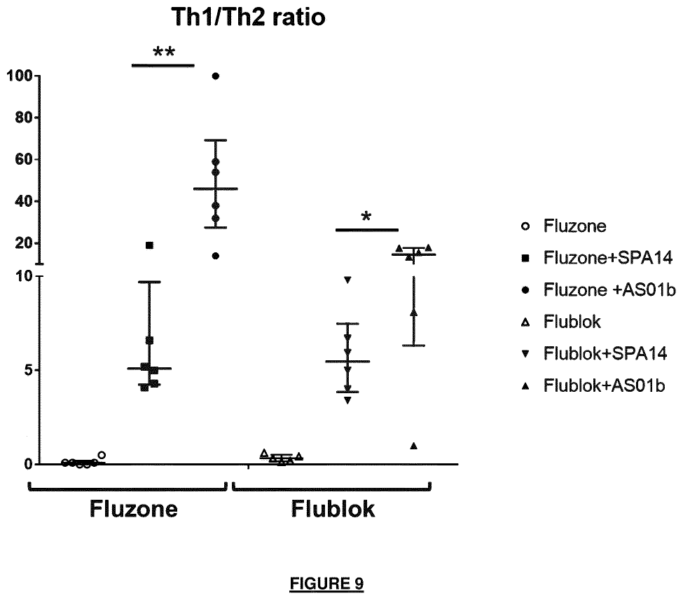

Th1 (IFNg)/Th2 (IL-5) cytokine secretion was assessed in splenocytes of immunized mice two weeks after boost immunization (Day 35). As measured by ELISPOT, Fluzone® or Flublok® alone immunized mice demonstrated low Th1/Th2 ratio, whereas addition of SPA14 significantly increased Th1/Th2 ratio in comparison with antigen alone groups. AS01B adjuvant significantly increased Th1/Th2 cytokine response ratio in comparison with the SPA14 groups (FIG. 9). Compared to AS01B, SPA14 induced a more balanced Th1/Th2 cytokine polarization.

### Example 7: Adjuvanting Effect of E6020-QS21-Containing Liposomes on Human Cytomegalovirus (hCMV) Antigens Administered to Mice and Comparison with Immune Response Profile Triggered by Glaxosmithkline's (GSK's) Adjuvant As01B

Immunogenicity studies using recombinant proteins from human cytomegalovirus (hCMV), extemporaneously mixed with SPA14, were performed to test the benefits of the adjuvant formulation in naïve C57BL/6 mice. GlaxoSmithKline's adjuvant AS01B (GSK AS01B) was used to compare the profile of the immune responses elicited by SPA14 and AS01B in a same study. The glycoprotein B (gB) and the pentamer containing gH/gL/UL128/UL130/UL131 proteins from hCMV were used as antigens.

The objective of this study was to evaluate the antibody and effector cell immune responses elicited by SPA14-adjuvanted gB plus pentamer vaccines (+/−SPA14) and compare with those obtained with AS01B-adjuvanted gB plus pentamer vaccine under the same protocol design in naïve C57Bl/6 mice and experimental conditions.

Groups of mice (n=8) were immunized on DO and D21 with SPA14-adjuvanted or AS01B-adjuvanted hCMV gB and pentamer and tested for serum neutralzing virus activity (SN) in epithelial ARP-19 and fibroblast MRC-5 cell lines, IgG1- and IgG2c-secreting B cells (ASC) specific to gB and pentamer, and T helper cell response (Th1/Th2) specific to gB and pentamer.

Blood and spleen cell samples were taken at 3 weeks post prime at D20 and 2 weeks after the boost at D35 for immune read-out analysis.

### I. Materials and Methods

Animal Information C57BL/6 female mice 6-8-week-old were provided by Charles River Laboratories, Saint Germain sur l'Arbresle, France and housed in Sanofi Pasteur facilities (Marcy L'Etoile, France) according to AAALAC accreditation conditions. This study was reviewed by the Ethics Committee of Sanofi Pasteur (Marcy L'Etoile, France). All experiments were conducted in accordance with the European Directive 2010/63/UE as published in the French Official Journal of Feb. 7, 2013.

Animals, with minimally 20 g of weight at day DO, were primed at DO and boosted at D21 via intramuscular route administration (IM.) into the right thigh muscle using a 28 g needle, 0.5 mL syringe (BD #329461). 50 μL of the tested compositions were administrated per mouse per injection.

An intermediate blood sampling was performed at D20 for each mouse and blood and spleen samples were taken after the boost at D35 from each mouse.

The adjuvant formulations, antigens and vaccine compositions were prepared as indicated in Table 6 below.

TABLE 6

| | | Active substances |
|---|---|---|
| Adjuvant | SPA14 | 20 μg/mLE6020, |
| | | 200 μg/mL QS21, |
| | | 4.0 mg/mL DOPC, |
| | | 1.0 mg/mL Cholesterol |
| | AS01b | 100 μg/mL MPL, |
| | | 100 μg/mL QS21, |
| | | 2.0 mg/ml DOPC, |
| | | 0.5 mg/mL Cholesterol |
| Antigens | hCMV gB | 0.619 mg/mL |
| | hCMV pentamer | 1.011 mg/mL |
| | (gH/gL/pUL128/ | |
| | pUL130/pUL131) | |
| | hCMV gB + hCMV | 2 μg hCMV gB |
| | pentamer | 2 μg hCMV Pentamer |
| Vaccines | hCMV gB + hCMV | 2 μg gB, |
| | pentamer formulated | 2 μg hCMV Pentamer |
| | in SPA14 | 0.5 μg E6020 |
| | | 5 μg QS21 |
| | | 100 μg DOPC |
| | | 25 μg Cholesterol |
| | hCMV gB + hCMV | 2 μg hCMV gB |
| | pentamer formulated | 2 μg Pentamer |
| | in AS01b | 5 μg MPL |
| | | 5 μgQS21 |
| | | 100 μg DOPC |
| | | 25 μg Cholesterol |

### Biological Sampling and Analytical Tests

### Blood Sampling for Serum Preparation

Intermediate blood samples (approximatively 200 μL) were collected post-prime at D20 from submandibular vein in slightly anesthetized mice with isoflurane. On D35, blood samples were collected by cardiac puncture (approximatively 1 mL), under deep anesthesia. Blood samples were collected into Vacutainer tubes with clotting activator and separator gel (BD, Meylan France). After overnight staying at 5±3° C., tubes were centrifuged at 2600 g for 20 min to separate serum from cells. Sera were transferred into deepwell plates in aliquots of 100 μL and heat-inactivated at 56° C. for 30 min. Samples were stored at −20° C. until use in ELISA and neutralizing assays

### Spleen Sampling for Spleen Cell Isolation

For B cell and T cell ELISpot assays, spleen from each immunized mouse was collected in sterile tube containing RPMI (Roswell park Memorial Institute medium). As soon as possible, spleens were mechanically dissociated using GentleMACS (Mylteni Biotech), centrifuged in 50 mL Falcon tubes at 500 g for 10 min at 4° C. and the supernatant was discarded. Each cell pellet corresponding to one mouse spleen was suspended with 1 mL of red blood cell lysis buffer (R 7757 Sigma-Aldrich) and gently mixed for 1 minute. The lysis reaction was stopped in ice and then, 20 mL of cooled RPMI buffer were added per pellet. Falcon tubes were centrifuged 500 g for 7 minutes and the supernatant was discarded. Spleen cells were diluted in RPMI complete buffer containing 10% FCS and prepared for cell counting before use in ELISpot assays.

hCMV Plaque Reduction Neutralizing Test (PRNT50) for the Detection of hCMV Neutralizing Antibodies in ARPE-19 Epithelial Cell Line.

Neutralizing antibodies in sera from immunized mice were titrated using plaque reduction seroneutralizing test. The assay was based on the ability of human Cytomegalovirus (hCMV) to infect human epithelial and fibroblast cells. Briefly, 2.5×10⁴ epithelial ARPE-19 cells were dispensed in 96-well dark plates the day before the microneutralization (MN) assay. Before their use, serum samples collected at D20 and D35 from each immunized mouse were first heat-inactivated at 56° C. for 30 min and stored at −20° C. The D-day of neutralizing assay, heat-inactivated sera were slowly thawed at room temperature (20-25° C.) and were serially two-fold diluted in DMEM/F12 1% FBS starting from 1/10 to 1/10240 in a 96-deep-well plate and incubated with 4.2 Log₁₀ FFU/mL of the BADrUL131-Y4 CMV virus strain (titer 5.63 log 10 FFU/ml) for 60 min at 37° C. in a 5% CO2 cell culture incubator. The different dilution of serum/virus mixtures were finally transferred onto ARPE-19 cell culture and incubated for 4 days at 37° C. in a 5% CO2. On D4, culture supernatants were removed, and ARPE-19 cells were fixed with 100 μL/well of 1% formol in PBS for 1 hour at 20-25° C. The plates were washed three times with PBS 1× and air dried at 20-25° C. before analysis on the Microvision fluorescent plate reader. Infected fluorescent cells were numerated in each well. As controls, cells were only incubated with culture medium without virus in duplicate. As control of infection in 6 wells per plate, cells were infected with half concentration of the initial of the viral dilution at 4.2 Log₁₀ FFU/mL. The mean of the two wells without infected cells defined the threshold of seroneutralization. The mean value of fluorescence in the 6 wells containing cells infected with ½ viral dose defined the 50% specific-signal of infection. For each serum, neutralizing end-point titers were defined as the reciprocal of the last dilution that fell below the calculated 50% specific-signal value (μPRNT₅₀), i.e. the last dilution that induced less infected cells than the calculated 50% specific-signal value. For each sample, titer was determined using a 4-parameter logistic curve. Geometric mean neutralizing antibody titers were calculated for each group

A similar protocol was used for PRNT₅₀ assay on fibroblast MRC-5 cell line. Briefly, 2.5×10⁴ MRC-5 cells were dispensed in 96-well dark plates the day before the microneutralization assay. Heat-inactivated sera were t serially two-fold diluted in DMEM/F12 1% FBS, starting from 1/10 to 1/10240 in a 96-deep-well plate and incubated with 4.2 Log₁₀ FFU/mL of the BADrUL131-Y4 CMV virus strain (titer 5.63 log 10 FFU/ml) in presence of 10% of baby rabbit complement for 60 min at 37° C. in a 5% CO2 cell culture incubator. The different dilutions of serum/virus mixtures were then transferred onto MRC-5 cell cultures and incubated at 37° C. in a 5% CO2 for 3 days. On D3, culture supernatants were removed, and MRC-5 cells were fixed with 100 μl/well of 1% formol in PBS for 1 hour at room temperature. Neutralizing antibody titers (μPRNT₅₀) were determined as previously described with above for ARPE-19 cell line.

Note: plaque reduction neutralizing assay on ARPE-19 cell line reflects the neutralizing antibody activity elicited by both antigens gB and pentamer whereas plaque reduction neutralizing assay on MRC-5 cell line rather reflects neutralizing antibody activity elicited by gB.

hCMV gB and Pentamer-Specific IgG1 and IgG2c-Secreting B Cells ELISpots

The fluorescent-linked immunospot (FLUOROSPOT) assay was used for detecting and enumerating individual B cell secreting antibodies (ASC) specific to hCMV gB and pentamer antigen (IgG1, IgG2c, IgG) and compared to ASC secreting total IgG, irrespective of the antigen specificity.

The fluorospot plates equipped with a low-fluorescent PVDF membrane were pre-wetted with 35% ethanol for 1 min, washed with sterile water, then with PBS 1× and coated overnight at 5±3° C. with either 10 μg/mL of hCMV gB or 10 μg/mL hCMV pentamer or a mix of capture mAb (10 μg/mL KDL)

The membrane of the 96-well IPFL-bottomed microplates (Multiscreen) was first pre-wetted with 25 μL of 35% ethanol at room temperature and removed after 1 min treatment. After washing with 200 μL/well of PBS 1×, the microplates were coated with either hCMVgB antigen or hCMV pentamer (10 μg/ml) or irrelevant mouse IgG antibody (10 μg/ml, KPL). Plates were washed with PBS and blocked with complete medium RPMI 10% FBS for 2 hours at 37° C. After washing with PBS, 5×10⁵ freshly isolated spleen cells from immunized mice were plated per well and incubated for 5h at 37° C. in a 5% CO2 incubator. Following 3 washes in PBS 1×Tween 20 0.05% and 6 washes in PBS 1×, the plates were incubated for 2 hours at 37° C. with 100 μL/well of anti-mouse IgG1 PE (4 μg/mL), anti-mouse IgG2c FITC (2 μg/mL) or anti-mouse total IgG (0.5 μg/mL) detection mAb. After washing with PBS, fluorescent spots were enumerated with an automatized spot reader (Microvision)

hCMV gB and Pentamer-Specific IFNγ- and IL-5-Secreting T Cell ELISpot

FluoroSpot assay was used for detection and enumeration of individual cells secreting either IFN-γ or IL-5 cytokines. Briefly, Multiscreen™ 96-well IPFL plates (Millipore) were pre-wetted with 25 μL of 35% ethanol for 1 minute at 20-25° C., washed with sterile water, washed twice with PBS 1×. Plates were then coated overnight at 5±3° C. with 100 μL per well of rat anti-mouse IFN-γ or rat anti-mouse IL-5 mAb (10 μg/ml, Pharmingen), diluted at 1/100 and 1/50, respectively. After 3 plate washing with 200 μL sterile PBS 1×per well, a saturation step was performed with 200 μL of complete medium (Roswell park Memorial Institute medium containing 10% fetal calf serum, 200 mM L-Glutamine, 100U/ml penicillin and 10 μg/ml) for 2 hours at 37° C. After plate washing, 5×10⁵ freshly isolated spleen cells were added per well and incubated overnight with hCMV gB antigen (0.1 μg/mL), hCMV pentamer antigen (0.1 μg/mL or concanavalin A (Con A, 2.5 μg/mL) as a positive control, in presence of murine IL-2 (10 U/ml).

After 6 washes in PBS 1×BSA 0.1% (200 μL per well), biotinylated anti-mouse IFN-γ or anti-mouse IL5 antibodies used at the concentration of 1 μg/mL in PBS 1×BSA 0.1% were added (100 μL per well) and incubated for 2 hours at 20-25° C. in the dark. After 3 washes in PBS 1×BSA 0.1%, 100 μL of streptavidin PE (1 μg/mL) in PBS 1×-BSA 0.1% were added per well and incubated at 20-25° C. for 1 hour in the dark. Plates were further washed 6 times with PBS 0.25% BSA. Finally, the back of plates was removed, and the well undersides quickly rinsed with sterile water. Plates were air-dried and stored in the dark until reading. Each spot corresponding to a single producer cell of IFNγ or IL-5 was enumerated with a fluorescent plate reader equipped with a filter for Cy3 and FITC fluorescences (Microvision). Results were expressed as number of IFN-γ- or IL-5-secreting cells per 10⁶ spleen cells.

### II. Results

SPA14 Formulated with hCMV gB and Pentamer Proteins Elicted Similar hCMV Neutralizing Antibody Titers than AS01B

The ability of SPA14 adjuvanted hCMV gB and pentamer proteins to induce seroneutralizing responses was evaluated in C57BL/6 mice. For that purpose, 3 groups of 8 female C57BL/6 mice were immunized twice, 3 weeks apart on day (D) 0 and D21, by IM route with 2 μg of each recombinant proteins, hCMV gB and pentamer. The proteins were administered without adjuvant or formulated either with SPA14 or AS01B, whose compositions have been described in example 3.

AS01B was used as benchmark adjuvant. The neutralizing antibody responses elicited in immunized animals 20 days post-first injection (D20) and 14 days post-second injection (D35) were measured using a plaque reduction seroneutralizing assay, based on the ability of human BADrUL131-Y4 CMV virus strain to infect both human fibrosblast and epithelial cells, with or without complement, respectively.

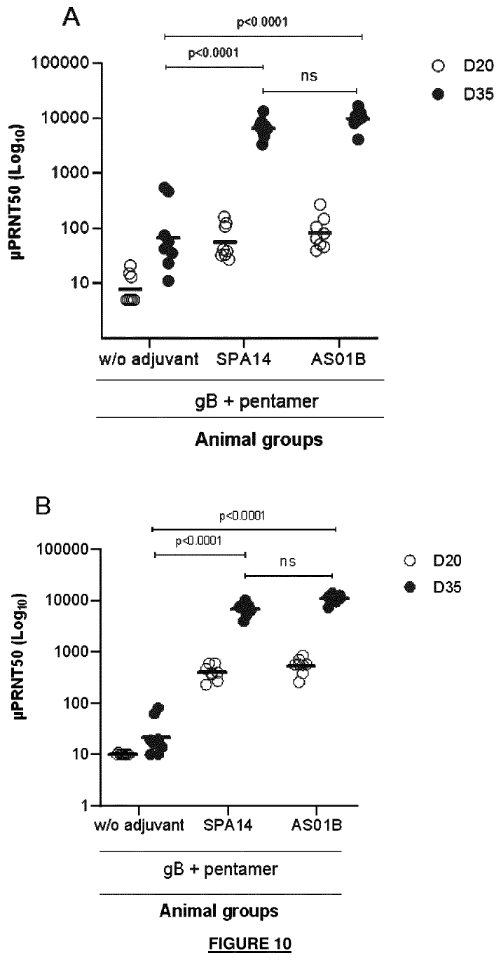

Results in FIGS. 10A and 10B showed that without adjuvant, hCMV gB plus pentamer proteins induced no or low levels of BADrUL131-Y4 CMV virus serum neutralizing antibodies (SN Ab) post-prime and post-boost measured both on fibroblast (MRC-5) or epithelial (ARPE-19) cell lines.

A significant adjuvant effect was measured with SPA14 and AS01B on BADrUL131-Y4 CMV virus neutralizing antibodies and was mainly observed post-boost (p-value<0.0001) compared to the non-adjuvanted group hCMV gB plus pentamer in both cell lines.

Moreover, a significant boost effect was measured in the adjuvanted groups with SPA14 and AS01B (p<0.0001), with approximatively a 2 Log-increase of neutralizing antibody titers from D20 to D35.

On ARPE-19 epithelial cell line without complement (FIG. 10A), representative of neutralizing antibodies induced by both gB and pentamer, geometric mean values (GMT) of neutralizing titers reached 3.75 Log₁₀ with SPA14 and 3.98 Log₁₀ with AS01B whereas a GMT of 1.82 Log₁₀ was determined in the non adjuvanted group. Similar results were obtained in MRC-5 fibroblast cell line in presence of complement (FIG. 10B), reflecting the neutralizing antibody titers mainly induced by gB., Geometric mean values (GMT) of neutralizing Ab titers of 3.84 Log₁₀ with SPA14 and 4.05 Log₁₀ were observed with AS01B whereas a GMT of 1.3 Log₁₀ was observed in the non adjuvanted group.

No difference (ns) was measured on neutralizing antibody titers induced by hCMV gB plus pentamer formulated with SPA14 and gB plus pentamer formulated with AS01B, post prime D21 and post-boost D35.

SPA14 Formulated with gB and Pentamer Proteins Triggered gB- and Pentamer-Specific IgG1 and IgG2c-Secreting B Cells (ASC) on D35 Compared to the Non Adjuvanted Group

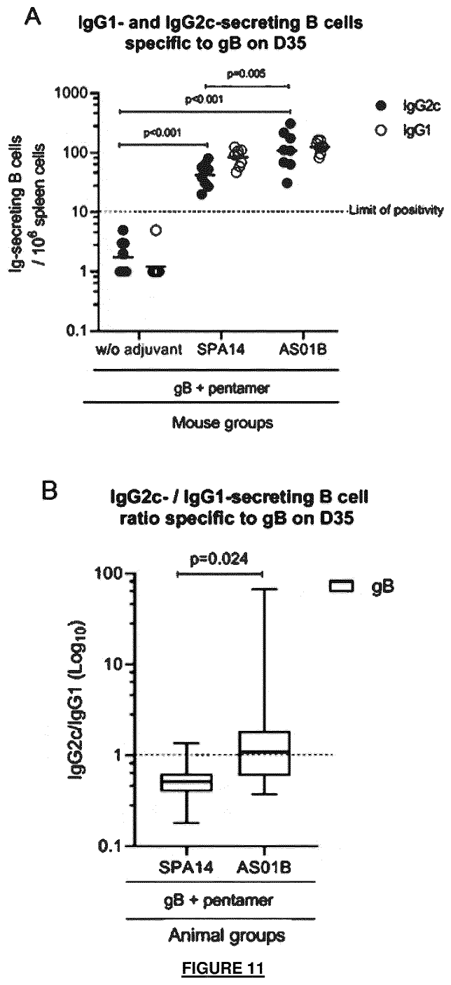

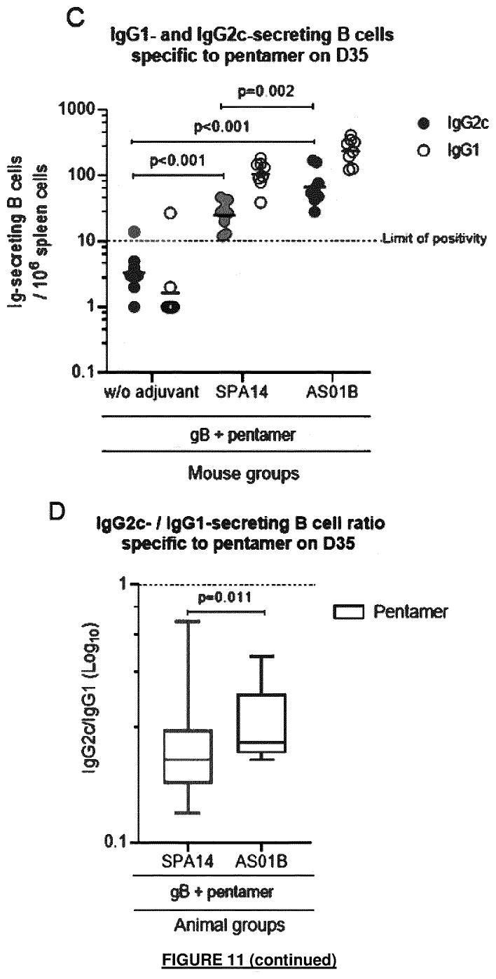

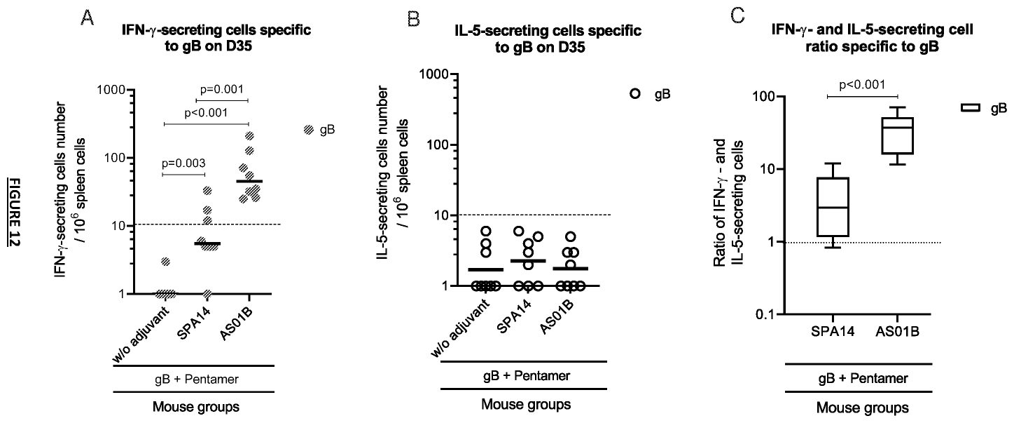

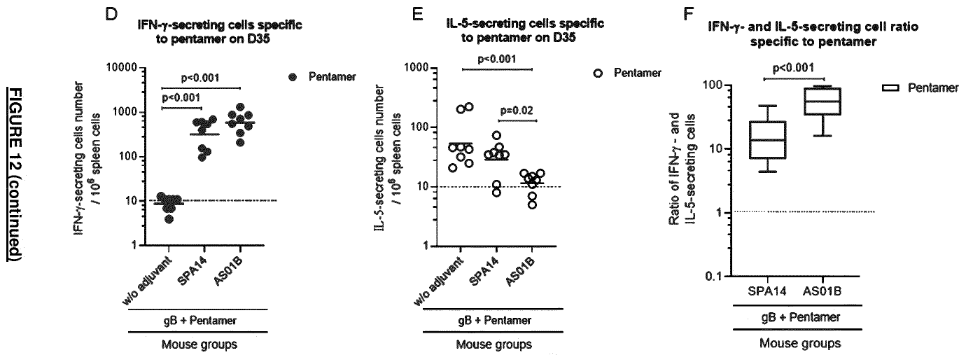

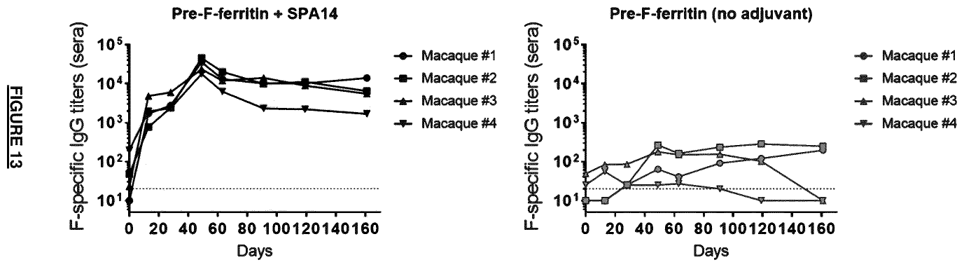

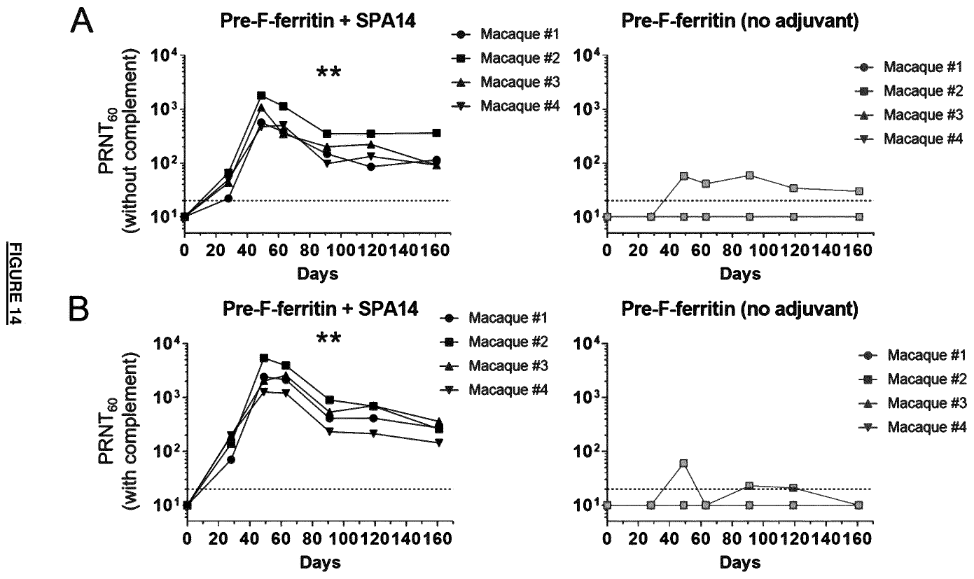

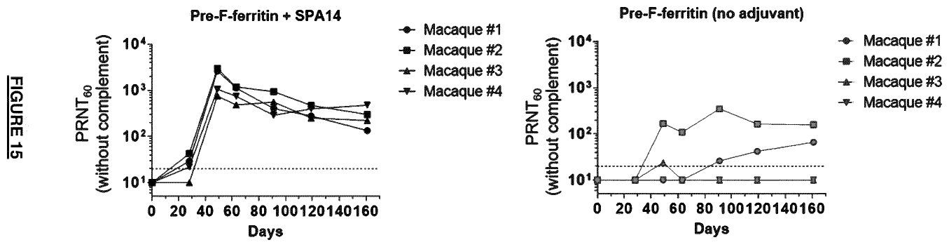

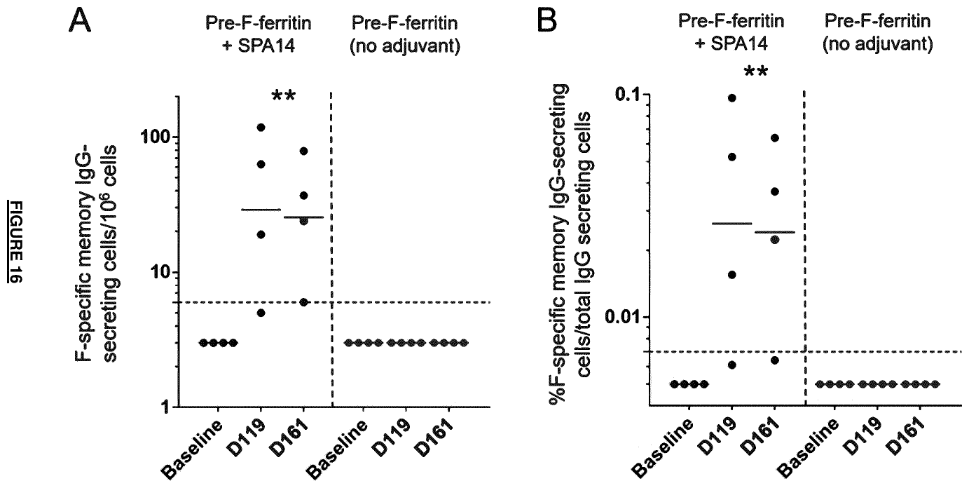

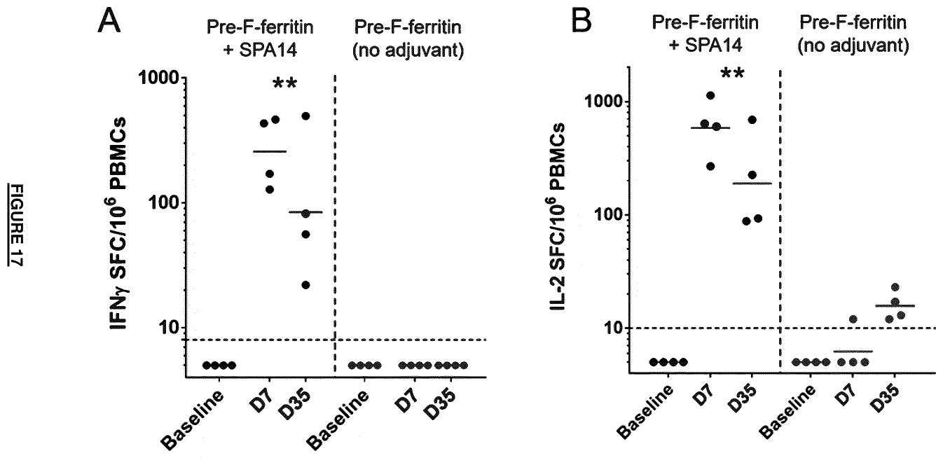

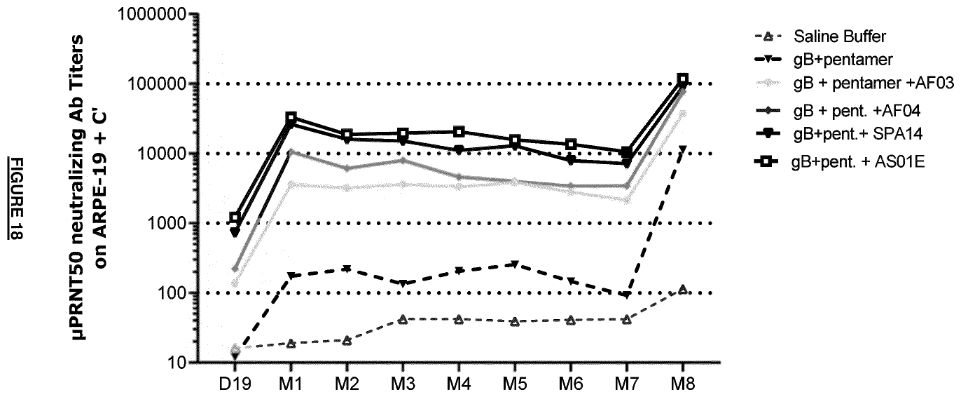

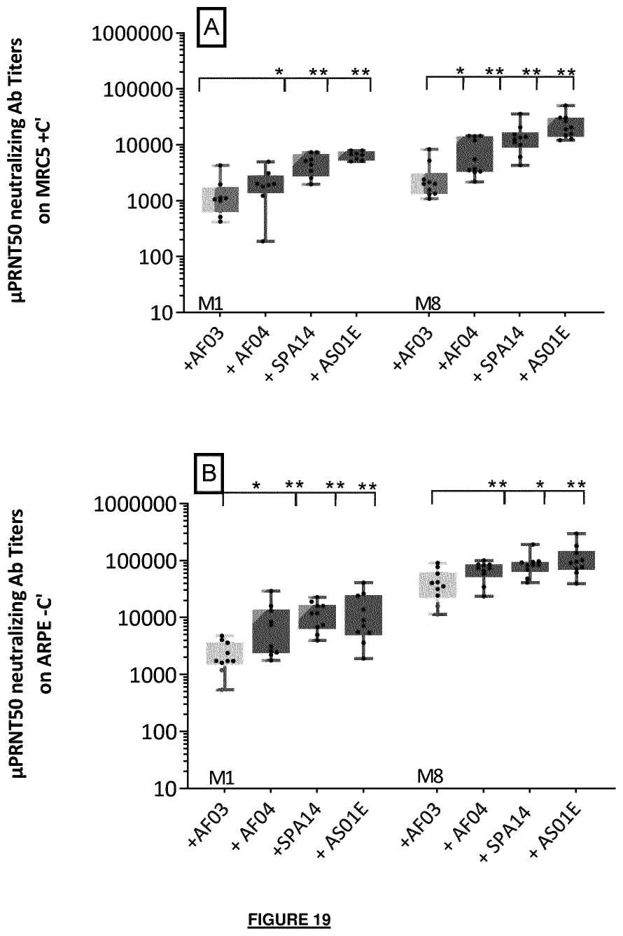

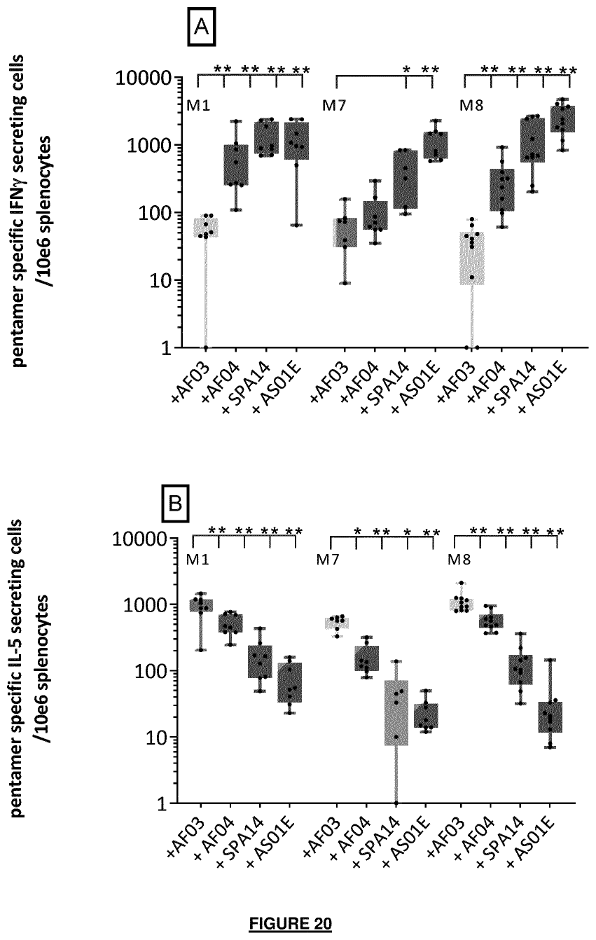

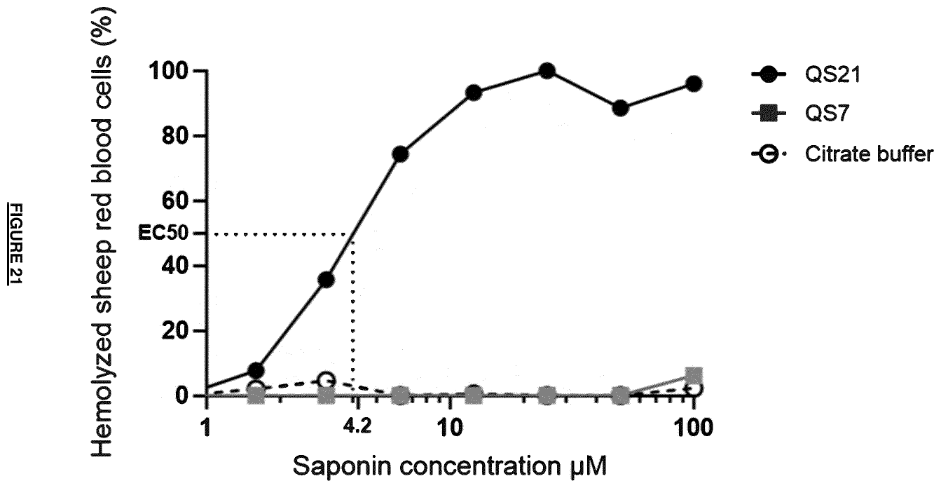

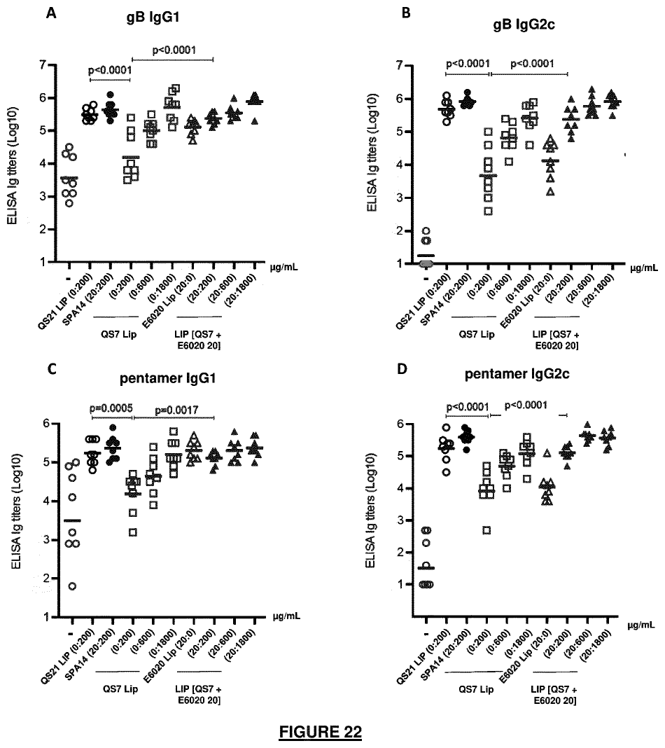

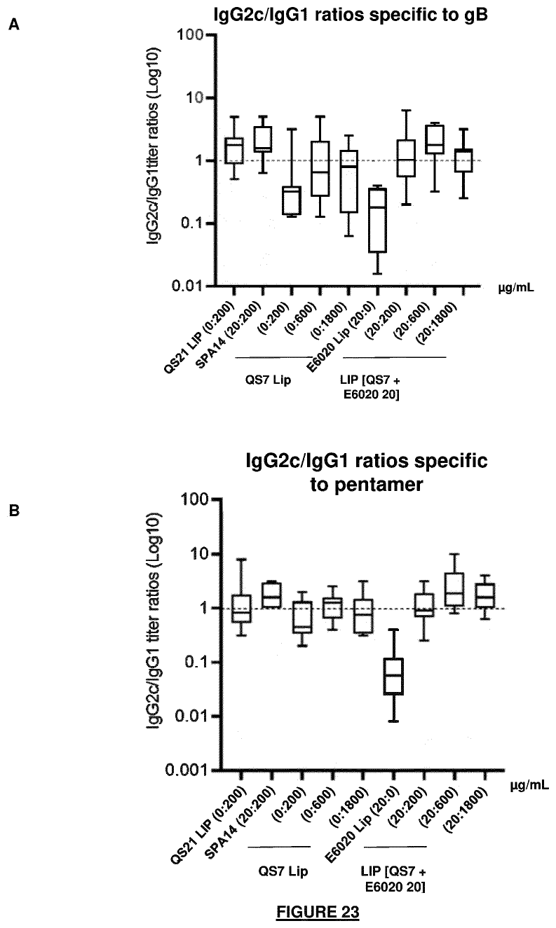

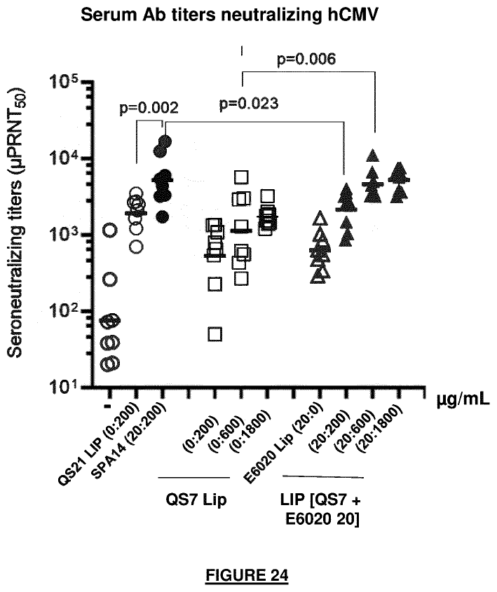

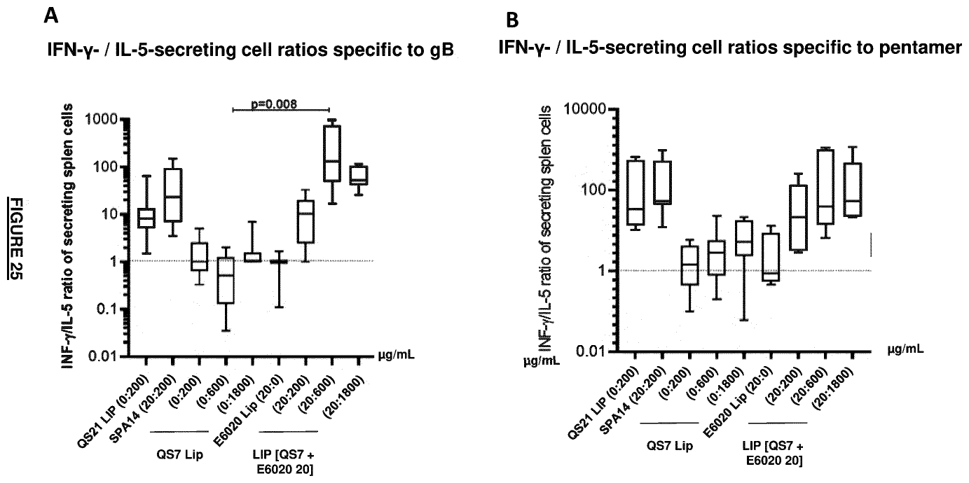

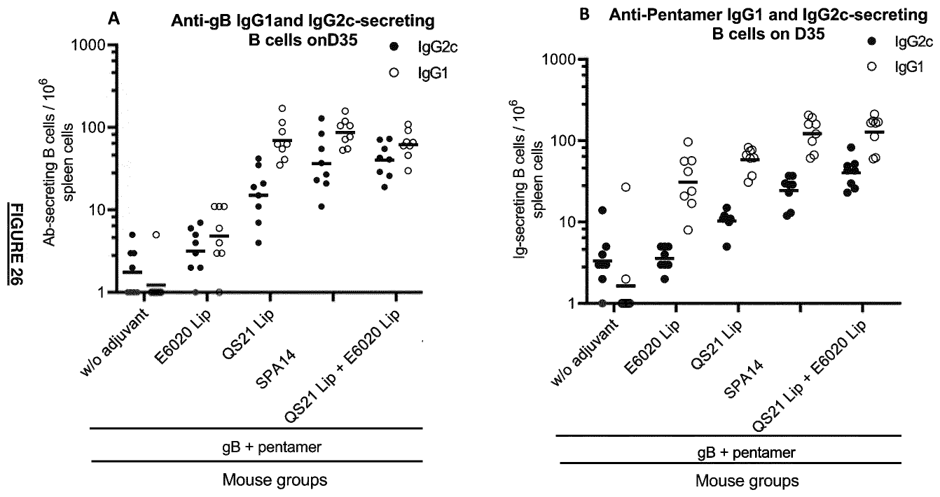

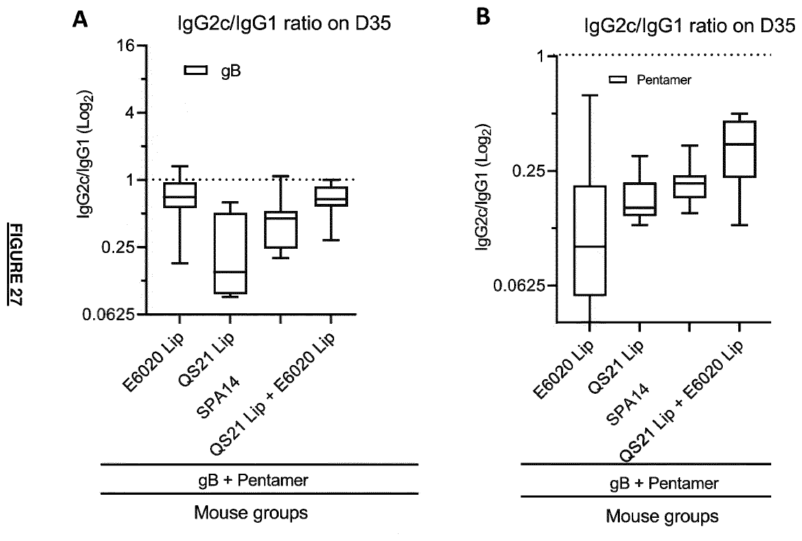

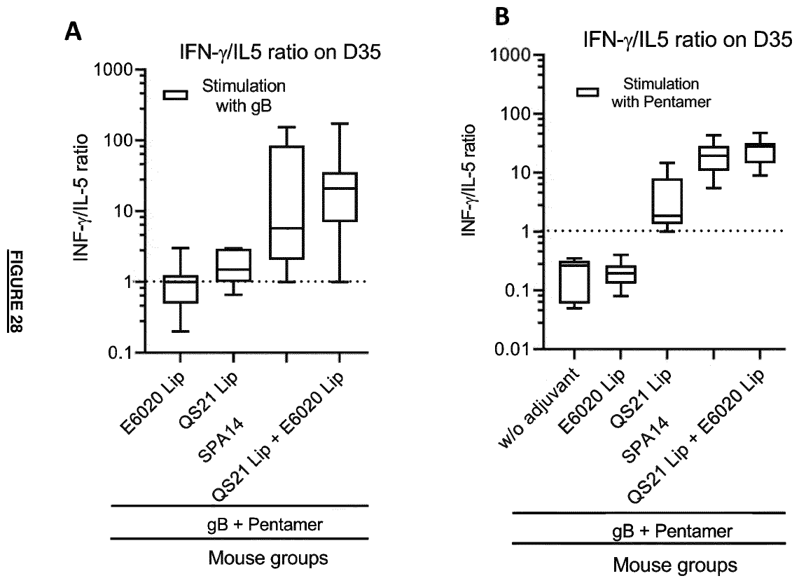

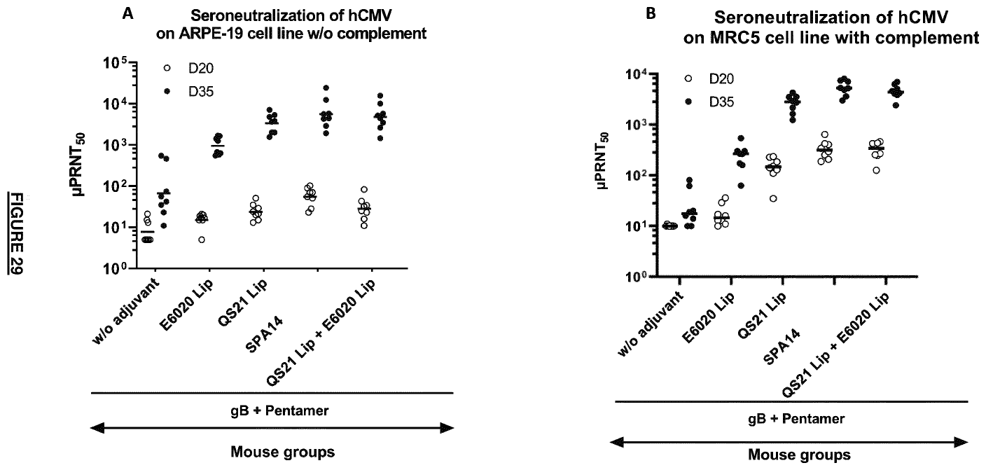

The ability of SPA14 adjuvanted hCMV gB and pentamer proteins to induce IgG1 and IgG2c-secreting B cells (ASC) specific to both hCMV antigens was evaluated in C57BL/6 mice. For that purpose, 3 groups of 8 female C57BL/6 mice were immunized twice, 3 weeks apart on day DO and D21, by IM route with 2 μg of each recombinant proteins, hCMV gB and pentamer. The recombinant proteins were administered without adjuvant or formulated either with SPA14 or AS01B. IgG1 and IgG2c-secreting B cells (ASC) specific to gB or pentamer were assessed by Fluorospot on spleen cells collected on D35 from immunized mice (FIG. 11A-D).

SPA14 induced significant higher IgG1 and IgG2c ASC frequencies specific to gB and pentamer, compared to the non adjuvanted group (p<0.001).

Indeed, specific ASC numbers measured in SPA14 adjuvanted group were moderate with a geometric mean value of 42 spots/10⁶ cells for IgG2c- and 83 spots spots/10⁶ cells for IgG1-secreting B cells specific to gB, 25 spots/10⁶ cells for IgG2c- and 105 spots spots/10⁶ cells for IgG1-secreting B cells specific to pentamer.

Similarly, AS01B induced moderate frequencies of IgG1- and IgG2c-ASC numbers specific to gB and pentamer but these cell frequencies were significantly higher than those measured in the non adjuvanted group (p<0.001).

Interestingly, data also showed that AS01B significantly improved the frequencies of anti-gB IgG2c-ASC (p=0.005) and anti-pentamer IgG2c-ASC (p=0.002) compared to SPA14, leading to a significant higher IgG2c-/IgG1-secreting B cells ratios against gB (p=0.024) and against pentamer (p=0.011).

In conclusion, SPA14 led to a more balanced Th1/Th2 antibody response than AS01B which significantly induced a more Th-1-skewed antibody response.

SPA14 Formulated with gB and Pentamer Proteins Elicited Low IFN-γ-Secreting Cells Specific to gB and but High IFN-γ-Secreting Cells Specific to Pentamer on D35 Compared to the Non Adjuvanted Group

The ability of SPA14 adjuvanted hCMV gB and pentamer proteins to induce IFN-γ- and IL-5-secreting cells specific to gB and pentamer was evaluated in C57BL/6 mice. For that purpose, 3 groups of 8 female C57BL/6 mice were immunized twice, 3 weeks apart on day (D) 0 and D21, by IM route with 2 μg of gB and pentamer. The recombinant proteins were administered without adjuvant or formulated either with SPA14 or AS01B.

IFN-γ- and IL-5-secreting cells were assessed by Fluorospot on fresh spleen cells collected on D35 from immunized mice (FIG. 12).

In mice immunized with non adjuvanted gB and pentamer, no IFN-γ- and IL-5-secreting cells specific to gB and no IFN-γ-secreting cells specific to pentamer were detected. In the non adjuvant group, low but measurable IL-5-secreting cell numbers specific to pentamer were detected (54 spots per 10⁶ spleen cells).

Data related to ex vivo spleen cell stimulation with gB described in FIGS. 12A, 12B and 12C showed that SPA14 induced significantly higher IFN-γ-secreting cell frequency (p=0.003) compared to the non adjuvanted group whereas no significant adjuvant effect of SPA14 was measured on IL-5-secreting cell number. Moreover, the increase of IFN-γ-secreting cell frequency did not have an impact on the ratio of IFN-γ- and IL-5 secreting cells (p=0.074; ns), a ratio>1 being representative of a skewed Th1 response (data not shown).

Data related to ex vivo spleen cell stimulation with pentamer described in FIGS. 12D, 12E and 12F showed that SPA14 induced significantly higher IFN-γ-secreting cell number, with a GM value of 321 spots per per 10⁶ spleen cells (p<0.001) compared to the non adjuvanted group with a GM value of 9 spots per 10⁶ spleen cells. No significant adjuvant effect of SPA14 was measured on IL-5-secreting cell number specific to pentamer. These results were correlated with a significant increase of IFN-γ- and IL-5 secreting cell ratio (p<0.001) compared to the non adjuvanted group reflecting a more Th-1-skewed immune responses directed to pentamer with SPA14 compared to the non adjuvanted group (ratio data with SPA14 vs non adjuvanted not shown).

In FIG. 12, results showed that AS01B induced significantly higher IFN-γ-secreting cell numbers, specific to both gB and to pentamer (p=<0.001) compared to the non adjuvanted group on D35. No significant adjuvant effect of AS01B was measured on IL-5-secreting cell number specific to gB but AS01B induced a significant reduction of IL-5-secreting cell number specific to pentamer with a GM value of 11 spots per 10⁶ cells compared with the non adjuvanted groups displaying a GM value of 54 spots 10⁶ cells (p<0.001).

Taken together, the data demonstrated a significant increase of IFN-γ- and IL-5 secreting cell ratios specific either to gB or pentamer compared to the non adjuvanted group (p<0.001) reflecting a more Th-1-skewed immune responses directed to hCMV antigens with AS01B compared to the non adjuvanted gB plus pentamer (ratio data with AS01B vs non adjuvanted group not shown).

The comparison of the 2 adjuvanted groups showed that SPA14 elicited significant lower IFN-γ-secreting cells number specific to gB compared to AS01B (p=0.001) but induced similar number of IFN-γ-secreting cells specific to pentamer. As observed with SPA14, AS01B did not induce specific IL-5-secreting cells specific to gB but interestingly SPA14 triggered significant higer IL-5-secreting cells specific to pentamer than AS01B (p=0.02).

Taken together, the data indicate that SPA14 induces a more balanced Th1/Th2 response than AS01B.

### III. Conclusion

In C57BL/6 mice, SPA14 formulated with hCMV gB and pentamer elicited high humoral and cellular immune response. SPA14 elicited high serum neutralizing antibody titers to hCMV on both epithelial (without complement) and fibroblast (with complement) cells, similar to those induced with AS01B, IgG1- and IgG2c-secreting effector cells specific to both hCMV antigens, IFN-γ- and IL-5-secreting cells specific to gB and mainly IFN-γ-secreting cells specific to pentamer.

Results demonstrated that SPA14 elicited a mixed Th1/Th2 immune profile to hCMV gB and pentamer whereas AS01B triggered a more Th-1-skewed immune response, eliciting higher specific IgG2c-secreting B cell and IFN-γ-secreting T cell frequencies

### Example 8: Adjuvanting Effect of E6020-Qs21-Containing Liposomes on RSV Pre-F Ferritin Antigens Administered to Cynomolgus Macaques

This study evaluated the immunogenicity of adjuvanted RSV pre-F-ferritin vs unadjuvanted RSV pre-F-ferritin vaccines in the naive cynomolgus macaque model (Macaca fascicularis). Pre-F-ferritin is a recombinant protein antigen particle produced in CHO cells and composed of the RSV-F glycoprotein blocked in the prefusion conformation and fused to a self-assembling Helicobacter pylori ferritin moiety (Swanson et al., Sci Immunol. 2020; 5(47)). One adjuvant was tested: SPA14 (liposomes+QS21+E6020).

Eight cynomolgus macaques (4 males and 4 females) were used and allocated in 2 groups of 4 animals (2 males and 2 females).

On Day 0 and Day 28, a total of two RSV vaccine candidates were assessed (one formulation per animal group) through an intramuscular injection in the deltoid muscle. Samples were taken (sera and PBMCs) at different time points before and after immunizations over 5 months. Only SPA14-adjuvanted pre-F-ferritin induced RSV-A2 neutralizing antibodies, that peaked at day 49 (three weeks post-dose 2). Importantly, the adjuvanted RSV pre-F-ferritin formulations induced cross-neutralizing antibodies to RSV-B strain (ATCC 18537). No differences were observed between male and females. Neutralizing antibody responses in sera remained detectable through 5 months post-dose 2, demonstrating response longevity.

SPA14-adjuvanted pre-F-ferritin was also shown to induce F-specific memory IgG secreting cells.

Higher levels of RSV F-specific T cell IFNγ/IL-2 ELISpots responses were significantly induced by SPA14-adjuvanted formulations as compared to the non-adjuvanted group, demonstrating T_{H}1-type responses.

### I. Materials and Methods

### Animal Information

Cynomolgus macaques of 24 to 30 months (Macaca fascicularis—Noveprim), were housed at Cynbiose, SA (Marcy l'Etoile—France). This study was reviewed by the Animal Welfare Body of Cynbiose and the Ethics Committee of VetAgro-Sup (1 avenue Bourgelat, 69280 Marcy l'Étoile, France) and approved under number 1633-V3 (MESR number: 2016071517212815).

All experiments were conducted in accordance with the European Directive 2010/63/UE as published in the French Official Journal of Feb. 7, 2013.

The tested compositions and dosages are provided in Table 7 below.

TABLE 7

| | Product under Test | | | | | | |
|---|---|---|---|---|---|---|---|
| | Active substance | | Adjuvant | | Dilution | Injection | |
| | | Quantity/ | | Quantity/ | Buffer | | Volume |
| Group | Name | Dose (unit) | Name | Dose (unit) | composition | Route | injected (unit) |
| A | Pre-F | 50 μg/dose | SPA14 | DOPC: 1 mg/dose | pH: 6.73 | IM | 500 μL |
| | ferritin | | | Chol: 0.25 mg/dose | 301 mOsm/kg | | |
| | | | | QS21: 50 μg/dose | | | |
| | | | | E6020: 1 μg/dose | | | |
| B | Pre-F | 50 μg/dose | None | N/A | Buffer: PBS | IM | 500 μL |
| | ferritin | | | | pH 7.37 | | |
| | | | | | 294 mOsm/kg | | |

### Biological Sampling and Analytical Tests

### Blood Sampling for Serum Preparation

Blood samples for serum preparation were collected by venipuncture from the femoral vessel on conscious animals. Approximately, 4 mL per animal were sampled into tubes with clotting activator and separator gel (Vacutainer 6 SST™ II Advance, ref: 367955, Becton Dickinson) as follows: D1 2 (baseline), D13, D28 (before dose 2), D49, D63, D91, D119, D149 and D163. After overnight storage of the blood samples at temperature ranging from +2° C. to +8° C., serum was extracted by centrifugation at 2000×g for 20 min at +4° C. At least four 300 μL aliquots of serum were prepared from each animal and conditioned in cryotubes under a type 2 laminar flow cabinet.

### Blood Sampling for Peripheral Blood Mononuclear Cell (PBMC) Isolation

Blood samples for PBMC isolation were collected by venipuncture from the femoral vessel. Approximately, 12 mL per animal were sampled into Sodium-Heparin tubes of 6 mL (Vacutainer @, ref: 367876, Becton Dickinson).

This operation was performed on conscious animals, as follows: D12 (baseline), D7, D35, D119 and D161.

### Systemic RSV F-Specific IgG ELISAs

RSV-F Specific antibody titers were tested by manual ELISA. Briefly, microtiter plates (Dynex, Nunc) were coated with 1 μg/mL of RSV F protein (SinoBiologicals, cat. 11049-V08B) in bicarbonate buffer (Sigma, cat. C3041-100CAP). Plates were incubated overnight at 4° C. and then blocked with PBS-Tween 0.05%-milk 5% for 1 h. Sera were serially diluted in PBS-Tween 0.05%-milk 5% in the coated plate. After 1 h30 incubation at 37° C., washes plates (with PBS-Tween 0.05%) were incubated for 1 h30 at 37° C. with a goat anti-monkey IgG-HRP diluted 1:10 000 (Biorad, cat. AAI42P). Plates were washed and incubated with tetramethylbenzidine (TMB) substrate (Tebu-bio, cat. TMB 100-1000) for 30 m in the dark at RT. Colorimetric reaction was stopped with 100 μL per well of HCl 1M (VWR Prolabo, cat 30024290) and measured at 450 and 650 nm on a Versamax plate reader (Molecular Devices).

### RSV Plaque Reduction Neutralizing Test (PRNT60) for Detection of RSV Neutralizing Antibodies (RSV-A2 & RSV-B Strains)

Vero cells are seeded at 70,000 cells/well under 500 μL one day prior to infection on 24-well plates. On the day of infection, serum samples are first inactiviated for 30 min at 56° C. then 4-fold diluted in a DMEM-Glutamax+2% FBS+1% PS medium and finally mixed with an equal volume of virus (70 PFU/well) with or without guinea pig complement (10%) for an incubation time of 1 h at 37° C.

The supernatant of Vero cell is removed and replaced by 100 μL of DMEM-Glutamax+2% FBS+1% PS and with 100 μL/well of serum/virus mixture. After an incubation time of 1.5 hours at 37° C., a methylcellulose overlay (0.75% in DMEM-2% FBS-1% PS) is added on wells. Plates are incubated at 37° C. 5% CO2 for 5 days.

Following incubation time, plates are fixed with absolute methanol (−20° C.) for 1 h at 4° C.

Washes plates were blocked with PBS-milk 5% for 1 h at room temperature and then an immunostaining was performed:
For RSV-A2 strain, a polyclonal anti-RSV-HRP antibody (abcam, cat. 20686) diluted at 1:2000 at least 2 h at room temperature.
For RSV-B strain, a monoclonal anti-RSV fusion protein antibody (abcam, cat. 24011) diluted at 1:2000 for 1h at room temperature and after washes an anti-mouse HRP (abcam, cat. ab6789) diluted at 1:2000 for 1 h at room temperature.

After washing, plates were incubated with True Blue substrate (SeraCare, Cat. 5510-0030) under agitation at room temperature for few minutes. When plaques are visible, the reaction is stopped by washing with water. Plaques are detected and counted in a multi-modal reader (Viruscope, Microvision) and the neutralizing antibody titers were determined at the 60% reduction endpoint.

### F-Specific IgG Memory B-Cells ELISpots

F-specific B cell memory responses were evaluated after 5 days of PBMC polyclonal stimulation (IL-2+R848) to induce quiescent memory B cells to differentiate into antibody secreting cells (ASC). ASC frequency was then measured by the Human IgG FluoroSpot kit from Mabtech (product Code #FS_05R24G-10) adapted to measure F (SinoBio ref #1149_V08B) specific responses.

Briefly, PBMCs were cultured in complete medium (Roswell park Memorial Institute medium containing 10% fetal calf serum, 200 mM L-Glutamine, 100 U/ml penicillin and 10 μg/ml streptomycin) supplemented with R848 (1 μg/ml) and recombinant human IL-2 (10 ng/ml). After 5 days of culture, the cells were recovered, washed and used in the FluoroSpot assay as described below.

The fluorospot plates equipped with a low-fluorescent PVDF membrane were pre-wetted with 35% ethanol for 1 min, washed with sterile water, then with PBS and coated overnight at 5±3° C. with either the F antigen (SinoBio 11049-V08B) or a mix of capture mAbs (MT91/145 and MT57). Plates were washed with PBS and blocked with complete medium for 1 hour at 37° C. After washing with PBS, PBMCs were plated and incubated for 5h at 37° C. Following 3 washes in PBS containing 0.05% Tween20 and 6 washes in PBS, the plates were incubated for 2h at 37° C. with anti-human IgG-550 (MT78/145) detection mAb. After washing with PBS, fluorescent spots were enumerated with a spot reader (Microvision).

### RSV F-Specific IFNγ/IL-2 Fluorospots

FluoroSpot assay was performed using monkey IFNγ/IL-2 FluoroSpot kit from Mabtech (product #52122-10) to detect and enumerate cells secreting one or both cytokines. Briefly, Multiscreen™ 96-well IPFL plates (Millipore) were first pre-wetted for 1 mn with 25 μL of 35% ethanol for 1 minute at room temperature, washed with sterile water, then twice with PBS and coated overnight at 5±3° C. with 100 μL of a mix of anti-monkey IFNγ and anti-IL-2 purified clones MT126L and MT2A91 respectively. Wells were washed 3 times with 200 μL sterile PBS per well, followed by a saturation step with 200 μL of complete medium (Roswell park Memorial Institute medium containing 10% fetal calf serum, 200 mM L-Glutamine, 100U/ml penicillin and 10 μg/ml) 2 hours at 37° C. After elimination of complete medium, 0.2 10⁶ PBMCs were then added to each well with anti-CD28 mAb (0.1 μg/mL) as co-stimulator factor and incubated overnight with 2 μg/mL of a pre-mixed peptide or pool of RSV F protein, 2 μg/mL of RSV F protein (Sino Biological #11049100_V08B) or a positive control (anti-CD3). After 6 washes in PBS 0.25% BSA, 100 μL of a mix of FITC-conjugated anti-IFNγ (7-B6-1-FS clone) and biotinylated anti-IL-2 (MT8G10 clone) were added for 2h at room temperature in the dark. After 3 washes in PBS 0.25% BSA, 100 μL of mix of anti-FITC Ab and SA-550 streptavidin were added to each well and incubated at room temperature for 1 h in the dark. Plates were further washed 6 times with PBS 0.25% BSA. Finally, the back of the plates was removed, and undersides of wells quickly rinsed with water. Plates were air-dried and stored in the dark until reading. Each spot corresponding to a single or double producer cell of IFNγ or/and II-2 was enumerated with a fluorescent plate reader equipped with a filter for Cy3 and FITC fluorescences (Microvision).

### II. Results

### Systemic Humoral Responses

### Pre-F-Ferritin+SPA14 Induced Enhanced F-Specific IgG Titers in Sera

This study was designed to evaluate the time course of immunological responses to repeat dose of RSV pre-F-ferritin (unadjuvanted group) vs RSV pre-F+SPA14 vaccination (adjuvanted group) in sero-naïve cynomolgus monkeys, with immunizations at study start (D0) and at D28.

The F antigen from Sinobiological, was used to do those ELISAs. RSV F-specific IgG titers were significantly greater in the SPA14 group vs the unadjuvanted group at all time points after immunization (FIG. 13). The F-specific IgG titers peaked at D49 (3 weeks post-dose 2) within the SPA14 group, approximately 200-fold greater than the unadjuvanted group (geometric mean of 20 000 versus 100).

Through-out the study, the SPA14 group sustained a greater F-specific IgG titer than the unadjuvanted group. Responders were defined as animals with a ≥4-fold rise over the assay LOD (Limit of Detection) of 20 (post-dose levels≥80). Applying this criterion, the SPA14 group reached a 100% response rate by D13 and F-specific IgG titers were long-lasting with 100% responders at the end of the study (D161=5 months post-dose 2). The unadjuvanted group reached a maximum of 50% response rate by D49 (3 weeks post-dose 2) and at D161.

### RSV Pre-F-Ferritin+SPA14 Induced RSV-A2 Neutralizing Antibodies

Serum virus neutralizing antibodies correlate with protection from RSV disease in humans.

Unlike F-specific IgG titers, RSV-A2 virus neutralizing antibodies (complement-dependent and complement independent) were only induced by SPA14-adjuvanted formulations (FIG. 14).

Similar to RSV F-specific IgG responses, RSV-A2 virus neutralizing antibodies peaked at D49 (3 weeks post-dose 2).

Group adjuvanted with SPA14 had significantly higher neutralizing titers than the non-adjuvanted group (p<0.01).

Responders were defined as animals with a ≥4-fold rise over the assay LOD of 20 (post-dose levels≥80). Applying this criterion, the SPA14 group reached a 75% (3 of 4 animals) response rate by D28 in presence of complement and 100% by D49. In contrast, the unadjuvanted group did not show any responders (FIG. 14B).

In absence of complement, SPA14 reached a 100% responder rate at D49 (FIG. 14A).

At the end of the study (D161=5 months post-dose 2), SPA14 group still had 100% responders (with and without complement).

### Pre-F-Ferritin+SPA14 Induces Cross-Neutralizing Antibodies to RSV B Strain (ATCC18537)

Multiple variants of RSV A and B strains circulate in the human population, so an effective RSV vaccine must induce cross-neutralizing antibodies. Thus, the ability of immune serum to cross-neutralize the RSV-B strain (ATCC18537) was evaluated.

Applying the responder criteria (>80), the unadjuvanted formulation induced only 25% responders (¼ NHPs) at day 49 against the RSV-B strain whereas the SPA14-adjuvanted pre-F-ferritin could induce cross-neutralizing titers in 100% of the NHPs at D49, with the adjuvanted group eliciting higher cross-neutralizing titers than the unadjuvanted group (FIG. 15).

Taken together, these results indicate that the adjuvanted RSV pre-F-ferritin vaccine induces greater levels of cross-neutralizing RSV antibodies compared to the unadjuvanted RSV pre-F-ferritin vaccine.

### Systemic Cellular Responses F-Specific Memory B Cells Responses

F-specific IgG memory B cells were assessed by Fluorospot at D119 (3 months post-dose 2) and at D161 (5 months post-dose 2) (FIG. 16A-B).

SPA14 induced F-specific IgG circulating memory B cells detectable at D119 and D161. Memory responses were low but measurable (<100 spots/10⁶ cells or 0.01% to 0.1% of total IgG secreting cells). SPA14 induced significantly higher memory IgG secreting cells compared to the non-adjuvanted group (P-value<0.01).

### F-Specific IFNγ and IL-2 T Cell ELISpots

CD4+ T cells exert essential helper functions and are critical for B-cell activation and differentiation. Interferon (IFN)-γ and interleukin (IL)-2 were selected to analyze Th1 immune responses. SPA14 vaccinated monkeys developed strong cellular immune responses to F compared to the non-adjuvanted groups (P-value<0.01), as shown by the results from the IFNγ and IL-2 ELISpot assays one week after the first and second injection (FIG. 17). On day 35, IFNγ spot-forming cells (SFC) ranged from 100 to 500 per million PBMCs (FIG. 17A) and IL-2 SFCs ranged from 200 to 1000 per million PBMCs (FIG. 17B).

### III. Conclusions

In the study, only adjuvanted pre-F-ferritin induced RSV-A2 neutralizing antibodies, that peaked at day 49 (three weeks post-dose 2) and lasted for at least 6 months. SPA14 induced significantly high neutralizing titers. Importantly, the adjuvanted RSV pre-F-ferritin formulations induced cross-neutralizing antibodies to RSV-B strain (ATCC 18537).

SPA14 induced F-specific memory IgG secreting cells (reactive memory) that could be of great interest to reactivate the production of neutralizing antibodies after RSV infection if the circulating neutralizing antibodies (constitutive memory) are not high enough to confer protection.

Significantly higher levels of RSV F-specific T cell IFNγ/IL-2 ELISpots responses (Th1) were induced by SPA14-adjuvanted formulation as compared to the antigen alone group.

By measuring cytokines in PBMC supernatants, SPA14 generated IFNγ-secreting cells (Th1).

### Example 9: Preparation of the CMV-Antigens and of the Vaccine Compositions

### Preparation of the CMV-Antigens and of the Vaccine Compositions for Examples 4, 10 and 11

### Materials

The antigens and adjuvants used in the following Examples are described in Table 8.

TABLE 8
Adjuvants formulations

| Product Name | Concentration | Source or composition |
|---|---|---|
| Pentamer HCMV | 80 μg/ml | Obtained as disclosed |
| gH/gL/pUL128/ | | hereinafter. |
| pUL130/pUL131 | | |
| (stock solution) | | |
| gB (HCMV) | 80 μg/ml | gBdTM obtained as |
| (stock solution) | | described in U.S. Pat. |
| | | No. 6,100,064, which is a |
| | | 806 amino acids long |
| | | polypeptide |
| AF03 (stock | Squalene: 50 mg/mL | AF03 was obtained as |
| solution) | Sorbitan trioleate: 7.4 | described in Klucker et |
| | mg/mL | al., Journal of |
| | Polyoxyethylene-12- | Pharmaceutical Sciences, |
| | cetostearyl ether: 9.5 | Volume 101, Issue |
| | mg/mL | 12, December 2012, |
| | Mannitol: 9.2 mg/mL | Pages 4490-4500 |
| | Phosphate buffered saline | |
| | solution q.s.p. 1 mL | |
| AF04 (stock | Squalene: 50 mg/mL | AF04 was obtained as |
| solutions) | Sorbitan trioleate: 7.4 | described in WO |
| | mg/mL | 2007/080308. |
| | Polyoxyethylene-12- | Quantity of E6020 was |
| | cetostearyl ether: 9.5 | adjusted to SPA14 |
| | mg/mL | intermediate low E6020 |
| | Mannitol: 9.2 mg/ml | dose. |
| | E6020: 4 or 40 μg/mL | |
| SPA14 (stock | QS21: 200 μg/mL | Liposomes obtained as |
| solutions) | E6020: 4, 8, 20, or 40 | described below |
| | μg/ml | |
| | 1,2-dioleoyl-sn-glycero-3- | |
| | phosphocholine (DOPC): | |
| | 4000 μg/ml | |
| | cholesterol: 1000 μg/ml | |
| AS01E (final | QS21: 50 μg/ml | Liposomes obtained from |
| conc.) | MPL: 50 μg/ml | Shingrix ® vaccine and |
| | 1,2-dioleoyl-sn-glycero-3- | half diluted vol/vol with |
| | phosphocholine (DOPC): | antigens |
| | 1000 μg/ml | |
| | Cholesterol: 250 μg/ml | |
| AS01B (final | QS21: 100 μg/ml | Liposomes obtained from |
| conc.) | MPL: 100 μg/ml | Shingrix ® vaccine |
| | 1,2-dioleoyl-sn-glycero-3- | |
| | phosphocholine (DOPC): | |
| | 2000 μg/ml | |
| | Cholesterol: 500 μg/ml | |

### Methods of Preparation

Antigens and adjuvants formulations were prepared as indicated in Table 8 or as disclosed below.

Except for AS01E and AS01B, the stock solutions of the adjuvants were mixed with the antigens volume at volume, and then the required dose: 50 or 500 μL (for mice or rabbits), was administered.

AS01B was obtained from Shingrix® and was used with a more concentrated antigens to maintain the initial concentration.

AS01E results from a mix volume:volume with the antigens (as for the other adjuvants).

### HCMV Pentamer

The HCMV pentamer gH/gL/pUL128/pUL130/pUL131 was obtained in CHO cell line transfected with 5 plasmids, each plasmid comprising the sequence coding for one of the 5 proteins constituting the HCMV pentamer. The sequences were from the strain BE/28/2011 (Genbank ID KP745669). The gH sequence was without the transmembrane domain for secretion of the recombinant pentamer. An example of expression of pentamer complex is given in Hofmann et al. (Biotechnology and Bioengineering, 2015, vol 112, issue 12, pages 2505-2515).

### SPA14

For example, the stock solution of SPA14 with E6020 at 20 μg/ml was prepared as follows.

The liposomes were prepared according to the solvent, e.g. ethanol, injection method as follows.

A solution of E6020 in ethanol was prepared at 2 mg/ml by dissolving 2.0 mg of E6020 powder in 0.998 ml of ethanol.

A 4-fold concentrated ethanol solution was prepared at 40 mg/ml of DOPC, 10 mg/ml of cholesterol and 0.200 mg/ml of E6020 by dissolving, in 0.850 ml of ethanol, 40 mg of DOPC and 10 mg of cholesterol and 0.100 ml of the previously prepared E6020 solution in ethanol.

The solution was stirred at room temperature (RT) until total dissolution of the product and obtaining a colorless solution.

In a 7 ml Lyo glass vial, 3.0 mL of CBS (Citrate Buffer Solution) pH 6.3 (citrate 10 mM, NaCl 140 mM pH 6.3) were stirred at 1000 rpm at room temperature. 1.0 ml of the lipid solution was slowly added using a Hamilton syringe with a 22 ga needle and a syringe pump at 0.1 ml/min to form liposomes. Liposomes were dialyzed (on 10000 MCWO dialysis cassettes) three times (half a day, one night and one day) against CBS pH 6.3.

Liposomes suspension was sterile filtered on Millex filter PVDF 0.22 μm of 33 mm diameter and stored at +4° C. under nitrogen.

Liposomes components concentrations were estimated according to the dilution factor of the dialysis. For a 1.6 dialysis dilution factor, liposomes components concentrations were at 6.25 mg/ml of DOPC, 1.56 mg/ml of Cholesterol and 0.031 mg/ml of E6020.

Under a flow hood, 3.0 mg of QS21 was re-suspended in 3.0 mL of CBS pH 6.3 to obtain a solution of QS21 at 1.0 mg/ml and sterile filtered on Pall Acrodisc 0.2 μm of 25 mm diameter. In sterile conditions, SPA14 (liposomes suspension) was formulated by addition of 1.563 ml of the solution of QS21 at 1.0 mg/ml in CBS pH 6.3 to 5.000 ml of the previous liposomal suspension and 1.250 ml of CBS pH 6.3. The mixture was stirred for 10 seconds using a vortex and stored at +4° C. under Nitrogen for a final SPA14 sterile suspension of 4 mg/ml DOPC, 1 mg/ml cholesterol, 0.020 mg/ml E6020 and 0.200 mg/ml QS21.

To do the mixture with antigens, the SPA14 adjuvant was gently turned upside down 5 times to homogenize the product prior mixing with antigen(s) twice concentrated. The immunogenic compositions were then stored at an appropriate temperature (2-8° C.) until further use.

The mixing with the antigen was done volume/volume and the resulting mixture was gently turned upside down 5 times. The mixtures were prepared just before injection or maximum 3 hours before the injection. In this later case, they had to be placed at 2-8° C. until injection.

For Example 10, a SPA14 stock solution was prepared as described above but with a final ratio DOPC:Cholesterol:QS21:E6020 at 4:1:0.2:0.04 mg/ml.

For Example 4 and 11, SPA14 stock solutions were prepared as described above at 4:1:0.2:X mg/ml of respectively DOPC/Chol/QS21/E6020. Four different concentrations X of E6020 were used: 0 mg/ml, 0.004 mg/ml, 0.008 mg/ml, and 0.02 mg/ml E6020 to obtain the doses of E6020 described in Table 8 in Example 9 (dilution v/v with the antigens and 500 μl injected).

### Example 10: Evaluation of Different Adjuvants

### Evaluation of Different Adjuvants

Adjuvants AF04, SPA14 and AS01E were compared to AF03 (AF03 is a squalene emulsion adjuvant, as was MF59 used in the clinical trials with gB antigen—see WO 2007/005583—that showed 50% efficacy in preventing HCMV acquisition of primary HCMV but with a rapidly declining level of neutralizing antibodies).

### Materials & Methods

In the present mouse study, groups of 37-week old naïve female C57BL/6J mice received three intra-muscular (IM) immunizations of CMV gB and CMV pentamer (2 μg each/dose-dose: 50 μL) formulated with AF03 (1.25 mg squalene/dose), AF04 (AF03 squalene-based emulsion containing 1 μg/dose E6020), SPA14 (DOPC-Chol liposomes containing 5 μg QS21 and 1 μg E6020/dose) or AS01E (half-dilution of AS01B obtained from the commercial vaccine Shingrix) adjuvants via the IM route on days 0, 21 and 221 (month 7). Blood samples were collected at months 1, 2, 3, 4, 5, 6, 7 and 8 for monitoring of the seroneutralizing antibody response. Around 1 mL of blood was collected in vials containing clot activator and serum separator (BD Vacutainer SST ref 367783). After a night at +4° C., blood was centrifuged at 3000 rpm during 20 minutes and serum was collected and stored at −20° C. until analysis. Additionally, at months 1, 7 and 8, blood and spleens were collected from 10 mice per group to monitor the CMV gB- and CMV pentamer specific IgG antibody subclasses, the Antibody Secreting Cell (ASC) frequencies as well as the IL-5 and IFN-γ secretions.

For cellular response assays, spleens were collected in sterile conditions and splenocytes were isolated as soon as possible after spleen sampling.

### Seroneutralization Assays

Briefly, 2.5×10⁴ MRC-5 fibroblasts or ARPE-19 cells were dispensed in 96-well dark plates the day before the microneutralization (MN) assay. On D0, sera were heat-inactivated at 56° C. for 30 min. Serum samples were serially two-fold diluted in DMEM/F12 1% FBS, starting from 1/10 to 1/10240 in a 96-deep-well plate and incubated with 4.2 log FFU/ml of the BADrUL131-Y4 CMV virus strain (as described in Wang et al., J Virol. 2005 August; 79(16):10330-8) for 60 min at 37° C. in a 5% C₀₂ cell culture incubator. The serum/virus mixtures were then transferred onto the MRC5 or the ARPE-19 cells and incubated at 37° C. in a 5% CO₂ cell culture incubator for 3 days for the MRC5 cells and for 4 days for the ARPE cells.

Culture supernatant was then removed, and cells were fixed with 100 μl of 1% formol in PBS for 1 hour at room temperature. The plates were then washed with PBS and air-dried at room temperature before analysis on the Microvision fluorescent plate reader to count infected cells in each well.

As control, two wells of cell control (without virus) and six wells with cells infected with half of the viral dilution containing the 4.2 log FFU/mL were present on each plate. The mean of these six wells defined the threshold of seroneutralization, determined as 50% of the specific-signal value. Neutralizing end-point titers were defined as the reciprocal of the last dilution that fell below the calculated 50% specific-signal value. Neutralizing titers (μPRNT50) were defined for each subject serum as the last dilution that induced 50% reduction of infected cells, i.e. the last dilution that induced lower infected cells than the calculated 50% specific-signal value. Geometric mean neutralizing antibody titers were calculated for each group.

### ELISA Assay

Serum IgG1 and IgG2c antibodies directed against CMV-gB antigen or against CMV-pentamer antigen were titrated by a robot ELISA assay according to the following procedure.

Dynex 96-well microplates were coated overnight at 4° C. with 1 μg/well of CMV-gB or CMV-pentamer, in 0.05 M carbonate/bicarbonate buffer, pH 9.6 (Sigma). Plates were then blocked at least 1 hour at 37° C. with 150 μL/well of PBS Tween-milk (PBS pH7.1, 0.05% Tween 20, 1% (w/v) powdered skim milk (DIFCO)). All next incubations were carried out in a final volume of 100 μL, followed by 3 washings with PBS pH 7.1, 0.05% Tween 20. Serial two-fold dilution of serum samples were performed in PBS-Tween-milk (starting from 1/1000 or 1/10000) and were added to the wells. Plates were incubated for 90 min at 37° C. After washings, goat anti-mouse IgG1 or IgG2c peroxydase conjugate antibodies (Southern Biotech) diluted in PBS-Tween-milk at 1/2000 were added to the wells and plates were incubated for 90 min at 37° C. Plates were further washed and incubated in the dark for 30 min at 20° C. with 100 μL/well of a ready-to-use Tetra Methyl Benzidine (TMB) substrate solution (TEBU). The reaction was stopped with 100 μL/well of HCl 1M (Prolabo).

Optical density (OD) was measured at 450 nm-650 nm with a plate reader (VersaMax—Molecular Devices). The IgG1 or IgG2c antibodies titers were calculated using the CodUnit software, for the OD value range of 0.2 to 3.0 from the titration curve (reference mouse hyper immune serum put on each plate). The IgG1 or IgG2c titer of this reference, expressed in arbitrary ELISA Units (EU) corresponds to the log 10 of the reciprocal dilution giving an OD of 1.0. The threshold of antibody detection was 10 ELISA units (1.0 log 10). All final titers were expressed in log 10 (Log).

IgG1/IgG2c ratios were calculated using the individual arithmetic values and the geometric mean of individual IgG1/IgG2c ratios was calculated for each group.

### Fluorospot

The fluorescent-linked immunospot (FLUOROSPOT) was used for detecting and enumerating individual cells secreting the IFN-γ and IL-5 cytokines.

The membrane of the 96-well IPFL-bottomed microplates (Multiscreen) was pre-wetted with 35% ethanol, then washed twice with PBS 1×. Microplates were then coated with a rat anti-mouse IFN-γ or rat anti-mouse IL-5 antibodies (10 μg/ml, Pharmingen) diluted at 1/100 and 1/50 respectively and were incubated overnight at 4° C.

On D1, plates were washed with PBS and then blocked at least 2h at 37° C. with RPMI 10% FBS. After plates washing, 5×10⁵ freshly isolated splenocytes/well were incubated overnight with the CMV-gB antigen (0.1 μg/ml), CMV-pentamer (0.1 μg/ml) or concanavalin A (Con A, 2.5 μg/mL) as a positive control, in presence of murine IL-2 (10 U/ml).

On D2, the plates were washed 6 times with PBS 1×-BSA 0.1% (200 μL/well). After the washing step, 100 μL/well of the biotinylated anti-mouse IFN-γ or anti-mouse IL5 antibodies were added at 1 μg/mL in PBS1×-BSA 0.1% for 2 hours at room temperature, in the dark. The plates were washed again 3 times with PBS 1×-BSA 0.1% (200 μL/well). Then, 100 μL/well of streptavidin-PE at 1 μg/mL in PBS 1×-BSA 0.1% was incubated for 1 hour at room temperature, in the dark.

The plates were further washed 6 times with PBS 1×-BSA 0.1% (200 μL/well). The plates were stored at 5° C.±3° C. in the dark until reading.

Each spot, corresponding to an IFN-γ or IL5 secreting cell (IFN-γ SC or IL5 SC), was enumerated with an automatic FLUOROSPOT plate reader (Microvision). Results were expressed as number of IFN-γ or IL-5 secreting cell per 10⁶ splenocytes.

### IgG, IgG1 and IgG2c FLUOROSPOT Assay

The fluorescent-linked immunospot (FLUOROSPOT) is used for detecting and enumerating individual B cells secreting antibodies irrespective of antigen specificity (IgG1, IgG2c or total IgG).

The membrane of the 96-well IPFL-bottomed microplates (Multiscreen) was pre-wetted with 35% ethanol, then washed twice with PBS 1×. Microplates were then coated with CMV-gB antigen (10 μg/ml, Sanofi), CMV-pentamer (10 μg/ml, NAC) or total IgG antibody (10 μg/ml, KPL) diluted at 1/68, 1/100 and 1/100 respectively and were incubated overnight at 4° C.

On D1, plates were washed with PBS and then blocked at least 2h at 37° C. with RPMI 10% FBS.

After plates washing, 5×10⁵ freshly isolated splenocytes/well for CMV-gB antigen or CMV-pentamer and 2.5·10⁵ freshly isolated splenocytes/well for total IgG antibody were incubated 5 hours.

After 5 hours, the plates were washed 3 times with PBS 1× and stored a 4° C. for the night.

On D2, the plates were washed 6 times with PBS 1×-BSA 0.1% (200 μL/well). After the washing step, 100 μL/well of the anti-mouse IgG1 PE or anti-mouse IgG2c FITC or anti-mouse total IgG antibodies were added respectively at 4, 2 or 0.5 μg/mL in PBS1×-BSA 0.1% for 2 hours at room temperature, in the dark. The plates were washed again 6 times with PBS 1×-BSA 0.1% (200 μL/well). The plates were stored at 5° C.±3° C. in the dark until reading.

Each spot, corresponding to an antibody secreting cell (ASC) (IgG1 ASC, IgG2c ASC or total IgG ACS), was enumerated with an automatic FLUOROSPOT plate reader (Microvision). Results were expressed as number of antibody secreting cell per 10⁶ splenocytes.

### Statistical Analysis

All analyses were performed on SAS v9.2® (SAS Institute, Cary, NC). Analysis Of Variance (ANOVA) one factor model with group as fixed factor or ANOVA, 2 factors with group and time as fixed factors were performed. For longitudinal analysis, Analysis of Covariance (ANCOVA), with group as category variable and time as continuous variable was performed. For comparison to AF03, Dunnett adjustment was used.

### Results

As shown on FIG. 18, an adjuvant effect was demonstrated on the CMV gB- and CMV pentamer-specific immune response for all tested formulations, whatever the timepoint. SPA14 elicited statistically significantly higher (≥3-fold) and more persistent neutralizing antibody titers compared to AF03 (test of superiority, bilateral Dunnet adjustment, all p_values<0.05). Also, SPA14 elicited a higher neutralizing antibody response than AF04. SPA14 and AS01E elicited neutralizing antibody response of similar amplitude.

As shown on FIGS. 19A and 19B, the analyses at month 1 (15 days after the 2^{nd} dose) and at month 8 (28 days after the last dose) confirmed the immune profiles observed for SPA14, AS01E, AF03 and AF04 at earlier time-points and the ability of SPA14 and AS01E adjuvants to induce higher and more persistent neutralizing antibody titers (FIGS. 19A and 19B, all p-values<0.05).

Furthermore, SPA14 and AS01E induced a higher IgG2c B memory cell frequencies as well as a more Th1-oriented cellular response than AF03. With respect to the profile of the immune response, the IgG1/IgG2c ratios showed a Th1 profile for the groups receiving SPA14 and AS01E, whereas a Th2 profile was observed with AF03 eliciting high IgG1 titers. It was confirmed by the cellular response analysis at month 1. The Th1-skewed profile of SPA14 and AS01E as compared to AF03 was exhibited by a decrease in IL-5 and an increase in IFN-γ production upon stimulation either with CMV gB or CMV pentamer. The analyses at month 7 and 8 confirmed the immune profiles observed for SPA14 and AS01E at earlier time-points and the ability of these adjuvants to induce a more Th1-oriented cellular response than AF03 (FIG. 20A-B). Moreover, the CMV gB- and CMV pentamer-specific IgG2c/IgG1 ASC ratio among plasmablasts and B memory cells, associated to a Th1 orientation, were higher with SPA14 and AS01E than with AF03. This early memory B cell response measured at day 35 for SPA14 was confirmed at month 7 (0.6% up to 1% IgG2c-ASC memory B cells specific to CMV gB and CMV pentamer detected). These IgG2c ASC frequencies were significantly higher than with AF03 (all p-values<0.001). Furthermore, it is observed that SPA14 elicited a more balanced Th1/Th2 response than AS01E. As shown in FIG. 20, and particularly at month 8, SPA14 induce lower increase of IFN-γ secreting cells (2.6-fold lower, not significant) and lower decrease of IL-5 secreting cells (2.9-fold higher, p-value<0.001) than AS01E, resulting to a lower and more balanced Th1/Th2 ratio as compared to AS01E.

Analyses conducted up to month 8 in the mouse model allow us to conclude that SPA14 and AS01E were able to fulfill the expected criteria (i) higher neutralizing antibodies titers than AF03, (ii) a Th1-biased profile compared to AF03 as evidenced by lower IgG1/IgG2c sub-class ratios and higher IFNγ/IL-5 ratio and (iii) a more persistent neutralizing antibodies response and higher frequencies of memory B cells compared to AF03, and could be suitable adjuvants to improve a CMV vaccine candidate.

When comparing SPA14 to AF04, we observed that AF04 elicited an intermediary response between AF03 and SPA14. For the same TLR4 agonist concentration evaluated either in emulsion (AF04) or in liposome (SPA14), AF04 was able to induce higher neutralizing antibodies than AF03 at months 1 and 8 (FIG. 18), however this increase of neutralizing titers was lower than the one obtained with SPA14. Similarly, for the Th1/Th2 profile, AF04 was able to increase IFN-γ and decrease IL-5 as compared to AF03 but these changes were enhanced and more pronounced with SPA14 formulation.

### Conclusion

In conclusion, SPA14 and AS01E were the adjuvants inducing the highest neutralizing antibody titers specific to both gB and Pentamer and the highest long-lasting immune response against gB and Pentamer. Both adjuvants induce a Th1 profile immune response. SPA14 induces a more balanced Th1/Th2 response.

### Example 11: Reactogenicity of a Composition Adjuvanted with SPA14

Reactogenicity of a Composition Adjuvanted with SPA14

The objective of the study was to investigate potential reactogenicity of CMV antigens-containing vaccine compositions containing as adjuvant either SPA14 or AS01B in the same New Zealand White rabbits groups of Example 4 following two intramuscular injections at three weeks intervals, and to evaluate the delayed occurrence and/or reversibility of any local reaction during a 2-weeks observation period.

The SPA14- and AS01B-adjuvanted immunogenic compositions containing CMV antigens were as described in Example 4.

### Materials and Methods

### Animals and Study Design

Animals and study design were the ones of Example 4.

### Blood Samples Collection

Blood samples were collected before initiating the study, and then at day 2, 3, 7, 23, 24, and 36. For fibrinogen analysis, blood samples were taken in vials containing trisodium acetate and for neutrophil counts, blood samples were taken with tubes containing EDTA-K2.

### Neutrophil Counts

Neutrophil counts were determined using the ADVIA (120 or 2120, Siemens) according to recommendations of manufacturer.

### Fibrinogen

The fibrinogen parameter was determined using the STAR Max (Stago) system according to recommendations of manufacturer.

### Globulin

The globulin parameter was determined using the AU680 (Beckman Coulter) system according to recommendations of manufacturer.

### C-Reactive Protein

The CRP was determined using an ELISA (CRP-10 Life Diagnostics). The samples were prepared as follows: the samples were centrifugated at 1 800 G for 10 minutes at about 4° C. The serum was then collected on ice. The ELISA kit was used according to manufacturer recommendations.

### Results

Two intramuscular administrations of immunogenic compositions comprising the CMV antigens gB+Pentamer (gH/gL/UL128/UL130/UL131A) adjuvanted either with AS01B or with SPA14 at 3-week intervals to NZW rabbits did not induce any systemic toxicity.

Slight increases were observed for neutrophil counts and fibrinogen levels, as well as an increase in globulin and CRP levels in animals receiving the AS01B-adjuvanted compositions compared to the animals receiving SPA14.

TABLE 9
AS01B and SPA14 induced neutrophil counts (G/L)
increases 24 h and 48 h post-injections

| | Group | | | | | | |
|---|---|---|---|---|---|---|---|
| Days | 1 | 2 | 3 | 4 | 5 | 6 | 7 |
| Pre-test | 0.96 | 1.18 | 0.99 | 0.92 | 0.99 | 0.91 | 0.95 |
| D2 | 1.34 | 1.17 | 3.05 | 1.45 | 1.72 | 2.77 | 3.00 |
| D3 | 1.19 | 0.99 | 1.43 | 1.03 | 1.34 | 1.46 | 1.70 |
| D7 | 1.83 | 1.21 | 1.54 | 1.38 | 1.36 | 1.28 | 1.71 |
| D23 | 1.20 | 1.39 | 2.70 | 1.30 | 1.25 | 1.38 | 1.71 |
| D24 | 1.06 | 0.99 | 1.46 | 0.85 | 1.08 | 0.92 | 1.10 |
| D36 | 1.03 | 0.92 | 1.08 | 0.77 | 1.04 | 1.00 | 1.06 |

After a 1^{st} injection, AS01B (Group 3) induced a neutrophil count increase compared to antigens alone (ab. 2-fold). While an increase was also observed for SPA14, it remained moderate, and was as strong as AS01B only at 5 μg of E6020.

AS01B induced also an increase after the 2^{nd} injection of the immunogenic composition, which was not observed, or only very moderately and lasting less long (SPA14 @ 5 μg/ml of E6020), with SPA14 formulations.

TABLE 10
AS01B and SPA14 induced fibrinogen levels (g/L)
increases 24 h and 48 h post-injections

| | Group | | | | | | |
|---|---|---|---|---|---|---|---|
| Days | 1 | 2 | 3 | 4 | 5 | 6 | 7 |
| Pre-test | 2.39 | 2.45 | 2.58 | 2.55 | 2.36 | 2.30 | 2.57 |
| D3 | 2.34 | 2.46 | 4.39 | 3.01 | 3.14 | 4.27 | 4.76 |
| D24 | 2.28 | 2.54 | 3.91 | 3.33 | 3.05 | 3.31 | 3.60 |
| D36 | 2.27 | 2.38 | 2.84 | 2.44 | 2.55 | 2.49 | 2.34 |

Forty-eight hours after the 1 st and 2nd injections, AS01B induced a fibrinogen level increase up to 88% compared to antigens alone. Again, if SPA14 induced fibrinogen increase 48h after 1^{st} injection, the fibrinogen level was increased to a lesser extent after the 2^{nd} injection (as compared to the increase induced by AS01B).

TABLE 11
AS01B and SPA14 induced globulin levels (g/L)
increases 24 h and 48 h post-injections

| | Group | | | | | | |
|---|---|---|---|---|---|---|---|
| Days | 1 | 2 | 3 | 4 | 5 | 6 | 7 |
| Pre-test | 12.55 | 12.10 | 12.94 | 12.69 | 11.61 | 11.71 | 13.23 |
| D2 | 13.58 | 12.80 | 14.89 | 13.6 | 12.91 | 14.61 | 14.84 |
| D3 | 12.73 | 11.94 | 15.50 | 13.86 | 13.56 | 14.15 | 14.74 |
| D7 | 12.96 | 12.00 | 13.89 | 13.09 | 13.08 | 13.21 | 13.58 |
| D23 | 12.88 | 11.81 | 13.99 | 12.86 | 12.86 | 12.85 | 13.54 |
| D24 | 13.34 | 12.98 | 15.63 | 14.39 | 13.76 | 13.70 | 14.64 |
| D36 | 13.08 | 12.58 | 15.53 | 14.08 | 14.00 | 14.03 | 13.60 |

AS01B induced an increase in globulin levels, as compared with antigens alone, slightly higher than what was observed with the SPA14-adjuvanted compositions, notably after the 2^{nd} injection.

TABLE 12
AS01B and SPA14 induced CRP concentrations (mg/L)
increases 24 h and 48 h post-injections

| | Group | | | | | | |
|---|---|---|---|---|---|---|---|
| Days | 1 | 2 | 3 | 4 | 5 | 6 | 7 |
| Pre-test | 3.0 | 3.6 | 5.4 | 4.8 | 5.7 | 2.5 | 5.9 |
| D2 | 7.8 | 5.0 | 140.8 | 43.6 | 43.3 | 170.5 | 182.0 |
| D3 | 4.8 | 4.2 | 107.2 | 26.7 | 26.0 | 93 | 93 |
| D7 | 6.3 | 6.3 | 10.1 | 8.3 | 5.5 | 5.5 | 8.2 |
| D23 | 7.1 | 12.0 | 161.2 | 75.9 | 62.9 | 100.6 | 117.1 |
| D24 | 5.9 | 4.8 | 80.8 | 30.0 | 23.1 | 36.3 | 42.1 |
| D36 | 2.1 | 2.5 | 3.8 | 3.2 | 4.6 | 5.3 | 2.0 |

AS01B induced an increase in CRP level higher than the one observed with SPA14-adjuvanted formulations. Further, the increase lasted longer than the ones observed with SPA14, up to 48h. Notably, 48h after the 2^{nd} injection, AS01B induced a CRP level that is double of the CRP level induced by SPA14 with the 5 μg/ml of E6020. CRP is a well-known reactogenic biomarker.

### Conclusions

Two IM administrations of CMV-gB+Pentamer adjuvanted with AS01B or SPA14 (containing E6020 from 0 to 5 μg), at 3-week intervals to NZW rabbits, were well tolerated and did not induce any systemic toxicity. Only local reactions were noted at injection sites (IS) evidenced mainly by oedema in all treated groups but that were not considered as adverse. Slight and reversible neutrophil count and fibrinogen level increases were observed that were comparable between adjuvanted treated groups but these increases were to a lesser extend with SPA14 than AS01B. An increase in globulin and CRP levels was also noted in all adjuvanted treated groups that with SPA14 formulations was lower and, for the reactogenic biomarker CRP, with a faster return to basal level, than with AS01B. These changes suggest a lower reactogenic profile with SPA14 formulations as compared to AS01B.

### Overall Conclusion of the Examples 4, 10 and 11

As shown by the above Examples 4, 10 and 11, immunogenic compositions containing CMV antigens, as exemplified with gB and pentamer (gH/gL/UL128/UL130/UL131) antigens, and adjuvanted with SPA14 adjuvant proved to elicit higher long lasting seroneutralizing antibodies as compared with other adjuvants containing TLR4 agonists such as AF04. Immune response elicited with SPA14 was comparable to the immune response elicited with AS01B.

Although inducing a Th1-oriented response, the immune response elicited by immunogenic compositions adjuvanted with SPA14 presented a more balanced Th1/Th2 profile as compared with AS01B.

Finally, as illustrated by the CRP, fibrinogen, or globulin responses, CMV-immunogenic compositions adjuvanted with SPA14 presented a lower reactogenic profile compared to AS01B-adjuvanted immunogenic compositions.

Immunogenic compositions containing CMV antigens, such as gB and pentamer (gH/gL/UL128/UL130/UL131) antigens, and adjuvanted with SPA14 present an excellent profile in terms of elicited immune responses and low reactogenicity to ensure a good adhesion to CMV vaccination program either by practicians or by intended recipients.

### Example 12: Qs21 and Qs7 Saponines in SPA14-Like Formulations

Comparison of QS21 v. QS7 in SPA14 Formulations

The objective of the study was to compare the haemolytic activity and the adjuvanting effect of SPA14 formulations containing either QS21 or QS7 as saponin.

### Materials and Methods

### Preparation of Liposomes

The liposomes were prepared with the hCMV antigens gB and pentamer, as described in Examples 1 and 4.

### Hemolysis Assay

Before use, red blood cells (Sheep red blood cells 10%, Rockland, ref R405-0050, lot BP30202 (stored+4° C.) were washed in cold PBS. Five mL of sheep red blood cells were transferred into a 15 mL Falcon tube and 7 mL of cold PBS were added. Cells were centrifuged for 10 min 700 g at 4° C. Supernatant was carefully removed and cell pellet was suspended in 12 mL of cold PBS. Then, cell suspension was centrifuged for 10 min, 700 g at 4° C. The cell suspension and centrifugation steps were repeated twice. Finally, cells were suspended in PBS in a final volume of 5 mL ready-to-use.

In a round bottom P96 plate, 100 μL per well of PBS were added. Then, 100 μL per well of saponin QS21 or QS7 solutions were added in serial 2-fold dilutions (1.6 μM to 200 μM-) in a citrate buffer (Citrate 10 mM, NaCl 140 mM, pH 6.3). Citrate buffer alone was used as control solution.

Twenty-five μL/well of 10% red blood cell solution were added, and the plates were incubated for 30 minutes at 37° C. and then were centrifuged for 5 min 700 g at room temperature. Eighty μL per well of surnageant were collected and transfered to a flat bottom plate for spectrophotometer reading (OD 540 nm). The percentage of cell hemolysis is calculated for each saponin concentration tested in μM according to the formula: 100×[(sample absorbance−negative control absorbance)+(positive control absorbance−negative control absorbance)]. Statistical analyses were carried out by Tukey adjustment+one-way ANOVA: p<0.05.

### Immune Response Study in C57BL/6 Mice

9 groups (n=8) of 6-8 weeks old C₅₇BL/6 mice received two IM immunizations (prime and boost: DO and D20) in the quadricep muscle with a final injection volume of 50 μL containing 2 μg of CMV gB and CMV pentamer (2 μg each/dose) formulated with:
DOPC-Chol liposomes (4000:1000 μg/ml) containing QS21 (5 μg) without E6020 (“QS21 LIP” (0:200 μg/mL)),
DOPC-Chol liposomes (4000:1000 μg/ml) containing E6020 without QS21 or QS7 (“E6020 LIP” (20:0 μg/mL)),
SPA14(DOPC-Chol liposomes (4000:1000 μg/ml) containing 5 μg QS21 and 0.5 μg E6020/dose (“SPA14” (20:200 μg/mL))
SPA14-like formulation containing QS7 (DOPC-Chol liposomes (4000:1000 μg/ml) containing 5, 15 or 45 μg QS7 and 0 or 0.5 μg of E6020/dose (“QS7 LIP” (0:200 μg/mL)”, (0:600 μg/mL) or (0:1800 μg/mL), “LIP [QS7+E6020 20]” (20:200 μg/mL), (20:600 μg/mL) or (20:1800 μg/mL))

TABLE 13
FORMULATIONS

| | | Active substances |
|---|---|---|
| Adjuvants | SPA14 | 20 μg/mLE6020, |
| | | 200 μg/mL QS21, |
| | | 4.0 mg/mL DOPC, |
| | | 1.0 mg/mL Cholesterol |
| | SPA-14 like | 20 μg/mLE6020, |
| | formulations | 200 μg/mL QS7, |
| | containing QS7 | 4.0 mg/mL DOPC, |
| | | 1.0 mg/mL Cholesterol |
| | | 20 μg/mLE6020, |
| | | 600 μg/mL QS7, |
| | | 4.0 mg/mL DOPC, |
| | | 1.0 mg/mL Cholesterol |
| | | 20 μg/mLE6020, |
| | | 1800 μg/mL QS7, |
| | | 4.0 mg/mL DOPC, |
| | | 1.0 mg/mL Cholesterol |
| | QS21 liposomes | 200 μg/mL QS21, |
| | | 4.0 mg/mL DOPC, |
| | | 1.0 mg/mLCholesterol |
| | QS7 liposomes | 200 μg/mL QS7, |
| | | 4.0 mg/mL DOPC, |
| | | 1.0 mg/mL Cholesterol |
| | | 600 μg/mL QS7, |
| | | 4.0 mg/mL DOPC, |
| | | 1.0 mg/mL Cholesterol |
| | | 1800 μg/mL QS7, |
| | | 4.0 mg/mL DOPC, |
| | | 1.0 mg/mL Cholesterol |
| | E6020 liposomes | 20 μg/mLE6020, |
| | | 4.0 mg/mL DOPC, |
| | | 1.0 mg/mL Cholesterol |
| Antigens | hCMV gB | 0.619 mg/mL |
| | hCMV pentamer | 1.011 mg/mL |
| | (gH/gL/pUL128/ | |
| | pUL130/pUL131) | |
| Vaccines | hCMV gB + hCMV | 2 μg hCMV gB |
| | pentamer | 2 μg hCMV |
| | | Pentamer |
| | hCMV gB + hCMV | 2 μg gB, |
| | pentamer formulated | 2 μg hCMV |
| | in SPA14 | Pentamer |
| | | 0.5 μg E6020 |
| | | 5 μg QS21 |
| | | 100 μg DOPC |
| | | 25 μg Cholesterol |
| | hCMV gB + hCMV | 2 μg hCMV gB |
| | pentamer formulated | 2 μg Pentamer |
| | in LIP [QS7 200 + | 0.5 μg E6020 |
| | E6020 20] | 5 μg QS7 |
| | | 100 μg DOPC |
| | | 25 μg Cholesterol |
| | hCMV gB + hCMV | 2 μg hCMV gB |
| | pentamer formulated | 2 μg Pentamer |
| | in LIP [QS7 600 + | 0.5 μg E6020 |
| | E6020 20] | 15 μg QS7 |
| | | 100 μg DOPC |
| | | 25 μg Cholesterol |
| | hCMV gB + hCMV | 2 μg hCMV gB |
| | pentamer formulated | 2 μg Pentamer |
| | in LIP [QS7 1800 + | 0.5 μg E6020 |
| | E6020 20] | 45 μg QS7 |
| | | 100 μg DOPC |
| | | 25 μg Cholesterol |

Blood samples and spleen cells were collected at 035 for measuring the seroneutralizing antibody response, the CMV gB- and CMV pentamer specific IgG antibody subclasses, as well as the IL-5 and IFN-γ secretions according to the methods described in Example 7.

### Results

### Hemolysis Assay

As shown on FIG. 21, QS21 elicits 100% of erythrocyte hemolysis from 25 μM to 100 μM and displays an effective concentration inducing hemolysis of 50% red blood cells (EC₅₀) at 4.2 μM while no hemolytic activity was detected with QS7 at similar concentrations.

### Immune Response Study in C57BL/6 Mice

The observed adjuvant potency effect was higher for QS21 LIP (0:200) than for QS7 LIP (0:200) for the hCMV gB and pentamer antigens. However, As shown on FIGS. 22A, 22B, 22C and 22D QS7 formulated in SPA14-like formulations (E62020:QS7 at 20:200; 600 or 1800) induced similar IgG1 and IgG2c responses compared to SPA14 (E6020:QS21 at 20:200).

As shown on FIGS. 23A and 23B, SPA14 formulations containing QS21 or QS7 induced comparable IgG2c/IgG1 ratios for the hCMV gB and pentamer antigens. Furthermore, as shown on FIG. 24, SPA14 (E6020:QS21 at 20:200) and LIP [QS7+E6020 20] with E6020:QS7 at 20:200, 600 or 1800 induced similar neutralizing antibody titers for the hCMV gB and pentamer antigens.

Although the level of secreted cytokines IFN-γ and IL-5 induced by LIP [E6020+QS7] were slightly lower than the levels induced by SPA14 (E62020:QS21 at 20:200), at every tested concentration, the ratios of IFN-γ/IL-5 were similar between SPA14 (E6020:QS21) and the different tested concentrations of LIP [E6020+QS7], as shown on FIGS. 25A and 25B.

It is known that adjuvanting response induced by Quillaja saponins is correlated with their acyl group which is also responsible for their toxicity (Fleck et al., Molecules. 2019; 24(1):171). QS7 has a shorter acyl chain compared to QS21 and has also a reduced toxicity (Wang et al., ACS Infect Dis. 2019; 5(6):974-981). The results here confirmed that QS7 has a good safety profile based on its hemolytic activity in vitro. Moreover, unexpectedly, when formulated in SPA14-like formulations, it was able to induce an adjuvanting effect comparable to SPA14 formulations containing QS21. Therefore, those results show that QS7 may advantageously be used as saponin in SPA14 formulations for inducing a good and safe adjuvanting effect.

### Example 13: Combined QS21-Liposomes and E6020-Liposomes Vs. SPA14 Formulation

Comparison of Combined Liposomes Containing QS21 or E6020 v. SPA14 Formulation

The objective of the study was to compare the adjuvanting effect of a combination of liposomes containing QS21 or E6020 with SPA14 formulation containing QS21 and E6020 on the immune response induced by hCMV antigens in a mouse model.

### Materials and Methods

### Preparation of Liposomes

The liposomes were prepared with the hCMV antigens gB and pentamer, as described in Examples 1 and 4.

### Immune Response Study in C57BL/6 Mice

5 groups of C₅₇BL/6 mice, 6-8 weeks old (n=8) received two IM immunizations (prime-boost: DO and D20) in the quadricep muscle with a final injection volume of 50 μL containing 2 μg of CMV gB and CMV pentamer (2 μg each/dose) formulated with:
DOPC-Chol liposomes (4000:1000 μg/ml) containing QS21 (200 μg/ml) without E6020 (“QS21 LIP”),
DOPC-Chol liposomes (4000:1000 μg/ml) containing E6020 (20 μg/ml) without QS21 (“E6020 LIP”),
SPA14 (DOPC-Chol liposomes (4000:1000 μg/ml) containing 200 μg/ml QS21 and 20 μg/ml E6020

A group of mice received the antigens without adjuvant (w/o adjuvant) and a group of mice received a combination of QS21 LIP and E6020 LIP. The amounts of administered antigens were identical for each group.

Blood samples and spleen cells were collected at D35 for measuring the CMV gB- and CMV pentamer specific IgG antibody subclasses, the IL-5 and IFN-γ secretions, and the neutralizing antibodies as indicated in Example 7.

Statistical analyses were carried out by Tukey adjustment+one-way ANOVA: p<0.05.

### Results

As shown on FIGS. 26A and 26B, the IgG1 and IgG2c responses observed with QS21 LIP (0:200) or E6020 LIP (20:0) were lower than the adjuvant potency effect observed with SPA14 (E6020:QS21 at 20:200) for the hCMV gB and pentamer antigens. However, the combined liposomes QS21 LIP (0:200) or E6020 LIP (20:0) induced similar IgG1 and IgG2c responses than SPA14.

As shown on FIGS. 27A and 27B, the IgG2c/IgG1 ratios were similar between the combined liposomes QS21 LIP and E6020 LIP and the SPA14 formulations.

The secreted levels of cytokines IFN-γ/IL-5 observed with QS21 LIP (0:200) or E6020 LIP (20:0) were lower than the secreted levels of IFN-γ/IL-5 observed with SPA14 (E6020:QS21 at 20:200) for the hCMV gB and pentamer antigens. Whereas, the combined liposomes QS21 LIP (0:200) or E6020 LIP (20:0) induced similar levels of cytokines IFN-γ/IL-5 than SPA14. As shown on FIGS. 28A and 28B, the ratio of cytokines IFN-γ/IL-5 secreted is similar between the combined liposomes QS21 LIP and E6020 LIP and the SPA14 formulations.

As shown on FIGS. 29A and 29B, the combined liposomes QS21 LIP (0:200) or E6020 LIP (20:0) induced similar serum neutralizing antibodies post-prime (D20) and post-boost (D35) measured both on fibroblast (MRC-5) or epithelial (ARPE-19) cell lines than SPA14. A significant adjuvant effect was measured with the combined liposomes QS21 LIP (0:200) or E6020 LIP (20:0).

In conclusion, the combined liposomes QS21 LIP and E6020 LIP can induce an adjuvanting effect similar to the effect obtained with the SPA14 formulations, i.e., liposomes containing simultaneously E6020 and a saponin such as QS21.

### REFERENCES

Albers J, Danzer C, Rechsteiner M, et al. A versatile modular vector system for rapid combinatorial mammalian genetics. J Clin Invest. 2015; 125(4):1603-1619. doi:10.1172/JC179743
Alderson M R, McGowan P, Baldridge J R, Probst P. TLR4 agonists as immunomodulatory agents. J Endotoxin Res. 2006; 12(5):313-9. doi: 10.1 179/096805106X118753. PMID: 17059695.
Althunian T A, de Boer A, Groenwold R H H, Klungel O H. 2017. Defining the noninferiority margin and analysing noninferiority: An overview. Br J Clin Pharmacol 83: 1636-42
Axelsson F, Adler S P, Lamarre A, Ohlin M. Humoral immunity targeting site I of antigenic domain 2 of glycoprotein B upon immunization with different cytomegalovirus candidate vaccines. Vaccine. 2007; 26(1):41-46. doi:1 0.101 6/j.vaccine.2007.10.048
Backovic M, Longnecker R, Jardetzky T S. Structure of a trimeric variant of the Epstein-Barr virus glycoprotein B. Proc Natl Acad Sci USA. 2009; 106(8):2880-2885. doi:10.1073/pnas.0810530106
Burke H G, Heldwein E E. Crystal Structure of the Human Cytomegalovirus Glycoprotein B [published correction appears in PLoS Pathog. 2015 November; 11(11):e1005300]. PLoS Pathog. 2015; 11(10):e1005227. Published 2015 Oct. 20. doi:10.1371/journal.ppat.1005227
Cheshenko N, Krougliak N, Eisensmith R C, Krougliak V A. A novel system for the production of fully deleted adenovirus vectors that does not require helper adenovirus. Gene Ther. 2001; 8(11):846-854. doi:10.1038/sj.gt.3301459
Ciferri C, Chandramouli S, Donnarumma D, et al. Structural and biochemical studies of HCMV gH/gL/gO and Pentamer reveal mutually exclusive cell entry complexes. Proc Natl Acad Sci USA. 2015; 112(6):1767-1772. doi:1 0.1073/pnas.1424818112
Collin M, McGovern N, Haniffa M. 2013. Human dendritic cell subsets. Immunology 140: 22-30
Deng K et al. Synthesis of QS-21-Xylose: Establishment of the Immunopotentiating Activity of Synthetic QS-21 Adjuvant with a Melanoma Vaccine, Angew Chem Int Ed Engl. 2008; 47(34): 6395-6398, doi: 10.1002/anie.200801885
Didierlaurent A M, Laupeze B, Di Pasquale A, Hergli N, Collignon C, Garcon N. 2017. Adjuvant system AS01: helping to overcome the challenges of modern vaccines. Expert Rev Vaccines 16: 55-63
Didierlaurent A M, Collignon C, Bourguignon P, Wouters S, Fierens K, Fochesato M, Dendouga N, Langlet C, Malissen B, Lambrecht B N, Garcon N, Van Mechelen M, Morel S. 2014. Enhancement of adaptive immunity by the human vaccine adjuvant AS01 depends on activated dendritic cells. J Immunol 193: 1920-30
Donald R. Drake III IS, Michael N. Nguyen, Anatoly Kachurin, Vaughan Wittman, Robert Parkhill O K, Janice M. Moser, Nicolas Burdin, Monique Moreau, Noelle Mistretta, Anthony M. Byers V D, Tenekua M. Tapia, Charlotte Vernhes, T. Kamala, Nithya Swaminathan, and William L. Warren, Abstract. 2012. In Vitro Biomimetic Model of the Human Immune System for Predictive Vaccine Assessments. DISRUPTIVE SCIENCE AND TECHNOLOGY 1: 28-40
Doyle S A, High Throughput Protein Expression and Purification: Methods and Protocols. Methods in Molecular Biology. 2008; vol. 498. Ed. Humana Press
EP 2 627 352—Novel antigen
Fleck J D, Betti A H, da Silva F P, et al. Saponins from Quillaja saponaria and Quillaja brasiliensis: Particular Chemical Characteristics and Biological Activities. Molecules. 2019; 24(1):171. Published 2019 Jan. 4. doi:10.3390/molecules24010171
Fox C B, Friede M, Reed S G, Ireton G C. Synthetic and natural TLR4 agonists as safe and effective vaccine adjuvants. Subcell Biochem. 2010; 53:303-21. doi: 10.1007/978-90-481-9078-2_14. PMID: 20593273.
Griffiths P D, Stanton A, McCarrell E, et al. Cytomegalovirus glycoprotein-B vaccine with MF59 adjuvant in transplant recipients: a phase 2 randomised placebo-controlled trial. Lancet. 2011; 377(9773):1256-1263. doi:10.1016/S0140-6736(11)60136-0
Habibi, Chiu et al. IgA B-cell memory in experimental infection of adults with RSV—Am J Resp Crit Care Med 2015
Haensler J, Probeck P, Su J, Piras F, Dalencon F, Cotte J F, Chambon V, Iqbal S M, Hawkins L, Burdin N. 2015. Design and preclinical characterization of a novel vaccine adjuvant formulation consisting of a synthetic TLR4 agonist in a thermoreversible squalene emulsion. Int J Pharm 486: 99-111
Henikoff S, Henikoff J G. Amino acid substitution matrices from protein blocks. Proc Natl Acad Sci USA. 1992; 89(22):10915-10919. doi:10.1073/pnas.89.22.10915
Heldwein E E, Lou H, Bender F C, Cohen G H, Eisenberg R J, Harrison S C. Crystal structure of glycoprotein B from herpes simplex virus 1. Science. 2006; 313(5784):217-220. doi:1 0.1 126/science.1126548
Hervé C, Laupeze B, Del Giudice G, Didierlaurent A M, Tavares Da Silva F. The how's and what's of vaccine reactogenicity. NPJ Vaccines. 2019; 4:39. Published 2019 Sep. 24. doi:10.1038/s41541-019-0132-6
Higbee R G, Byers A M, Dhir V, Drake D, Fahlenkamp H G, Gangur J, Kachurin A, Kachurina O, Leistritz D, Ma Y, Mehta R, Mishkin E, Moser J, Mosquera L, Nguyen M, Parkhill R, Pawar S, Poisson L, Sanchez-Schmitz G, Schanen B, Singh I, Song H, Tapia T, Warren W, Wittman V. 2009. An immunologic model for rapid vaccine assessment—a clinical trial in a test tube. Altern Lab Anim 37 Suppl 1: 19-27
Hofmann I, Wen Y, Ciferri C, et al. Expression of the human cytomegalovirus pentamer complex for vaccine use in a CHO system. Biotechnol Bioeng. 2015; 112(12):2505-2515. doi:10.1002/bit.25670
Institute of Medicine (US) Committee to Study Priorities for Vaccine Development, Stratton K R, Durch J S, Lawrence R S, eds. Vaccines for the 21st Century: A Tool for Decision making. Washington (DC): National Academies Press (US); 2000.
Ishizaka, Sally & Hawkins, Lynn. (2007). E6020: A synthetic Toll-like receptor 4 agonist as a vaccine adjuvant. Expert review of vaccines. 6. 773-84. 10.1586/14760584.6.5.773
Katzen F, Chang G, Kudlicki W. The past, present and future of cell-free protein synthesis. Trends Biotechnol. 2005; 23(3):150-156. doi:10.1016/j.tibtech.2005.01.003
Kim Y J et al. Synthetic Studies of Complex Immunostimulants from Quillaja saponaria: Synthesis of the Potent Clinical Immunoadjuvant QS-21Aapi, J Am Chem Soc, 2006; 128:11906-11915, doi: 10.1021/ja062364i
Klucker M F, Dalengon F, Probeck P, Haensler J. AF03, an alternative squalene emulsion-based vaccine adjuvant prepared by a phase inversion temperature method. J Pharm Sci. 2012 December; 101(12):4490-500. doi: 10.1002/jps.23311. Epub 2012 Aug. 31. PMID: 22941944.
Liposomes: A practical approach. Edited by RRC New. Oxford University Press, 1990
Loignon M, Perret S, Kelly J, et al. Stable high volumetric production of glycosylated human recombinant IFNalpha2b in HEK293 cells. BMC Biotechnol. 2008; 8:65. Published 2008 Aug. 27. doi:10.1186/1472-6750-8-65
Luna et al. “Evaluation of the innate immunostimulatory potential of originator and non-originator copies of insulin glargine in an in vitro human immune model.” PloS one vol. 13,6 e0197478. 6 Jun. 2018, doi:10.1371/journal.pone.01 97478
Ma Y, Poisson L, Sanchez-Schmitz G, Pawar S, Qu C, Randolph G J, Warren W L, Mishkin E M, Higbee R G. 2010. Assessing the immunopotency of Toll-like receptor agonists in an in vitro tissue-engineered immunological model. Immunology 130: 374-87
Macagno A, Bernasconi N L, Vanzetta F, et al. Isolation of human monoclonal antibodies that potently neutralize human cytomegalovirus infection by targeting different epitopes on the gH/gL/UL128-131A complex. J Virol. 2010; 84(2):1005-1013. doi:10.1128/JVI.01809-09
Mastelic B, Lewis D J, Golding H, Gust I, Sheets R, Lambert P H. 2013. Potential use of inflammation and early immunological event biomarkers in assessing vaccine safety. Biologicals 41: 115-24
Merrifield R. B., Solid Phase Peptide Synthesis. I. The Synthesis of a Tetrapeptide. J. Am. Chem. Soc., 1963, 85(14), 2149-2154
Morihara K., Using proteases in peptide synthesis. Trends in Biotechnology, 1987, 5(6), 164-170
Needleman S B, Wunsch C D. A general method applicable to the search for similarities in the amino acid sequence of two proteins. J Mol Biol. 1970; 48(3):443-453. doi:10.1016/0022-2836(70)90057-4
Patrone M, Secchi M, Fiorina L, Ierardi M, Milanesi G, Gallina A. Human cytomegalovirus UL130 protein promotes endothelial cell infection through a producer cell modification of the virion. J Virol. 2005; 79(13):8361-8373. doi:10.1128/JVI.79.13.8361-8373.2005
Pass R F, Zhang C, Evans A, et al. Vaccine prevention of maternal cytomegalovirus infection. N Engl J Med. 2009; 360(12):1191-1199. doi:10.1056/NEJMoa0804749
Peri F, Calabrese V. Toll-like receptor 4 (TLR4) modulation by synthetic and natural compounds: an update. J Med Chem. 2014; 57(9):3612-3622. doi:1 0.1021/jm401006s
Permar S R, Schleiss M R, Plotkin S A. Advancing Our Understanding of Protective Maternal Immunity as a Guide for Development of Vaccines To Reduce Congenital Cytomegalovirus Infections. J Virol. 2018 Mar. 14; 92(7):e00030-18. doi: 10.1128/JVI.00030-18. PMID: 29343580; PMCID: PMC5972872.
Plotkin et al., Vaccines, 6th edition, Ed. Elsevier, 2013, Schleiss et al., Cytomegalovirus vaccines, pages 1032-1041
Remington's Pharmaceutical Sciences (18th edition), ed. A. Gennaro, 1990, Mack Publishing Company, Easton, Pa
Ryckman B J, Rainish B L, Chase M C, et al. Characterization of the human cytomegalovirus gH/gL/UL128-131 complex that mediates entry into epithelial and endothelial cells. J Virol. 2008; 82(1):60-70. doi:10.1128/JVI.01910-07
Sharma S, Wisner T W, Johnson D C, Heldwein E E. HCMV gB shares structural and functional properties with gB proteins from other herpesviruses. Virology. 2013; 435(2):239-249. doi:10.1016/j.virol.2012.09.024
Sambrook, J., Molecular Cloning: A Laboratory Manual. 3rd. Cold Spring Harbor Laboratory Press, 2000
Schoppel K, Hassfurther E, Britt W, Ohlin M, Borrebaeck C A, Mach M. Antibodies specific for the antigenic domain 1 of glycoprotein B (gpUL55) of human cytomegalovirus bind to different substructures. Virology. 1996; 216(1):133-145. doi:1 0.1 006/viro.1996.0040
Scott, M. T., Goss-Sampson, M. and Bomford, R., 1985, Adjuvant activity of saponin: Antigen localisation studies, Int. Archs. Allergy Appl. Immun., 77: 409.
Swanson K A, Rainho-Tomko J N, Williams Z P, Lanza L, Peredelchuk M, Kishko M, et al. A respiratory syncytial virus (RSV) F protein nanoparticle vaccine focuses antibody responses to a conserved neutralization domain. Sci Immunol. 2020; 5(47)
Szymczak-Workman A L, Vignali K M, Vignali D A. Design and construction of 2A peptide-linked multicistronic vectors. Cold Spring Harb Protoc. 2012; 2012(2):199-204. Published 2012 Feb. 1. doi:10.1101/pdb.ip067876
US 2002/0102562—Recombinant CMV neutralizing proteins
U.S. Pat. No. 5,057,540B2
U.S. Pat. No. 5,547,834—Recombinant CMV neutralizing proteins
U.S. Pat. No. 5,608,143—External regulation of gene expression
U.S. Pat. No. 5,693,506—Process for protein production in plants
U.S. Pat. No. 6,100,064—Secreted viral proteins useful for vaccines and diagnostics
Veseli A, Žakelj S, Kristl A. A review of methods for solubility determination in biopharmaceutical drug characterization. Drug Dev Ind Pharm. 2019 November; 45(11):1717-1724. doi: 10.1080/03639045.2019.1665062. Epub 2019 Sep. 12. PMID: 31512934
Wagner A, Platzgummer M, Kreismayr G, Quendler H, Stiegler G, Ferko B, Vecera G, Vorauer-Uhl K, Katinger H. GMP production of liposomes—a new industrial approach. J Liposome Res. 2006; 16(3):311-9. doi: 10.1080/08982100600851086. PMID: 16952884.
Wagner A, Vorauer-Uhl K. Liposome technology for industrial purposes. J Drug Deliv. 2011; 2011:591325. doi: 10.1155/2011/591325. Epub 2010 Dec. 5. PMID: 21490754; PMCID: PMC3065896.
Wang D, Shenk T. Human cytomegalovirus UL131 open reading frame is required for epithelial cell tropism. J Virol. 2005 August; 79(16):10330-8. doi: 10.1128/JVI.79.16.10330-10338.2005. PMID: 16051825; PMCID: PMC1182637.
Wang P, Ding X, Kim H, Michalek S M, Zhang P. Structural Effect on Adjuvanticity of Saponins. J Med Chem. 2020 Mar. 26; 63(6):3290-3297. doi: 10.1021/acs.jmedchem.9b02063. Epub 2020 Feb. 26. PMID: 32101001
Wang P, et al., Synthesis of QS-21 based immunoadjuvants, J Org Chem, 2013 Nov. 15; 78(22): 11525-11534, doi: 10.1021/jo402118j
Wang P, Škalamera D, Sui X, Zhang P, Michalek S M. Synthesis and Evaluation of QS-7-Based Vaccine Adjuvants. ACS Infect Dis. 2019; 5(6):974-981. doi:10.1021/acsinfecdis.9b00039
Wen Y, Monroe J, Linton C, et al. Human cytomegalovirus gH/gL/UL128/UL130/UL131A complex elicits potently neutralizing antibodies in mice. Vaccine. 2014; 32(30):3796-3804. doi:10.1016/j.vaccine.2014.05.004
WO 2007/005583 A1
WO 2009/037359 A1—Vaccine composition for the prevention of CMV infections
WO 2014/005959—Complexes of cytomegalovirus proteins
WO 2014/160463 A1
WO 2016/092460—Cytomegalovirus antigens
WO 2017/070613A1—Human cytomegalovirus vaccine
WO 2019/052975—Human cytomegalovirus immunogenic composition
WO 2019/106192 A1—Saponin purification
WO 2019/157509 A1
WO 2019/195316 A1
Zenk M H, 6. Chasing the enzymes of secondary metabolism: Plant cell cultures as a pot of gold. Phytochemistry. 1991, 30(12):3861-3863. doi.org/10.1016/0031-9422(91)83424-J

1. A liposome comprising a saponin, a sterol, a phospholipid and a Toll-like receptor 4 (TLR4) agonist, or
a combination of liposomes comprising at least two types of liposomes, wherein a first type of liposome comprises a saponin, a sterol, and a phospholipid and a second type of liposome comprises a sterol, a phospholipid, and a Toll-like receptor 4 (TLR4) agonist,
wherein the Toll-like receptor 4 (TLR4) agonist is of formula (I): 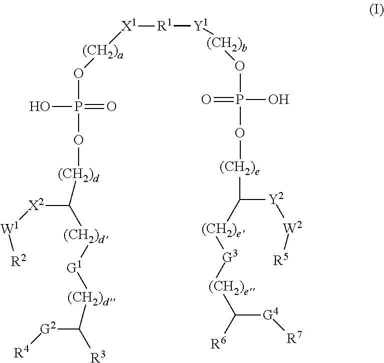
wherein R¹ is selected from the group consisting of:
a) C(O);
b) C(O)—(C₁-C₁₄ alkyl)-C(O), in which said C₁-C₁₄ alkyl is optionally substituted with a hydroxyl, a C₁-C₅ alkoxy, a C₁-C₅ alkylenedioxy, a (C₁-C₅ alkyl)amino or a (C₁-C₅ alkyl)aryl, in which said aryl moiety of said (C₁-C₅ alkyl)aryl is optionally substituted with a C₁-C₅ alkoxy, a (C₁-C₅ alkyl)amino, a (C₁-C₅ alkoxy)amino, a (C₁-C₅ alkyl)-amino(C₁-C₅ alkoxy), —O—(C₁-C₅ alkyl)amino(C₁-C₅ alkoxy), —O—(C₁-C₅ alkyl)amino-C(O)—(C₁-C₅ alkyl)-C(O)OH, or —O—(C₁-C₅ alkyl)amino-C(O)—(C₁-C₅ alkyl)-C(O)—(C₁-C₅)alkyl;
c) an alkyl comprising a C₂-C₁₅ linear or branched chain, optionally substituted with a hydroxyl or an alkoxy; and
d) —C(O)—(C₆-C₁₂ arylene)-C(O)— in which said arylene is optionally substituted with a hydroxyl, a halogen, a nitro or an amino;
a and b are independently 0, 1, 2, 3 or 4;
d, d′, d″, e, e′ and e″ are independently 0, 1, 2, 3 or 4;
X₁, X₂, Y₁ and Y₂ are independently selected from the group consisting of null, an oxygen, —NH— and —N(C(O)(C₁-C₄ alkyl))-, and —N(C₁-C₄ alkyl)-;
W₁ and W₂ are independently selected from the group consisting of a carbonyl, a methylene, a sulfone and a sulfoxide;
R² and R⁵ are independently selected from the group consisting of:
a) a C₂ to C₂₀ straight chain or branched chain alkyl, which is optionally substituted with an oxo, a hydroxyl or an alkoxy;
b) a C₂ to C₂₀ straight chain or branched chain alkenyl or dialkenyl, which is optionally substituted with an oxo, a hydroxyl or an alkoxy;
c) a C₂ to C₂₀ straight chain or branched chain alkoxy, which is optionally substituted with an oxo, a hydroxyl or an alkoxy;
d) —NH—(C₂ to C₂₀ straight chain or branched chain alkyl), in which said alkyl group is optionally substituted with an oxo, a hydroxy or an alkoxy; and
e) 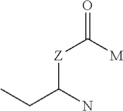
in which Z is selected from the group consisting of an O and NH, and M and N are independently selected from the group consisting of an alkyl, an alkenyl, an alkoxy, an acyloxy, an alkylamino and an acylamino comprising a C₂-C₂₀ linear or branched chain;
R³ and R⁶ are independently selected from the group consisting of a C₂ to C₂₀ straight chain or branched chain alkyl or alkenyl, optionally substituted with an oxo or a fluoro;
R⁴ and R⁷ are independently selected from the group consisting of a C(O)—(C₂ to C₂₀ straight chain or branched chain alkyl or alkenyl), a C₂ to C₂₀ straight chain or branched chain alkyl, a C₂ to C₂₀ straight chain or branched chain alkoxy, and a C₂ to C₂₀ straight chain or branched chain alkenyl; in which said alkyl, alkenyl or alkoxy groups can be independently and optionally substituted with a hydroxyl, a fluoro or a C₁-C₅ alkoxy;
G¹, G², G³ and G⁴ are independently selected from the group consisting of an oxygen, a methylene, an amino, a thiol, —C(O)NH—, —NHC(O)—, and —N(C(O)(C₁-C₄ alkyl))-;
or G²R⁴ or G⁴R⁷ can together be a hydrogen atom or a hydroxyl;
or a pharmaceutically acceptable salt of this compound;
wherein the TLR4 agonist and the saponin are present in a weight:weight ratio of TLR4 agonist:saponin ranging from about 1:1 to about 1:50, or from about 1:25 to about 1:35, or in a weight ratio of TLR4 agonist:saponin of about 1:10.

2. The liposome or the combination of liposomes according to claim 1, wherein the TLR4 agonist has a solubility parameter in ethanol, measured at 25° C., of at least about 0.2 mg/ml.

3. The liposome or the combination of liposomes according to claim 1 or 2, wherein the TLR4 agonist is of formula (II): 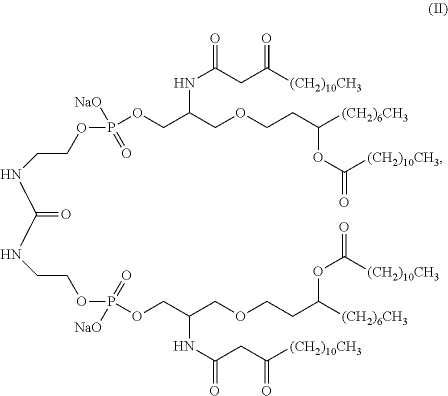
in particular the TLR4 agonist is E6020 of formula (III):

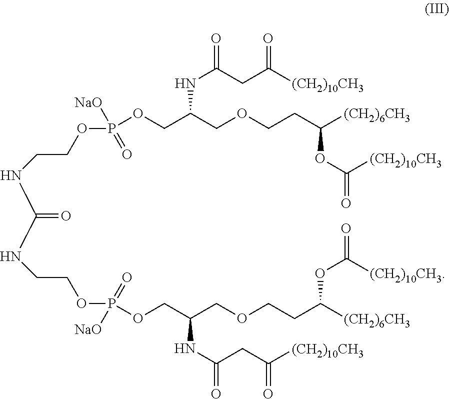

4. The liposome or the combination of liposomes according to any one of claims 1 to 3, wherein the saponin is a Quillaja saponaria saponin, in particular is extracted from the bark of Quillaja Saponaria Molina.

5. The liposome or the combination of liposomes according to any one of claims 1 to 4, wherein the saponin is selected among QS-7, QS-17, QS-18, QS-21, and combinations thereof, preferably the saponin is QS-7 or QS-21.

6. The liposome or the combination of liposomes according to any one of claims 1 to 5, wherein the sterol is selected from cholesterol or its derivatives, ergosterol, desmosterol (3β-hydroxy-5,24-cholestadiene), stigmasterol (stigmasta-5,22-dien-3-ol), lanosterol (8,24-lanostadien-3b-ol), 7-dehydrocholesterol (Δ5,7-cholesterol), dihydrolanosterol (24,25-dihydrolanosterol), zymosterol (5α-cholesta-8,24-dien-3β-ol), lathosterol (5α-cholest-7-en-3β-ol), diosgenin ((3β,25R)-spirost-5-en-3-ol), sitosterol (22,23-dihydrostigmasterol), sitostanol, campesterol (campest-5-en-3β-ol), campestanol (5a-campestan-3b-ol), 24-methylene cholesterol (5,24(28)-cholestadien-24-methylen-3β-ol), cholesteryl margarate (cholest-5-en-3β-yl heptadecanoate), cholesteryl oleate, cholesteryl stearate, and mixtures thereof, in particular the sterol is selected from cholesterol or its derivatives, and in particular the sterol is cholesterol.

7. The liposome or the combination of liposomes according to any one of claims 1 to 6, wherein the saponin and the sterol are present in a weight:weight ratio of saponin:sterol ranging from 1:100 to 1:1, ranging from 1:50 to 1:2, or ranging from 1:10 to 1:5, or in a weight:weight ratio of saponin:sterol of about 1:2, or in a weight:weight ratio of saponin:sterol of about 1:5.

8. The liposome or the combination of liposomes according to any one of claims 1 to 7, wherein the phospholipid is selected from phosphatidylcholines, phosphatidic acids, phosphatidylethanolamines, phosphatidylglycerols, phosphatidylserines, phosphatidylinositols, and mixtures thereof, in particular the phospholipid is a phosphatidylcholine selected from DSPC (1,2-distearoyl-sn-glycero-3-phosphocholine), DPPC (1,2-dipalmitoyl-sn-glycero-3-phosphocholine), DMPC (1,2-dimyristoyl-sn-glycero-3-phosphocholine), POPC (1-palmitoyl-2-oleoyl-sn-glycero-3-phosphocholine), DOPC (1,2-dioleoyl-sn-glycero-3-phosphocholine), SOPC (1-stearoyl-2-oleoyl-sn-glycero-3-phosphocholine), and mixtures thereof.

9. A method for manufacturing a liposome comprising at least the steps of:
(a) solubilizing, in an organic water-miscible solvent, a TLR4 agonist of formula (I) having a solubility parameter in ethanol, measured at 25° C., of at least about 0.2 mg/ml, a sterol, and a phospholipid,
(b) processing the mixture obtained at step (a) into a liposome,
wherein a saponin is added either at step (a), at step (b), or after step b), and
wherein the TLR4 agonist and the saponin are present in a weight:weight ratio of TLR4-agonist:saponin ranging from about 1:1 to about 1:400, ranging from about 1:2 to about 1:200, ranging from about 1:2.5 to about 1:100, ranging from about 1:3 to about 1:40, or ranging from about 1:5 to about 1:25.

10. The method according to claim 9, comprising a step, prior to step (a), of selecting a TLR4 agonist of formula (I) having a solubility parameter in ethanol, measured at 25° C., of at least about 0.2 mg/ml.

11. The method according to any one of claims 9 or 10, wherein step (b) of processing the mixture obtained at step (a) into a liposome is carried out by using the solvent injection method.

12. The method according to any one of claims 9 to 11, wherein step (b) of processing the mixture obtained at step (a) into a liposome includes the steps of:
(b1) injecting and/or diluting the solution obtained at step (a) into an aqueous buffer, and
(b2) removing the organic water-miscible solvent.

13. The method according to any one of claims 9 to 12, wherein the organic water-miscible solvent is selected from ethanol, isopropanol, or mixtures thereof, or is ethanol.

14. The method according to any one of claims 9 to 13, further comprising a step (c) of filtering the liposomes obtained in step (b) and recovering the liposomes having an average diameter lower than 200 nm.

15. An adjuvant composition comprising at least either one liposome or one combination of liposomes according to any one of claims 1 to 8 or at least one liposome obtained according to the method of any one of claims 9 to 14.

16. An immunogenic composition comprising at least either one liposome or one combination of liposomes according to any one of claims 1 to 8, or at least one liposome obtained according to the method of any one of claims 9 to 14, or an adjuvant composition according to claim 15, and at least one antigen.

17. An immunogenic composition comprising at least:
one CMV gB antigen;
one CMV gH/gL/UL128/UL130/UL131 pentameric complex antigen; and
one adjuvant comprising either at least one liposome comprising a saponin, a sterol, a phospholipid and a Toll-like receptor 4 (TLR4) agonist or at least a combination of liposomes comprising at least two types of liposomes, wherein a first type of liposome comprises a saponin, a sterol, and a phospholipid and a second type of liposome comprises a sterol, a phospholipid, and a Toll-like receptor 4 (TLR4) agonist.

18. The immunogenic composition according to claim 17, wherein said CMV gB antigen is selected in a group comprising a full length CMV gB antigen, a truncated CMV gB antigen deleted from at least a part of the transmembrane domain, a truncated CMV gB antigen substantially deleted from all the transmembrane domain, a truncated CMV gB antigen deleted from at least a part of the intracellular domain, a truncated CMV gB antigen substantially deleted from all the intracellular domain, and a truncated CMV gB antigen deleted substantially from both the transmembrane domain and the intracellular domain, and in particular said CMV gB antigen is gBdTM.

19. The immunogenic composition according to claim 17 or 18, wherein said gH is deleted from at least a part of the transmembrane domain or from substantially all the transmembrane domain or wherein said gH comprises the ectodomain of the full length gH polypeptide encoded by UL75 gene.

20. The immunogenic composition according to any one of claims 17 to 19, wherein the CMV gB antigen and the CMV gH/gL/UL128/UL130/UL131 pentameric complex antigen are the only CMV antigens.

21. The immunogenic composition according to any one of claims 17 to 20, wherein the TLR4 agonist is according to any one of claims 1 to 3.

22. The immunogenic composition according to any one of claims 17 to 21, wherein the saponin is according to claim 4 or 5.

23. The immunogenic composition according to any one of claims 17 to 22, wherein the sterol is according to claim 6 or 7.

24. The immunogenic composition according to any one of claims 17 to 23, wherein the phospholipid is according to claim 8.

25. An immunogenic composition according to any one of claims 17 to 24, for use as a CMV vaccine.

26. A liposome or a combination of liposomes according to any one of claims 1 to 8, a liposome obtained according to the method of any one of claims 9 to 14, an adjuvant composition according to claim 15, an immunogenic composition according to claim 16, for their use in the prevention and/or the treatment of a infectious diseases, allergies, autoimmune diseases, rare blood disorders, rare metabolic diseases, rare neurologic diseases, and tumour or cancer diseases.


SEQUENCE LISTING (6 sequences)

SEQ ID NO: 1 - protein, 805 aa, Cytomegalovirus
STRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNTTQTSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKRLCMQPLQNLFPYLVSADGTTVTSGNTKDTSLQAPPSYEESVYNSGRKGPGPPSSDASTAAPPYTNEQAYQMLLALVRLDAEQRAQQNGTDSLDGQTGTQDKGQKPNLLDRLRHRKNGYRHLKDSDEEENV

SEQ ID NO: 2 - protein, 694 aa, Cytomegalovirus
RYGAEAVSEPLDKAFHLLLNTYGRPIRFLRENTTQCTYNNSLRNSTVVRENAISFNFFQSYNQYYVFHMPRCLFAGPLAEQFLNQVDLTETLERYQQRLNTYALVSKDLASYRSFSQQLKAQDSLGEQPTTVPPPIDLSIPHVWMPPQTTPHGWTESHTTSGLHRPHFNQTCILFDGHDLLFSTVTPCLHQGFYLIDELRYVKITLTEDFFVVTVSIDDDTPMLLIFGHLPRVLFKAPYQRDNFILRQTEKHELLVLVKKDQLNRHSYLKDPDFLDAALDFNYLDLSALLRNSFHRYAVDVLKSGRCQMLDRRTVEMAFAYALALFAAARQEEAGAQVSVPRALDRQAALLQIQEFMITCLSQTPPRTTLLLYPTAVDLAKRALWTPNQITDITSLVRLVYILSKQNQQHLIPQWALRQIADFALKLHKTHLASFLSAFARQELYLMGSLVHSMLVHTTERREIFIVETGLCSLAELSHFTQLLAHPHHEYLSDLYTPCSSSGRRDHSLERLTRLFPDATVPATVPAALSILSTMQPSTLETFPDLFCLPLGESFSALTVSEHVSYVVTNQYLIKGISYPVSTTVVGQSLIITQTDSQTKCELTRNMHTTHSITAALNISLENCAFCQSALLEYDDTQGVINIMYMHDSDDVLFALDPYNEVVVSSPRTHYLMLLKNGTVLEVTDVVVDATDSR

SEQ ID NO: 3 - protein, 248 aa, Cytomegalovirus
AAVSVAPTAAEKVPAECPELTRRCLLGEVFQGDKYESWLRPLVNVTGRDGPLSQLIRYRPVTPEAANSVLLDEAFLDTLALLYNNPDQLRALLTLLSSDTAPRWMTVMRGYSECGDGSPAVYTCVDDLCRGYDLTRLSYERSIFTEHVLGFELVPPSLFNVVVAIRNEATRTNRAVRLPVSTAAAPEGITLFYGLYNAVKEFCLRHQLDPPLLRHLDKYYAGLPPELKQTRVNLPAHSRYGPQAVDAR

SEQ ID NO: 4 - protein, 144 aa, Cytomegalovirus
EECCEFINVNHPPERCYDFKMCNRFTVALRCPDGEVCYSPEKTAEIRGIVTTMTHSLTRQVVHNKLTSCNYNPLYLEADGRIRCGKVNDKAQYLLGAAGSVPYRWINLEYDKITRIVGLDQYLESVKKHKRLDVCRAKMGYMLQ

SEQ ID NO: 5 - protein, 189 aa, Cytomegalovirus
SPWSTLTANQNPSPLWSKLTYSKPHDAATFYCPFIYPSPPRSPLQFSGFQRVLTGPECRNETLYLLYNREGQTLVERSSTWVKKVIWYLSGRNQTILQRMPRTASKPSDGNVQISVEDAKIFGAHMVPKQTKLLRFVVNDGTRYQMCVMKLESWAHVFRDYSVSFQVRLTFTEANNQTYTFCTHPNLIV

SEQ ID NO: 6 - protein, 111 aa, Cytomegalovirus
QCQRETAEKNDYYRVPHYWDACSRALPDQTRYKYVEQLVDLTLNYHYDASHGLDNFDVLKRINVTEVSLLISDFRRQNRRGGTNKRTTFNAAGSLAPHARSLEFSVRLFAN